SEQUENCE LISTING (290 sequences)

SEQ ID NO: 1 - DNA, 45 bp, synthetic construct
  source 1..45: mol_type = other DNA
ggtggtattgcccggagtagaagcacctgtgactaactgtgccaa

SEQ ID NO: 2 - DNA, 49 bp, synthetic construct
  source 1..49: mol_type = other DNA
ttttttgatggccagctccgaggatctagcgtactgagtcgcccagtct

SEQ ID NO: 3 - DNA, 73 bp, synthetic construct
  source 1..73: mol_type = other DNA
  variation 72: replace = c
  variation 2: replace = g
  variation 71: replace = c
  variation 6: replace = g
  variation 67: replace = c
  variation 64: replace = g
  variation 13: replace = c
  variation 22: replace = g
  variation 15: replace = g
  variation 28: replace = c
  variation 42: replace = g
  variation 31: replace = a
  variation 39: replace = t
  variation 37: replace = g
  variation 44: replace = a
  variation 50: replace = c
gccccagtggcctaatggataaggcactggccttcaaagccagggattgtgggttcgagtcccacctggggtg

SEQ ID NO: 4 - DNA, 73 bp, synthetic construct
  source 1..73: mol_type = other DNA
gccccagtggcctaatggataaggcactggccttcaaagccagggattgtgggttcgagtcccacctggggta

SEQ ID NO: 5 - DNA, 73 bp, synthetic construct
  source 1..73: mol_type = other DNA
gccccggtggcctaatggataaggcattggccttcaaagccagggattgtgggttcgagtcccacccggggta

SEQ ID NO: 6 - DNA, 73 bp, synthetic construct
  source 1..73: mol_type = other DNA
  variation 65: replace = c
  variation 23: replace = g
  variation 28: replace = c
  variation 50: replace = t
  variation 4: replace = c
  variation 27: replace = c
  variation 42: replace = g
  variation 49: replace = g
  variation 64: replace = a
  variation 69: replace = g
  variation 12: replace = c
  variation 43: replace = g
  variation 40: replace = c
  variation 31: replace = c
  variation 39: replace = g
  variation 44: replace = g
  variation 46: replace = a
  variation 2: replace = c
ggctcwgtggcgcaatggatagcgcattggacttcaaattcaaaggttccgggttcgagtcccggcwgagscg

SEQ ID NO: 7 - DNA, 73 bp, synthetic construct
  source 1..73: mol_type = other DNA
gccccagtggcctaatggataaggcattggccttcaaagccagggattgtgggttcgagtcccatctggggtg

SEQ ID NO: 8 - DNA, 73 bp, synthetic construct
  source 1..73: mol_type = other DNA
gccccagtggcctgatggataaggtactggccttcaaagccagggattgtgggttcgagttccacctggggta

SEQ ID NO: 9 - DNA, 73 bp, synthetic construct
  source 1..73: mol_type = other DNA
ggctctgtggcgcaatggatagcgcattggacttcaaattcaaaggttgtgggttcgaatcccaccagagtcg

SEQ ID NO: 10 - DNA, 73 bp, synthetic construct
  source 1..73: mol_type = other DNA
ggctctgtggcgcaatggatagcgcattggacttcaaattcaaaggttgtgggttcgagtcccaccagagtcg

SEQ ID NO: 11 - DNA, 74 bp, synthetic construct
  source 1..74: mol_type = other DNA
gtctctgtggcgcaatggacgagcgcgctggacttcaaatccagaggttccgggttcgagtcccggcagagatg

SEQ ID NO: 12 - DNA, 73 bp, synthetic construct
  source 1..73: mol_type = other DNA
ggctctgtggcgcaatggatagcgcattggacttcaaattcaaaggttgcgggttcgagtccctccagagtcg

SEQ ID NO: 13 - DNA, 73 bp, synthetic construct
  source 1..73: mol_type = other DNA
ggccgcgtggcctaatggataaggcgtctgacttcagatcagaagattgcaggttcgagtcctgccgcggtcg

SEQ ID NO: 14 - DNA, 73 bp, synthetic construct
  source 1..73: mol_type = other DNA
gaccacgtggcctaatggataaggcgtctgacttcagatcagaagattgagggttcgaatccctccgtggtta

SEQ ID NO: 15 - DNA, 73 bp, synthetic construct
  source 1..73: mol_type = other DNA
gaccgcgtggcctaatggataaggcgtctgacttcagatcagaagattgagggttcgagtcccttcgtggtcg

SEQ ID NO: 16 - DNA, 73 bp, synthetic construct
  source 1..73: mol_type = other DNA
gaccacgtggcctaatggataaggcgtctgacttcagatcagaagattgagggttcgaatcccttcgtggtta

SEQ ID NO: 17 - DNA, 73 bp, synthetic construct
  source 1..73: mol_type = other DNA
gaccacgtggcctaatggataaggcgtctgacttcagatcagaagattgagggttcgaatcccttcgtggttg

SEQ ID NO: 18 - DNA, 73 bp, synthetic construct
  source 1..73: mol_type = other DNA
ggccgtgtggcctaatggataaggcgtctgacttcagatcaaaagattgcaggtttgagttctgccacggtcg

SEQ ID NO: 19 - DNA, 73 bp, synthetic construct
  source 1..73: mol_type = other DNA
ggccgcgtggcctaatggataaggcgtctgatttcagatcagaagattgagggttcgagtcccttcgtggtcg

SEQ ID NO: 20 - DNA, 73 bp, synthetic construct
  source 1..73: mol_type = other DNA
gacccagtggcctaatggataaggcatcagccttcagagctggggattgtgggttcgagtcccatctgggtcg

SEQ ID NO: 21 - DNA, 73 bp, synthetic construct
  source 1..73: mol_type = other DNA
gggccagtggcgcaatggataacgcgtctgacttcagatcagaagattccaggttcgactcctggctggctcg

SEQ ID NO: 22 - DNA, 73 bp, synthetic construct
  source 1..73: mol_type = other DNA
gggccagtggcgcaatggataacgcgtctgacttcagatcagaagattctaggttcgactcctggctggctcg

SEQ ID NO: 23 - DNA, 73 bp, synthetic construct
  source 1..73: mol_type = other DNA
gccccagtggcctaatggataaggcactggccttcaaagccagggattgtgggttcgagtcccacctggggcg

SEQ ID NO: 24 - DNA, 73 bp, synthetic construct
  source 1..73: mol_type = other DNA
ggcccagtggcctaatggataaggcactggccttcaaagccagggattgtgggttcgagtcccacctgggctg

SEQ ID NO: 25 - DNA, 73 bp, synthetic construct
  source 1..73: mol_type = other DNA
gccccggtggcctaatggataaggcactggccttcaaagccagggattgtgggttcgagtcccacccggggtg

SEQ ID NO: 26 - DNA, 73 bp, synthetic construct
  source 1..73: mol_type = other DNA
gccccagtggcccaatggatagggcactggccttcaaagccagggattgtgggttcgagtcccacctggggtg

SEQ ID NO: 27 - DNA, 73 bp, synthetic construct
  source 1..73: mol_type = other DNA
gccccagtggcctagtggataaggcactggccttcaaagccagggattgtgggttcgagtcccacctggggtg

SEQ ID NO: 28 - DNA, 73 bp, synthetic construct
  source 1..73: mol_type = other DNA
gccccagtggcctaatggataaggcaccggccttcaaagccggggattgtgggttcgagtcccacctggggtg

SEQ ID NO: 29 - DNA, 73 bp, synthetic construct
  source 1..73: mol_type = other DNA
gccccagtggcctaatggataaggcactggacttcaaatccagggattgtgggttcgagtcccacctggggtg

SEQ ID NO: 30 - DNA, 73 bp, synthetic construct
  source 1..73: mol_type = other DNA
gccccagtggcctaatggataaggcactggccttcagagccagggattgtgggttcgagtcccacctggggtg

SEQ ID NO: 31 - DNA, 73 bp, synthetic construct
  source 1..73: mol_type = other DNA
gccccagtggcctaatggataaggcactggccttcaaagccagagattgtgggttcgagtcccacctggggtg

SEQ ID NO: 32 - DNA, 73 bp, synthetic construct
  source 1..73: mol_type = other DNA
gccccagtggcctaatggataaggcactggccttcaaagccagggattgcgggttcgagtcccgcctggggtg

SEQ ID NO: 33 - DNA, 73 bp, synthetic construct; identical to SEQ ID NO: 3
  source 1..73: mol_type = other DNA

SEQ ID NO: 34 - DNA, 73 bp, synthetic construct
  source 1..73: mol_type = other DNA
gccccggtggcctaatggataaggcattggccttcaaagccagggattgtgggttcgagtcccacccggggtg

SEQ ID NO: 35 - DNA, 73 bp, synthetic construct
  source 1..73: mol_type = other DNA
ggctctgtggcgcaatggatagcgcattggacttcaaattcaaaggttccgggttcgagtcccggcagagtcg

SEQ ID NO: 36 - DNA, 73 bp, synthetic construct
  source 1..73: mol_type = other DNA
ggctccgtggcgcaatggatagcgcattggacttcaaattcaaaggttgcgggttcgagtcccgccggagtcg

SEQ ID NO: 37 - DNA, 73 bp, synthetic construct
  source 1..73: mol_type = other DNA
ggctccgtggcgcaatggatagcgcattggacttcaaattcaaaggttctgggttcgagtcccagcggagtcg

SEQ ID NO: 38 - DNA, 73 bp, synthetic construct
  source 1..73: mol_type = other DNA
ggctccgtggcgcaatggatagcgcattggacttcaaattcaaaggttccgggttcgagtcccggcggagccg

SEQ ID NO: 39 - DNA, 73 bp, synthetic construct
  source 1..73: mol_type = other DNA
gcctccgtggcgcaatggatagcgcattggacttcaaattcaaaggttccgggttcgagtcccggcggaggcg

SEQ ID NO: 40 - DNA, 73 bp, synthetic construct
  source 1..73: mol_type = other DNA
ggccccgtggcgcaatggatagcgcattggacttcaaattcaaaggttccgggttcgagtcccggcggggtcg

SEQ ID NO: 41 - DNA, 73 bp, synthetic construct
  source 1..73: mol_type = other DNA
ggctcagtggcgcaatggatagcgcattggacttcaaattcaaaggttccgggttcgagtcccggctgagtcg

SEQ ID NO: 42 - DNA, 73 bp, synthetic construct
  source 1..73: mol_type = other DNA
ggctccgtggcccaatggatagggcattggacttcaaattcaaaggttccgggttcgagtcccggcggagtcg

SEQ ID NO: 43 - DNA, 73 bp, synthetic construct
  source 1..73: mol_type = other DNA
ggctccgtggcgcaatggatagcgcactggacttcaaattcagaggttccgggttcgagtcccggcggagtcg

SEQ ID NO: 44 - DNA, 73 bp, synthetic construct
  source 1..73: mol_type = other DNA
ggctccgtggcgcaatggatagcgcatcggacttcaaattcgaaggttccgggttcgagtcccggcggagtcg

SEQ ID NO: 45 - DNA, 73 bp, synthetic construct
  source 1..73: mol_type = other DNA
ggctccgtggcgcaatggatagcgcattggacttcaaatccaaaggttccgggttcgagtcccggcggagtcg

SEQ ID NO: 46 - DNA, 73 bp, synthetic construct
  source 1..73: mol_type = other DNA
ggctccgtggcgcaatggatagcgcattggccttcaaagtcaaaggttccgggttcgagtcccggcggagtcg

SEQ ID NO: 47 - DNA, 73 bp, synthetic construct
  source 1..73: mol_type = other DNA
ggctccgtggcgcaatggatagcgcattggacttcaaattcaagggttccgggttcgagtcccggcggagtcg

SEQ ID NO: 48 - DNA, 73 bp, synthetic construct
  source 1..73: mol_type = other DNA
ggctccgtggcgcaatggatagcgcattggacttcaaattcaaagattccgggttcgagtcccggcggagtcg

SEQ ID NO: 49 - DNA, 72 bp, synthetic construct
  source 1..72: mol_type = other DNA
ggttccatggtgtaatggttagcactctggactttaaatccagcgatccgagttcaaatctcggtggaacct

SEQ ID NO: 50 - DNA, 72 bp, synthetic construct
  source 1..72: mol_type = other DNA
ggttccatggtgtaatggttagcactctggactttaaatccagcgatccgagttcaagtctcggtggaacct

SEQ ID NO: 51 - DNA, 72 bp, synthetic construct
  source 1..72: mol_type = other DNA
ggttccatggtgtaatggtgagcactctggactttaaatccagcgatccgagttcgagtctcggtggaacct

SEQ ID NO: 52 - DNA, 72 bp, synthetic construct; identical to SEQ ID NO: 51
  source 1..72: mol_type = other DNA

SEQ ID NO: 53 - DNA, 72 bp, synthetic construct
  source 1..72: mol_type = other DNA
ggttccatggtgtaatggttagcactctggactttaaatccggtaatccgagttcaaatctcggtggaacct

SEQ ID NO: 54 - DNA, 72 bp, synthetic construct
  source 1..72: mol_type = other DNA
ggccccatggtgtaatggtcagcactctggactttaaatccagcgatccgagttcaaatctcggtgggaccc

SEQ ID NO: 55 - DNA, 72 bp, synthetic construct
  source 1..72: mol_type = other DNA
ggttccatggtgtaatggtaagcactctggactttaaatccagccatctgagttcgagtctctgtggaacct

SEQ ID NO: 56 - DNA, 72 bp, synthetic construct
  source 1..72: mol_type = other DNA
ggttccatggtgtaatggtgagcactttggactttaaatacagtgatcagagttcaagtctcactgggacct

SEQ ID NO: 57 - DNA, 72 bp, synthetic construct
  source 1..72: mol_type = other DNA
ggttccatgggttaatggtgagcaccctggactttaaatcaagcgatccgagttcaaatctcggtggtacct

SEQ ID NO: 58 - DNA, 72 bp, synthetic construct
  source 1..72: mol_type = other DNA
ggttccatggtgtaatggtgaccactttggactttaaatacagtgatcagagttcaagtctcactggaacct

SEQ ID NO: 59 - DNA, 72 bp, synthetic construct
  source 1..72: mol_type = other DNA
ggttccatggtgtaatggtgagggctttggactttaactacagtgatcagagttcaagtctcagtgggacct

SEQ ID NO: 60 - DNA, 73 bp, synthetic construct
  source 1..73: mol_type = other DNA
ggttccatggtgtaatggtaagcaccctggactttaaatccagcaaccagagttccagtctcagcgtggacct

SEQ ID NO: 61 - DNA, 72 bp, synthetic construct
  source 1..72: mol_type = other DNA
ggtagtgtagtctactggttaaacgcttgggctttaacattaacgtcctgggttcaaatcccagctttgtca

SEQ ID NO: 62 - DNA, 72 bp, synthetic construct
  source 1..72: mol_type = other DNA
ggtcccatggtgtaatggttagcactctggactttaaatccagcgatccgagttcaaatctcggtgggacct

SEQ ID NO: 63 - DNA, 72 bp, synthetic construct
  source 1..72: mol_type = other DNA
ggtcccatggtgtaatggttagcactctggactttaaatccagcaatccgagttcgaatctcggtgggacct

SEQ ID NO: 64 - DNA, 72 bp, synthetic construct
  source 1..72: mol_type = other DNA
ggccccatggtgtaatggttagcactctggactttaaatccagcgatccgagttcaaatctcggtgggacct

SEQ ID NO: 65 - DNA, 72 bp, synthetic construct
  source 1..72: mol_type = other DNA
ggtcccatggtgtaatggttagcactctgggctttaaatccagcaatccgagttcgaatcttggtgggacct

SEQ ID NO: 66 - DNA, 73 bp, synthetic construct
  source 1..73: mol_type = other DNA
ggctgtgtacctcagtgggcaagggtatggactttaaagccagactatttgggttcaaatcccagcttggcct

SEQ ID NO: 67 - DNA, 73 bp, synthetic construct
  source 1..73: mol_type = other DNA
gaccatgtggcctaagggaaaagacatctcactttaggtcagaagattgagggttcaagtcctttcatggtca

SEQ ID NO: 68 - DNA, 74 bp, synthetic construct
  source 1..74: mol_type = other DNA
ggtacagtgttaaaggggagaaaaattgctgactttaaatacagtagacctaggtttgaatcctggctttacca

SEQ ID NO: 69 - DNA, 72 bp, synthetic construct
  source 1..72: mol_type = other DNA
ggttccatggtgtaatggttagcactctggactctaaatccagcgatccgagttcaaatctcggtggaacct

SEQ ID NO: 70 - DNA, 72 bp, synthetic construct
  source 1..72: mol_type = other DNA
ggttccatggtgtaatggttagcactctggactctaaatccagcgatccgagttcaagtctcggtggaacct

SEQ ID NO: 71 - DNA, 72 bp, synthetic construct
  source 1..72: mol_type = other DNA
ggttccatggtgtaatggtgagcactctggactctaaatccagcgatccgagttcgagtctcggtggaacct

SEQ ID NO: 72 - DNA, 72 bp, synthetic construct
  source 1..72: mol_type = other DNA
ggttccatggtgtaatggtaagcactctggactctaaatccagcgatccgagttcgagtctcggtggaacct

SEQ ID NO: 73 - DNA, 72 bp, synthetic construct
  source 1..72: mol_type = other DNA
ggttccatggtgtaatggttagcactctggactctaaatccggtaatccgagttcaaatctcggtggaacct

SEQ ID NO: 74 - DNA, 72 bp, synthetic construct
  source 1..72: mol_type = other DNA
ggccccatggtgtaatggtcagcactctggactctaaatccagcgatccgagttcaaatctcggtgggaccc

SEQ ID NO: 75 - DNA, 72 bp, synthetic construct
  source 1..72: mol_type = other DNA
ggttccatggtgtaatggtaagcactctggactctaaatccagccatctgagttcgagtctctgtggaacct

SEQ ID NO: 76 - DNA, 72 bp, synthetic construct
  source 1..72: mol_type = other DNA
ggttccatggtgtaatggtgagcactttggactctaaatacagtgatcagagttcaagtctcactgggacct

SEQ ID NO: 77 - DNA, 72 bp, synthetic construct
  source 1..72: mol_type = other DNA
ggttccatgggttaatggtgagcaccctggactctaaatcaagcgatccgagttcaaatctcggtggtacct

SEQ ID NO: 78 - DNA, 72 bp, synthetic construct
  source 1..72: mol_type = other DNA
ggttccatggtgtaatggtgaccactttggactctaaatacagtgatcagagttcaagtctcactggaacct

SEQ ID NO: 79 - DNA, 72 bp, synthetic construct
  source 1..72: mol_type = other DNA
ggttccatggtgtaatggtgagggctttggactctaactacagtgatcagagttcaagtctcagtgggacct

SEQ ID NO: 80 - DNA, 73 bp, synthetic construct
  source 1..73: mol_type = other DNA
ggttccatggtgtaatggtaagcaccctggactctaaatccagcaaccagagttccagtctcagcgtggacct

SEQ ID NO: 81 - DNA, 72 bp, synthetic construct
  source 1..72: mol_type = other DNA
ggtagtgtagtctactggttaaacgcttgggctctaacattaacgtcctgggttcaaatcccagctttgtca

SEQ ID NO: 82 - DNA, 72 bp, synthetic construct
  source 1..72: mol_type = other DNA
ggtcccatggtgtaatggttagcactctggactctaaatccagcgatccgagttcaaatctcggtgggacct

SEQ ID NO: 83 - DNA, 72 bp, synthetic construct
  source 1..72: mol_type = other DNA
ggtcccatggtgtaatggttagcactctggactctaaatccagcaatccgagttcgaatctcggtgggacct

SEQ ID NO: 84 - DNA, 72 bp, synthetic construct
  source 1..72: mol_type = other DNA
ggccccatggtgtaatggttagcactctggactctaaatccagcgatccgagttcaaatctcggtgggacct

SEQ ID NO: 85 - DNA, 72 bp, synthetic construct
  source 1..72: mol_type = other DNA
ggtcccatggtgtaatggttagcactctgggctctaaatccagcaatccgagttcgaatcttggtgggacct

SEQ ID NO: 86 - DNA, 73 bp, synthetic construct
  source 1..73: mol_type = other DNA
ggctgtgtacctcagtgggcaagggtatggactctaaagccagactatttgggttcaaatcccagcttggcct

SEQ ID NO: 87 - DNA, 73 bp, synthetic construct
  source 1..73: mol_type = other DNA
gaccatgtggcctaagggaaaagacatctcactctaggtcagaagattgagggttcaagtcctttcatggtca

SEQ ID NO: 88 - DNA, 74 bp, synthetic construct
  source 1..74: mol_type = other DNA
ggtacagtgttaaaggggagaaaaattgctgactctaaatacagtagacctaggtttgaatcctggctttacca

SEQ ID NO: 89 - protein, 1008 aa, synthetic construct
MAAAAAAAPSGGGGGGEEERLEEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVSORWITHREFERENCETOSEQIDNOMVAGMLGLREEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 90 - protein, 486 aa, synthetic construct
MVAGMLGLREEKSEDQDLQGLKDKPLKFKKVKKDKKEEKEGKHEPVQPSAHHSAEPAEAGKAETSEGSGSAPAVPEASASPKQRRSIIRDRGPMYDDPTLPEGWTRKLKQRKSGRSAGKYDVYLINPQGKAFRSKVELIAYFEKVGDTSLDPNDFDFTVTGRGSPSRREQKPPKKPKSPKAPGTGRGRGRPKGSGTTRPKAATSEGVQVKRVLEKSPGKLLVKMPFQTSPGGKAEGGGATTSTQVMVIKRPGRKRKAEADPQAIPKKRGRKPGSVVAAAAAEAKKKAVKESSIRSVQETVLPIKKRKTRETVSIEVKEVVKPLLVSTLGEKSGKGLKTCKSPGRKSKESSPKGRSSSASSPPKKEHHHHHHHSESPKAPVPLLPPLPPPPPEPESSEDPTSPPEPQDLSSSVCKEEKMPRGGSLESDGCPKEPAKTQPAVATAATAAEKYKHRGEGERKDIVSSSMPRPNREEPVDSRTPVTERVS

SEQ ID NO: 91-100 - skipped (10 entries)

SEQ ID NO: 101 - RNA, 45 nt, synthetic construct
  source 1..45: mol_type = other RNA
  tRNA 1..45: note = tRNA
ggtggtattgcccggagtagaagcacctgtgactaactgtgccaa

SEQ ID NO: 102 - RNA, 49 nt, synthetic construct
  source 1..49: mol_type = other RNA
  tRNA 1..49: note = tRNA
ttttttgatggccagctccgaggatctagcgtactgagtcgcccagtct

SEQ ID NO: 103 - RNA, 73 nt, synthetic construct
  source 1..73: mol_type = other RNA
  tRNA 1..73: note = tRNA
gccccagtggcctaatggataaggcactggccttcaaagccagggattgtgggttcgagtcccacctggggtg

SEQ ID NO: 104 - RNA, 73 nt, synthetic construct
  source 1..73: mol_type = other RNA
  tRNA 1..73: note = tRNA
gccccagtggcctaatggataaggcactggccttcaaagccagggattgtgggttcgagtcccacctggggta

SEQ ID NO: 105 - RNA, 73 nt, synthetic construct
  source 1..73: mol_type = other RNA
  tRNA 1..73: note = tRNA
gccccggtggcctaatggataaggcattggccttcaaagccagggattgtgggttcgagtcccacccggggta

SEQ ID NO: 106 - RNA, 73 nt, synthetic construct
  source 1..73: mol_type = other RNA
  tRNA 1..73: note = tRNA
ggctccgtggcgcaatggatagcgcattggacttcaaattcaaaggttccgggttcgagtcccggcggagtcg

SEQ ID NO: 107 - RNA, 73 nt, synthetic construct
  source 1..73: mol_type = other RNA
  tRNA 1..73: note = tRNA
gccccagtggcctaatggataaggcattggccttcaaagccagggattgtgggttcgagtcccatctggggtg

SEQ ID NO: 108 - RNA, 73 nt, synthetic construct
  source 1..73: mol_type = other RNA
  tRNA 1..73: note = tRNA
gccccagtggcctgatggataaggtactggccttcaaagccagggattgtgggttcgagttccacctggggta

SEQ ID NO: 109 - RNA, 73 nt, synthetic construct
  source 1..73: mol_type = other RNA
  tRNA 1..73: note = tRNA
ggctctgtggcgcaatggatagcgcattggacttcaaattcaaaggttgtgggttcgaatcccaccagagtcg

SEQ ID NO: 110 - RNA, 73 nt, synthetic construct
  source 1..73: mol_type = other RNA
  tRNA 1..73: note = tRNA
ggctctgtggcgcaatggatagcgcattggacttcaaattcaaaggttgtgggttcgagtcccaccagagtcg

SEQ ID NO: 111 - RNA, 74 nt, synthetic construct
  source 1..74: mol_type = other RNA
  tRNA 1..74: note = tRNA
gtctctgtggcgcaatggacgagcgcgctggacttcaaatccagaggttccgggttcgagtcccggcagagatg

SEQ ID NO: 112 - RNA, 73 nt, synthetic construct
  source 1..73: mol_type = other RNA
  tRNA 1..73: note = tRNA
ggctctgtggcgcaatggatagcgcattggacttcaaattcaaaggttgcgggttcgagtccctccagagtcg

SEQ ID NO: 113 - RNA, 73 nt, synthetic construct
  source 1..73: mol_type = other RNA
  tRNA 1..73: note = tRNA
ggccgcgtggcctaatggataaggcgtctgacttcagatcagaagattgcaggttcgagtcctgccgcggtcg

SEQ ID NO: 114 - RNA, 73 nt, synthetic construct
  source 1..73: mol_type = other RNA
  tRNA 1..73: note = tRNA
gaccacgtggcctaatggataaggcgtctgacttcagatcagaagattgagggttcgaatccctccgtggtta

SEQ ID NO: 115 - RNA, 73 nt, synthetic construct
  source 1..73: mol_type = other RNA
  tRNA 1..73: note = tRNA
gaccgcgtggcctaatggataaggcgtctgacttcagatcagaagattgagggttcgagtcccttcgtggtcg

SEQ ID NO: 116 - RNA, 73 nt, synthetic construct
  source 1..73: mol_type = other RNA
  tRNA 1..73: note = tRNA
gaccacgtggcctaatggataaggcgtctgacttcagatcagaagattgagggttcgaatcccttcgtggtta

SEQ ID NO: 117 - RNA, 73 nt, synthetic construct
  source 1..73: mol_type = other RNA
  tRNA 1..73: note = tRNA
gaccacgtggcctaatggataaggcgtctgacttcagatcagaagattgagggttcgaatcccttcgtggttg

SEQ ID NO: 118 - RNA, 73 nt, synthetic construct
  source 1..73: mol_type = other RNA
  tRNA 1..73: note = tRNA
ggccgtgtggcctaatggataaggcgtctgacttcagatcaaaagattgcaggtttgagttctgccacggtcg

SEQ ID NO: 119 - RNA, 73 nt, synthetic construct
  source 1..73: mol_type = other RNA
  tRNA 1..73: note = tRNA
ggccgcgtggcctaatggataaggcgtctgatttcagatcagaagattgagggttcgagtcccttcgtggtcg

SEQ ID NO: 120 - RNA, 73 nt, synthetic construct
  source 1..73: mol_type = other RNA
  tRNA 1..73: note = tRNA
gacccagtggcctaatggataaggcatcagccttcagagctggggattgtgggttcgagtcccatctgggtcg

SEQ ID NO: 121 - RNA, 73 nt, synthetic construct
  source 1..73: mol_type = other RNA
  tRNA 1..73: note = tRNA
gggccagtggcgcaatggataacgcgtctgacttcagatcagaagattccaggttcgactcctggctggctcg

SEQ ID NO: 122 - RNA, 73 nt, synthetic construct
  source 1..73: mol_type = other RNA
  tRNA 1..73: note = tRNA
gggccagtggcgcaatggataacgcgtctgacttcagatcagaagattctaggttcgactcctggctggctcg

SEQ ID NO: 123 - RNA, 73 nt, synthetic construct
  source 1..73: mol_type = other RNA
  tRNA 1..73: note = tRNA
gccccagtggcctaatggataaggcactggccttcaaagccagggattgtgggttcgagtcccacctggggcg

SEQ ID NO: 124 - RNA, 73 nt, synthetic construct
  source 1..73: mol_type = other RNA
  tRNA 1..73: note = tRNA
ggcccagtggcctaatggataaggcactggccttcaaagccagggattgtgggttcgagtcccacctgggctg

SEQ ID NO: 125 - RNA, 73 nt, synthetic construct
  source 1..73: mol_type = other RNA
  tRNA 1..73: note = tRNA
gccccggtggcctaatggataaggcactggccttcaaagccagggattgtgggttcgagtcccacccggggtg

SEQ ID NO: 126 - RNA, 73 nt, synthetic construct
  source 1..73: mol_type = other RNA
  tRNA 1..73: note = tRNA
gccccagtggcccaatggatagggcactggccttcaaagccagggattgtgggttcgagtcccacctggggtg

SEQ ID NO: 127 - RNA, 73 nt, synthetic construct
  source 1..73: mol_type = other RNA
  tRNA 1..73: note = tRNA
gccccagtggcctagtggataaggcactggccttcaaagccagggattgtgggttcgagtcccacctggggtg

SEQ ID NO: 128 - RNA, 73 nt, synthetic construct
  source 1..73: mol_type = other RNA
  tRNA 1..73: note = tRNA
gccccagtggcctaatggataaggcaccggccttcaaagccggggattgtgggttcgagtcccacctggggtg

SEQ ID NO: 129 - RNA, 73 nt, synthetic construct
  source 1..73: mol_type = other RNA
  tRNA 1..73: note = tRNA
gccccagtggcctaatggataaggcactggacttcaaatccagggattgtgggttcgagtcccacctggggtg

SEQ ID NO: 130 - RNA, 73 nt, synthetic construct
  source 1..73: mol_type = other RNA
  tRNA 1..73: note = tRNA
gccccagtggcctaatggataaggcactggccttcagagccagggattgtgggttcgagtcccacctggggtg

SEQ ID NO: 131 - RNA, 73 nt, synthetic construct
  source 1..73: mol_type = other RNA
  tRNA 1..73: note = tRNA
gccccagtggcctaatggataaggcactggccttcaaagccagagattgtgggttcgagtcccacctggggtg

SEQ ID NO: 132 - RNA, 73 nt, synthetic construct
  source 1..73: mol_type = other RNA
  tRNA 1..73: note = tRNA
gccccagtggcctaatggataaggcactggccttcaaagccagggattgcgggttcgagtcccgcctggggtg

SEQ ID NO: 133 - RNA, 73 nt, synthetic construct; identical to SEQ ID NO: 103
  source 1..73: mol_type = other RNA
  tRNA 1..73: note = tRNA

SEQ ID NO: 134 - RNA, 73 nt, synthetic construct
  source 1..73: mol_type = other RNA
  tRNA 1..73: note = tRNA
gccccggtggcctaatggataaggcattggccttcaaagccagggattgtgggttcgagtcccacccggggtg

SEQ ID NO: 135 - RNA, 73 nt, synthetic construct
  source 1..73: mol_type = other RNA
  tRNA 1..73: note = tRNA
ggctctgtggcgcaatggatagcgcattggacttcaaattcaaaggttccgggttcgagtcccggcagagtcg

SEQ ID NO: 136 - RNA, 73 nt, synthetic construct
  source 1..73: mol_type = other RNA
  tRNA 1..73: note = tRNA
ggctccgtggcgcaatggatagcgcattggacttcaaattcaaaggttgcgggttcgagtcccgccggagtcg

SEQ ID NO: 137 - RNA, 73 nt, synthetic construct
  source 1..73: mol_type = other RNA
  tRNA 1..73: note = tRNA
ggctccgtggcgcaatggatagcgcattggacttcaaattcaaaggttctgggttcgagtcccagcggagtcg

SEQ ID NO: 138 - RNA, 73 nt, synthetic construct
  source 1..73: mol_type = other RNA
  tRNA 1..73: note = tRNA
ggctccgtggcgcaatggatagcgcattggacttcaaattcaaaggttccgggttcgagtcccggcggagccg

SEQ ID NO: 139 - RNA, 73 nt, synthetic construct
  source 1..73: mol_type = other RNA
  tRNA 1..73: note = tRNA
gcctccgtggcgcaatggatagcgcattggacttcaaattcaaaggttccgggttcgagtcccggcggaggcg

SEQ ID NO: 140 - RNA, 73 nt, synthetic construct
  source 1..73: mol_type = other RNA
  tRNA 1..73: note = tRNA
ggccccgtggcgcaatggatagcgcattggacttcaaattcaaaggttccgggttcgagtcccggcggggtcg

SEQ ID NO: 141 - RNA, 73 nt, synthetic construct
  source 1..73: mol_type = other RNA
  tRNA 1..73: note = tRNA
ggctcagtggcgcaatggatagcgcattggacttcaaattcaaaggttccgggttcgagtcccggctgagtcg

SEQ ID NO: 142 - RNA, 73 nt, synthetic construct
  source 1..73: mol_type = other RNA
  tRNA 1..73: note = tRNA
ggctccgtggcccaatggatagggcattggacttcaaattcaaaggttccgggttcgagtcccggcggagtcg

SEQ ID NO: 143 - RNA, 73 nt, synthetic construct
  source 1..73: mol_type = other RNA
  tRNA 1..73: note = tRNA
ggctccgtggcgcaatggatagcgcactggacttcaaattcagaggttccgggttcgagtcccggcggagtcg

SEQ ID NO: 144 - RNA, 73 nt, synthetic construct
  source 1..73: mol_type = other RNA
  tRNA 1..73: note = tRNA
ggctccgtggcgcaatggatagcgcatcggacttcaaattcgaaggttccgggttcgagtcccggcggagtcg

SEQ ID NO: 145 - RNA, 73 nt, synthetic construct
  source 1..73: mol_type = other RNA
  tRNA 1..73: note = tRNA
ggctccgtggcgcaatggatagcgcattggacttcaaatccaaaggttccgggttcgagtcccggcggagtcg

SEQ ID NO: 146 - RNA, 73 nt, synthetic construct
  source 1..73: mol_type = other RNA
  tRNA 1..73: note = tRNA
ggctccgtggcgcaatggatagcgcattggccttcaaagtcaaaggttccgggttcgagtcccggcggagtcg

SEQ ID NO: 147 - RNA, 73 nt, synthetic construct
  source 1..73: mol_type = other RNA
  tRNA 1..73: note = tRNA
ggctccgtggcgcaatggatagcgcattggacttcaaattcaagggttccgggttcgagtcccggcggagtcg

SEQ ID NO: 148 - RNA, 73 nt, synthetic construct
  source 1..73: mol_type = other RNA
  tRNA 1..73: note = tRNA
ggctccgtggcgcaatggatagcgcattggacttcaaattcaaagattccgggttcgagtcccggcggagtcg

SEQ ID NO: 149 - DNA, 40 bp, synthetic construct
  source 1..40: mol_type = other DNA
tccctatcagtgatagagatctccctatcagtgatagaga

SEQ ID NO: 150 - DNA, 19 bp, synthetic construct
  source 1..19: mol_type = other DNA
tccctatcagtgatagaga

SEQ ID NO: 151 - RNA, 73 nt, synthetic construct
  source 1..73: mol_type = other RNA
  tRNA 1..73: note = tRNA
gccccagtggcctaatggataaggcactggcctcctaagccagggattgcgggttcgagtcccgcctggggtg

SEQ ID NO: 152 - RNA, 71 nt, synthetic construct
  source 1..71: mol_type = other RNA
  tRNA 1..71: note = tRNA
ggtcccatggtgtaatggttagcactctggactctaaatccagcgatccgagttcaaatctcggtgggacc

SEQ ID NO: 153 - RNA, 71 nt, synthetic construct
  source 1..71: mol_type = other RNA
  tRNA 1..71: note = tRNA
ggtcccatggtgtaatggttagcactctggactttaaatccagcgatccgagttcaaatctcggtgggacc

SEQ ID NO: 154 - DNA, 7327 bp, synthetic construct
  source 1..7327: mol_type = other DNA
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 155 - DNA, 7327 bp, synthetic construct
  source 1..7327: mol_type = other DNA
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctaaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctaaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 156 - DNA, 7287 bp, synthetic construct
  source 1..7287: mol_type = other DNA
gcgcgccgatatcgttaacgccccgcgccggccgctctagaactagtggatcccccggaagatcagaagttcctattccgaagttcctattctctagaaagtataggaacttctgatctgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccacgggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtagttgcttgttaatcaatagaccgtttagttcgtttcagttgaactttggtctctgcgtatttctttcttatctagtttccatggctacgtagataagtagcatggcgggttaatcattaactacagcccgggcgtttaaacagcgggcggaggggtggagtcgtgacgtgaattacgtcatagggttagggaggtcctgtattagaggtcacgtgagtgttttgcgacattttgcgacaccatgtggtctcgctgggggggggggcccgagtgagcacgcagggtctccattttgaagcgggaggtttgaacgagcgctggcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcatttttttaaccaataggccgaaatcggcaaaatcccttataaatcaaaagaatagaccgagatagggttgagtgttgttccagtttggaacaagagtccactattaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaa

SEQ ID NO: 157 - DNA, 73 bp, synthetic construct; identical to SEQ ID NO: 8
  source 1..73: mol_type = other DNA

SEQ ID NO: 158 - DNA, 105 bp, synthetic construct
  source 1..105: mol_type = other DNA
tccctatcagtgatagagatgcagaagccccagtggcctaatggataaggcactggcctcctaagccagggattgcgggttcgagtcccgcctggggtgtttttt

SEQ ID NO: 159 - DNA, 110 bp, synthetic construct
  source 1..110: mol_type = other DNA
tccctatcagtgatagagaagagttgcagaagccccagtggcctaatggataaggcactggccttcaaagccagggattgcgggttcgagtcccgcctggggtgtttttt

SEQ ID NO: 160 - DNA, 118 bp, synthetic construct
  source 1..118: mol_type = other DNA
tccctatcagtgatagagaatcagataagagttgcagaagccccagtggcctaatggataaggcactggccttcaaagccagggattgcgggttcgagtcccgcctggggtgtttttt

SEQ ID NO: 161 - DNA, 128 bp, synthetic construct
  source 1..128: mol_type = other DNA
tccctatcagtgatagagaatagccgttgatcagataagagttgcagaagccccagtggcctaatggataaggcactggccttcaaagccagggattgcgggttcgagtcccgcctggggtgtttttt

SEQ ID NO: 162 - DNA, 143 bp, synthetic construct
  source 1..143: mol_type = other DNA
tccctatcagtgatagagatggcaattccacgttatagccgttgatcagataagagttgcagaagccccagtggcctaatggataaggcactggccttcaaagccagggattgcgggttcgagtcccgcctggggtgtttttt

SEQ ID NO: 163 - DNA, 98 bp, synthetic construct
  source 1..98: mol_type = other DNA
tccctatcagtgatagagagccccagtggcctaatggataaggcactggccttcaaagccagggattgcgggttcgagtcccgcctggggtgtttttt

SEQ ID NO: 164 - DNA, 106 bp, synthetic construct
  source 1..106: mol_type = other DNA
tgcagaagccccagtggcctaatggataaggcactggccttcaaagccagggattgcgggttcgagtcccgcctggggtgttttttttccctatcagtgatagaga

SEQ ID NO: 165 - DNA, 111 bp, synthetic construct
  source 1..111: mol_type = other DNA
tgcagaagccccagtggcctaatggataaggcactggccttcaaagccagggattgcgggttcgagtcccgcctggggtgtttttttatctgtccctatcagtgatagaga

SEQ ID NO: 166 - DNA, 119 bp, synthetic construct
  source 1..119: mol_type = other DNA
tgcagaagccccagtggcctaatggataaggcactggccttcaaagccagggattgcgggttcgagtcccgcctggggtgtttttttatctgcaagcctgtccctatcagtgatagaga

SEQ ID NO: 167 - DNA, 129 bp, synthetic construct
  source 1..129: mol_type = other DNA
tgcagaagccccagtggcctaatggataaggcactggccttcaaagccagggattgcgggttcgagtcccgcctggggtgtttttttatctgcaagcctggcaattccactccctatcagtgatagaga

SEQ ID NO: 168 - DNA, 144 bp, synthetic construct
  source 1..144: mol_type = other DNA
tgcagaagccccagtggcctaatggataaggcactggccttcaaagccagggattgcgggttcgagtcccgcctggggtgtttttttatctgcaagcctggcaattccacgttatagccgttgattccctatcagtgatagaga

SEQ ID NO: 169 - DNA, 86 bp, synthetic construct
  source 1..86: mol_type = other DNA
tgcagaagccccagtggcctaatggataaggcactggccttcaaagccagggattgcgggttcgagtcccgcctggggtgtttttt

SEQ ID NO: 170 - DNA, 86 bp, synthetic construct
  source 1..86: mol_type = other DNA
tgcagaagccccagtggcctaatggataaggcactggcctcctaagccagggattgcgggttcgagtcccgcctggggtgtttttt

SEQ ID NO: 171 - DNA, 126 bp, synthetic construct
  source 1..126: mol_type = other DNA
tccctatcagtgatagagatctccctatcagtgatagagatgcagaagccccagtggcctaatggataaggcactggccttcaaagccagggattgcgggttcgagtcccgcctggggtgtttttt

SEQ ID NO: 172 - DNA, 131 bp, synthetic construct
  source 1..131: mol_type = other DNA
tccctatcagtgatagagatctccctatcagtgatagagaagagttgcagaagccccagtggcctaatggataaggcactggccttcaaagccagggattgcgggttcgagtcccgcctggggtgtttttt

SEQ ID NO: 173 - DNA, 139 bp, synthetic construct
  source 1..139: mol_type = other DNA
tccctatcagtgatagagatctccctatcagtgatagagaatcagataagagttgcagaagccccagtggcctaatggataaggcactggccttcaaagccagggattgcgggttcgagtcccgcctggggtgtttttt

SEQ ID NO: 174 - DNA, 149 bp, synthetic construct
  source 1..149: mol_type = other DNA
tccctatcagtgatagagatctccctatcagtgatagagaatagccgttgatcagataagagttgcagaagccccagtggcctaatggataaggcactggccttcaaagccagggattgcgggttcgagtcccgcctggggtgtttttt

SEQ ID NO: 175 - DNA, 164 bp, synthetic construct
  source 1..164: mol_type = other DNA
tccctatcagtgatagagatctccctatcagtgatagagatggcaattccacgttatagccgttgatcagataagagttgcagaagccccagtggcctaatggataaggcactggccttcaaagccagggattgcgggttcgagtcccgcctggggtgtttttt

SEQ ID NO: 176 - DNA, 119 bp, synthetic construct
  source 1..119: mol_type = other DNA
tccctatcagtgatagagatctccctatcagtgatagagagccccagtggcctaatggataaggcactggccttcaaagccagggattgcgggttcgagtcccgcctggggtgtttttt

SEQ ID NO: 177 - DNA, 127 bp, synthetic construct
  source 1..127: mol_type = other DNA
tgcagaagccccagtggcctaatggataaggcactggccttcaaagccagggattgcgggttcgagtcccgcctggggtgttttttttccctatcagtgatagagatctccctatcagtgatagaga

SEQ ID NO: 178 - DNA, 132 bp, synthetic construct
  source 1..132: mol_type = other DNA
tgcagaagccccagtggcctaatggataaggcactggccttcaaagccagggattgcgggttcgagtcccgcctggggtgtttttttatctgtccctatcagtgatagagatctccctatcagtgatagaga

SEQ ID NO: 179 - DNA, 140 bp, synthetic construct
  source 1..140: mol_type = other DNA
tgcagaagccccagtggcctaatggataaggcactggccttcaaagccagggattgcgggttcgagtcccgcctggggtgtttttttatctgcaagcctgtccctatcagtgatagagatctccctatcagtgatagaga

SEQ ID NO: 180 - DNA, 150 bp, synthetic construct
  source 1..150: mol_type = other DNA
tgcagaagccccagtggcctaatggataaggcactggccttcaaagccagggattgcgggttcgagtcccgcctggggtgtttttttatctgcaagcctggcaattccactccctatcagtgatagagatctccctatcagtgatagaga

SEQ ID NO: 181 - DNA, 165 bp, synthetic construct
  source 1..165: mol_type = other DNA
tgcagaagccccagtggcctaatggataaggcactggccttcaaagccagggattgcgggttcgagtcccgcctggggtgtttttttatctgcaagcctggcaattccacgttatagccgttgattccctatcagtgatagagatctccctatcagtgatagaga

SEQ ID NO: 182 - DNA, 329 bp, synthetic construct
  source 1..329: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtccctatcagtgatagagattatatatcttccctatcagtgatagagacgccccagtggcctaatggataaggcactggccttcaaagccagggattgcgggttcgagtcccgcctggggtgtttttt

SEQ ID NO: 183 - DNA, 329 bp, synthetic construct
  source 1..329: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtccctatcagtgatagagattatatatcttccctatcagtgatagagacgccccagtggcctaatggataaggcattggccttcaaagccagggattgtgggttcgagtcccatctggggtgtttttt

SEQ ID NO: 184 - DNA, 329 bp, synthetic construct
  source 1..329: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtccctatcagtgatagagattatatatctttccctatcagtgatagagagccccagtggcctaatggataaggcactggccttcaaagccagggattgcgggttcgagtcccgcctggggtgtttttt

SEQ ID NO: 185 - DNA, 329 bp, synthetic construct
  source 1..329: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtccctatcagtgatagagattatatatctttccctatcagtgatagagagccccagtggcctaatggataaggcattggccttcaaagccagggattgtgggttcgagtcccatctggggtgtttttt

SEQ ID NO: 186 - DNA, 328 bp, synthetic construct
  source 1..328: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttccctatcagtgatagagacgccccagtggcctaatggataaggcactggccttcaaagccagggattgcgggttcgagtcccgcctggggtgtttttt

SEQ ID NO: 187 - DNA, 328 bp, synthetic construct
  source 1..328: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttccctatcagtgatagagacgccccagtggcctaatggataaggcattggccttcaaagccagggattgtgggttcgagtcccatctggggtgtttttt

SEQ ID NO: 188 - DNA, 328 bp, synthetic construct
  source 1..328: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatctttccctatcagtgatagagagccccagtggcctaatggataaggcactggccttcaaagccagggattgcgggttcgagtcccgcctggggtgtttttt

SEQ ID NO: 189 - DNA, 328 bp, synthetic construct
  source 1..328: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatctttccctatcagtgatagagagccccagtggcctaatggataaggcattggccttcaaagccagggattgtgggttcgagtcccatctggggtgtttttt

SEQ ID NO: 190 - DNA, 348 bp, synthetic construct
  source 1..348: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatctccctatcagtgatagagatcgtccctatcagtgatagagagccccagtggcctaatggataaggcattggccttcaaagccagggattgtgggttcgagtcccatctggggtgtttttt

SEQ ID NO: 191 - DNA, 348 bp, synthetic construct
  source 1..348: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttccctatcagtgatagagatctccctatcagtgatagagacttaccgtaacttgaaagtatttcgatttcttggctttatatatctccctatcagtgatagagatcgtccctatcagtgatagagagccccagtggcctaatggataaggcattggccttcaaagccagggattgtgggttcgagtcccatctggggtgtttttt

SEQ ID NO: 192 - DNA, 369 bp, synthetic construct
  source 1..369: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttccctatcagtgatagagacttaccgtaacttgaaagtatttcgatttcttggctttatatatcttgtggaaaggacgaaacaccgtccctatcagtgatagagatctccctatcagtgatagagagccccagtggcctaatggataaggcattggccttcaaagccagggattgtgggttcgagtcccatctggggtgtttttt

SEQ ID NO: 193 - DNA, 369 bp, synthetic construct
  source 1..369: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtccctatcagtgatagagatctccctatcagtgatagagagccccagtggcctaatggataaggcattggccttcaaagccagggattgtgggttcgagtcccatctggggtgtttttt

SEQ ID NO: 194 - DNA, 79 bp, synthetic construct
  source 1..79: mol_type = other DNA
gccccagtggcctaatggataaggcattggcctcctaagccagggattgtgggttcgagtcccatctggggtgtttttt

SEQ ID NO: 195 - DNA, 99 bp, synthetic construct
  source 1..99: mol_type = other DNA
tccctatcagtgatagagacgccccagtggcctaatggataaggcactggccttcaaagccagggattgcgggttcgagtcccgcctggggtgtttttt

SEQ ID NO: 196 - DNA, 105 bp, synthetic construct
  source 1..105: mol_type = other DNA
tccctatcagtgatagagatgcagaagccccagtggcctaatggataaggcactggccttcaaagccagggattgcgggttcgagtcccgcctggggtgtttttt

SEQ ID NO: 197 - DNA, 98 bp, synthetic construct
  source 1..98: mol_type = other DNA
tccctatcagtgatagagagccccagtggcctaatggataaggcattggccttcaaagccagggattgtgggttcgagtcccatctggggtgtttttt

SEQ ID NO: 198 - DNA, 119 bp, synthetic construct
  source 1..119: mol_type = other DNA
tccctatcagtgatagagatctccctatcagtgatagagagccccagtggcctaatggataaggcattggccttcaaagccagggattgtgggttcgagtcccatctggggtgtttttt

SEQ ID NO: 199 - DNA, 328 bp, synthetic construct
  source 1..328: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccccagtggcctaatggataaggcactggccttcaaagccagggattgcgggttcgagtcccgcctggggtgtttttt

SEQ ID NO: 200 - DNA, 328 bp, synthetic construct
  source 1..328: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccccagtggcctaatggataaggcactggcctcctaagccagggattgcgggttcgagtcccgcctggggtgtttttt

SEQ ID NO: 201 - DNA, 328 bp, synthetic construct
  source 1..328: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccccagtggcctaatggataaggcattggccttcaaagccagggattgtgggttcgagtcccatctggggtgtttttt

SEQ ID NO: 202 - DNA, 328 bp, synthetic construct
  source 1..328: mol_type = other DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccccagtggcctaatggataaggcattggcctcctaagccagggattgtgggttcgagtcccatctggggtgtttttt

SEQ ID NO: 203 - DNA, 2242 bp, synthetic construct
  source 1..2242: mol_type = other DNA
tctgcgcgctcgctcgctcactgaggccgggcgaccaaaggtcgcccgacgcccgggctttgcccgggcggcctcagtgagcgagcgagcgcgcagaggatcctatcaagaaccaacctagaacaggataccacagtgaatatcactgttagatccctatcagtgatagagatggcaattccacgttatagccgttgatcagataagagttgcagaagccccagtggcctaatggataaggcactggccttcaaagccagggattgcgggttcgagtcccgcctggggtgttttttttggaccgttataaccggtattatcttcgtgttattgccgtcgtagcagacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcacaagcggcgctcactgtagaccttgacacaccactgatcggcagattccttaactgtaatcaagagttcttagaggagacaatccaactccttggactcctcgaataactctactcggttcacagtggaacactattgattgtagattacggtgaggccgtgctcagacttcgagatcgatcacaagattgtatccaagcggaagacttagaacaatctgcaagctaataccacggcagatcgtattggagtataggattctacctaatacggtattcagtcaggagattaagattgcgcagtgcagattctgaacttgtcaagttccttactcgcagcgtactggcctagctatcgtgctcacactgctatcttggtgcgactattgttctgttgcagtcaccgtgtgtagtgtatctgcagtcagactactctctacggaggctgcttaagtatcagcgctgagttgtcgcttgattgtgctggtgactataagtcctgaagtccgattgttgcctcaattgttactgtagcgatacgattaagcgacgatataggcgacggaagatacagcttgctattgagtgcgcgttctatcgtgacagattattagaagtgtagaccggaggatcagatagatcggcctaccgcacacctgagatcaatacgtcggtaagagcgctgtaaggactaatacaatagtaataagacctattatagagggtactaagattgcggcctggccactgactaagatagaagctgtaacttgcctcagattgtggccgctgatctgcagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 204 - DNA, 2242 bp, synthetic construct
  source 1..2242: mol_type = other DNA
tctgcgcgctcgctcgctcactgaggccgggcgaccaaaggtcgcccgacgcccgggctttgcccgggcggcctcagtgagcgagcgagcgcgcagaggatcctatcaagaaccaacctagaacaggataccacagtgaatatcactgttagatccctatcagtgatagagatggcaattccacgttatagccgttgatcagataagagttgcagaagccccagtggcctaatggataaggcactggccttcaaagccagggattgcgggttcgagtcccgcctggggtgttttttcgataggctgttgtaaggcgattggtgaaggaatacgcggtacaagtacactctccctatcagtgatagagaactgttaacaagacacagtgtgttgtctattcggttggtgcagaagccccagtggcctaatggataaggcactggccttcaaagccagggattgcgggttcgagtcccgcctggggtgttttttgaatagttacagcgattaccgcctattaccaacacggaagcactagactaatatccctatcagtgatagagactgactggtacaggaagtatctgatctagacgaattggtgcagaagccccagtggcctaatggataaggcactggccttcaaagccagggattgcgggttcgagtcccgcctggggtgttttttttggaccgttataaccggtattatcttcgtgttattgccgtcgtagcagacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcacaagcggcgctcactgtagaccttgacacaccactgatcggcagattccttaactgtaatcaagagttcttagaggagacaatccaactccttggactcctcgaataactctactcggttcacagtggaacactattgattgtagattacggtgaggccgtgctcagacttcgagatcgatcacaagattgtatccaagcggaagacttagaacaatctgcaagctaataccacggcagatcgtattggagtataggattctacctaatacggtattcagtcaggagattaagattgcgcagtgcagattctgaacttgtcaagttccttagtactaagattgcggcctggccactgactaagatagaagctgtaacttgcctcagattgtggccgctgatctgcagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 205 - DNA, 2242 bp, synthetic construct
  source 1..2242: mol_type = other DNA
tctgcgcgctcgctcgctcactgaggccgggcgaccaaaggtcgcccgacgcccgggctttgcccgggcggcctcagtgagcgagcgagcgcgcagaggatcctatcaagaaccaacctagaacaggataccacagtgaatatcactgttagatccctatcagtgatagagagccccagtggcctaatggataaggcactggccttcaaagccagggattgcgggttcgagtcccgcctggggtgttttttttggaccgttataaccggtattatcttcgtgttattgccgtcgtagcagacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcacaagcggcgctcactgtagaccttgacacaccactgatcggcagattccttaactgtaatcaagagttcttagaggagacaatccaactccttggactcctcgaataactctactcggttcacagtggaacactattgattgtagattacggtgaggccgtgctcagacttcgagatcgatcacaagattgtatccaagcggaagacttagaacaatctgcaagctaataccacggcagatcgtattggagtataggattctacctaatacggtattcagtcaggagattaagattgcgcagtgcagattctgaacttgtcaagttccttactcgcagcgtactggcctagctatcgtgctcacactgctatcttggtgcgactattgttctgttgcagtcaccgtgtgtagtgtatctgcagtcagactactctctacggaggctgcttaagtatcagcgctgagttgtcgcttgattgtgctggtgactataagtcctgaagtccgattgttgcctcaattgttactgtagcgatacgattaagcgacgatataggcgacggaagatacagcttgctattgagtgcgcgttctatcgtgacagattattagaagtgtagaccggaggatcagatagatcggcctaccgcacacctgagatcaatacgtcggtaagagcgctgtaaggactaatacaatagtaataagacctattatagaggctgactggtacaggaagtatctgatctagacgaattggcacacaagtactaagattgcggcctggccactgactaagatagaagctgtaacttgcctcagattgtggccgctgatctgcagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 206 - DNA, 2242 bp, synthetic construct
  source 1..2242: mol_type = other DNA
tctgcgcgctcgctcgctcactgaggccgggcgaccaaaggtcgcccgacgcccgggctttgcccgggcggcctcagtgagcgagcgagcgcgcagaggatcctatcaagaaccaacctagaacaggataccacagtgaatatcactgttagatccctatcagtgatagagagccccagtggcctaatggataaggcactggccttcaaagccagggattgcgggttcgagtcccgcctggggtgttttttcgataggctgttgtaaggcgattggtgaaggaatacgcggtacaagtacactctccctatcagtgatagagagccccagtggcctaatggataaggcactggccttcaaagccagggattgcgggttcgagtcccgcctggggtgttttttgaatagttacagcgattaccgcctattaccaacacggaagcactagactaatatccctatcagtgatagagagccccagtggcctaatggataaggcactggccttcaaagccagggattgcgggttcgagtcccgcctggggtgttttttttggaccgttataaccggtattatcttcgtgttattgccgtcgtagcagacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcacaagcggcgctcactgtagaccttgacacaccactgatcggcagattccttaactgtaatcaagagttcttagaggagacaatccaactccttggactcctcgaataactctactcggttcacagtggaacactattgattgtagattacggtgaggccgtgctcagacttcgagatcgatcacaagattgtatccaagcggaagacttagaacaatctgcaagctaataccacggcagatcgtattggagtataggattctacctaatacggtattcagtcaggagattaagattgcgcagtgcagattctgaacttgtcaagttccttactcgcagcgtactggcctagctatcgtgctcacactgctatcttggtgcgactattgttctgttgcagtcaccgtgtgtagtgtatctgcagtcagactactctctacggaggctgcttaagtatcagcgctgaggtactaagattgcggcctggccactgactaagatagaagctgtaacttgcctcagattgtggccgctgatctgcagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 207 - DNA, 2242 bp, synthetic construct
  source 1..2242: mol_type = other DNA
tctgcgcgctcgctcgctcactgaggccgggcgaccaaaggtcgcccgacgcccgggctttgcccgggcggcctcagtgagcgagcgagcgcgcagaggatcctatcaagaaccaacctagaacaggataccacagtgaatatcactgttagatccctatcagtgatagagagccccagtggcctaatggataaggcactggcctcctaagccagggattgcgggttcgagtcccgcctggggtgttttttcgataggctgttgtaaggcgattggtgaaggaatacgcggtacaagtacactctccctatcagtgatagagagccccagtggcctaatggataaggcactggcctcctaagccagggattgcgggttcgagtcccgcctggggtgttttttgaatagttacagcgattaccgcctattaccaacacggaagcactagactaatatccctatcagtgatagagagccccagtggcctaatggataaggcactggcctcctaagccagggattgcgggttcgagtcccgcctggggtgttttttttggaccgttataaccggtattatcttcgtgttattgccgtcgtagcagacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcacaagcggcgctcactgtagaccttgacacaccactgatcggcagattccttaactgtaatcaagagttcttagaggagacaatccaactccttggactcctcgaataactctactcggttcacagtggaacactattgattgtagattacggtgaggccgtgctcagacttcgagatcgatcacaagattgtatccaagcggaagacttagaacaatctgcaagctaataccacggcagatcgtattggagtataggattctacctaatacggtattcagtcaggagattaagattgcgcagtgcagattctgaacttgtcaagttccttactcgcagcgtactggcctagctatcgtgctcacactgctatcttggtgcgactattgttctgttgcagtcaccgtgtgtagtgtatctgcagtcagactactctctacggaggctgcttaagtatcagcgctgaggtactaagattgcggcctggccactgactaagatagaagctgtaacttgcctcagattgtggccgctgatctgcagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 208 - DNA, 2242 bp, synthetic construct
  source 1..2242: mol_type = other DNA
tctgcgcgctcgctcgctcactgaggccgggcgaccaaaggtcgcccgacgcccgggctttgcccgggcggcctcagtgagcgagcgagcgcgcagaggatcctatcaagaaccaacctagaacaggataccacagtgaatatcactgttagatccctatcagtgatagagatctccctatcagtgatagagagccccagtggcctaatggataaggcactggccttcaaagccagggattgcgggttcgagtcccgcctggggtgttttttttggaccgttataaccggtattatcttcgtgttattgccgtcgtagcagacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcacaagcggcgctcactgtagaccttgacacaccactgatcggcagattccttaactgtaatcaagagttcttagaggagacaatccaactccttggactcctcgaataactctactcggttcacagtggaacactattgattgtagattacggtgaggccgtgctcagacttcgagatcgatcacaagattgtatccaagcggaagacttagaacaatctgcaagctaataccacggcagatcgtattggagtataggattctacctaatacggtattcagtcaggagattaagattgcgcagtgcagattctgaacttgtcaagttccttactcgcagcgtactggcctagctatcgtgctcacactgctatcttggtgcgactattgttctgttgcagtcaccgtgtgtagtgtatctgcagtcagactactctctacggaggctgcttaagtatcagcgctgagttgtcgcttgattgtgctggtgactataagtcctgaagtccgattgttgcctcaattgttactgtagcgatacgattaagcgacgatataggcgacggaagatacagcttgctattgagtgcgcgttctatcgtgacagattattagaagtgtagaccggaggatcagatagatcggcctaccgcacacctgagatcaatacgtcggtaagagcgctgtaaggactaatacaatagtaataagacctattatagaggctgactggtacaggaagtatctgagtactaagattgcggcctggccactgactaagatagaagctgtaacttgcctcagattgtggccgctgatctgcagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 209 - DNA, 2305 bp, synthetic construct
  source 1..2305: mol_type = other DNA
tctgcgcgctcgctcgctcactgaggccgggcgaccaaaggtcgcccgacgcccgggctttgcccgggcggcctcagtgagcgagcgagcgcgcagaggatcctatcaagaaccaacctagaacaggataccacagtgaatatcactgttagatccctatcagtgatagagatctccctatcagtgatagagagccccagtggcctaatggataaggcactggccttcaaagccagggattgcgggttcgagtcccgcctggggtgttttttcgataggctgttgtaaggcgattggtgaaggaatacgcggtacaagtacactctccctatcagtgatagagatctccctatcagtgatagagagccccagtggcctaatggataaggcactggccttcaaagccagggattgcgggttcgagtcccgcctggggtgttttttgaatagttacagcgattaccgcctattaccaacacggaagcactagactaatatccctatcagtgatagagatctccctatcagtgatagagagccccagtggcctaatggataaggcactggccttcaaagccagggattgcgggttcgagtcccgcctggggtgttttttttggaccgttataaccggtattatcttcgtgttattgccgtcgtagcagacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcacaagcggcgctcactgtagaccttgacacaccactgatcggcagattccttaactgtaatcaagagttcttagaggagacaatccaactccttggactcctcgaataactctactcggttcacagtggaacactattgattgtagattacggtgaggccgtgctcagacttcgagatcgatcacaagattgtatccaagcggaagacttagaacaatctgcaagctaataccacggcagatcgtattggagtataggattctacctaatacggtattcagtcaggagattaagattgcgcagtgcagattctgaacttgtcaagttccttactcgcagcgtactggcctagctatcgtgctcacactgctatcttggtgcgactattgttctgttgcagtcaccgtgtgtagtgtatctgcagtcagactactctctacggaggctgcttaagtatcagcgctgaggtactaagattgcggcctggccactgactaagatagaagctgtaacttgcctcagattgtggccgctgatctgcagaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 210 - DNA, 3867 bp, synthetic construct
  source 1..3867: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggatcctatcaagaaccaacctagaacaggataccacagtgaatatcactgttagatccctatcagtgatagagatggcaattccacgttatagccgttgatcagataagagttgcagaagccccagtggcctaatggataaggcactggccttcaaagccagggattgcgggttcgagtcccgcctggggtgttttttcgataggctgttgtaaggcgattggtgaaggaatacgcggtacaagtacactctccctatcagtgatagagaactgttaacaagacacagtgtgttgtctattcggttggtgcagaagccccagtggcctaatggataaggcactggccttcaaagccagggattgcgggttcgagtcccgcctggggtgttttttgaatagttacagcgattaccgcctattaccaacacggaagcactagactaatatccctatcagtgatagagactgactggtacaggaagtatctgatctagacgaattggtgcagaagccccagtggcctaatggataaggcactggccttcaaagccagggattgcgggttcgagtcccgcctggggtgttttttttggaccgttataaccggtattatcttcgtgttattgccgtcgtagcagacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcacaagcggcgctcactgtagaccttgacacaccactgatcggcagattccttaactgtaatcaagagttcttagaggagacaatccaactccttggactcctcgaataactctactcggttcacagtggaacactattgattgtagattacggtgaggccgtgctcagacttcgagatcgatcacaagattgtatccaagcggaagacttagaacaatctgcaagctaataccacggcagatcgtattggagtataggattctacctaatacggtattcagtcaggagattaagattgcgcagtgcagattctgaacttgtcaagttccttactcgcagcgtactggcctagctatcgtgctcacactgctatcttggtgcgactattgttctgttgcagtcaccgtgtgtagtgtatctgcagtcagactactctctacggaggctgcttaagtatcagcgctgagttgtcgcttgattgtgctggtgactataagtcctgaagtccgattgttgcctcaattgttactgtagcgatacgattaagcgacgatataggcgacggaagatacagcttgctattgagtgcgcgttctatcgtgacagattattagaagtgtagaccggaggatcagatagatcggcctaccgcacacctgagatcaatacgtcggtaagagcgctgtaaggactaatacaatagtaataagacctattatagaggattcgttgtcgagtagaattattcctatacaatcgcaggttcgattgagattctacttcgcgttcttgacagctgaagtccttgtgaacaatagcgcggcgcaattgaagacgtacttgacaagcgtatcgatctctgtgaagcaccgaattggcaagaagtattgtattataattaccgtctggttaacagagtgttcactactccgccaatcgaggttattacttgaatcaaccgtcagtacaacttcgacacactaactactagccaatcgagatacgtaagacacgtgtgtatacctctcctatcagtgcgttcaagtgttacacgatagttcgcttaattgactcggtcaagattatcacgtagctattgttacaatacttgtagaatctaagccgtatcgctatcggaattagccgctgtcacctatgacactatccgcactgtaagatcgctgcagttcgcagcctccgaacgattgaggtgaataaggttattcttgcagttgcaccaggaacctgcgttataattagtccttggcgtcacttgcgaagattaggatcagattgcagcactcgtaggacttgacttcactacagtgagcagtatctagacgccaaggtagcttccagtacgcctcgttacttcggagttacgtatactcacgcgtaagttgccgaataggtgcactatgactggagtgcttagcgcgtgattactgctggaggattggaattggcgattcttacgcggaaccacgataacgagataacgttaagtcgctagacttaaggcggcacgcgatcgagcaactcctcactacgataggtgagcgcagtccagtgtagctcagtcggccaattacggttctattagacagttgcacgttgacatcacactcgttcctatagtgtatagactcaattgtagttcaccgctgagtatcactcacagcacgatctactcagacttaatcgcactactggacggaatcgcgtcgtcggccaataagcctgtgtccagagctgcagtagtaactccactgtagctgttagacaataagtactaagattgcggcctggccactgactaagatagaagctgtaacttgcctcagattgtggccgctgatctgcaggaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 211 - DNA, 3867 bp, synthetic construct
  source 1..3867: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggatcctatcaagaaccaacctagaacaggataccacagtgaatatcactgttagatccctatcagtgatagagagccccagtggcctaatggataaggcactggccttcaaagccagggattgcgggttcgagtcccgcctggggtgttttttcgataggctgttgtaaggcgattggtgaaggaatacgcggtacaagtacactctccctatcagtgatagagagccccagtggcctaatggataaggcactggccttcaaagccagggattgcgggttcgagtcccgcctggggtgttttttgaatagttacagcgattaccgcctattaccaacacggaagcactagactaatatccctatcagtgatagagagccccagtggcctaatggataaggcactggccttcaaagccagggattgcgggttcgagtcccgcctggggtgttttttttggaccgttataaccggtattatcttcgtgttattgccgtcgtagcagacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcacaagcggcgctcactgtagaccttgacacaccactgatcggcagattccttaactgtaatcaagagttcttagaggagacaatccaactccttggactcctcgaataactctactcggttcacagtggaacactattgattgtagattacggtgaggccgtgctcagacttcgagatcgatcacaagattgtatccaagcggaagacttagaacaatctgcaagctaataccacggcagatcgtattggagtataggattctacctaatacggtattcagtcaggagattaagattgcgcagtgcagattctgaacttgtcaagttccttactcgcagcgtactggcctagctatcgtgctcacactgctatcttggtgcgactattgttctgttgcagtcaccgtgtgtagtgtatctgcagtcagactactctctacggaggctgcttaagtatcagcgctgagttgtcgcttgattgtgctggtgactataagtcctgaagtccgattgttgcctcaattgttactgtagcgatacgattaagcgacgatataggcgacggaagatacagcttgctattgagtgcgcgttctatcgtgacagattattagaagtgtagaccggaggatcagatagatcggcctaccgcacacctgagatcaatacgtcggtaagagcgctgtaaggactaatacaatagtaataagacctattatagaggattcgttgtcgagtagaattattcctatacaatcgcaggttcgattgagattctacttcgcgttcttgacagctgaagtccttgtgaacaatagcgcggcgcaattgaagacgtacttgacaagcgtatcgatctctgtgaagcaccgaattggcaagaagtattgtattataattaccgtctggttaacagagtgttcactactccgccaatcgaggttattacttgaatcaaccgtcagtacaacttcgacacactaactactagccaatcgagatacgtaagacacgtgtgtatacctctcctatcagtgcgttcaagtgttacacgatagttcgcttaattgactcggtcaagattatcacgtagctattgttacaatacttgtagaatctaagccgtatcgctatcggaattagccgctgtcacctatgacactatccgcactgtaagatcgctgcagttcgcagcctccgaacgattgaggtgaataaggttattcttgcagttgcaccaggaacctgcgttataattagtccttggcgtcacttgcgaagattaggatcagattgcagcactcgtaggacttgacttcactacagtgagcagtatctagacgccaaggtagcttccagtacgcctcgttacttcggagttacgtatactcacgcgtaagttgccgaataggtgcactatgactggagtgcttagcgcgtgattactgctggaggattggaattggcgattcttacgcggaaccacgataacgagataacgttaagtcgctagacttaaggcggcacgcgatcgagcaactcctcactacgataggtgagcgcagtccagtgtagctcagtcggccaattacggttctattagacagttgcacgttgacatcacactcgttcctatagtgtatagactcaattgtagttcaccgctgagtatcactcacagcacgatctactcagacttaatcgcactactggacggaatcgcgtcgtcggccaataagcctgtgtccagagctgcagtagtaactccactgtagctgttagacaataagacgagtgatactggctagcgaggagcgcgaggacacttagatccacagtctaactgacaaggacgagtgagcaactagcagtctgcggtgattgagtctcgcggccgctgttatcgccagtacggaataggtcggtactaagattgcggcctggccactgactaagatagaagctgtaacttgcctcagattgtggccgctgatctgcaggaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 212 - DNA, 3867 bp, synthetic construct
  source 1..3867: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcggatcctatcaagaaccaacctagaacaggataccacagtgaatatcactgttagatccctatcagtgatagagagccccagtggcctaatggataaggcactggcctcctaagccagggattgcgggttcgagtcccgcctggggtgttttttcgataggctgttgtaaggcgattggtgaaggaatacgcggtacaagtacactctccctatcagtgatagagagccccagtggcctaatggataaggcactggcctcctaagccagggattgcgggttcgagtcccgcctggggtgttttttgaatagttacagcgattaccgcctattaccaacacggaagcactagactaatatccctatcagtgatagagagccccagtggcctaatggataaggcactggcctcctaagccagggattgcgggttcgagtcccgcctggggtgttttttttggaccgttataaccggtattatcttcgtgttattgccgtcgtagcagacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcacaagcggcgctcactgtagaccttgacacaccactgatcggcagattccttaactgtaatcaagagttcttagaggagacaatccaactccttggactcctcgaataactctactcggttcacagtggaacactattgattgtagattacggtgaggccgtgctcagacttcgagatcgatcacaagattgtatccaagcggaagacttagaacaatctgcaagctaataccacggcagatcgtattggagtataggattctacctaatacggtattcagtcaggagattaagattgcgcagtgcagattctgaacttgtcaagttccttactcgcagcgtactggcctagctatcgtgctcacactgctatcttggtgcgactattgttctgttgcagtcaccgtgtgtagtgtatctgcagtcagactactctctacggaggctgcttaagtatcagcgctgagttgtcgcttgattgtgctggtgactataagtcctgaagtccgattgttgcctcaattgttactgtagcgatacgattaagcgacgatataggcgacggaagatacagcttgctattgagtgcgcgttctatcgtgacagattattagaagtgtagaccggaggatcagatagatcggcctaccgcacacctgagatcaatacgtcggtaagagcgctgtaaggactaatacaatagtaataagacctattatagaggattcgttgtcgagtagaattattcctatacaatcgcaggttcgattgagattctacttcgcgttcttgacagctgaagtccttgtgaacaatagcgcggcgcaattgaagacgtacttgacaagcgtatcgatctctgtgaagcaccgaattggcaagaagtattgtattataattaccgtctggttaacagagtgttcactactccgccaatcgaggttattacttgaatcaaccgtcagtacaacttcgacacactaactactagccaatcgagatacgtaagacacgtgtgtatacctctcctatcagtgcgttcaagtgttacacgatagttcgcttaattgactcggtcaagattatcacgtagctattgttacaatacttgtagaatctaagccgtatcgctatcggaattagccgctgtcacctatgacactatccgcactgtaagatcgctgcagttcgcagcctccgaacgattgaggtgaataaggttattcttgcagttgcaccaggaacctgcgttataattagtccttggcgtcacttgcgaagattaggatcagattgcagcactcgtaggacttgacttcactacagtgagcagtatctagacgccaaggtagcttccagtacgcctcgttacttcggagttacgtatactcacgcgtaagttgccgaataggtgcactatgactggagtgcttagcgcgtgattactgctggaggattggaattggcgattcttacgcggaaccacgataacgagataacgttaagtcgctagacttaaggcggcacgcgatcgagcaactcctcactacgataggtgagcgcagtccagtgtagctcagtcggccaattacggttctattagacagttgcacgttgacatcacactcgttcctatagtgtatagactcaattgtagttcaccgctgagtatcactcacagcacgatctactcagacttaatcgcactactggacggaatcgcgtcgtcggccaataagcctgtgtccagagctgcagtagtaactccactgtagctgttagacaataagacgagtgatactggctagcgaggagcgcgaggacacttagatccacagtctaactgacaaggacgagtgagcaactagcagtctgcggtgattgagtctcgcggccgctgttatcgccagtacggaataggtcggtactaagattgcggcctggccactgactaagatagaagctgtaacttgcctcagattgtggccgctgatctgcaggaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 213 - DNA, 3760 bp, synthetic construct
  source 1..3760: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtggatcctatcaagaaccaacctagaacaggataccacagtgaatatcactgttagagttcagagaccatgacactgaggtctcaggccttggaccgttataaccggtattatcttcgtgttattgccgtcgtagcagacctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcggggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctcagtactaagattgcggcctggccactgactaagatagaagctgtaacttgcctcagattgtggccgctgatctgcagctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 214 - DNA, 615 bp, synthetic construct
  source 1..615: mol_type = other DNA
atgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaa

SEQ ID NO: 215 - DNA, 1882 bp, synthetic construct
  source 1..1882: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcggacaggtaccaaaacaaatgttctcgtcacgtgggcatgaatctgatgctgtttccctgcagacaatgcgagagactgaatcagaattcaaatatctgcttcactcacggtgtcaaagactgtttagagtgctttcccgtgtcagaatctcaacccgtttctgtcgtcaaaaaggcgtatcagaaactgtgctacattcatcacatcatgggaaaggtgccagacgcttgcactgcttgcgacctggtcaatgtggacttggatgactgtgtttctgaacaataaatgacttaaaccaggt

SEQ ID NO: 216 - DNA, 531 bp, synthetic construct
  source 1..531: mol_type = other DNA
atggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatga

SEQ ID NO: 217 - DNA, 1491 bp, synthetic construct
  source 1..1491: mol_type = other DNA
atggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattga

SEQ ID NO: 218 - DNA, 1590 bp, synthetic construct
  source 1..1590: mol_type = other DNA
ttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccat

SEQ ID NO: 219 - DNA, 684 bp, synthetic construct
  source 1..684: mol_type = other DNA
atgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactga

SEQ ID NO: 220 - DNA, 2424 bp, synthetic construct
  source 1..2424: mol_type = other DNA
atggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtag

SEQ ID NO: 221 - DNA, 1882 bp, synthetic construct; identical to SEQ ID NO: 215
  source 1..1882: mol_type = other DNA

SEQ ID NO: 222 - DNA, 657 bp, synthetic construct
  source 1..657: mol_type = other DNA
atgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaa

SEQ ID NO: 223 - protein, 218 aa, synthetic construct
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSAYSGSREFRSY

SEQ ID NO: 224 - DNA, 952 bp, synthetic construct
  source 1..952: mol_type = other DNA
cctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagtagagccgccaccatggccacaaccatgggtcttttctgcaatcaccgtcctcgaggcaccatgaacccagccatcagcgtcgctctcctgctctcagtcttgcgggtgtcccgagggcagaaggtgaccagcctgacagcctgcctggtgaaccaaaaccttcgcctggactgccgccatgagaataacaccaaggataactccatccagcatgagttcagcctgacccgagagaagaggaagcacgtgctctcaggcacccttgggatacccgagcacacgtaccgctcccgcgtcaccctctccaaccagccctatatcaaggtccttaccctagccaacttcaccaccaaggatgagggcgactacttttgtgagcttcgcgtctcgggcgcgaatcccatgagctccaataaaagtatcagtgtgtatagagacaagctggtcaagtgtggcggcataagcctgctggttcagaacacatcctggatgctgctgctgctgctttccctctccctcctccaagccctggacttcatttctctgtaaaataaaggaaatttattttcattgcaatagtgtgttggaattttttgtgtctctca

SEQ ID NO: 225 - DNA, 97 bp, synthetic construct
  source 1..97: mol_type = other DNA
tctgcgcgctcgctcgctcactgaggccgggcgaccaaaggtcgcccgacgcccgggctttgcccgggcggcctcagtgagcgagcgagcgcgcaga

SEQ ID NO: 226 - DNA, 141 bp, synthetic construct
  source 1..141: mol_type = other DNA
ccttaattaggctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 227 - DNA, 145 bp, synthetic construct
  source 1..145: mol_type = other DNA
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 228 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 229-248 - skipped (20 entries)

SEQ ID NO: 249 - RNA, 72 nt, synthetic construct
  source 1..72: mol_type = other RNA
  tRNA 1..72: note = tRNA
ggttccatggtgtaatggttagcactctggactttaaatccagcgatccgagttcaaatctcggtggaacct

SEQ ID NO: 250 - RNA, 72 nt, synthetic construct
  source 1..72: mol_type = other RNA
  tRNA 1..72: note = tRNA
ggttccatggtgtaatggttagcactctggactttaaatccagcgatccgagttcaagtctcggtggaacct

SEQ ID NO: 251 - RNA, 72 nt, synthetic construct
  source 1..72: mol_type = other RNA
  tRNA 1..72: note = tRNA
ggttccatggtgtaatggtgagcactctggactttaaatccagcgatccgagttcgagtctcggtggaacct

SEQ ID NO: 252 - RNA, 72 nt, synthetic construct; identical to SEQ ID NO: 251
  source 1..72: mol_type = other RNA
  tRNA 1..72: note = tRNA

SEQ ID NO: 253 - RNA, 72 nt, synthetic construct
  source 1..72: mol_type = other RNA
  tRNA 1..72: note = tRNA
ggttccatggtgtaatggttagcactctggactttaaatccggtaatccgagttcaaatctcggtggaacct

SEQ ID NO: 254 - RNA, 72 nt, synthetic construct
  source 1..72: mol_type = other RNA
  tRNA 1..72: note = tRNA
ggccccatggtgtaatggtcagcactctggactttaaatccagcgatccgagttcaaatctcggtgggaccc

SEQ ID NO: 255 - RNA, 72 nt, synthetic construct
  source 1..72: mol_type = other RNA
  tRNA 1..72: note = tRNA
ggttccatggtgtaatggtaagcactctggactttaaatccagccatctgagttcgagtctctgtggaacct

SEQ ID NO: 256 - RNA, 72 nt, synthetic construct
  source 1..72: mol_type = other RNA
  tRNA 1..72: note = tRNA
ggttccatggtgtaatggtgagcactttggactttaaatacagtgatcagagttcaagtctcactgggacct

SEQ ID NO: 257 - RNA, 72 nt, synthetic construct
  source 1..72: mol_type = other RNA
  tRNA 1..72: note = tRNA
ggttccatgggttaatggtgagcaccctggactttaaatcaagcgatccgagttcaaatctcggtggtacct

SEQ ID NO: 258 - RNA, 72 nt, synthetic construct
  source 1..72: mol_type = other RNA
  tRNA 1..72: note = tRNA
ggttccatggtgtaatggtgaccactttggactttaaatacagtgatcagagttcaagtctcactggaacct

SEQ ID NO: 259 - RNA, 72 nt, synthetic construct
  source 1..72: mol_type = other RNA
  tRNA 1..72: note = tRNA
ggttccatggtgtaatggtgagggctttggactttaactacagtgatcagagttcaagtctcagtgggacct

SEQ ID NO: 260 - RNA, 73 nt, synthetic construct
  source 1..73: mol_type = other RNA
  tRNA 1..73: note = tRNA
ggttccatggtgtaatggtaagcaccctggactttaaatccagcaaccagagttccagtctcagcgtggacct

SEQ ID NO: 261 - RNA, 72 nt, synthetic construct
  source 1..72: mol_type = other RNA
  tRNA 1..72: note = tRNA
ggtagtgtagtctactggttaaacgcttgggctttaacattaacgtcctgggttcaaatcccagctttgtca

SEQ ID NO: 262 - RNA, 72 nt, synthetic construct
  source 1..72: mol_type = other RNA
  tRNA 1..72: note = tRNA
ggtcccatggtgtaatggttagcactctggactttaaatccagcgatccgagttcaaatctcggtgggacct

SEQ ID NO: 263 - RNA, 72 nt, synthetic construct
  source 1..72: mol_type = other RNA
  tRNA 1..72: note = tRNA
ggtcccatggtgtaatggttagcactctggactttaaatccagcaatccgagttcgaatctcggtgggacct

SEQ ID NO: 264 - RNA, 72 nt, synthetic construct
  source 1..72: mol_type = other RNA
  tRNA 1..72: note = tRNA
ggccccatggtgtaatggttagcactctggactttaaatccagcgatccgagttcaaatctcggtgggacct

SEQ ID NO: 265 - RNA, 72 nt, synthetic construct
  source 1..72: mol_type = other RNA
  tRNA 1..72: note = tRNA
ggtcccatggtgtaatggttagcactctgggctttaaatccagcaatccgagttcgaatcttggtgggacct

SEQ ID NO: 266 - RNA, 73 nt, synthetic construct
  source 1..73: mol_type = other RNA
  tRNA 1..73: note = tRNA
ggctgtgtacctcagtgggcaagggtatggactttaaagccagactatttgggttcaaatcccagcttggcct

SEQ ID NO: 267 - RNA, 73 nt, synthetic construct
  source 1..73: mol_type = other RNA
  tRNA 1..73: note = tRNA
gaccatgtggcctaagggaaaagacatctcactttaggtcagaagattgagggttcaagtcctttcatggtca

SEQ ID NO: 268 - RNA, 74 nt, synthetic construct
  source 1..74: mol_type = other RNA
  tRNA 1..74: note = tRNA
ggtacagtgttaaaggggagaaaaattgctgactttaaatacagtagacctaggtttgaatcctggctttacca

SEQ ID NO: 269 - RNA, 72 nt, synthetic construct
  source 1..72: mol_type = other RNA
  tRNA 1..72: note = tRNA
ggttccatggtgtaatggttagcactctggactctaaatccagcgatccgagttcaaatctcggtggaacct

SEQ ID NO: 270 - RNA, 72 nt, synthetic construct
  source 1..72: mol_type = other RNA
  tRNA 1..72: note = tRNA
ggttccatggtgtaatggttagcactctggactctaaatccagcgatccgagttcaagtctcggtggaacct

SEQ ID NO: 271 - RNA, 72 nt, synthetic construct
  source 1..72: mol_type = other RNA
  tRNA 1..72: note = tRNA
ggttccatggtgtaatggtgagcactctggactctaaatccagcgatccgagttcgagtctcggtggaacct

SEQ ID NO: 272 - RNA, 72 nt, synthetic construct
  source 1..72: mol_type = other RNA
  tRNA 1..72: note = tRNA
ggttccatggtgtaatggtaagcactctggactctaaatccagcgatccgagttcgagtctcggtggaacct

SEQ ID NO: 273 - RNA, 72 nt, synthetic construct
  source 1..72: mol_type = other RNA
  tRNA 1..72: note = tRNA
ggttccatggtgtaatggttagcactctggactctaaatccggtaatccgagttcaaatctcggtggaacct

SEQ ID NO: 274 - RNA, 72 nt, synthetic construct
  source 1..72: mol_type = other RNA
  tRNA 1..72: note = tRNA
ggccccatggtgtaatggtcagcactctggactctaaatccagcgatccgagttcaaatctcggtgggaccc

SEQ ID NO: 275 - RNA, 72 nt, synthetic construct
  source 1..72: mol_type = other RNA
  tRNA 1..72: note = tRNA
ggttccatggtgtaatggtaagcactctggactctaaatccagccatctgagttcgagtctctgtggaacct

SEQ ID NO: 276 - RNA, 72 nt, synthetic construct
  source 1..72: mol_type = other RNA
  tRNA 1..72: note = tRNA
ggttccatggtgtaatggtgagcactttggactctaaatacagtgatcagagttcaagtctcactgggacct

SEQ ID NO: 277 - RNA, 72 nt, synthetic construct
  source 1..72: mol_type = other RNA
  tRNA 1..72: note = tRNA
ggttccatgggttaatggtgagcaccctggactctaaatcaagcgatccgagttcaaatctcggtggtacct

SEQ ID NO: 278 - RNA, 72 nt, synthetic construct
  source 1..72: mol_type = other RNA
  tRNA 1..72: note = tRNA
ggttccatggtgtaatggtgaccactttggactctaaatacagtgatcagagttcaagtctcactggaacct

SEQ ID NO: 279 - RNA, 73 nt, synthetic construct
  source 1..73: mol_type = other RNA
  tRNA 1..73: note = tRNA
ggttccatggtgtaatggtgagggctttggactctaactacagtgatcagagttcaagtctcagtgggacctt

SEQ ID NO: 280 - RNA, 73 nt, synthetic construct
  source 1..73: mol_type = other RNA
  tRNA 1..73: note = tRNA
ggttccatggtgtaatggtaagcaccctggactctaaatccagcaaccagagttccagtctcagcgtggacct

SEQ ID NO: 281 - RNA, 72 nt, synthetic construct
  source 1..72: mol_type = other RNA
  tRNA 1..72: note = tRNA
ggtagtgtagtctactggttaaacgcttgggctctaacattaacgtcctgggttcaaatcccagctttgtca

SEQ ID NO: 282 - RNA, 73 nt, synthetic construct
  source 1..73: mol_type = other RNA
  tRNA 1..73: note = tRNA
ggtcccatggtgtaatggttagcactctggactctaaatccagcgatccgagttcaaatctcggtgggacctt

SEQ ID NO: 283 - RNA, 73 nt, synthetic construct
  source 1..73: mol_type = other RNA
  tRNA 1..73: note = tRNA
ggtcccatggtgtaatggttagcactctggactctaaatccagcaatccgagttcgaatctcggtgggacctt

SEQ ID NO: 284 - RNA, 73 nt, synthetic construct
  source 1..73: mol_type = other RNA
  tRNA 1..73: note = tRNA
ggccccatggtgtaatggttagcactctggactctaaatccagcgatccgagttcaaatctcggtgggacctt

SEQ ID NO: 285 - RNA, 73 nt, synthetic construct
  source 1..73: mol_type = other RNA
  tRNA 1..73: note = tRNA
ggtcccatggtgtaatggttagcactctgggctctaaatccagcaatccgagttcgaatcttggtgggacctt

SEQ ID NO: 286 - RNA, 73 nt, synthetic construct
  source 1..73: mol_type = other RNA
  tRNA 1..73: note = tRNA
ggctgtgtacctcagtgggcaagggtatggactctaaagccagactatttgggttcaaatcccagcttggcct

SEQ ID NO: 287 - RNA, 73 nt, synthetic construct
  source 1..73: mol_type = other RNA
  tRNA 1..73: note = tRNA
gaccatgtggcctaagggaaaagacatctcactctaggtcagaagattgagggttcaagtcctttcatggtca

SEQ ID NO: 288 - RNA, 74 nt, synthetic construct
  source 1..74: mol_type = other RNA
  tRNA 1..74: note = tRNA
ggtacagtgttaaaggggagaaaaattgctgactctaaatacagtagacctaggtttgaatcctggctttacca

SEQ ID NO: 289 - DNA, 63 bp, synthetic construct
  source 1..63: mol_type = other DNA
acctgactcgtaatctgtaattgcttgttaatcaataaaccgtttaattcgtttcagttgaac

SEQ ID NO: 290 - DNA, 63 bp, synthetic construct
  source 1..63: mol_type = other DNA
acctgactcgtaatctgtagttgcttgttaatcaatagaccgtttagttcgtttcagttgaac

(19) United States
(12) Patent Application Publication
(10) Pub. No.: US 2025/0059538 A1
(43) Pub. Date: Feb. 20, 2025

(54) COMPOSITIONS AND METHODS OF tRNA SILENCING

(71) Applicant: SHAPE THERAPEUTICS INC., Seattle, WA (US)

(72) Inventors: Sandhya Pande, Seattle, WA (US); Anupama Lakshmanan, Seattle, WA (US); Lauriel Earley, Seattle, WA (US); Stephen Michael Burleigh, Seattle, WA (US); Collin Hauskins, Seattle, WA (US); Adrian Wrangham Briggs, Seattle, WA (US); Kevin Stein, Seattle, WA (US)

(21) Appl. No.: 18/723,276

(22) PCT Filed: Dec. 23, 2022
(86) PCT No.: PCT/US2022/082330

§ 371 Date: Jun. 21, 2024

Related U.S. Application Data
(60) Provisional application No. 63/320,883, filed on Mar. 17, 2022.
(60) Provisional application No. 63/293,367, filed on Dec. 23, 2021.

(51) Int. Cl. C12N 15/113

(52) U.S. Cl. CPC C12N 15/113; C12N 2310/14; C12N 2310/531

(57) ABSTRACT

Provided are compositions and methods for silencing suppressor tRNAs. Further provided are compositions and methods for silencing suppressor tRNAs during AAV production for producing virions comprising sequences coding for suppressor tRNAs.

### CLAIM OF PRIORITY

This application claims the benefit of U.S. Provisional Application No. 63/293,367, filed on Dec. 23, 2021, and 63/320,883, filed on Mar. 17, 2022, each of which is incorporated by reference in its entirety.

### BACKGROUND

Premature stop codons leading to mutations in proteins have been implicated in many severe diseases and disorders. Translation of an mRNA molecule that contains a premature stop codon can cause premature termination of the translation process to produce a truncated polypeptide or protein, which can cause these severe diseases or disorders.

There is a need for effective treatments for diseases and disorders associated with premature stop codons, including effective production and delivery of these treatments.

### SUMMARY

One example treatment for diseases and disorders associated with premature stop codons is administration of transfer RNA (tRNA) molecules that suppress premature stop codons to enable readthrough of the premature stop codon in a template messenger RNA (mRNA), which are referred to herein as a “suppressor tRNA” or a “suppressor tRNA molecule”, which are used interchangeably throughout this disclosure. These suppressor tRNAs can comprise an engineered anticodon for recognition of a premature stop codon relative to a wild type tRNA and at least partially transform translation of a premature stop codon into a sense codon, such as, for example by adding a corrective (e.g., non-disease-causing) amino acid to the growing peptide.

Often, vectors are used to deliver the suppressor tRNAs to a subject in need thereof, for example a subject suffering from a disease or disorder associated with premature stop codon(s). The vectors can be viral vectors. The suppressor tRNA molecules or vectors encoding the suppressor tRNA molecules can be packaged into a virus for virus particle-mediated delivery of the suppressor tRNA molecules to a target cell or tissue in vivo. In some cases, the virus can be an adeno-associated virus (AAV).

However, as demonstrated herein, a significant and surprising loss in titer was observed when producing AAV encapsidated suppressor tRNA coding sequences. As also demonstrated herein, when expression of suppressor tRNA sequences is repressed during AAV production, a resulting increase in titer is observed. Therefore, one advantage of the compositions and methods described herein is increased titer during production of AAV encapsidated suppressor tRNA coding sequence(s). Having the ability to increase the titer during production of AAV encapsidated suppressor tRNA coding sequences allows clinicians, medical professionals, laboratory personnel, and researchers to produce amounts of AAV encapsidated suppressor tRNA coding sequence(s) for administration to a patient in need thereof.

As further described herein, in a therapeutic context, the compositions and methods described herein allow for expression of the conditionally repressible suppressor tRNA coding sequences (e.g., in a diseased cell that comprises a premature stop codon, such as Rett syndrome, Dravet syndrome, or Duchenne Muscular Dystrophy). Therefore, in some cases, another advantage of the compositions and methods described herein is that, while in the production environment expression of the suppressor tRNA is repressed, in a therapeutic context, the suppressor tRNA is expressed, resulting in read-through of the premature stop codon(s) and treatment of the disease.

Alternatively to repression of suppressor tRNA during AAV production, sequences coding for one or more viral proteins needed for viral production can be engineered from the stop codon recognized by the suppressor tRNA to a different stop codon (e.g., from an opal stop codon recognized by an opal suppressor tRNA to an amber stop codon). Therefore, in some cases, the effect of expression of suppressor tRNA sequences during AAV production on the one or more viral proteins needed for viral production is reduced, resulting in an increase in titer. This alternative approach, and the associated compositions and methods described herein, also can advantageously increase titer during production of AAV encapsidated suppressor tRNA coding sequence(s). Additionally, this approach allows for encapsidation of the suppressor tRNA not in the context of a conditionally repressible suppressor tRNA. Having the ability to increase the titer during production of AAV encapsidated suppressor tRNA coding sequences allows clinicians, medical professionals, laboratory personnel, and researchers to produce amounts of AAV encapsidated suppressor tRNA coding sequence(s) for administration to a patient in need thereof.

Therefore, among other things, provided here are polynucleotides comprising, from 5′ to 3′: a 5′ AAV inverted terminal repeat (ITR) sequence; a polynucleotide sequence comprising one or more conditionally repressible suppressor tRNA coding sequence(s); and a 3′ AAV inverted terminal repeat (ITR) sequence.

In some embodiments, the conditionally repressible suppressor tRNA coding sequence(s) each comprise sequence(s) encoding repressor element(s) and sequence(s) encoding suppressor tRNA(s).

In some embodiments, the sequence encoding the repressor element is upstream of the sequence encoding the suppressor tRNA. In some embodiments, the sequence encoding the repressor element is downstream of the sequence encoding the suppressor tRNA. In some embodiments, the sequence coding for the repressor element is positioned at +1, −7, −12, −20, −30, or −45 of the sequence coding for the suppressor tRNA. In some embodiments, the sequence encoding the repressor element is positioned at +7, +12, +20, +30, or +45 of the sequence encoding the suppressor tRNA.

In some embodiments, the repressor element comprises a sequence encoding an operator. In some embodiments, the operator is selected from the group consisting of a tetracycline operator (TetO) and a lac operator (LacO). In some embodiments, the tetracycline operator comprises SEQ ID NO: 150. In some embodiments, the repressor element comprises a sequence encoding a tetracycline response element (TRE). In some embodiments, the sequence encoding the TRE comprises TetO sequence(s). In some embodiments, the TetO sequence(s) comprises a concatemer of TetO sequences. In some embodiments, the TetO sequence(s) comprises 2, 3, 4, 5, 6, 7, 8, 9, or 10 TetO sequences. In some embodiments, two or more TetO sequences in the concatemer of TetO sequences are separated by a spacer sequence. In some embodiments, the spacer sequence is TC. In some embodiments, the concatemer of TetO sequences consists of 2 TetO sequences. In some embodiments, the concatemer of TetO sequences comprises 2 TetO sequences separated by a spacer sequence. In some embodiments, the concatemer of TetO sequences comprises 2 TetO sequences separated by a TC spacer sequence. In some embodiments, the concatemer of TetO sequences consists of SEQ ID NO: 149.

In some embodiments, the polynucleotide sequence comprising one or more conditionally repressible suppressor tRNA coding sequence(s) further comprises a promoter sequence. In some embodiments, the promoter sequence comprises a RNA polymerase type III promoter, optionally a U6 promoter sequence, optionally a hU6 promoter sequence. In some embodiments, the promoter sequence comprises a tRNA promoter sequence or fragment thereof. In some embodiments, the promoter sequence is upstream of the conditionally repressible suppressor tRNA coding sequence(s). In some embodiments, the promoter sequence is downstream of the conditionally repressible suppressor tRNA coding sequence(s). In some embodiments, the promoter sequence is interspersed within the conditionally repressible tRNA coding sequence(s). In some embodiments, the promoter sequence is an hU6 promoter sequence. In some embodiments, the hU6 promoter sequence comprises one or more promoter sequence element(s) selected from the group consisting of a SPH element, an Oct-1 element, a proximal sequence element (PSE), and a TATA element. In some embodiments, the promoter sequence element(s) are interspersed between repressor element sequence(s) and suppressor tRNA sequence(s). In some embodiments, the repressor element sequence(s) are downstream of the SPH and Oct-1 element(s). In some embodiments, the repressor element sequence(s) are before the PSE element, between the PSE and TATA elements, and/or after the TATA element.

In some embodiments, the sequence(s) encoding the suppressor tRNA(s) are sequence(s) encoding for opal suppressor tRNA(s). In some embodiments, the sequence(s) encoding the suppressor tRNA(s) are sequence(s) encoding for ochre suppressor tRNA(s). In some embodiments, the sequence(s) encoding the suppressor tRNA(s) are sequence(s) encoding for amber suppressor tRNA(s). In some embodiments, the sequences encoding the suppressor tRNAs are sequence(s) encoding for opal suppressor tRNA(s), sequence(s) encoding for ochre suppressor tRNA(s), sequence(s) encoding for amber suppressor tRNA(s), or any combination thereof. In some embodiments, the sequence(s) encoding the suppressor tRNA(s) are each selected from the group consisting of any one of SEQ ID NOs: 3-48, 49-68, and 69-88. In some embodiments, the sequence(s) encoding the suppressor tRNA(s) are each selected from the group consisting of any one of SEQ ID NOs: 103-148, 249-268, and 269-288. In some embodiments, the sequence encoding the polynucleotide sequence comprising one or more conditionally repressible suppressor tRNA coding sequence(s) comprises at least 80%, 85%, 90%, 95%, 97%, 99%, or 100% sequence identity to any one of SEQ ID NOs: 158-162, 164-168, 171-175, 177-181, 182-194, or 199; optionally wherein the sequence encoding the polynucleotide sequence comprises multiple copies of the one or more conditionally repressible suppressor tRNA coding sequence(s) comprising at least 80%, 85%, 90%, 95%, 97%, 99%, or 100% sequence identity to any one of SEQ ID NOs: 158-162, 164-168, 171-175, 177-181, 182-194, or 199; optionally wherein the sequence encoding the polynucleotide sequence comprises one, two, three, four, five, six, seven, eight, nine, or ten of the one or more conditionally repressible suppressor tRNA coding sequence(s) comprising at least 80%, 85%, 90%, 95%, 97%, 99%, or 100% sequence identity to any one of SEQ ID NOs: 158-162, 164-168, 171-175, 177-181, 182-194, or 199. In some embodiments, the sequence encoding the polynucleotide sequence comprising one or more conditionally repressible suppressor tRNA coding sequence(s) comprises or consists of at least 80%, 85%, 90%, 95%, 97%, 99%, or 100% sequence identity any one of SEQ ID NOs: 203-206 or 208-211, with or without the transduction marker; optionally, wherein the transduction marker comprises at least 80%, 85%, 90%, 95%, 97%, 99%, or 100% sequence identity to SEQ ID NO: 224.

In some embodiments, the conditionally repressible suppressor tRNA comprises a guide RNA binding sequence and sequence encoding a suppressor tRNA. In some embodiments, the conditionally repressible suppressor tRNA coding sequence further comprises a protospacer adjacent motif (PAM). In some embodiments, the guide RNA binding sequence is from 15 to 30 nucleotides in length. In some embodiments, the guide RNA binding sequence is positioned at +1 or −7 of the sequence encoding the suppressor tRNA. In some embodiments, the repressor is a guide RNA complexed to a Cas protein, wherein the Cas protein is linked or fused to a repressor domain. In some embodiments, the Cas protein is a catalytically dead Cas protein. In some embodiments, the repressor domain is a krab domain. In some embodiments, the polynucleotide sequence comprising one or more conditionally repressible suppressor tRNA coding sequence(s) comprises two or more conditionally repressible suppressor tRNA coding sequences.

In some embodiments, the polynucleotide sequence comprising one or more conditionally repressible suppressor tRNA coding sequence(s) comprises two, three, four, five, or six conditionally repressible suppressor tRNA coding sequences. In some embodiments, the polynucleotide sequence comprising one or more conditionally repressible suppressor tRNA coding sequence(s) comprises two conditionally repressible suppressor tRNA coding sequences. In some embodiments, the polynucleotide sequence comprising one or more conditionally repressible suppressor tRNA coding sequence(s) comprises three conditionally repressible suppressor tRNA coding sequences. In some embodiments, the polynucleotide sequence comprising one or more conditionally repressible suppressor tRNA coding sequence(s) comprises six conditionally repressible suppressor tRNA coding sequences.

In some embodiments, the conditionally repressible suppressor tRNA coding sequences are the same. In some embodiments, the conditionally repressible suppressor tRNA coding sequences are different.

In some embodiments, the conditionally repressible suppressor tRNA coding sequences are separated by filler sequence(s). In some embodiments, the filler sequence(s) are each, independently, from 1 to 500 nucleotides in length. In some embodiments, the filler sequence(s) are 100 nucleotides in length.

In some embodiments, the polynucleotide sequence comprising one or more conditionally repressible suppressor tRNA coding sequence(s) further comprises quantification tag sequence(s). In some embodiments, the polynucleotide comprises a quantification tag sequence 5′ of the conditionally repressible suppressor tRNA coding sequence(s). In some embodiments, the polynucleotide comprises a quantification tag sequence 3′ of the conditionally repressible suppressor tRNA coding sequence(s). In some embodiments, the polynucleotide sequence comprising one or more conditionally repressible suppressor tRNA coding sequence(s) further comprises a transduction marker sequence. In some embodiments, the transduction marker sequence encodes a fluorescent protein.

In some embodiments, the 5′ and 3′ ITR sequences are selected from AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68 5 and 3 ITR sequences, or any combination thereof; optionally, wherein the 5′ ITR sequence comprises at least 80%, 85%, 90%, 95%, 97%, 99%, or 100% sequence identity to any one of SEQ ID NOs: 225-226; optionally, wherein the 3′ ITR comprises at least 80%, 85%, 90%, 95%, 97%, 99%, or 100% sequence identity to any one of SEQ ID NOs: 227-228.

Also provided herein are vectors comprising any of the polynucleotides described herein.

Also provided herein are systems comprising any of the the vectors described herein; and one or more vectors comprising polynucleotide sequences(s) encoding for one or more proteins selected from the group of: a Rep protein, a Cap protein, a Helper protein, VA RNA, an AAP protein, an MAAP protein, a Protein X protein, and combinations thereof.

Also provided herein are cell(s) comprising any of the vectors or systems described herein. In some embodiments, the cell(s) are engineered to express a repressor configured to bind to the repressor element of the polynucleotide. In some embodiments, the repressor is Tet Repressor protein; optionally, wherein the Tet Repressor protein is TetR; further optionally, wherein the Tet Repressor protein comprises at least 80%, 85%, 90%, 95%, 97%, 99%, or 100% sequence identity to SEQ ID NO: 223. In some embodiments, the cell(s) comprise adenovirus gene E1; optionally, wherein the adenovirus gene E1 comprises E1A gene and/or E1B gene.

In some embodiments, binding of the repressor to the repressor element represses expression of the suppressor tRNA, relative to a control, optionally wherein the repressor element represses expression of the suppressor tRNA at least 40%, at least 45%, at least 50%, at least 55%, at least 60%, at least 65%, at least 70%, at least 75%, at least 80%, at least 85%, or at least 90% relative to a control. In some embodiments, the control is: a) expression of the suppressor tRNA in the cell(s) when the repressor is inhibited from binding to the repressor element, optionally wherein the repressor is inhibited from binding to the repressor element by binding to a switching agent; or b) expression of the suppressor tRNA from reference cell(s) comprising the vector, wherein the reference cell(s) do not express the repressor.

Also provided herein are methods for producing AAV genomes, comprising: culturing the cell(s) described herein under conditions effective to replicate the polynucleotide sequence comprising one or more conditionally repressible suppressor tRNA coding sequence(s).

Also provided herein are methods for producing virion encapsidating a polynucleotide coding for a suppressor tRNA, comprising: culturing the cell(s) described herein under conditions effective to replicate the polynucleotide sequence comprising one or more conditionally repressible suppressor tRNA coding sequence(s) and express the Cap protein.

Also provided herein are composition comprising one or more stop codon engineered AAV nucleotide sequence(s).

In some embodiments, the composition further comprises one or more suppressor tRNA coding sequence(s), optionally flanked by 5′ and 3′ ITR sequences.

In some embodiments, the suppressor tRNA coding sequence(s) independently encode a suppressor tRNA that is capable of suppressing an opal stop codon, an ochre stop codon, or an amber stop codon. In some embodiments, the suppressor tRNA coding sequence comprises 100%, at least 99%, at least 98%, at least 95%, or at least 90% sequence identity to any one of SEQ ID NO: 3-SEQ ID NO: 48, SEQ ID NO: 103-SEQ ID NO: 148, SEQ ID NO: 49-SEQ ID NO: 88, or SEQ ID NO: 249-288; optionally, wherein the anticodon is engineered to bind to an ochre stop codon, an amber stop codon, or an opal stop codon.

In some embodiments, the stop codon engineered AAV nucleotide sequence(s) are selected from the group consisting of a sequence coding for a Rep protein, a sequence coding for a Cap protein, a sequence coding for a helper protein, a sequence encoding VA RNA, a sequence encoding AAP, a sequence encoding MAAP, a sequence encoding Protein X, and any combinations thereof. In some embodiments, the stop codon engineered AAV nucleotide sequence(s) are selected from the group consisting of 23K Endoprotease, VP1, E1B 19K, E1B 55K, E2A, Hexon-associated precursor, Hexon assembly, Rep2, and combinations thereof.

In some embodiments, one or more opal stop codons of the AAV nucleotide sequence(s) is engineered to an amber stop codon or ochre stop codon. In some embodiments, one or more ochre stop codons of the AAV nucleotide sequence(s) is engineered to an amber stop codon or opal stop codon. In some embodiments, the one or more stop codon engineered AAV nucleotide sequence(s) comprises one or more sequences selected from SEQ ID NOs: 155-156, with one or more of the stop codons engineered to a different stop codon. In some embodiments, the one or more stop codon engineered AAV nucleotide sequence(s) comprises one or more sequences selected from SEQ ID NOs: 214-221, with one or more of the stop codons engineered to a different stop codon.

Also provided herein are cell(s) comprising the any of the compositions described herein. Also provided herein are methods for producing AAV genomes, comprising: culturing the cell(s) described herein under conditions effective to replicate the polynucleotide sequence comprising one or more conditionally repressible suppressor tRNA coding sequence(s).

Also provided herein are methods for producing virion encapsidating a polynucleotide coding for a suppressor tRNA, comprising: culturing the cell(s) described herein under conditions effective to replicate the polynucleotide sequence comprising one or more conditionally repressible suppressor tRNA coding sequence(s) and express the Cap protein.

Also provided herein are virion produced by any of the methods described herein.

In some embodiments, a capsid of the AAV virion is selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combination thereof.

Also provided herein are methods of treating a subject having a disease associated with a premature stop codon comprising administering a virion described herein to the subject. In some embodiments, the disease associated with a premature stop codon is Rett syndrome, Dravet syndrome, or Duchenne Muscular Dystrophy.

Also provided herein are methods of treating a subject having Rett syndrome comprising administering a virion described herein to the subject. In some embodiments, the subject is a human.

Also provided herein are methods for producing encapsidated virion, comprising: culturing the cell(s) described herein under conditions effective (a) to replicate the polynucleotide comprising one or more conditionally repressible suppressor tRNA coding sequence(s) and (b) express the Cap protein; wherein expression of the suppressor tRNA is repressed compared to a control; optionally wherein the expression of the suppressor tRNA is repressed at least 40%, at least 45%, at least 50%, at least 55%, at least 60%, at least 65%, at least 70%, at least 75%, at least 80%, at least 85%, or at least 90% relative to a control; optionally wherein the control is: a) expression of the suppressor tRNA in the cell(s) when the repressor is inhibited from binding to the repressor element, optionally wherein the repressor is inhibited from binding to the repressor element by a switching agent that binds to the repressor; or b) expression of the suppressor tRNA from reference cell(s) comprising a vector described herein, wherein the reference cell(s) do not express the repressor; optionally, wherein the switching agent is doxycycline.

Also provided herein are methods for readthrough of a premature stop codon in a subject having a disease associated with a premature stop codon, comprising: producing an encapsidated virion according to any of the methods described herein; and administering the virion to a subject having a disease associated with a premature stop codon under conditions effective for expression of the suppressor tRNA and readthrough of the premature stop codon. In some embodiments, the disease associated with a premature stop codon is Rett syndrome, Dravet syndrome, or Duchenne Muscular Dystrophy. In some embodiments, the disease associated with a premature stop codin is Rett syndrome. In some embodiments, subject is a human.

Provided herein are, among other things, compositions comprising a conditionally repressible suppressor tRNA coding sequence. In some embodiments, the conditionally repressible suppressor tRNA coding sequence comprises sequence coding for a repressor element and a sequence coding for a suppressor tRNA. In some embodiments, the conditionally repressible suppressor tRNA coding sequence comprises a sequence coding for a repressor element upstream or downstream of a sequence coding for a suppressor tRNA. In some embodiments, binding of a repressor to the repressor element represses expression of the suppressor tRNA. In some embodiments, the repressor is a Tet repressor protein. In some embodiments, the sequence coding for the repressor element is positioned at −7 of the sequence coding for the suppressor tRNA. In some embodiments, the sequence coding for the repressor element is positioned at +1 of the sequence coding for the suppressor tRNA. In some embodiments, the repressor element comprises an operon. In some embodiments, the repressor element comprises a sequence of SEQ ID NO: 149 or SEQ ID NO: 150. In some embodiments, the operon comprises a Tet operator (TetO) or Lac operator (LacO). In some embodiments, the operon comprises a sequence of SEQ ID NO: 150. In some embodiments, the repressor element comprises a tetracycline response element (TRE).

In some embodiments, the TRE comprises a TetO sequence. In some embodiments, the TRE comprises a concatemer of TetO sequences. In some embodiments, the concatemer of TetO sequences comprises 2, 3, 4, 5, 6, 7, 8, 9, or 10 TetO sequences. In some embodiments, two or more TetO sequences in the concatemer of TetO sequences are separated by a spacer sequence. In some embodiments, the spacer sequence is TC. In some embodiments, the concatemer of TetO sequences is 2 TetO sequences. In some embodiments, the concatemer of TetO sequences comprises 2 TetO sequences separated by a spacer sequence. In some embodiments, the concatemer of TetO sequences comprises 2 TetO sequences separated by a TC spacer sequence. In some embodiments, the concatemer of TetO sequences is SEQ ID NO: 149.

In some embodiments, the TRE further comprises a promoter. In some embodiments, the TRE is a fusion of the TetO sequence and a promoter. In some embodiments, the TRE is a fusion of the concatemer of TetO sequence and a promoter. In some embodiments, the promoter is a tRNA promoter or a fragment thereof. In some embodiments, the promoter is downstream of the TetO sequence or the concatemer of TetO sequences. In some embodiments, the promoter is upstream of the TetO sequence or the concatemer of TetO sequences.

In some embodiments, the conditionally repressible suppressor tRNA coding sequence is flanked by ITRs.

In some embodiments, the composition comprises one or more conditionally repressible suppressor tRNA coding sequences. In some embodiments, the composition comprises two conditionally repressible suppressor tRNA coding sequences. In some embodiments, the composition comprises three conditionally repressible suppressor tRNA coding sequences. In some embodiments, the composition comprises six conditionally repressible suppressor tRNA coding sequences. In some embodiments, the one or more conditionally repressible suppressor tRNA coding sequence are flanked by inverted terminal repeats (ITRs). In some embodiments, the two conditionally repressible suppressor tRNA coding sequences, the three conditionally repressible suppressor tRNA coding sequences, or the six conditionally repressible suppressor tRNA coding sequences are flanked by ITRs.

In some embodiments, the conditionally repressible suppressor tRNA coding sequence comprises a guide RNA binding sequence and encodes a suppressor tRNA. In some embodiments, the conditionally repressible suppressor tRNA coding sequence further comprises a protospacer adjacent motif (PAM). In some embodiments, the guide RNA binding sequence is from 15 to 30 nucleotides in length. In some embodiments, the guide RNA binding sequence is positioned at +1 or −7 of the sequence coding for the suppressor tRNA. In some embodiments, binding of the guide RNA binding sequence to a guide RNA complexed to a Cas protein that is linked or fused to a repressor domain represses expression of the suppressor tRNA.

In some embodiments, the repressor is a guide RNA complexed to a Cas protein, wherein the Cas protein is linked or fused to a repressor domain. In some embodiments, the Cas protein is a catalytically dead Cas protein. In some embodiments, the repressor domain is a krab domain. In some embodiments, the conditionally repressible suppressor tRNA coding sequence is flanked by ITRs. In some embodiments, the composition comprises one or more conditionally repressible suppressor tRNA coding sequences. In some embodiments, the composition comprises two conditionally repressible suppressor tRNA coding sequences. In some embodiments, the composition comprises three conditionally repressible suppressor tRNA coding sequences. In some embodiments, the composition comprises six conditionally repressible suppressor tRNA coding sequences. In some embodiments, the one or more conditionally repressible suppressor tRNA coding sequence are flanked by ITRs. In some embodiments, the two conditionally repressible suppressor tRNA coding sequences, the three conditionally repressible suppressor tRNA coding sequences, or the six conditionally repressible suppressor tRNA coding sequences are flanked by ITRs.

In some embodiments, the suppressor tRNA are capable of suppressing an opal stop codon, an ochre stop codon, or an amber stop codon. In some embodiments, a sequence of the suppressor tRNA comprises 100%, at least 99%, at least 98%, at least 95%, or at least 90% sequence identity to any one of SEQ ID NO: 3-SEQ ID NO: 48 or SEQ ID NO: 103-SEQ ID NO: 148; optionally, wherein the anticodon is engineered to bind to an ochre stop codon or an amber stop codon. In some embodiments, the suppressor tRNA are capable of suppressing an opal stop codon, an ochre stop codon, or an amber stop codon. In some embodiments, a sequence of the suppressor tRNA comprises 100%, at least 99%, at least 98%, at least 95%, or at least 90% sequence identity to any one of SEQ ID NO: 49-SEQ ID NO: 68 or SEQ ID NO: 249-SEQ ID NO: 268; optionally, wherein the anticodon is engineered to bind to an opal stop codon or an amber stop codon. In some embodiments, the suppressor tRNA are capable of suppressing an opal stop codon, an ochre stop codon, or an amber stop codon. In some embodiments, a sequence of the suppressor tRNA comprises 100%, at least 99%, at least 98%, at least 95%, or at least 90% sequence identity to any one of SEQ ID NO: 69-SEQ ID NO: 88 or SEQ ID NO: 269-SEQ ID NO: 288; optionally, wherein the anticodon is engineered to bind to an ochre stop codon or an opal stop codon. In some embodiments, the conditionally repressible suppressor tRNA coding sequence comprises a sequence selected from SEQ ID NOs: 158-202.

Also provided herein are compositions comprising one or more short interfering RNA (siRNA), one or more short hairpin RNA (shRNA), one or more dicer-substrate RNAs (DsRNA), or any combination thereof, wherein the one or more siRNA, the one or more shRNA, or the one or more DsRNA bind to a suppressor tRNA. In some embodiments, upon binding of the one or more siRNA, the one or more shRNA, or the one or more DsRNA, the suppressor tRNA is targeted for degradation. In some embodiments, upon binding of the one or more siRNA, the one or more shRNA, or the one or more DsRNA, the suppressor tRNA is degraded by an RNA induced silencing complex. In some embodiments, the one or more short interfering RNA (siRNA), one or more short hairpin RNA (shRNA), one or more dicer-substrate RNAs (DsRNA), or any combination thereof, bind to an anticodon region of the suppressor tRNA. In some embodiments, the one or more siRNA are from 15 nucleotides to 25 nucleotides in length. In some embodiments, the one or more siRNA are from 19 nucleotides in length. In some embodiments, the one or more DsRNA are from 25 to 35 nucleotides in length. In some embodiments, the one or more DsRNA are 27 nucleotides in length.

In some embodiments, the composition further comprises the suppressor tRNA. In some embodiments, the suppressor tRNA is capable of suppressing an opal stop codon, an ochre stop codon, or an amber stop codon. In some embodiments, the suppressor tRNA comprises 100%, at least 99%, at least 98%, at least 95%, or at least 90% sequence identity to any one of SEQ ID NO: 3-SEQ ID NO: 48 or SEQ ID NO: 103-SEQ ID NO: 148; optionally, wherein the anticodon is engineered to bind to an ochre stop codon or an amber stop codon. In some embodiments, the suppressor tRNA comprises 100%, at least 99%, at least 98%, at least 95%, or at least 90% sequence identity to any one of SEQ ID NO:49-SEQ ID NO: 68 or SEQ ID NO: 249-SEQ ID NO: 268; optionally, wherein the anticodon is engineered to bind to an opal stop codon or an amber stop codon. In some embodiments, the suppressor tRNA comprises 100%, at least 99%, at least 98%, at least 95%, or at least 90% sequence identity to any one of SEQ ID NO: 69-SEQ ID NO: 88 or SEQ ID NO: 269-SEQ ID NO: 288; optionally, wherein the anticodon is engineered to bind to an ochre stop codon or an opal stop codon.

Also provided herein are compositions comprising one or more stop codon engineered AAV nucleotide sequence(s). In some embodiments, the composition further comprises one or more suppressor tRNA coding sequence(s). In some embodiments, the composition further comprises one or more conditionally repressible suppressor tRNA coding sequence(s); optionally, wherein the one or more conditionally repressible suppressor tRNA coding sequences are any of those provided herein. In some embodiments, the suppressor tRNA coding sequence(s) or the conditionally repressible suppressor tRNA coding sequence(s) are flanked by ITRs. In some embodiments, the suppressor tRNA coding sequence(s) or the conditionally repressible suppressor tRNA coding sequence(s) independently encode a suppressor tRNA that is capable of suppressing an opal stop codon, an ochre stop codon, or an amber stop codon. In some embodiments, the suppressor tRNA coding sequence comprises 100%, at least 99%, at least 98%, at least 95%, or at least 90% sequence identity to any one of SEQ ID NO: 3-SEQ ID NO: 48 or SEQ ID NO: 103-SEQ ID NO: 148; optionally, wherein the anticodon is engineered to bind to an ochre stop codon or an amber stop codon. In some embodiments, the suppressor tRNA comprises 100%, at least 99%, at least 98%, at least 95%, or at least 90% sequence identity to any one of SEQ ID NO:49-SEQ ID NO: 68 or SEQ ID NO: 249-SEQ ID NO: 268; optionally, wherein the anticodon is engineered to bind to an opal stop codon or an amber stop codon. In some embodiments, the suppressor tRNA comprises 100%, at least 99%, at least 98%, at least 95%, or at least 90% sequence identity to any one of SEQ ID NO: 69-SEQ ID NO: 88 or SEQ ID NO: 269-SEQ ID NO: 288; optionally, wherein the anticodon is engineered to bind to an ochre stop codon or an opal stop codon.

In some embodiments, the stop codon engineered AAV nucleotide sequence(s) are selected from the group consisting of a sequence coding for a Rep protein, a sequence coding for a Cap protein, a sequence coding for a helper protein, a sequence encoding VA RNA, a sequence encoding AAP, a sequence encoding MAAP, a sequence encoding Protein X, and any combinations thereof. In some embodiments, the Cap protein(s) are selected from the group consisting of VP1, VP2, VP3, and any combinations thereof. In some embodiments, the Rep protein(s) are selected from the group consisting of Rep78, Rep52, Rep68, Rep40, and any combinations thereof. In some embodiments, the helper protein(s) are selected from the group consisting of E1a, E1b, E4, E2a, and any combinations thereof. In some embodiments, one or more opal stop codons of the AAV nucleotide sequence(s) is engineered to an amber stop codon or ochre stop codon. In some embodiments, one or more ochre stop codons of the AAV nucleotide sequence(s) is engineered to an amber stop codon or opal stop codon. In some embodiments, one or more amber stop codons of the AAV nucleotide sequence(s) is engineered to an ochre stop codon or opal stop codon. In some embodiments, the one or more stop codon engineered AAV nucleotide sequence(s) comprises one or more sequences comprising 100%, at least 99%, at least 98%, at least 95%, or at least 90% sequence identity to SEQ ID NOs: 214-221, with one or more stop codons engineered to a different stop codon.

Also provided herein are cell(s) comprising the composition(s) described herein. In some embodiments, the cell further comprises a sequence coding for a Rep protein.

In some embodiments of the cell(s) or composition(s) provided herein, the Rep protein is Rep78, Rep52, Rep68, Rep40, or any combination thereof. In some embodiments, an opal stop codon of the sequence coding for the Rep68 is changed to an amber stop codon or an ochre stop codon. In some embodiments, an opal stop codon of the sequence coding for the Rep40 is changed to an amber stop codon or an ochre stop codon. In some embodiments, an ochre stop codon of the sequence coding for the Rep78 is changed to an amber stop codon or an opal stop codon. In some embodiments, an ochre stop codon of the sequence coding for the Rep52 is changed to an amber stop codon or an opal stop codon.

In some embodiments the cell(s) or composition(s) provided herein further comprises a sequence coding for a Cap protein. In some embodiments, the Cap protein is VP1, VP2, VP3, or any combination thereof. In some embodiments, an ochre stop codon of the sequence coding for VP1 is changed to an amber stop codon or an opal stop codon. In some embodiments, an ochre stop codon of the sequence coding for VP2 is changed to an amber stop codon or an opal stop codon. In some embodiments, an ochre stop codon of the sequence coding for VP3 is changed to an amber stop codon or an opal stop codon.

In some embodiments the cell(s) or composition(s) provided herein further comprise a sequence coding for a Helper protein or encoding VA RNA. In some embodiments, the Helper protein is E1a, E1b, E4, E2a, or any combination thereof.

In some embodiments, the cell(s) or composition(s) provided herein further comprise a sequence coding for AAP. In some embodiments, an opal stop codon of the sequence coding for AAP is changed to an amber stop codon or an ochre stop codon.

In some embodiments, the cell(s) or composition(s) provided herein further comprise a sequence coding for MAAP. In some embodiments, an amber stop codon of the sequence coding for MAAP is changed to an opal stop codon or an ochre stop codon.

In some embodiments, the cell(s) or composition(s) provided herein further comprise a sequence coding for Protein X. In some embodiments, an opal stop codon of the sequence coding for Protein X is changed to an amber stop codon or an ochre stop codon.

In some embodiments of the cell(s) or composition(s) provided herein, the AAV nucleotide sequence, the conditionally repressible tRNA sequence, the suppressor tRNA coding sequence, the sequence coding for a Rep protein, the sequence coding for a Cap protein, a sequence coding for a helper protein, a sequence encoding VA RNA, a sequence coding for AAP, a sequence coding for MAAP, a sequence coding for Protein X, a sequence coding for a repressor, or any combination of these sequences are in one or more plasmids.

In some embodiments of the cell(s) or composition(s) provided herein, the AAV nucleotide sequence, the conditionally repressible tRNA sequence, the suppressor tRNA coding sequence, the sequence coding for a Rep protein, the sequence coding for a Cap protein, a sequence coding for a helper protein, a sequence encoding VA RNA, a sequence coding for AAP, a sequence coding for MAAP, a sequence coding for Protein X, a sequence coding for a repressor, or any combination of these sequences are stably integrated into the nuclear genome of the cell.

In some embodiments, the cell is a mammalian cell. In some embodiments, the cell is an HEK293 cell or a CHO cell. In some embodiments, the cell is an insect cell. In some embodiments, the cell is an Sf9 cell.

In some embodiments, the cell is capable of producing a virion encapsidating the suppressor tRNA. In some embodiments, the cell is capable of producing a virion encapsidating the conditionally repressible suppressor tRNA coding sequence.

In some embodiments, the cell(s) or composition(s) provided herein further comprise the repressor or a sequence coding for the repressor. In some embodiments, the repressor is a Tet repressor protein or a guide RNA complexed to a catalytically dead Cas protein that is linked or fused to a repressor domain. In some embodiments, the repressor domain is a krab domain.

Also provided herein are methods of silencing a suppressor tRNA comprising: providing a cell comprising a repressor; and transfecting the cell with a composition comprising a conditionally repressible suppressor tRNA coding sequence, as provided herein, wherein the repressor binds to the repressor element, thereby silencing expression of the suppressor tRNA.

Also provided herein are methods of silencing a suppressor tRNA comprising: providing a cell comprising the suppressor tRNA; and transfecting the cell with a composition comprising one or more short interfering RNA (siRNA), one or more short hairpin RNA (shRNA), one or more dicer-substrate RNAs (DsRNA), or any combination thereof, wherein the one or more siRNA, the one or more shRNA, or the one or more DsRNA bind to a suppressor tRNA, as provided herein; wherein the siRNA, shRNA, DsRNA or any combination thereof binds to the suppressor tRNA and targets the suppressor tRNA for degradation, thereby silencing the tRNA.

Also provided herein are methods of silencing suppressor tRNA for virion production comprising: providing a cell comprising a repressor; transfecting the cell with a sequence coding for a helper protein, a sequence encoding VA RNA, a sequence coding for a Rep protein, and a sequence coding for a Cap protein; transfecting the cell with a composition comprising a conditionally repressible suppressor tRNA coding sequence, as provided herein, wherein the conditionally repressible suppressor tRNA coding sequence is flanked by ITRs; and culturing the cell under conditions suitable for expression and encapsidation of the suppressor tRNA, thereby producing virions encapsidating the conditionally repressible suppressor tRNA coding sequence, wherein the repressor binds to the repressor element, thereby silencing expression of the suppressor tRNA for virion production.

Also provided herein are methods of silencing suppressor tRNA for virion production comprising: providing a cell comprising a suppressor tRNA flanked by ITRs; transfecting the cell with a sequence coding for a helper protein, a sequence encoding VA RNA, a sequence coding for a Rep protein, and a sequence coding for a Cap protein; transfecting the cell with a composition comprising one or more short interfering RNA (siRNA), one or more short hairpin RNA (shRNA), one or more dicer-substrate RNAs (DsRNA), or any combination thereof, wherein the one or more siRNA, the one or more shRNA, or the one or more DsRNA bind to a suppressor tRNA, as provided herein; and culturing the cell under conditions suitable for expression and encapsidation of the suppressor tRNA, thereby producing virions encapsidating the conditionally repressible suppressor tRNA coding sequence, wherein the siRNA, shRNA, DsRNA or any combination thereof binds to the suppressor tRNA and targets the suppressor tRNA for degradation, thereby silencing the suppressor tRNA for virion production.

Also provided herein are methods of producing virion encapsidating a sequence coding for a suppressor tRNA comprising providing a cell comprising a repressor; transfecting the cell with a sequence coding for a helper protein, a sequence coding for a Rep protein, and a sequence coding for a Cap protein; transfecting the cell with the composition comprising a conditionally repressible suppressor tRNA coding sequence, as provided herein, wherein the conditionally repressible suppressor tRNA coding sequence is flanked by ITRs; and culturing the cell under conditions suitable for expression and encapsidation of the suppressor tRNA, thereby producing virions encapsidating the sequence coding for a suppressor tRNA, wherein the repressor binds to the repressor element, thereby silencing expression of the suppressor tRNA.

Also provided herein are methods of producing virion that encapsidates a sequence coding for a suppressor tRNA comprising providing a cell comprising a suppressor tRNA coding sequence flanked by ITRs; transfecting the cell with a sequence coding for a helper protein, a sequence coding for a Rep protein, and a sequence coding for a Cap protein; transfecting the cell with a composition comprising one or more short interfering RNA (siRNA), one or more short hairpin RNA (shRNA), one or more dicer-substrate RNAs (DsRNA), or any combination thereof, wherein the one or more siRNA, the one or more shRNA, or the one or more DsRNA bind to a suppressor tRNA, as provided herein; and culturing the cell under conditions suitable for expression and encapsidation of the suppressor tRNA, thereby producing virions encapsidating the conditionally repressible suppressor tRNA coding sequence, wherein the siRNA, shRNA, DsRNA or any combination thereof binds to the suppressor tRNA and targets the suppressor tRNA for degradation, thereby silencing the suppressor tRNA.

Also provided herein are methods of producing virion encapsidating a sequence coding for a suppressor tRNA, the method comprising: a) providing a cell comprising: a sequence encoding a suppressor tRNA flanked by ITRs, a sequence encoding a helper protein, a sequence encoding a VA RNA, a sequence encoding a Rep protein, a sequence encoding a Cap protein, and optionally a sequence coding for AAP, a sequence coding for MAAP, and/or a sequence coding for Protein X, wherein one or more of the sequence encoding the helper protein, the sequence encoding the VA RNA, the sequence encoding the Rep protein, the sequence coding for the Cap protein, the sequence coding for AAP, the sequence coding for MAAP, and/or the sequence coding for Protein X is stop codon engineered; and b) culturing the cell under conditions suitable for expression and encapsidation of the suppressor tRNA.

Also provided herein are methods of increasing titer of virions encapsidating a sequence coding for a suppressor tRNA by silencing the suppressor tRNA comprising providing a cell comprising a repressor; transfecting the cell with a sequence coding for a helper protein, a sequence coding for a Rep protein, and a sequence coding for a Cap protein; transfecting the cell with a composition comprising a conditionally repressible suppressor tRNA coding sequence, as provided herein, wherein the conditionally repressible suppressor tRNA coding sequence is flanked by ITRs; and producing virions encapsidating the sequence coding for a suppressor tRNA, wherein the repressor binds to the repressor element, thereby silencing expression of the suppressor tRNA for virion production, and wherein the titer of virions encapsidating the sequence coding for the suppressor tRNA is increased compared to titer of virions encapsidating the sequence for the suppressor tRNA produced by steps b)-d) or produced by transfecting the cell with a sequence coding for a suppressor tRNA and transfecting the cell with a sequence coding for a helper protein, a sequence coding for a Rep protein, and a sequence coding for a Cap protein.

Also provided herein are methods of increasing titer of virions encapsidating a sequence coding for a suppressor tRNA by silencing the suppressor tRNA comprising transfecting a cell with the sequence coding for the suppressor tRNA flanked by ITRs; transfecting the cell with a sequence coding for a helper protein, a sequence coding for a Rep protein, and a sequence coding for a Cap protein; transfecting the cell with a composition comprising one or more short interfering RNA (siRNA), one or more short hairpin RNA (shRNA), one or more dicer-substrate RNAs (DsRNA), or any combination thereof, wherein the one or more siRNA, the one or more shRNA, or the one or more DsRNA bind to a suppressor tRNA, as provided herein; and culturing the cell under conditions suitable for expression and encapsidation of the suppressor tRNA, thereby producing virions encapsidating the sequence coding for the suppressor tRNA, wherein the siRNA, shRNA, DsRNA or any combination thereof binds to the suppressor tRNA and targets the suppressor tRNA for degradation, thereby silencing the suppressor tRNA, and wherein the titer of virions encapsidating the sequence coding for the suppressor tRNA is increased compared to titer of virions encapsidating the sequence for the suppressor tRNA produced by steps a), b), and d).

Also provided herein are methods of increasing titer of virions encapsidating a sequence coding for a suppressor tRNA, the method comprising: a) providing a cell comprising: a sequence encoding a suppressor tRNA flanked by ITRs, a sequence encoding a helper protein, a sequence encoding a VA RNA, a sequence encoding a Rep protein, a sequence encoding a Cap protein, and optionally a sequence coding for AAP, a sequence coding for MAAP, and/or a sequence coding for Protein X, wherein one or more of the sequence encoding the helper protein, the sequence encoding the VA RNA, the sequence encoding the Rep protein, the sequence coding for the Cap protein, the sequence coding for AAP, the sequence coding for MAAP, and/or the sequence coding for Protein X is stop codon engineered; and b) culturing the cell under conditions suitable for expression and encapsidation of the suppressor tRNA.

In some embodiments of the methods described herein, the repressor is a Tet repressor protein or a guide RNA complexed to a catalytically dead Cas protein that is linked or fused to a repressor domain.

In some embodiments of the methods provided herein, the repressor domain is a krab domain. In some embodiments, the Rep protein is Rep78, Rep52, Rep68, Rep40, or any combination thereof. In some embodiments, an opal stop codon of the sequence coding for the Rep68 is changed to an amber stop codon or an ochre stop codon. In some embodiments, an opal stop codon of the sequence coding for the Rep40 is changed to an amber stop codon or an ochre stop codon. In some embodiments, an ochre stop codon of the sequence coding for the Rep78 is changed to an amber stop codon or an opal stop codon. In some embodiments, an ochre stop codon of the sequence coding for the Rep52 is changed to an amber stop codon or an opal stop codon.

In some embodiments of the methods provided herein, the Cap protein is VP1, VP2, VP3, or any combination thereof. In some embodiments, an ochre stop codon of the sequence coding for VP1 is changed to an amber stop codon or an opal stop codon. In some embodiments, an ochre stop codon of the sequence coding for VP2 is changed to an amber stop codon or an opal stop codon. In some embodiments, an ochre stop codon of the sequence coding for VP3 is changed to an amber stop codon or an opal stop codon.

In some embodiments of the methods provided herein, the helper protein is E1a, E1b, E4, E2a, or any combination thereof.

In some embodiments of the methods provided herein, the cell is a mammalian cell. In some embodiments, the cell is an HEK293 cell or a CHO cell. In some embodiments, the cell is an insect cell. In some embodiments, the cell is an Sf9 cell.

In some embodiments of the methods provided herein, the virion is an AAV virion. In some embodiments, a capsid of the AAV virion is selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combinations thereof.

Also provided herein are virion produced by any of the methods provided herein.

Also provided herein are virion encapsidating the sequence coding for the conditionally repressible suppressor tRNA provided herein. In some embodiments, the virion is an AAV virion. In some embodiments, a capsid of the AAV virion is selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combinations thereof.

Also provided herein are methods of treating a subject having a disease associated with a premature stop codon comprising administering the virion provided herein to the subject. In some cases, the disease associated with a premature stop codon is Rett syndrome, Dravet syndrome, or Duchenne Muscular Dystrophy.

Also provided herein are methods of treating a subject having Rett syndrome, the method comprising administering the virion provided herein to the subject.

In some embodiments of the methods of treating a subject provided herein, the subject is a human.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1D are schematics of examples of methods of silencing suppressor tRNA. FIG. 1A depicts the silencing of a suppressor tRNA using repressors that bind to a repressor element upstream of the sequence coding for the suppressor tRNA sequence. Examples of repressor systems are a Tet Repressor system as shown in FIG. 1B and a CRISPRi system shown in FIG. 1C. FIG. 1D depicts the use of siRNA and/or shRNA and/or DsRNA that silence the suppressor tRNA by binding to suppressor tRNA (e.g., covering the anti-codon region of the suppressor tRNA), thereby targeting the suppressor tRNA for degradation by RISC and subsequently silencing the suppressor tRNA.

FIG. 1E shows decrease in AAV titer produced by cells expressing suppressor tRNA CCT2.10 (1-8 and 12-17) as compared to controls (10 and 11 (no suppressor tRNA); and 18 (GFP)). One control also had low titer (9 (no suppressor tRNA)).

FIGS. 2A-2B show an example AAV backbone plasmid (FIG. 2A) and a schematic of a sequence between ITRs (FIG. 2B) comprising three conditionally repressible suppressor tRNA coding sequences (3× a sequence comprising the repressor element (TRE sequence) and the sequence coding for the suppressor tRNA), synthetic filler sequences (100 bps), an EF1a_core-mCherry sequence (transduction marker), strawberry sequences (quantification tags for ddPCR quantification of episomes/AAV transcripts) inserted into the AAV backbone plasmid. The suppressor tRNAs can be CCT4, CCT2.10, or CCT5 (control tRNA; non-functional). The TRE sequence comprises two repeats of the Tet operator (19 bp) with a TC spacer: TCCCTATCAGTGATAGAGATCTCCCTATCAGTGATAGAGA (SEQ ID NO: 149).

FIGS. 3A-3B show sequences coding for suppressor tRNAs downstream of a repressor element are silenced in cells expressing the corresponding repressor compared to cells not expressing the corresponding repressor. Cells not expressing the Tet repressor (no repressor) (FIG. 3A) or cells expressing the Tet repressor (+repressor) (FIG. 3B) were transfected with a first plasmid comprising either a R74X premature stop codon or a R97X premature stop codon inserted into a GFP sequence (broken GFP) and a second plasmid comprising three copies of a repressor element-suppressor tRNA sequence and an intact mCherry sequence. The suppressor tRNA of the second plasmid was 1: 3×CCT4-TRE+1; 2: 3×CCT4-TRE −7; 3: 3×CCT2.10-TRE +1; or 4: 3×CCT5-TRE+1 (3× indicates the plasmid had three copies of the repressor element-suppressor tRNA sequence, TRE indicates the repressor element, which was a sequence comprising two TetO sequences separated by a TC spacer (SEQ ID NO: 149); +1 or −7 indicates the position of the repressor element with respect to the suppressor tRNA sequence). CCT4 and CCT2.10 are suppressor tRNAs. CCT5 is a control tRNA that is non-functional. Detection of a GFP signal indicates the suppressor tRNA was expressed and there was subsequent readthrough of the premature stop codon to produce detectable GFP. Detection of mCherry indicates successful transfection of the plasmid comprising repressor element upstream of the suppressor tRNA coding sequence. FIG. 3A shows a high ratio of GFP/mCherry MFI indicating readthrough of the broken GFP and therefore, expression of the suppressor tRNAs in cells that did not express the repressor. FIG. 3B shows a low ratio of GFP/mCherry MFI indicating low to no readthrough of the broken GFP and therefore, silencing of expression of the suppressor tRNAs in cells that expressed the repressor.

FIG. 4 shows increased AAV titer produced by cells expressing a repressor and transfected with a plasmid comprising a repressor element upstream of the suppressor tRNA compared to cells transfected with a plasmid lacking a repressor element upstream of the suppressor tRNA. Cells that express a Tet repressor were transfected with plasmids comprising AAV helper protein sequences, plasmids comprising AAV Rep protein sequences and Cap protein sequences, and plasmids comprising a suppressor tRNA sequence. The plasmids comprising sequence coding for a suppressor tRNA were 1A: AAV2, CCT4 3×, +1; 2A: AAV2, CCT4 3×, −7; 3A: AAV2, CCT2.10 3×, +1; 4B: AAV2, CCT5 3×, +1; STX650: AAV2, CMV-GFP control; STX0607: AAV2, CCT2.10 3×, no repressor element; AAV9-3A: AAV9, CCT2.10 3×, +1; or AAV2-3A: AAV2, CCT2.10 3×, +1 (3× indicates the plasmid had three copies of the repressor element-suppressor tRNA sequence (or three copies of the suppressor tRNA sequence if no repressor element), +1 or −7 indicates the position of the repressor element relative to the suppressor tRNA sequence, in which repressor element was a TRE sequence comprising two TetO sequences separated by a TC spacer (SEQ ID NO: 149)). CCT4 and CCT2.10 are suppressor tRNAs. CCT5 is a control tRNA that is non-functional. The AAV serotypes tested were AAV2 or AAV9 as indicated. Production was scaled up for AAV9-3A as shown for the right most bar on the graph.

FIG. 5 shows AAV titer measured by ddPCR from cells transfected with Rep/Cap plasmids (pRC), helper plasmids (pHelper), and plasmids encoding different tRNAs (no tRNA (GFP only); Pyl-TGA; CCT2.10 Arg; CCT2.10; TCT1.11; TTG1-ochre; or TTG1-amber, independently); from cells transfected with pHelper and no tRNA (GFP only) plasmids (No RNA, no pRC); or from cells that were not transfected (no trfn).

FIG. 6 shows capsid titer measured by ELISA (left bars), AAV titer without benzonase treatment (“No nuclease”) measured by ddPCR (middle bars), and encapsidated AAV titer after benzonase treatment (“with nuclease”) measured by ddPCR from cells transfected with Rep/Cap plasmids (pRC), helper plasmids (pHelper), and plasmids encoding different tRNAs (no tRNA (GFP only); Pyl-TGA; CCT2.10 Arg; CCT2.10; TCT1.11; TTG1-ochre; or TTG1-amber, independently); from cells transfected with helper plasmids and no tRNA (GFP only) plasmids (No tRNA, no pRC); or from cells that were not transfected (no trfn).

FIG. 7 shows comparable total genomes (middle bars), encapsidated genomes (right bars), and capsid titer (left bars) produced by cells transfected with a helper plasmid (pHelper) and: (1) pRC2+614: a Rep/Cap plasmid (pRC2) and a payload plasmid comprising a sequence between two ITRs encoding mCherry (614); (2) pRC2+606: a Rep/Cap plasmid (pRC2) and a payload plasmid comprising a sequence between two ITRs encoding three TCT suppressor tRNAs and mCherry (606); (3) Opal+614: a Rep/Cap plasmid encoding TGA stop codons engineered to TAA stop codons (Opal) and a payload plasmid comprising a sequence between two ITRs encoding mCherry (614); (4) Opal+606: a Rep/Cap plasmid encoding TGA stop codons engineered to TAA stop codons (Opal) and a payload plasmid comprising a sequence between two ITRs encoding three TCT suppressor tRNAs and mCherry (606); (5) Arg+614: a Rep/Cap plasmid encoding TGA stop codons engineered to arginine codons (Arg) and a payload plasmid comprising a sequence between two ITRs encoding mCherry (614); (6) Arg+606: a Rep/Cap plasmid encoding TGA stop codons engineered to arginine codons (Arg) and a payload plasmid comprising a sequence between two ITRs encoding three TCT suppressor tRNAs and mCherry (606); (7) AAP-TAA+614: a Rep/Cap plasmid encoding the TGA stop codon of AAP engineered to a TAA stop codon (AAP-TAA) and a payload plasmid comprising a sequence between two ITRs encoding mCherry (614); or (8) AAP-TAA+606: a Rep/Cap plasmid encoding the TGA stop codon of AAP engineered to a TAA stop codon (AAP-TAA) and a payload plasmid comprising a sequence between two ITRs encoding three TCT suppressor tRNAs and mCherry (606).

FIG. 8 shows total genomes titer (without benzonase treatment (“Nuclease-”; left bars) and encapsidated genomes titer (after benzonase treatment (“Nuclease+”; right bars) produced by cells transfected with a helper plasmid (pHelper) and: (1) pRC2 and STX894: a Rep/Cap plasmid (pRC2) and a payload plasmid comprising a sequence between two ITRs encoding TTG1-ochre suppressor tRNA (STX894); (2) pRC2-Ochre and STX894: a Rep/Cap plasmid encoding TAA stop codons engineered to TAG stop codons (pRC2-Ochre) and a payload plasmid comprising a sequence between two ITRs encoding TTG1-ochre suppressor tRNA (STX894); (3) pRC2 and STX956: a Rep/Cap plasmid (pRC2) and a payload plasmid comprising a sequence between two ITRs encoding a CCT5 tRNA (control tRNA that is non-functional; STX956); or (4) pRC2-Ochre and STX956: a Rep/Cap plasmid encoding TAA stop codons engineered to TAG stop codons (pRC2-Ochre) and a payload plasmid comprising a sequence between two ITRs encoding a CCT5 tRNA (control tRNA that is non-functional; STX956).

FIG. 9 shows increased AAV titer produced by cells transfected with a plasmid comprising a repressor element (TRE sequence comprising two TetO sequences separated by a TC spacer (SEQ ID NO: 149)) upstream of a sequence coding for a suppressor tRNA in the presence of a repressor (Tet repressor; expressed by the transfected cells) compared to cells transfected with a plasmid comprising a repressor element (TRE sequence comprising two TetO sequences separated by a TC spacer (SEQ ID NO: 149)) upstream of a sequence coding for a suppressor tRNA in the presence of a repressor (Tet repressor; expressed by the transfected cells) and doxycycline, which competes with the repressor element upstream of the suppressor tRNA for binding to the repressor, and thus reduces the repression of the downstream suppressor tRNA. In the presence of doxycycline, repression is less and therefore AAV titer is reduced (3A+Dox Cells that express a repressor were transfected with plasmids comprising AAV helper protein sequences, plasmids comprising AAV Rep protein sequences and Cap protein sequences, and a 3A plasmid comprising a sequence coding for suppressor tRNA or a control STX650 plasmid as follows-3A plasmid: AAV2, CCT2.10 3×, +1; or STX650 plasmid: AAV2, CMV-GFP control (3× indicates the plasmid had three copies of the repressor element-suppressor tRNA sequence, +1 indicates the position of the repressor element relative to the suppressor tRNA sequence, in which the repressor element was a TRE sequence comprising two TetO sequences separated by a TC spacer (SEQ ID NO: 149), and CCT2.10 indicates the suppressor tRNA encoded by the suppressor tRNA sequence). Titer was assessed from transfected cells in the presence of doxycycline (3A+Dox) or in the absence of doxycycline (3A or STX650)

FIGS. 10A-10D show schematic depiction of example repressor element-suppressor tRNA constructs described herein. FIG. 10A shows a schematic depiction of an example repressor element-suppressor tRNAconstruct comprising the repressor element (two TetO sequences (TetO), e.g., two TetO sequences separated by a TC spacer (e.g., SEQ ID NO: 149)) placed upstream of the suppressor tRNA sequence (CCT2.10). FIG. 10B shows a schematic depiction of an example repressor element-suppressor tRNAconstruct comprising the repressor element (two TetO sequences (TetO), e.g., two TetO sequences separated by a TC spacer (e.g., SEQ ID NO: 149)) placed downstream of the suppressor tRNA sequence (CCT2.10). The numbers +1, −7, −12, −20, −30 and −45 indicate the different positions of the repressor element (two TetO sequences (TetO), e.g., two TetO sequences separated by a TC spacer (e.g., SEQ ID NO: 149)) relative to the suppressor tRNA sequence in FIG. 10A and FIG. 10B. FIG. 10C shows a schematic depiction of an example repressor element-suppressor tRNAconstruct comprising the repressor element (one TetO sequence (TetO); e.g., SEQ ID NO: 150) placed upstream of the tRNA sequence (CCT2.10). FIG. 10D shows a schematic depiction of an example repressor element-suppressor tRNA construct comprising the repressor element (one TetO sequence (TetO); e.g., SEQ ID NO: 150) placed downstream of the tRNA sequence (CCT2.10).

FIG. 11 shows a graph of % GFP+ cells of transfected cells (Thy 1.1+ cells) after independent transfection of either HEK293 cells (Repressor-) stably expressing a GFP reporter comprising a R74X premature stop codon (left bars) or HEK293 cells expressing a Tet repressor (Repressor+) and stably expressing a GFP reporter comprising a R74X premature stop codon (right bars) with plasmids from a series of plasmids designed to assess the number and the placement of the TetO sequences of the repressor element. The percent GFP+ cells were normalized to the percent GFP+ cells of the control noTET_CCT2.10-WT.

FIG. 12 shows a graph of % GFP+ cells of transfected cells (Thy 1.1+ cells) after independent transfection of either HEK293 cells (Repressor-) stably expressing a GFP reporter comprising a R97X premature stop codon or HEK293 cells expressing a Tet repressor (Repressor+) and stably expressing a GFP reporter comprising a R97X premature stop codon with plasmids from a series of plasmids designed to assess the number and the placement of the TetO sequences of the repressor element. The percent GFP+ cells were normalized to the percent GFP+ cells of the control noTET_CCT2.10-WT.

FIG. 13 shows a schematic depiction of exemplary variations of hU6-repressor element-suppressor tRNAconstructs. SPH, OCT-1, PSE and TATA are elements of the hU6 promoter. Variations of the placement of the repressor elements with the elements of the hU6 promoter are shown, with the repressor element (“TetO”) placed in one or more of the three positions downstream of the SPH and OCT-1 elements-either before the PSE element, between the PSE and TATA box, or after the TATA box. As indicated, the repressor element may be present as one TetO sequence (as indicated by 1×; e.g., SEQ ID NO: 150) or two TetO sequences (as indicated by 2×; e.g., SEQ ID NO: 149). TSS indicates the transcriptional start site of the sequence coding for a suppressor tRNA (e.g., CCT4 or CCT2.10).

FIG. 14A shows a graph of % GFP+ cells of transfected cells (Thy 1.1+ cells) after independent transfection of either HEK293 cells (Repressor−) stably expressing a GFP reporter comprising a R74X premature stop codon (left bars) or HEK293 cells expressing a Tet repressor (Repressor+) that stably express a GFP reporter comprising a R74X premature stop codon (right bars) with plasmids comprising the hU6-repressor element-suppressor tRNA constructs or control constructs of TABLE 8 to assess the number of the TetO sequences of the repressor element and the placement of repressor element relative to elements of the hU6 promoter and the sequence coding for the suppressor tRNA. The % GFP+ cells were normalized to the % GFP+ cells of the control noTetCCT2.10.

FIG. 14B shows a graph of % GFP+ cells of transfected cells (Thy 1.1+ cells) after independent transfection of either HEK293 cells (Repressor−) stably expressing a GFP reporter comprising a R97X premature stop codon or HEK293 cells expressing a Tet repressor (Repressor+) that stably express a GFP reporter comprising a R97X premature stop codon with plasmids comprising the hU6-repressor element-suppressor tRNA constructs or control constructs of TABLE 8 to assess the number of the TetO sequences of the repressor element and the placement of repressor element relative to elements of the hU6 promoter and the sequence coding for the suppressor tRNA. The % GFP+ cells were normalized to the % GFP+ cells of the control noTetCCT2.10.

FIG. 15 shows a schematic depiction of an example a repressor element-suppressor tRNA AAV construct, e.g., comprising three copies of a repressor element-suppressor tRNA sequence inserted between inverted terminal repeats (ITRs) of scAAV plasmid or ssAAV plasmid. The repressor element (TET) can comprise a TRE sequence comprising two TetO sequences separated by a TC spacer (e.g., SEQ ID NO: 149) or a TetO sequence (e.g., SEQ ID NO: 150) upstream of a sequence coding for a suppressor tRNA. The suppressor tRNA sequence can encode CCT2.10 as indicated. A synthetic filler sequence (syn. filler) can be downstream of a repressor element-suppressor tRNA sequence, which together with the repressor element-suppressor tRNA sequence produces a repeat (e.g., a repressor element-suppressor tRNA sequence-synthetic filler sequence). As shown, there can be three repeats between ITRs in the repressor element-suppressor tRNA AAV construct. CMV-350 is constitutive promoter that can be upstream of a sequence coding for Thy 1.1, in which expression of Thy 1.1 can be used as a tranfection marker or transduction marker, and can also be included, e.g., downstream of the last repressor element-suppressor tRNA sequence-synthetic filler sequence repeat, between ITRs in the repressor element-suppressor tRNA AAV construct. The repressor element-suppressor tRNA AAV construct can be cloned into an AAV plasmid.

FIGS. 16A-16C show data from testing of various repressor element-suppressor tRNA AAV constructs in AAV plasmids. FIGS. 16A-16B show graphs of GFP mean fluorescence intensity (MFI) relative to the control vector (STX0607) detected in HEK 293 cells (Repressor−) (FIG. 16A) stably expressing a broken GFP reporter (having a R74X premature stop codon or R97X premature stop codon) or HEK293 cells expressing a Tet repressor (Repressor+) (FIG. 16B) that also stably express a broken GFP reporter (having a R74X premature stop codon or R97X premature stop codon). These cells were independently transfected with the AAV plasmids having various repressor element-suppressor tRNA AAV constructs as indicated, which were designed to assess the number and the placement of the repressor element on suppressor tRNA function either in the presence or absence of the repressor. FIG. 16C shows a plot of the GFP mean fluorescence intensity (MFI) relative to the control vector (STX0607) in HEK293 cells expressing a Tet repressor (Repressor+) that also stably express a broken GFP reporter (having a R74X premature stop codon) vs. viral titer (total vgs) from HEK293 cells expressing a Tet repressor (Repressor+). For GFP MFI, HEK293 cells expressing a Tet repressor (Repressor+) that also stably express a broken GFP reporter (having a R74X premature stop codon or R97X premature stop codon) were independently transfected with scAAV plasmids having various repressor element-suppressor tRNA constructs as indicated, in which GFP expression indicates readthrough of the broken GFP reporter. For viral titer, HEK293 cells expressing a Tet repressor (Repressor+) were triple transfected with an AAV helper plasmid, an AAV Rep/Cap plasmid, and an scAAV plasmid comprising the repressor element-suppressor tRNA construct as indicated to produce AAV virion.

FIGS. 17A-17B show data from testing of various repressor element-suppressor tRNA AAV constructs in plasmids that can be used to produce either scAAV or ssAAV. FIGS. 17A-17B show graphs of percent GFP positive cells detected in HEK 293 cells (Repressor-) stably expressing a broken GFP reporter (having a R74X premature stop codon or R97X premature stop codon) (FIG. 17A) or percent GFP positive cells detected in HEK293 cells expressing a Tet repressor (Repressor+) that also stably express a broken GFP reporter (having a R74X premature stop codon or R97X premature stop codon) (FIG. 17B) after independent transfection with either scAAV plasmids or ssAAV plasmids having various repressor element-suppressor tRNA constructs as indicated.

FIG. 18 shows a graph of viral titer of cells transfected with AAV plasmids comprising different repressor element-suppressor tRNA AAV constructs.

FIG. 19A shows a graph of percent GFP positive HEK293 cells stably expressing the broken GFP reporter (having an R74X premature stop codon or R97X premature stop codon) that were transduced with AAV plasmids comprising different repressor element-suppressor tRNA constructs.

FIG. 19B shows a graph of GFP MFI of HEK293 cells stably expressing the broken GFP reporter (having an R74X premature stop codon or R97X premature stop codon) that were transduced with AAV plasmids comprising different repressor element-suppressor tRNA constructs.

FIG. 20 is a graph showing the percentage of transduced R168X primary neuron cells (Thy 1.1+) that are MeCP2+ after transduction with AAV comprising repressor element-suppressor tRNA AAV constructs or controls.

FIGS. 21A-21B show a schematic depiction of a portion of the sequence of a pRC2 plasmid compared to the corresponding portion of a pRC2-Ochre plasmid, and data showing that the pRC2-Ochre plasmid improves AAV virion production by reducing suppressor-tRNA mediated readthrough of the AAV capsid protein stop codon. The pRC2 plasmid comprises the sequence coding for the AAV capsid protein VP1 having an intact ochre stop codon and two alternative downstream in-frame ochre stop codons. The pRC2-ochre plasmid comprises the sequence coding for the AAV capsid protein VP1 having an ochre stop codon engineered to an amber stop codon and two alternative downstream in-frame ochre stop codons engineered to amber stop codons.

### DETAILED DESCRIPTION

Translation of a mRNA molecule that contains a premature stop codon can cause premature termination of the translation process to produce a truncated polypeptide or protein. These premature stop codons and subsequent truncated proteins are associated with many severe diseases and disorders, such as Rett Syndrome, Dravet Syndrome, and muscular dystrophies, such as Duchenne Muscular Dystrophy. Certain mutant mRNAs having premature stop codons can also be degraded, which can lead to complete absence or an insufficiency of the resultant protein involved in essential cellular function. There is a need for effective treatments for these diseases and disorders associated with premature stop codons.

One example treatment for diseases and disorders associated with premature stop codons is administration of transfer RNA (tRNA) molecules that suppress premature stop codons to enable readthrough of the premature stop codon in a template messenger RNA (mRNA), which are referred to herein as a “suppressor tRNA” or a “suppressor tRNA molecule”, which are used interchangeably throughout this disclosure. These suppressor tRNAs can comprise an engineered anticodon for recognition of a premature stop codon relative to a wild type tRNA and at least partially transform translation of a premature stop codon into a sense codon, such as, for example by adding a corrective (e.g., non-disease-causing) amino acid to the growing peptide.

Often, vectors are used to deliver the suppressor tRNAs to a subject in need thereof, for example a subject suffering from a disease or disorder associated with premature stop codon(s). The vectors can be viral vectors. The suppressor tRNA molecules or vectors encoding the suppressor tRNA molecules can be packaged into a virus for virus particle-mediated delivery of the suppressor tRNA molecules to a target cell or tissue in vivo. In some cases, the virus can be an adeno-associated virus (AAV).

However, as demonstrated herein, a significant and surprising loss in titer was observed when producing AAV encapsidated suppressor tRNA coding sequences. As also demonstrated herein, when expression of suppressor tRNA sequences is repressed during AAV production, a resulting increase in titer is observed. Therefore, one advantage of the compositions and methods described herein is increased titer during production of AAV encapsidated suppressor tRNA coding sequence(s). Having the ability to increase the titer during production of AAV encapsidated suppressor tRNA coding sequences allows clinicians, medical professionals, laboratory personnel, and researchers to produce amounts of AAV encapsidated suppressor tRNA coding sequence(s) for administration to a patient in need thereof.

As further described herein, in a therapeutic context, the compositions and methods described herein allow for expression of the conditionally repressible suppressor tRNA coding sequences (e.g., in a diseased cell that comprises a premature stop codon, such as Rett syndrome, Dravet syndrome, or Duchenne Muscular Dystrophy). Therefore, in some cases, another advantage of the compositions and methods described herein is that, while in the production environment expression of the suppressor tRNA is repressed, in a therapeutic context, the suppressor tRNA is expressed, resulting in read-through of the premature stop codon(s) and treatment of the disease.

Alternatively to repression of suppressor tRNA during AAV production, sequences coding for one or more proteins needed for viral production can be engineered from the stop codon recognized by the suppressor tRNA to a different stop codon (e.g., from an opal stop codon recognized by an opal suppressor tRNA to an amber stop codon). Therefore, in some cases, the effect of expression of suppressor tRNA sequences during AAV production on the one or more proteins needed for viral production is reduced, resulting in an increase in titer. This alternative approach, and the associated compositions and methods described herein, also can advantageously increase titer during production of AAV encapsidated suppressor tRNA coding sequence(s). Additionally, this approach allows for encapsidation of the suppressor tRNA not in the context of a conditionally repressible suppressor tRNA. Having the ability to increase the titer during production of AAV encapsidated suppressor tRNA coding sequences allows clinicians, medical professionals, laboratory personnel, and researchers to produce amounts of AAV encapsidated suppressor tRNA coding sequence(s) for administration to a patient in need thereof.

The compositions and methods described herein are useful, for example, for silencing tRNA for viral vector production. In some cases, the compositions and methods described herein find use in silencing tRNA for viral vector production using DNA viruses. In some cases, the compositions and methods described herein find use in silencing tRNA for viral vector production, wherein for example, the viral vector is an adeno-associated virus (AAV), adenovirus, herpes virus (e.g., HSV, CMV), or parvovirus (e.g., bocavirus).

Compositions described herein comprising polynucleotides encoding for the suppressor tRNA molecule(s), the suppressor tRNA molecule(s) or the vector(s) encoding the suppressor tRNA molecule(s) can employ an AAV vector for delivery to a subject. Compositions described herein comprising polynucleotides encoding for the suppressor tRNA molecule(s), the suppressor tRNA molecule(s) or the vector(s) encoding the suppressor tRNA molecule(s) can employ an AAV (IV/CNS) vector for delivery to a subject. AAV vector delivery can achieve long-term benefit with single dose and can provide opportunity for multiplexed targeting. Methods can include identifying AAV serotypes that can promote tissue specific-tropism and biodistribution with systemic dosing. Methods can include identifying AAV serotypes that can promote central neuronal tropism and biodistribution with CNS/IV dosing. Also disclosed herein are various constructs for packaging polynucleotides encoding suppressor tRNA molecules in viral vectors, including packaging of: a polynucleotide encoding multiple suppressor tRNA payloads, markers such as GFP or mCherry, stuffer sequences, or any combination thereof. However, more efficient packaging of suppressor tRNA is needed. Disclosed herein are compositions and methods for increasing packaging efficiency of suppressor tRNA in viral vectors, such as AAV, by silencing the suppressor tRNA during packaging. Silencing suppressor tRNA during packaging can include reducing expression of the suppressor tRNA, suppressing the function of expressed suppressor tRNA, or the combination thereof. Disclosed herein are compositions and methods for increasing packaging efficiency of suppressor tRNA in viral vectors, such as AAV, by engineering nucleotide sequences encoding viral proteins to comprise a stop codon that is not the stop codon that the packaged suppressor tRNA binds to. The engineered viral proteins can be viral proteins directly or indirectly involved in AAV production.

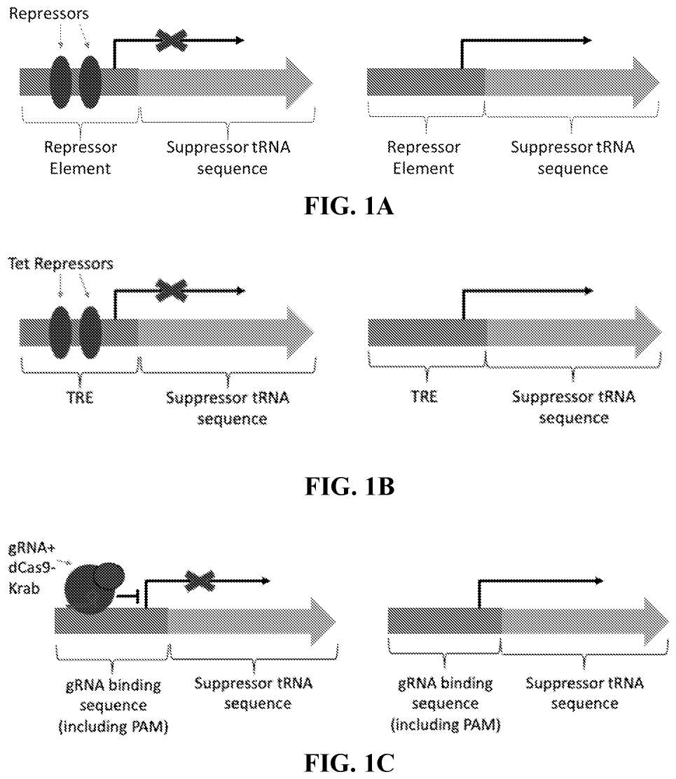

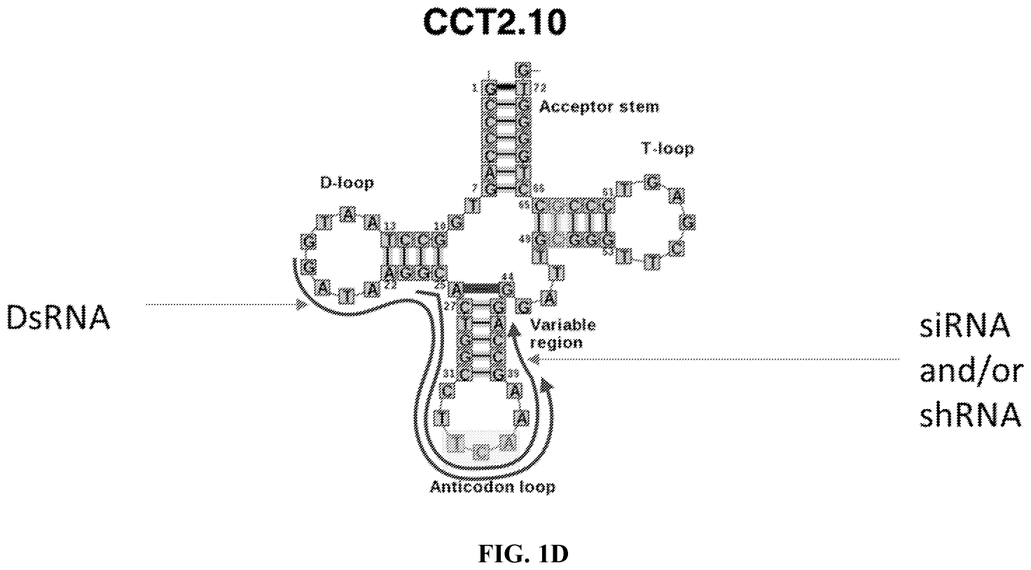

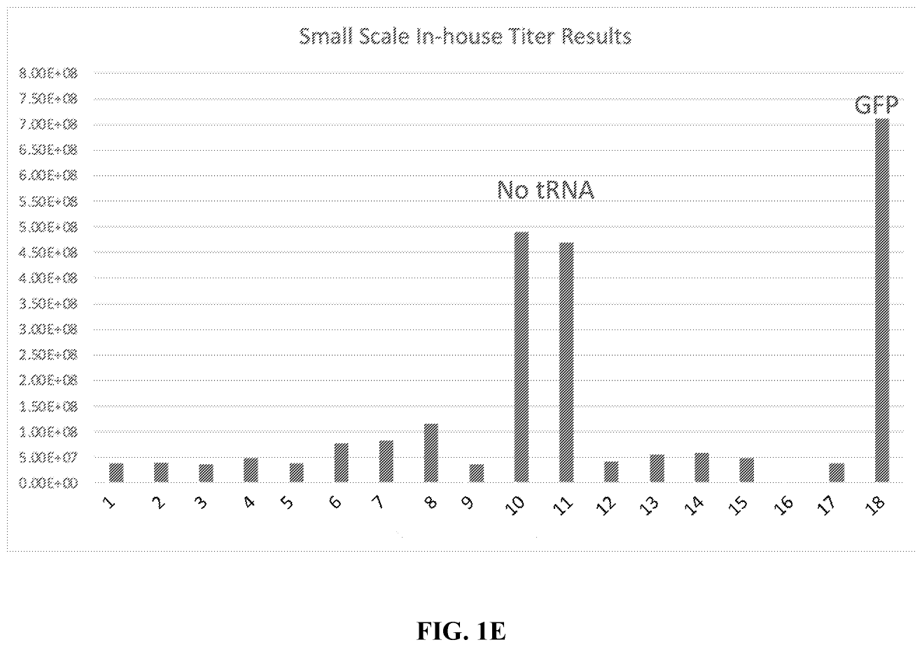

AAV is a non-enveloped virus that can be engineered to deliver nucleic acids, e.g., polynucleotides encoding suppressor tRNA(s), to target cells. The nucleic acids (payload, e.g., a polynucleotide sequence coding for suppressor tRNA(s)) is encapsidated in an AAV capsid protein for delivery to a subject. As described herein, an example discovery is that AAV production of virions comprising polynucleotides encoding suppressor tRNA(s) results in a dramatic decrease in titer of encapsidated genomes relative to virions comprising non-suppressor tRNA nucleic acid payload(s) (e.g., as depicted in FIG. 1E; additionally, suppressor tRNA titration data during AAV production showed that as the amount of suppressor tRNA increased, the produced AAV titer decreased), but that conditional silencing of suppressor tRNA expression or function (e.g., by degradation) and/or engineering stop codons (e.g., by changing one stop codon to another stop codon that is not bound by the suppressor tRNA, e.g., relative to the wild-type sequence) within the AAV genome can rescue this loss in titer during AAV production.

Thus, described herein, are, among other things, compositions and methods for the production and/or packaging of polynucleotide(s) encoding suppressor tRNA molecule(s) in a viral vector, e.g., for AAV mediated delivery to a patient. The compositions and methods for the production and/or packaging of polynucleotide(s) encoding suppressor tRNA molecule(s) in a viral vector, e.g., for AAV mediated delivery to a patient, includes compositions and methods allowing for conditional compositions and methods for selectively mediating production and/or packaging of polynucleotide(s) encoding suppressor tRNA molecule(s) in a viral vector that do not subsequently negatively affect suppressor tRNA molecule(s) function once delivered to a patient. In some cases, the compositions and methods are useful for silencing suppressor tRNA molecule(s), e.g., via repressor elements, e.g., as shown in FIGS. 1A, 1B, or 1C. In some cases, the compositions and methods are useful for silencing suppressor tRNA molecules(s) via knockdown or degradation of functional suppressor tRNA molecule(s), e.g., as shown in FIG. 1D. In some cases, the compositions and methods are useful for increasing titer of encapsidated genomes, e.g., by stop codon engineering, e.g., by changing in-frame stop codon(s) of the same type as the suppressor tRNA molecule(s) to another type of stop codon in viral molecules, e.g., viral proteins, involved in vector production, e.g., AAV production.

Compositions and methods of silencing a suppressor tRNA are disclosed herein. A suppressor tRNA can be conditionally silenced using the disclosed compositions and methods. A composition as disclosed herein can be a polynucleotide comprising a conditionally repressible suppressor tRNA coding sequence. The conditionally repressible suppressor tRNA coding sequence can comprise a sequence coding for a repressor element and a sequence coding for a suppressor tRNA, wherein a repressor can bind to the repressor element and subsequently repress expression of the suppressor tRNA, thus silencing the expression of the suppressor tRNA. The suppressor tRNA is therefore conditionally silenced based upon the presence of the repressor. A composition as disclosed herein can comprise short interference RNA (siRNA), short hairpin RNA (shRNA), dicer-substrate RNA (DsRNA), or any combination thereof, that bind to a suppressor tRNA and subsequently target the bound suppressor tRNA for degradation, thus silencing the suppressor tRNA. The function of the suppressor tRNA is therefore conditionally silenced based upon the presence of the siRNA, shRNA, DsRNA, or any combination thereof.

The compositions as described herein can be used in methods of silencing suppressor tRNA, e.g., via silencing of expression or silencing of function. The suppressor tRNA can be conditionally silenced. For example, the suppressor tRNA can be conditionally silenced using the compositions described herein during production of a virion, wherein the virion encapsidates the polynucleotide comprising the sequence coding for the suppressor tRNA. The titer of virion comprising suppressor tRNA can be higher using the methods as described herein for silencing suppressor tRNA during virion production compared to titer of virion comprising suppressor tRNA wherein the suppressor tRNA is not silenced during virion production. The virion can encapsidate a polynucleotide comprising a conditionally repressible suppressor tRNA coding sequence. The titer of virion encapsidating a polynucleotide comprising a sequence coding for suppressor tRNA can be higher using the methods as described herein for conditionally silencing suppressor tRNA during virion production compared to titer of virion encapsidating a polynucleotide comprising a sequence coding for suppressor tRNA wherein the suppressor tRNA is not silenced during virion production. The virions produced using the methods as described herein can be used to treat a subject having a disease associated with a premature stop codon, such as Rett Syndrome, Dravet Syndrome, and Duchenne Muscular Dystrophy. The virion can encapsidate a polynucleotide comprising a conditionally repressible suppressor tRNA coding sequence, in which the conditions needed for conditional silencing of the suppressor tRNA are absent once delivered to the subject having the disease associated with a premature stop codon, allowing for expression and/or function of the suppressor tRNA in the subject.

The compositions of polynucleotides comprising stop codon engineered nucleotide sequences coding for viral molecule, e.g., a viral protein, as described herein can be used in methods of AAV production, wherein the produced virion encapsidates the polynucleotide comprising a sequence coding for the suppressor tRNA. The titer of virion comprising polynucleotide(s) encoding suppressor tRNA(s) can be higher using the methods as described herein by using polynucleotides comprising stop codon engineered nucleotide sequences coding for viral RNA or protein for virion production (e.g., polynucleotides comprising nucleotide sequences engineered by changing in-frame stop codon(s) of the same type as the suppressor tRNA molecule(s) to another type of stop codon) compared to titer of virion comprising polynucleotide(s) coding for a suppressor tRNA(s) wherein polynucleotides comprising sequences coding for the viral RNA or protein used for virion production are not stop codon engineered (e.g., the polynucleotides comprising nucleotide sequences coding for viral RNA or protein comprise in-frame stop codon(s) of the same type as the suppressor tRNA molecule(s)). The virion can encapsidate polynucleotide comprising the suppressor tRNA coding sequence. The virions produced using the methods as described herein can be used to treat a subject having a disease associated with a premature stop codon, such as Rett Syndrome, Dravet Syndrome, and Duchenne Muscular Dystrophy.

### Stop Codons

In the standard genetic code, there are three different termination codons, as follows:

Stop Codon

| DNA | RNA | Name(s) |
|---|---|---|
| TAG | UAG | “amber” |
| TAA | UAA | “ochre” |
| TGA | UGA | “opal” or “umber” |

In some cases, these stop codons are present within a coding sequence as a result of mutation in the polynucleotide comprising the coding sequence. In some cases, the mutation is a single nucleotide variant, e.g., that changes an in-frame codon for an amino acid to an in-frame stop codon. In some cases, the mutation is an insertion or deletion polymorphism, e.g., that causes a frame shift such that the stop codon is in-frame with the translation initiation site.

Supressor tRNAs

A suppressor tRNA encoded by a sequence in a polynucleotide as described herein is capable of premature stop codon readthrough. For example, a suppressor tRNA is capable of premature stop codon readthrough of an Arg-to-opal stop codon mutation in the MECP2 gene. The suppressor tRNA can enable premature stop codon readthrough of the R168X mutation in the MECP2 gene. In some embodiments, the suppressor tRNA enables premature stop codon readthrough of the R255X mutation in the MECP2 gene. In some embodiments, the suppressor tRNA enables premature stop codon readthrough of the R270X mutation in the MECP2 gene. In some embodiments, the suppressor tRNA enables premature stop codon read-through of the R294X mutation in the MECP2 gene. In some embodiments, the suppressor tRNA enables premature stop codon read-through of the R198X mutation in the MECP2 gene. In some embodiments, the suppressor tRNA enables premature stop codon readthrough of the R453X mutation in the MECP2 gene.

A suppressor tRNA encoded by a sequence in a polynucleotide as described herein can comprise an engineered anticodon for recognition of a premature stop codon. A suppressor tRNA sequence can comprise mutations relative to a parent tRNA sequence that can stabilize or improve one or more regions of the tRNA structure. For example, the suppressor tRNA can have mutations in the acceptor stem, the D-loop, the D-stem, the T-loop, the T-stem, the variable loop, the anticodon stem, the anticodon loop, or any combination thereof.

In some cases, after expression from the polynucleotide comprising a sequence coding for a suppressor tRNA as disclosed herein, the expressed suppressor tRNA is acylated with a canonical amino acid. This expression can occur in a viral production cell, e.g., HEK293 cell. This expression can occur in a cell after delivery, e.g., after AAV delivery to a cell of a subject, e.g., a subject having a disease associated with a premature stop codon, such as Rett Syndrome, Dravet Syndrome, and Duchenne Muscular Dystrophy. In some cases, the expressed suppressor tRNA is acylated with a non-canonical amino acid. A non-canonical amino acid can comprise p-Acetylphenylalanine, p-Propargyloxyphenylalanine, p-Azidophenylalanine, O-methyltyrosine, p-Iodophenylalanine, 3-Iodotyrosine, Biphenylalanine, 2-Aminocaprylic acid, p-Benzoylphenylalanine, o-Nitrobenzylcysteine, o-Nitrobenzylserine, 4,5-Dimethoxy-2-nitrobenzylserine, o-Nitrobenzyllysine, Dansylalanine, Acetyllysine, Methylhistidine, 2-Aminononanoic acid, 2-Aminodecanoic acid, 2-Aminodecanoic acid, Cbz-lysine, Boc-lysine, or Allyloxycarbonyllysine.

In some embodiments, the expressed suppressor tRNA is acylated with an amino acid selected provided in TABLE 1.

TABLE 1
AMINO ACIDS, ONE AND THREE LETTER CODES

| Amino acid | Three letter code | One letter code |
|---|---|---|
| alanine | ala | A |
| arginine | arg | R |
| asparagine | asr | N |
| aspartic acid | asp | D |
| asparagine or aspartic acid | asx | B |
| cysteine | cys | C |
| glutamic acid | glu | E |
| glutamine | gln | Q |
| glutamine or glutamic acid | glx | Z |
| glycine | gly | G |
| histidine | his | H |
| isoleucine | ile | I |
| leucine | leu | L |
| lysine | lys | K |
| methionine | met | M |
| phenylalanine | phe | F |
| proline | pro | P |
| serine | ser | S |
| threonine | thr | T |
| tryptophan | trp | W |
| tyrosine | tyr | Y |
| valine | val | V |

In some cases, the expressed suppressor tRNA is acylated with a lysine. In some cases, the expressed suppressor tRNA is acylated with an arginine. In some cases, the expressed suppressor tRNA is acylated with a histidine. In some cases, the expressed suppressor tRNA is acylated with a glycine. In some cases, the expressed suppressor tRNA is acylated with an alanine. In some cases, the expressed suppressor tRNA is acylated with a valine. In some cases, the expressed suppressor tRNA is acylated with a leucine. In some cases, the expressed suppressor tRNA is acylated with an isoleucine. In some cases, the expressed suppressor tRNA is acylated with a methionine. In some cases, the expressed suppressor tRNA is acylated with a phenylalanine. In some cases, the expressed suppressor tRNA is acylated with a tryptophan. In some cases, the expressed suppressor tRNA is acylated with a proline. In some cases, the expressed suppressor tRNA is acylated with a serine. In some cases, the expressed suppressor tRNA is acylated with a threonine. In some cases, the expressed suppressor tRNA is acylated with a cysteine. In some cases, the expressed suppressor tRNA is acylated with a tyrosine. In some cases, the expressed suppressor tRNA is acylated with an asparagine. In some cases, the expressed suppressor tRNA is acylated with a glutamine. In some cases, the expressed suppressor tRNA is acylated with an aspartate. In some cases, the expressed suppressor tRNA is acylated with a glutamate. In some cases, the expressed suppressor tRNA is acylated with a pyrolysine. In some cases, the expressed suppressor tRNA is acylated with a selenocysteine. In one preferred embodiment, the expressed suppressor tRNA is acylated with an arginine. In another preferred embodiment, the expressed suppressor tRNA is acylated with glutamine.

In some cases, the acylated suppressor tRNA is a lysyl-tRNA, an arginyl-tRNA, a histidyl-tRNA, a glycyl-tRNA, an alanyl-tRNA, a valyl-tRNA, a leucyl-tRNA, an isoleucyl-tRNA, methionyl-tRNA, a phenylalanyl-tRNA, a tryptophanyl-tRNA, a prolyl-tRNA, a seryl-tRNA, a threonyl-tRNA, a cysteinyl-tRNA, a tyrosyl-tRNA, an asparaginyl tRNA, a glutaminyl-tRNA, an aspartyl-tRNA, a pyrrolysyl tRNA, a selenocytstyl tRNA, or a glutamyl-tRNA. In one preferred embodiment, the acylated suppressor tRNA is a nucleotide sequence encoding an arginyl-tRNA. In another preferred embodiment, the acylated suppressor tRNA is a nucleotide sequence encoding a glutaminyl-tRNA.

This disclosure contemplates incorporating specific hybrid tRNAs or orthogonal tRNA/tRNA synthetase pairs. In some cases, a polynucleotide comprises a sequence coding for a suppressor tRNA that is a designer tRNA (such as a hybrid tRNA made from two different naturally occurring tRNAs) or an orthogonal tRNA from other species. In some cases, a polynucleotide comprises a sequence coding for a synthetic or chimeric orthogonal tRNA-tRNA synthetase pair, either together or separately, is used or included. In some cases, polynucleotide(s) comprising sequence(s) coding for synthetic tRNA(s) that interact with naturally occurring tRNA synthetase(s) are included or used. In some cases, a polynucleotide comprising a sequence coding for a pyrrolysyl tRNA or a selenocytstyl tRNA is used in genetic code expansion to incorporate non-canonical amino acids into a suppressor tRNA.

In some cases, the suppressor tRNA is a lysine-tRNA, an arginine-tRNA, a histidine-tRNA, a glycine-tRNA, an alanine-tRNA, a valine-tRNA, a leucine-tRNA, an isoleucine-tRNA, methionine-tRNA, a phenylalanine-tRNA, a tryptophan-tRNA, a proline-tRNA, a serine-tRNA, a threonine-tRNA, a cysteine-tRNA, a tyrosine-tRNA, an asparagine tRNA, an aspartic acid-tRNA, or a glutamine-tRNA. In some cases, the suppressor tRNA is an arginine-tRNA. In some cases, the suppressor tRNA is a glutamine-tRNA.

In some cases, the suppressor tRNA that is encoded by a polynucleotide of the present disclosure is derived from a human tRNA. In some cases, the suppressor tRNA is derived from a non-human tRNA. In some cases, the suppressor tRNA is derived from a tRNA that can be orthogonal to a human tRNA. The suppressor tRNA can be acylated by an amino-acyl synthetase that can recognize the suppressor tRNA and acylate the suppressor tRNA with an amino acid. In some cases, the suppressor tRNA that is encoded by a polynucleotide of the present disclosure is a pre-tRNA. Such pre-tRNA or variant thereof can comprise an intronic sequence. In some cases, an intronic sequence is spliced to produce a mature suppressor tRNA after expression from the polynucleotide comprising a sequence encoding a pre-tRNA. In some cases, an intronic sequence is spliced within a cell containing the suppressor tRNA expressed from the polynucleotide comprising the sequence encoding the pre-tRNA. In some cases, once the intronic sequence is spliced from the suppressor tRNA expressed from the polynucleotide comprising the sequence encoding the pre-tRNA, the mature suppressor tRNA can at least partially enable translation of a premature stop codon into a sense codon. In some cases, a suppressor tRNA with an intron (e.g., suppressor tRNA expressed from the polynucleotide comprising the sequence encoding the pre-tRNA) is more efficient at enabling translation of a premature stop codon into a sense codon as compared to a suppressor tRNA without an intron. The efficiency can be measured by transfecting a vector encoding a suppressor pre-tRNA with an intron into a primary cell line comprising a premature stop codon to which the suppressor pre-tRNA recognizes and comparing the level of premature stop codon readthrough against another comparable cell that has been transfected with a vector encoding a suppressor pre-tRNA without an intron. In some cases, determining the amount of full-length protein can be used to measure premature stop codon readthrough.

A suppressor tRNA that is encoded by a polynucleotide of the present disclosure can be engineered with an anticodon sequence that base pairs with a stop codon, instead of a codon encoding for the amino acid of interest. For example, a suppressor tRNA targeting a premature stop codon at a position in the growing polypeptide in which an Arginine (amino acid of interest) can be normal (e.g., not causing disease). In some cases, an Arg suppressor tRNA with an anticodon sequence that base pairs with the premature stop codon can base pair with the stop codon enabling the suppressor tRNA charged with the Arg to add the Arg to the growing polypeptide molecule, thus, effecting readthrough of the premature stop codon. As another example, a suppressor tRNA targeting a premature stop codon at a position in the growing polypeptide in which a Glutamine (amino acid of interest) can be normal (e.g., not causing disease). In some cases, a Gln suppressor tRNA with an anticodon sequence that base pairs with the premature stop codon can base pair with the stop codon enabling the suppressor tRNA charged with the Gln to add the Gln to the growing polypeptide molecule, thus, effecting readthrough of the premature stop codon.

Suppressor tRNAs that are encoded by the polynucleotides of the present disclosure can recognize and suppress an amber stop codon (UAG), an ochre stop codon (UAA), or an opal stop codon (UGA), or a combination thereof. In some cases, a suppressor tRNA suppresses an amber stop codon (UAG). In some cases, a suppressor tRNA suppresses an ochre stop codon (UAA). In some cases, a suppressor tRNA suppresses an opal stop codon (UGA). In some cases, the present disclosure provides for compositions encoding multiple suppressor tRNAs capable of recognizing and suppressing more than one type of stop codon, e.g., the amber stop codon, the ochre stop codon, and the opal stop codon. A suppressor tRNA expressed by a polynucleotide of as described herein that can recognize and suppress an amber stop codon can comprise an Arginine (Arg) tRNA isodecoder. A suppressor tRNA expressed by a polynucleotide of as described herein can comprise an Arginine (Arg) tRNA isoacceptor that has been engineered to recognize and suppress an amber stop codon. A suppressor tRNA expressed by a polynucleotide of as described herein that can recognize and suppress an amber stop codon can comprise a Glutamine (Gln) tRNA isodecoder. A suppressor tRNA expressed by a polynucleotide of as described herein that can recognize and suppress an ochre stop codon can comprise an Arginine (Arg) tRNA isodecoder. A suppressor tRNA expressed by a polynucleotide of as described herein that can recognize and suppress an ochre stop codon can comprise a Glutamine (Gln) tRNA isodecoder. A suppressor tRNA expressed by a polynucleotide of as described herein that can recognize and suppress an opal stop codon can comprise an Arginine (Arg) tRNA isodecoder.

In some embodiments, a suppressor tRNA expressed by a polynucleotide of as described herein that can recognize and suppress an amber stop codon can have a nucleic acid sequence similarity of about 70% to about 100% to a naturally occurring tRNA isodecoder nucleic acid sequence. In some embodiments, a suppressor tRNA expressed by a polynucleotide of as described herein that can recognize and suppress an amber stop codon can have a nucleic acid sequence similarity of about 70% to about 100% to a naturally occurring Gln tRNA isodecoder nucleic acid sequence. In some embodiments, a suppressor tRNA expressed by a polynucleotide of as described herein that can recognize and suppress an amber stop codon can have an amino acid sequence similarity of about 70% to about 100% to a naturally occurring tRNA isodecoder amino acid sequence. In some embodiments, a suppressor tRNA expressed by a polynucleotide of as described herein that can recognize and suppress an amber stop codon can have an amino acid sequence similarity of about 70% to about 100% to a naturally occurring Gln tRNA isodecoder amino acid sequence. In some embodiments, a suppressor tRNA expressed by a polynucleotide of as described herein that can recognize and suppress an ochre stop codon can have a nucleic acid sequence similarity of about 70% to about 100% to a naturally occurring tRNA isodecoder nucleic acid. In some embodiments, a suppressor tRNA expressed by a polynucleotide of as described herein that can recognize and suppress an ochre stop codon can have a nucleic acid sequence similarity of about 70% to about 100% to a naturally occurring Gln tRNA isodecoder nucleic acid. In some embodiments, a suppressor tRNA expressed by a polynucleotide of as described herein that can recognize and suppress an ochre stop codon can have an amino acid sequence similarity of about 70% to about 100% to a naturally occurring tRNA isodecoder amino acid. In some embodiments, a suppressor tRNA expressed by a polynucleotide of as described herein that can recognize and suppress an ochre stop codon can have an amino acid sequence similarity of about 70% to about 100% to a naturally occurring Gln tRNA isodecoder amino acid. In some embodiments, a suppressor tRNA expressed by a polynucleotide of as described herein that can recognize and suppress an amber stop codon can have a nucleic acid sequence similarity of about 70% to about 100% to a naturally occurring Arg tRNA isodecoder nucleic acid. In some embodiments, a suppressor tRNA expressed by a polynucleotide of as described herein that can recognize and suppress an amber stop codon can have an amino acid sequence similarity of about 70% to about 100% to a naturally occurring Arg tRNA isodecoder amino acid. In some embodiments, a suppressor tRNA expressed by a polynucleotide of as described herein that can recognize and suppress an ochre stop codon can have a nucleic acid sequence similarity of about 70% to about 100% to a naturally occurring Arg tRNA isodecoder nucleic acid. In some embodiments, a suppressor tRNA expressed by a polynucleotide of as described herein that can recognize and suppress an ochre stop codon can have an amino acid sequence similarity of about 70% to about 100% to a naturally occurring Arg tRNA isodecoder amino acid. In some embodiments, a suppressor tRNA expressed by a polynucleotide of as described herein that can recognize and suppress an opal stop codon can have a nucleic acid sequence similarity of about 70% to about 100% to a naturally occurring tRNA isodecoder nucleic acid. In some embodiments, a suppressor tRNA expressed by a polynucleotide of as described herein that can recognize and suppress an opal stop codon can have a nucleic acid sequence similarity of about 70% to about 100% to a naturally occurring Arg tRNA isodecoder nucleic acid. In some embodiments, a suppressor tRNA expressed by a polynucleotide of as described herein that can recognize and suppress an opal stop codon can have an amino acid sequence similarity of about 70% to about 100% to a naturally occurring tRNA isodecoder amino acid. In some embodiments, a suppressor tRNA expressed by a polynucleotide of as described herein that can recognize and suppress an opal stop codon can have an amino acid sequence similarity of about 70% to about 100% to a naturally occurring Arg tRNA isodecoder amino acid.

A suppressor tRNA expressed by a polynucleotide of as described herein with an anticodon configured to base pair with any one of the stop codons described herein can be charged with any one of the amino acids (canonical or noncanonical) described herein. A suppressor tRNA expressed by a polynucleotide of as described herein comprising an anticodon engineered to base pair with any one of the stop codons described herein can be charged with any one of the amino acids (canonical or noncanonical) described herein.

In some embodiments, an mRNA targeted by a suppressor tRNA can comprise one, two, three, four, or five premature stop codons. Accordingly, a suppressor tRNA expressed by a polynucleotide as described herein can produce readthrough of the one or more premature stop codons, at least partially restoring a substantially full-length polypeptide. In some cases, at least partially restoring a substantially full-length polypeptide can comprise at least partially treating a disease or condition. In some cases, a suppressor tRNA expressed by a polynucleotide as described herein can restore about 30% to about 99% of PTC readthroughs. In some cases, a suppressor tRNA can restore about 40% to about 45%, about 40% to about 50%, about 40% to about 55%, about 40% to about 60%, about 40% to about 65%, about 40% to about 70%, about 40% to about 75%, about 40% to about 80%, about 40% to about 85%, about 40% to about 90%, about 40% to about 95%, about 45% to about 50%, about 45% to about 55%, about 45% to about 60%, about 45% to about 65%, about 45% to about 70%, about 45% to about 75%, about 45% to about 80%, about 45% to about 85%, about 45% to about 90%, about 45% to about 95%, about 50% to about 55%, about 50% to about 60%, about 50% to about 65%, about 50% to about 70%, about 50% to about 75%, about 50% to about 80%, about 50% to about 85%, about 50% to about 90%, about 50% to about 95%, about 55% to about 60%, about 55% to about 65%, about 55% to about 70%, about 55% to about 75%, about 55% to about 80%, about 55% to about 85%, about 55% to about 90%, about 55% to about 95%, about 60% to about 65%, about 60% to about 70%, about 60% to about 75%, about 60% to about 80%, about 60% to about 85%, about 60% to about 90%, about 60% to about 95%, about 65% to about 70%, about 65% to about 75%, about 65% to about 80%, about 65% to about 85%, about 65% to about 90%, about 65% to about 95%, about 70% to about 75%, about 70% to about 80%, about 70% to about 85%, about 70% to about 90%, about 70% to about 95%, about 75% to about 80%, about 75% to about 85%, about 75% to about 90%, about 75% to about 95%, about 80% to about 85%, about 80% to about 90%, about 80% to about 95%, about 85% to about 90%, about 85% to about 95%, or about 90% to about 95% of PTC readthroughs. In some cases, a suppressor tRNA expressed by a polynucleotide as described herein can restore at least about 40% of PTC readthroughs. In some cases, two or more stop codons can be the same stop codon. In some cases, two or more stop codons can be different stop codons. In some instances, one type of suppressor tRNA expressed by a polynucleotide as described herein can be used to at least partially restore a full-length polypeptide when a target mRNA contains two or more stop codons that are the same stop codon. In some instances, more than one type of a suppressor tRNA expressed by a polynucleotide as described herein can be used to at least partially restore a full-length polypeptide when a target mRNA contains two or more stop codons that are different stop codons. In some embodiments, a suppressor tRNA can reduce or prevent nonsense-mediated decay of the target mRNA.

The suppressor tRNAs sequences encoded in a polynucleotide disclosed herein can be modified. For example, the suppressor tRNAs sequences encoded in a polynucleotide disclosed herein can be modified relative to a reference tRNA sequence. In some cases, the modification can be a mutation in the sequence of the suppressor tRNA, such as an insertion or a substitution of a nucleotide. In some cases, the sequence that can be mutated can be a DNA sequence, a tRNA sequence, or a pre-tRNA sequence.

The reference tRNA sequence can be a wild-type tRNA sequence or a previously identified suppressor tRNA sequence, such that the new suppressor tRNA sequence has more than one mutation. In this context, the wild-type tRNA sequence or the previously identified suppressor tRNA sequence can be referred to as a “backbone” or “parental” tRNA sequence.

A reference tRNA can be a wild-type tRNA or a previously identified suppressor tRNA, such that the new suppressor tRNA has more than one mutation. In this context, the wild-type tRNA or the previously identified suppressor tRNA sequence can be referred to as a “backbone” or “parental” tRNA.

Mutations can be made in any region of the sequence coding for the suppressor tRNA including, but not limited to, the acceptor stem, anticodon stem, D-loop, D stem, and T-loop, T-stem, or the variable region or loop to produce the suppressor tRNAs encoded in a polynucleotide of the present disclosure. For example, substitutions of nucleotides in the acceptor stem and anticodon stem to increase Watson-Crick base pairing can stabilize the acceptor and anticodon stems of the suppressor tRNA. Non-limiting examples of mutations to the sequences of suppressor tRNAs encoded by polynucleotides disclosed herein are provided in Table 2.

TABLE 2
EXAMPLE MUTATIONS TO POLYNUCLEOTIDES ENDCODING
SUPPRESSOR tRNAS TO PRODUCE SUPPRESSOR tRNAS

| Modifications to SEQ ID NO: 3 | | | Modifications to SEQ ID NO: 6 | | | |
|---|---|---|---|---|---|---|
| 72T > C | 13T > C | 31C > A | 6C > T | 67G > A | 49C > G | 31A > C |
| 2C > G | 22A > G | 39G > T | 65G > C | 50C > T | 64G > A | 39T > G |
| 71G > C | 15A > G | 37A > G | 71T > G | 4T > C | 69A > G | 44A > G |
| 6A > G | 28T > C | 44G > A | 6C > A | 67G > T | 12G > C | 46G > A |
| 67T > C | 42A > G | 50T > C | 23C > G | 27T > C | 43A > G | 2G > C |
| 64A > G | | | 28T > C | 42A > G | 40T > C | 71T > C |

*N>N′ means that N can be substituted for N′. The number preceding N>N′ indicates the nucleotide position with reference to the DNA sequence encoding the engineered tRNA. Thymine (“T”) can be only present in the DNA context, and when in reference to the tRNA sequence, should be understood to refer to uracil (“U”).

The suppressor tRNA sequence encoded in a polynucleotide disclosed herein can comprise a mutation in a T-loop, a T-stem, a D-loop, a D-stem, a variable loop, an anticodon stem, or an anticodon loop. The mutation can be relative to the nucleic acid sequence of any one of SEQ ID NOS: 3-22 or 103-122. In some embodiments, the mutation can comprise 1, 2, 3, 4, 5, 6, 7, 8, 9, 10, 11, 12, 13, 14, or 15 mutations, or a range of mutations defined by any two of the aforementioned numbers of mutations. In some embodiments, the mutation can comprise at least 1, at least 2, at least 3, at least 4, at least 5, at least 6, at least 7, at least 8, at least 9, at least 10, at least 11, at least 12, at least 13, at least 14, or at least 15 mutations. In some embodiments, the mutation can comprise no more than 1, no more than 2, no more than 3, no more than 4, no more than 5, no more than 6, no more than 7, no more than 8, no more than 9, no more than 10, no more than 11, no more than 12, no more than 13, no more than 14, or no more than 15 mutations. In some embodiments, the mutation can comprise a mutation in a T-loop, a T-stem, a D-loop, a D-stem, a variable loop, an anticodon stem, an anticodon loop, or a combination thereof. In some embodiments, the mutation can comprise a mutation in a T-loop. In some embodiments, the mutation can comprise a mutation in a T-stem. In some embodiments, the mutation can comprise a mutation in a D-loop. In some embodiments, the mutation can comprise a mutation in a D-stem. In some embodiments, the mutation can comprise a mutation in a variable loop. In some embodiments, the mutation can comprise a mutation in an anticodon stem. In some embodiments, the mutation can comprise a mutation in an anticodon loop. In some embodiments, the mutation can comprise a mutation in a T-loop and a T-stem. In some embodiments, the mutation can comprise a mutation in a T-loop and a D-loop. In some embodiments, the mutation can comprise a mutation in a T-loop and a D-stem. In some embodiments, the mutation can comprise a mutation in a T-loop and a variable loop. In some embodiments, the mutation can comprise a mutation in a T-loop and an anticodon stem. In some embodiments, the mutation can comprise a mutation in a T-loop and an anticodon loop. In some embodiments, the mutation can comprise a mutation in a T-stem and a D-loop. In some embodiments, the mutation can comprise a mutation in a T-stem and a D-stem. In some embodiments, the mutation can comprise a mutation in a T-stem and a variable loop. In some embodiments, the mutation can comprise a mutation in a T-stem and an anticodon stem. In some embodiments, the mutation can comprise a mutation in a T-stem and an anticodon stem. In some embodiments, the mutation can comprise a mutation in a D-loop and a D-stem. In some embodiments, the mutation can comprise a mutation in a D-loop and a variable loop. In some embodiments, the mutation can comprise a mutation in a D-loop and an anticodon stem. In some embodiments, the mutation can comprise a mutation in a D-loop and an anticodon stem. In some embodiments, the mutation can comprise a mutation in a D-stem and a variable loop. In some embodiments, the mutation can comprise a mutation in a D-stem and an anticodon stem. In some embodiments, the mutation can comprise a mutation in a D-stem and an anticodon stem. In some embodiments, the mutation can comprise a mutation in an anticodon stem and an anticodon stem. In some embodiments, the mutation can comprise a mutation in an anticodon stem and an anticodon stem.

In some cases, the suppressor tRNA sequence encoded in a polynucleotide disclosed herein can comprise 72C, 2G, 71C, 6G, 67C, 64G, 13C, 22G, 15G, 28C, 42G, 31A, 39T, 37G, 44A, or 50C, or any combination thereof, in relation to SEQ ID NO: 3. In some cases, the suppressor tRNA sequence encoded in a polynucleotide can comprise 72C. In some cases, the suppressor tRNA sequence encoded in a polynucleotide can comprise 2G. In some cases, the suppressor tRNA sequence encoded in a polynucleotide can comprise 71G. In some cases, the suppressor tRNA sequence encoded in a polynucleotide can comprise 6G. In some cases, the suppressor tRNA sequence encoded in a polynucleotide can comprise 67C. In some cases, the suppressor tRNA sequence encoded in a polynucleotide can comprise 64G. In some cases, the suppressor tRNA sequence encoded in a polynucleotide can comprise 13C. In some cases, the suppressor tRNA sequence encoded in a polynucleotide comprise 22G. In some cases, the suppressor tRNA sequence encoded in a polynucleotide can comprise 15G. In some cases, the suppressor tRNA sequence encoded in a polynucleotide can comprise 28C. In some cases, the suppressor tRNA sequence encoded in a polynucleotide can comprise 42G. In some cases, the suppressor tRNA sequence encoded in a polynucleotide can comprise 31A. In some cases, the suppressor tRNA sequence encoded in a polynucleotide can comprise 39T. In some cases, the suppressor tRNA sequence encoded in a polynucleotide can comprise 37G. In some cases, the suppressor tRNA sequence encoded in a polynucleotide can comprise 44A. In some cases, the suppressor tRNA sequence encoded in a polynucleotide can comprise 50C. In some cases, the suppressor tRNA sequence encoded in a polynucleotide can have a sequence identity of about 70% to about 100% to SEQ ID NO: 3. In some cases, the suppressor tRNA sequence encoded in a polynucleotide can comprise the sequence of SEQ ID NO: 23, 24, 25, 25, 27, 28, 29, 30, 31 or 32. In some cases, the suppressor tRNA sequence encoded in a polynucleotide can have a sequence identity of about 70% to about 100% to a suppressor tRNA comprising the sequence of SEQ ID NO: 23, 24, 25, 25, 27, 28, 29, 30, 31 or 32.

In some cases, the suppressor tRNA sequence encoded in a polynucleotide as disclosed herein can be a variant of the engineered tRNA TCT1 comprising 2C, 6T, 65C, 71G, 71C, 6A, 23G, 28C, 67A, 50T, 4C, 67T, 27C, 42G, 49G, 64A, 69G, 12C, 43G, 40C, 31C, 39G, 44G or 46A, or any combination thereof, relative to the sequence of SEQ ID NO: 6. In some cases, the suppressor tRNA sequence encoded in a polynucleotide can comprise 6T. In some cases, the suppressor tRNA sequence encoded in a polynucleotide can comprise 65C. In some cases, the suppressor tRNA sequence encoded in a polynucleotide can comprise 2C. In some cases, the suppressor tRNA sequence encoded in a polynucleotide can comprise 71C. In some cases, the suppressor tRNA sequence encoded in a polynucleotide comprise 46A. In some cases, the suppressor tRNA sequence encoded in a polynucleotide can comprise 71G. In some cases, the suppressor tRNA sequence encoded in a polynucleotide can comprise 6A. In some cases, the suppressor tRNA sequence encoded in a polynucleotide can comprise 23G. In some cases, the suppressor tRNA sequence encoded in a polynucleotide can comprise 28C. In some cases, the suppressor tRNA sequence encoded in a polynucleotide can comprise 67A. In some cases, the suppressor tRNA sequence encoded in a polynucleotide can comprise 50T. In some cases, the suppressor tRNA sequence encoded in a polynucleotide can comprise 4C. In some cases, the suppressor tRNA sequence encoded in a polynucleotide can comprise 67T. In some cases, the suppressor tRNA sequence encoded in a polynucleotide can comprise 27C. In some cases, the suppressor tRNA sequence encoded in a polynucleotide can comprise 42G. In some cases, the suppressor tRNA sequence encoded in a polynucleotide can comprise 49G. In some cases, the suppressor tRNA sequence encoded in a polynucleotide can comprise 64A. In some cases, the suppressor tRNA sequence encoded in a polynucleotide can comprise 69G. In some cases, the suppressor tRNA sequence encoded in a polynucleotide can comprise 12C. In some cases, the suppressor tRNA sequence encoded in a polynucleotide can comprise 43G. In some cases, the suppressor tRNA sequence encoded in a polynucleotide can comprise 40C. In some cases, the suppressor tRNA sequence encoded in a polynucleotide comprise 31C. In some cases, the suppressor tRNA sequence encoded in a polynucleotide can comprise 39G. In some cases, the suppressor tRNA sequence encoded in a polynucleotide can comprise 44G. In some cases, the suppressor tRNA sequence encoded in a polynucleotide can have a sequence identity of about 70% to about 100% to the sequence of SEQ ID NO: 6. In some cases, the suppressor tRNA sequence encoded in a polynucleotide can comprise a sequence of any one of SEQ ID NO: 35-SEQ ID NO: 48. In some cases, the suppressor tRNA sequence encoded in a polynucleotide can comprise a sequence selected from SEQ ID NO: 3, SEQ ID NO: 32, SEQ ID NO: 6, or SEQ ID NO: 45. In some cases, the suppressor tRNA sequence encoded in a polynucleotide includes SEQ ID NO: 3, SEQ ID NO: 32, SEQ ID NO: 6, or SEQ ID NO: 45, and SEQ ID NOS: 1 and 2. In some cases, the suppressor tRNA sequence encoded in a polynucleotide can comprise SEQ ID NO: 3. In some cases, the suppressor tRNA sequence encoded in a polynucleotide can comprise SEQ ID NO: 32. In some cases, the suppressor tRNA sequence encoded in a polynucleotide can comprise SEQ ID NO: 6. In some cases, the suppressor tRNA sequence encoded in a polynucleotide can comprise SEQ ID NO: 45. In some cases, the suppressor tRNA sequence encoded in a polynucleotide can have a sequence identity of about 70% to about 100% to any of the suppressor tRNAs comprising a sequence of any one of SEQ ID NO: 35-SEQ ID NO: 48. In some cases, the suppressor tRNA sequence encoded in a polynucleotide can have a sequence identity of 70%, 75%, 80%, 85%, 90%, 95%, or 100% compared to any of the suppressor tRNAs comprising a sequence of any one of SEQ ID NO: 35-SEQ ID NO: 48.

In some cases, the suppressor tRNA sequence encoded in a polynucleotide as disclosed herein can comprise more than or equal to 1, 2, 3, 4, 5, 6, 7, 8, 9, 10, 11, 12, 13, 14, 15, 16, 17, 18, 19, or 20 amino acid substitutions. In some cases, the suppressor tRNA sequence encoded in a polynucleotide can comprise no more than 1, 2, 3, 4, 5, 6, 7, 8, 9, 10, 11, 12, 13, 14, 15, 16, 17, 18, 19, or 20 amino acid substitutions. In some cases, the suppressor tRNA sequence encoded in a polynucleotide does not comprise a substitute in the anticodon. In some cases, the suppressor tRNA sequence encoded in a polynucleotide described herein can be provided in TABLE 3, TABLE 4, or TABLE 5. In some embodiments, the suppressor tRNA sequence encoded in a polynucleotide can be flanked by a 5′ end sequence comprising the sequence of SEQ ID NO: 1 and/or a 3′ end sequence comprising the sequence of SEQ ID NO: 2. In some embodiments, the 5′ end sequence can be the leader sequence of the suppressor tRNA sequence or a variant thereof. In some embodiments, the 3′ end sequence can be the poly-T termination signal and a trailer sequence of the suppressor tRNA sequence or a variant thereof. In some embodiments, the suppressor tRNA sequence encoded in a polynucleotide of any sequence of TABLE 3, TABLE 4, or TABLE 5 can be flanked by a 5′ end sequence comprising the sequence of SEQ ID NO: 1 and/or a 3′ end sequence comprising the sequence of SEQ ID NO: 2.

TABLE 3
EXEMPLARY TRNA SEQUENCES FOR SUPPRESSION OF OPAL STOP
CODONS

| | SEQ | | SEQ | |
|---|---|---|---|---|
| | ID | 5′ to 3′ tRNA sequence | ID | 5′ to 3′ tRNA sequence |
| Name | NO: | (DNA) | NO: | (RNA) |
| 5′ end | 1 | GGTGGTATTGCCCGGAGTAGAA | 101 | GGUGGUAUUGCCCGGAGUAGAAG |
| of | | GCACCTGTGACTAACTGTGCCA | | CACCUGUGACUAACUGUGCCAA |
| tRNA | | A | | |
| 3′ end | 2 | TTTTTTGATGGCCAGCTCCGAG | 102 | UUUUUUGAUGGCCAGCUCCGAGG |
| of | | GATCTAGCGTACTGAGTCGCCC | | AUCUAGCGUACUGAGUCGCCCAG |
| tRNA | | AGTCT | | UCU |
| CCT2 | 3 | GCCCCAGTGGCCTAATGGATAA | 103 | GCCCCAGUGGCCUAAUGGAUAAG |
| | | GGCACTGGCCTTCAAAGCCAGG | | GCACUGGCCUUCAAAGCCAGGGA |
| | | GATTGTGGGTTCGAGTCCCACC | | UUGUGGGUUCGAGUCCCACCUGG |
| | | TGGGGTG | | GGUG |
| CCT1 | 4 | GCCCCAGTGGCCTAATGGATAA | 104 | GCCCCAGUGGCCUAAUGGAUAAG |
| | | GGCACTGGCCTTCAAAGCCAGG | | GCACUGGCCUUCAAAGCCAGGGA |
| | | GATTGTGGGTTCGAGTCCCACC | | UUGUGGGUUCGAGUCCCACCUGG |
| | | TGGGGTA | | GGUA |
| CCT3 | 5 | GCCCCGGTGGCCTAATGGATAA | 105 | GCCCCGGUGGCCUAAUGGAUAAG |
| | | GGCATTGGCCTTCAAAGCCAGG | | GCAUUGGCCUUCAAAGCCAGGGA |
| | | GATTGTGGGTTCGAGTCCCACC | | UUGUGGGUUCGAGUCCCACCCGG |
| | | CGGGGTA | | GGUA |
| TCT1 | 6 | GGCTCCGTGGCGCAATGGATAG | 106 | GGCUCCGUGGCGCAAUGGAUAGC |
| | | CGCATTGGACTTCAAATTCAAA | | GCAUUGGACUUCAAAUUCAAAGG |
| | | GGTTCCGGGTTCGAGTCCCGGC | | UUCCGGGUUCGAGUCCCGGCGGA |
| | | GGAGTCG | | GUCG |
| CCT4 | 7 | GCCCCAGTGGCCTAATGGATAA | 107 | GCCCCAGUGGCCUAAUGGAUAAG |
| | | GGCATTGGCCTTCAAAGCCAGG | | GCAUUGGCCUUCAAAGCCAGGGA |
| | | GATTGTGGGTTCGAGTCCCATC | | UUGUGGGUUCGAGUCCCAUCUGG |
| | | TGGGGTG | | GGUG |
| CCT5 | 8 | GCCCCAGTGGCCTGATGGATAA | 108 | GCCCCAGUGGCCUGAUGGAUAAG |
| | | GGTACTGGCCTTCAAAGCCAGG | | GUACUGGCCUUCAAAGCCAGGGA |
| | | GATTGTGGGTTCGAGTTCCACC | | UUGUGGGUUCGAGUUCCACCUGG |
| | | TGGGGTA | | GGUA |
| TCT2 | 9 | GGCTCTGTGGCGCAATGGATAG | 109 | GGCUCUGUGGCGCAAUGGAUAGC |
| | | CGCATTGGACTTCAAATTCAAA | | GCAUUGGACUUCAAAUUCAAAGG |
| | | GGTTGTGGGTTCGAATCCCACC | | UUGUGGGUUCGAAUCCCACCAGA |
| | | AGAGTCG | | GUCG |
| TCT3 | 10 | GGCTCTGTGGCGCAATGGATAG | 110 | GGCUCUGUGGCGCAAUGGAUAGC |
| | | CGCATTGGACTTCAAATTCAAA | | GCAUUGGACUUCAAAUUCAAAGG |
| | | GGTTGTGGGTTCGAGTCCCACC | | UUGUGGGUUCGAGUCCCACCAGA |
| | | AGAGTCG | | GUCG |
| TCT4 | 11 | GTCTCTGTGGCGCAATGGACGA | 111 | GUCUCUGUGGCGCAAUGGACGAG |
| | | GCGCGCTGGACTTCAAATCCAG | | CGCGCUGGACUUCAAAUCCAGAG |
| | | AGGTTCCGGGTTCGAGTCCCGG | | GUUCCGGGUUCGAGUCCCGGCAG |
| | | CAGAGATG | | AGAUG |
| TCT5 | 12 | GGCTCTGTGGCGCAATGGATAG | 112 | GGCUCUGUGGCGCAAUGGAUAGC |
| | | CGCATTGGACTTCAAATTCAAA | | GCAUUGGACUUCAAAUUCAAAGG |
| | | GGTTGCGGGTTCGAGTCCCTCC | | UUGCGGGUUCGAGUCCCUCCAGA |
| | | AGAGTCG | | GUCG |
| TCG1 | 13 | GGCCGCGTGGCCTAATGGATAA | 113 | GGCCGCGUGGCCUAAUGGAUAAG |
| | | GGCGTCTGACTTCAGATCAGAA | | GCGUCUGACUUCAGAUCAGAAGA |
| | | GATTGCAGGTTCGAGTCCTGCC | | UUGCAGGUUCGAGUCCUGCCGCG |
| | | GCGGTCG | | GUCG |
| TCG2 | 14 | GACCACGTGGCCTAATGGATAA | 114 | GACCACGUGGCCUAAUGGAUAAG |
| | | GGCGTCTGACTTCAGATCAGAA | | GCGUCUGACUUCAGAUCAGAAGA |
| | | GATTGAGGGTTCGAATCCCTCC | | UUGAGGGUUCGAAUCCCUCCGUG |
| | | GTGGTTA | | GUUA |
| TCG3 | 15 | GACCGCGTGGCCTAATGGATAA | 115 | GACCGCGUGGCCUAAUGGAUAAG |
| | | GGCGTCTGACTTCAGATCAGAA | | GCGUCUGACUUCAGAUCAGAAGA |
| | | GATTGAGGGTTCGAGTCCCTTC | | UUGAGGGUUCGAGUCCCUUCGUG |
| | | GTGGTCG | | GUCG |
| TCG4 | 16 | GACCACGTGGCCTAATGGATAA | 116 | GACCACGUGGCCUAAUGGAUAAG |
| | | GGCGTCTGACTTCAGATCAGAA | | GCGUCUGACUUCAGAUCAGAAGA |
| | | GATTGAGGGTTCGAATCCCTTC | | UUGAGGGUUCGAAUCCCUUCGUG |
| | | GTGGTTA | | GUUA |
| TCG5 | 17 | GACCACGTGGCCTAATGGATAA | 117 | GACCACGUGGCCUAAUGGAUAAG |
| | | GGCGTCTGACTTCAGATCAGAA | | GCGUCUGACUUCAGAUCAGAAGA |
| | | GATTGAGGGTTCGAATCCCTTC | | UUGAGGGUUCGAAUCCCUUCGUG |
| | | GTGGTTG | | GUUG |
| TCG6 | 18 | GGCCGTGTGGCCTAATGGATAA | 118 | GGCCGUGUGGCCUAAUGGAUAAG |
| | | GGCGTCTGACTTCAGATCAAAA | | GCGUCUGACUUCAGAUCAAAAGA |
| | | GATTGCAGGTTTGAGTTCTGCC | | UUGCAGGUUUGAGUUCUGCCACG |
| | | ACGGTCG | | GUCG |
| CCG1 | 19 | GGCCGCGTGGCCTAATGGATAA | 119 | GGCCGCGUGGCCUAAUGGAUAAG |
| | | GGCGTCTGATTTCAGATCAGAA | | GCGUCUGAUUUCAGAUCAGAAGA |
| | | GATTGAGGGTTCGAGTCCCTTC | | UUGAGGGUUCGAGUCCCUUCGUG |
| | | GTGGTCG | | GUCG |
| CCG2 | 20 | GACCCAGTGGCCTAATGGATAA | 120 | GACCCAGUGGCCUAAUGGAUAAG |
| | | GGCATCAGCCTTCAGAGCTGGG | | GCAUCAGCCUUCAGAGCUGGGGA |
| | | GATTGTGGGTTCGAGTCCCATC | | UUGUGGGUUCGAGUCCCAUCUGG |
| | | TGGGTCG | | GUCG |
| ACG1 | 21 | GGGCCAGTGGCGCAATGGATAA | 121 | GGGCCAGUGGCGCAAUGGAUAAC |
| | | CGCGTCTGACTTCAGATCAGAA | | GCGUCUGACUUCAGAUCAGAAGA |
| | | GATTCCAGGTTCGACTCCTGGC | | UUCCAGGUUCGACUCCUGGCUGG |
| | | TGGCTCG | | CUCG |
| ACG2 | 22 | GGGCCAGTGGCGCAATGGATAA | 122 | GGGCCAGUGGCGCAAUGGAUAAC |
| | | CGCGTCTGACTTCAGATCAGAA | | GCGUCUGACUUCAGAUCAGAAGA |
| | | GATTCTAGGTTCGACTCCTGGC | | UUCUAGGUUCGACUCCUGGCUGG |
| | | TGGCTCG | | CUCG |
| CCT2.1 | 23 | GCCCCAGTGGCCTAATGGATAA | 123 | GCCCCAGUGGCCUAAUGGAUAAG |
| | | GGCACTGGCCTTCAAAGCCAGG | | GCACUGGCCUUCAAAGCCAGGGA |
| | | GATTGTGGGTTCGAGTCCCACC | | UUGUGGGUUCGAGUCCCACCUGG |
| | | TGGGGCG | | GGCG |
| CCT2.2 | 24 | GGCCCAGTGGCCTAATGGATAA | 124 | GGCCCAGUGGCCUAAUGGAUAAG |
| | | GGCACTGGCCTTCAAAGCCAGG | | GCACUGGCCUUCAAAGCCAGGGA |
| | | GATTGTGGGTTCGAGTCCCACC | | UUGUGGGUUCGAGUCCCACCUGG |
| | | TGGGCTG | | GCUG |
| CCT2.3 | 25 | GCCCCGGTGGCCTAATGGATAA | 125 | GCCCCGGUGGCCUAAUGGAUAAG |
| | | GGCACTGGCCTTCAAAGCCAGG | | GCACUGGCCUUCAAAGCCAGGGA |
| | | GATTGTGGGTTCGAGTCCCACC | | UUGUGGGUUCGAGUCCCACCCGG |
| | | CGGGGTG | | GGUG |
| CCT2.4 | 26 | GCCCCAGTGGCCCAATGGATAG | 126 | GCCCCAGUGGCCCAAUGGAUAGG |
| | | GGCACTGGCCTTCAAAGCCAGG | | GCACUGGCCUUCAAAGCCAGGGA |
| | | GATTGTGGGTTCGAGTCCCACC | | UUGUGGGUUCGAGUCCCACCUGG |
| | | TGGGGTG | | GGUG |
| CCT2.5 | 27 | GCCCCAGTGGCCTAGTGGATAA | 127 | GCCCCAGUGGCCUAGUGGAUAAG |
| | | GGCACTGGCCTTCAAAGCCAGG | | GCACUGGCCUUCAAAGCCAGGGA |
| | | GATTGTGGGTTCGAGTCCCACC | | UUGUGGGUUCGAGUCCCACCUGG |
| | | TGGGGTG | | GGUG |
| CCT2.6 | 28 | GCCCCAGTGGCCTAATGGATAA | 128 | GCCCCAGUGGCCUAAUGGAUAAG |
| | | GGCACCGGCCTTCAAAGCCGGG | | GCACCGGCCUUCAAAGCCGGGGA |
| | | GATTGTGGGTTCGAGTCCCACC | | UUGUGGGUUCGAGUCCCACCUGG |
| | | TGGGGTG | | GGUG |
| CCT2.7 | 29 | GCCCCAGTGGCCTAATGGATAA | 129 | GCCCCAGUGGCCUAAUGGAUAAG |
| | | GGCACTGGACTTCAAATCCAGG | | GCACUGGACUUCAAAUCCAGGGA |
| | | GATTGTGGGTTCGAGTCCCACC | | UUGUGGGUUCGAGUCCCACCUGG |
| | | TGGGGTG | | GGUG |
| CCT2.8 | 30 | GCCCCAGTGGCCTAATGGATAA | 130 | GCCCCAGUGGCCUAAUGGAUAAG |
| | | GGCACTGGCCTTCAGAGCCAGG | | GCACUGGCCUUCAGAGCCAGGGA |
| | | GATTGTGGGTTCGAGTCCCACC | | UUGUGGGUUCGAGUCCCACCUGG |
| | | TGGGGTG | | GGUG |
| CCT2.9 | 31 | GCCCCAGTGGCCTAATGGATAA | 131 | GCCCCAGUGGCCUAAUGGAUAAG |
| | | GGCACTGGCCTTCAAAGCCAGA | | GCACUGGCCUUCAAAGCCAGAGA |
| | | GATTGTGGGTTCGAGTCCCACC | | UUGUGGGUUCGAGUCCCACCUGG |
| | | TGGGGTG | | GGUG |
| CCT2.10 | 32 | GCCCCAGTGGCCTAATGGATAA | 132 | GCCCCAGUGGCCUAAUGGAUAAG |
| | | GGCACTGGCCTTCAAAGCCAGG | | GCACUGGCCUUCAAAGCCAGGGA |
| | | GATTGCGGGTTCGAGTCCCGCC | | UUGCGGGUUCGAGUCCCGCCUGG |
| | | TGGGGTG | | GGUG |
| CCT1.1 | 33 | GCCCCAGTGGCCTAATGGATAA | 133 | GCCCCAGUGGCCUAAUGGAUAAG |
| | | GGCACTGGCCTTCAAAGCCAGG | | GCACUGGCCUUCAAAGCCAGGGA |
| | | GATTGTGGGTTCGAGTCCCACC | | UUGUGGGUUCGAGUCCCACCUGG |
| | | TGGGGTG | | GGUG |
| CCT3.1 | 34 | GCCCCGGTGGCCTAATGGATAA | 134 | GCCCCGGUGGCCUAAUGGAUAAG |
| | | GGCATTGGCCTTCAAAGCCAGG | | GCAUUGGCCUUCAAAGCCAGGGA |
| | | GATTGTGGGTTCGAGTCCCACC | | UUGUGGGUUCGAGUCCCACCCGG |
| | | CGGGGTG | | GGUG |
| TCT1.1 | 35 | GGCTCTGTGGCGCAATGGATAG | 135 | GGCUCUGUGGCGCAAUGGAUAGC |
| | | CGCATTGGACTTCAAATTCAAA | | GCAUUGGACUUCAAAUUCAAAGG |
| | | GGTTCCGGGTTCGAGTCCCGGC | | UUCCGGGUUCGAGUCCCGGCAGA |
| | | AGAGTCG | | GUCG |
| TCT1.2 | 36 | GGCTCCGTGGCGCAATGGATAG | 136 | GGCUCCGUGGCGCAAUGGAUAGC |
| | | CGCATTGGACTTCAAATTCAAA | | GCAUUGGACUUCAAAUUCAAAGG |
| | | GGTTGCGGGTTCGAGTCCCGCC | | UUGCGGGUUCGAGUCCCGCCGGA |
| | | GGAGTCG | | GUCG |
| TCT1.3 | 37 | GGCTCCGTGGCGCAATGGATAG | 137 | GGCUCCGUGGCGCAAUGGAUAGC |
| | | CGCATTGGACTTCAAATTCAAA | | GCAUUGGACUUCAAAUUCAAAGG |
| | | GGTTCTGGGTTCGAGTCCCAGC | | UUCUGGGUUCGAGUCCCAGCGGA |
| | | GGAGTCG | | GUCG |
| TCT1.4 | 38 | GGCTCCGTGGCGCAATGGATAG | 138 | GGCUCCGUGGCGCAAUGGAUAGC |
| | | CGCATTGGACTTCAAATTCAAA | | GCAUUGGACUUCAAAUUCAAAGG |
| | | GGTTCCGGGTTCGAGTCCCGGC | | UUCCGGGUUCGAGUCCCGGCGGA |
| | | GGAGCCG | | GCCG |
| TCT1.5 | 39 | GCCTCCGTGGCGCAATGGATAG | 139 | GCCUCCGUGGCGCAAUGGAUAGC |
| | | CGCATTGGACTTCAAATTCAAA | | GCAUUGGACUUCAAAUUCAAAGG |
| | | GGTTCCGGGTTCGAGTCCCGGC | | UUCCGGGUUCGAGUCCCGGCGGA |
| | | GGAGGCG | | GGCG |
| TCT1.6 | 40 | GGCCCCGTGGCGCAATGGATAG | 140 | GGCCCCGUGGCGCAAUGGAUAGC |
| | | CGCATTGGACTTCAAATTCAAA | | GCAUUGGACUUCAAAUUCAAAGG |
| | | GGTTCCGGGTTCGAGTCCCGGC | | UUCCGGGUUCGAGUCCCGGCGGG |
| | | GGGGTCG | | GUCG |
| TCT1.7 | 41 | GGCTCAGTGGCGCAATGGATAG | 141 | GGCUCAGUGGCGCAAUGGAUAGC |
| | | CGCATTGGACTTCAAATTCAAA | | GCAUUGGACUUCAAAUUCAAAGG |
| | | GGTTCCGGGTTCGAGTCCCGGC | | UUCCGGGUUCGAGUCCCGGCUGA |
| | | TGAGTCG | | GUCG |
| TCT1.8 | 42 | GGCTCCGTGGCCCAATGGATAG | 142 | GGCUCCGUGGCCCAAUGGAUAGG |
| | | GGCATTGGACTTCAAATTCAAA | | GCAUUGGACUUCAAAUUCAAAGG |
| | | GGTTCCGGGTTCGAGTCCCGGC | | UUCCGGGUUCGAGUCCCGGCGGA |
| | | GGAGTCG | | GUCG |
| TCT1.9 | 43 | GGCTCCGTGGCGCAATGGATAG | 143 | GGCUCCGUGGCGCAAUGGAUAGC |
| | | CGCACTGGACTTCAAATTCAGA | | GCACUGGACUUCAAAUUCAGAGG |
| | | GGTTCCGGGTTCGAGTCCCGGC | | UUCCGGGUUCGAGUCCCGGCGGA |
| | | GGAGTCG | | GUCG |
| TCT1.10 | 44 | GGCTCCGTGGCGCAATGGATAG | 144 | GGCUCCGUGGCGCAAUGGAUAGC |
| | | CGCATCGGACTTCAAATTCGAA | | GCAUCGGACUUCAAAUUCGAAGG |
| | | GGTTCCGGGTTCGAGTCCCGGC | | UUCCGGGUUCGAGUCCCGGCGGA |
| | | GGAGTCG | | GUCG |
| TCT1.11 | 45 | GGCTCCGTGGCGCAATGGATAG | 145 | GGCUCCGUGGCGCAAUGGAUAGC |
| | | CGCATTGGACTTCAAATCCAAA | | GCAUUGGACUUCAAAUCCAAAGG |
| | | GGTTCCGGGTTCGAGTCCCGGC | | UUCCGGGUUCGAGUCCCGGCGGA |
| | | GGAGTCG | | GUCG |
| TCT1.12 | 46 | GGCTCCGTGGCGCAATGGATAG | 146 | GGCUCCGUGGCGCAAUGGAUAGC |
| | | CGCATTGGCCTTCAAAGTCAAA | | GCAUUGGCCUUCAAAGUCAAAGG |
| | | GGTTCCGGGTTCGAGTCCCGGC | | UUCCGGGUUCGAGUCCCGGCGGA |
| | | GGAGTCG | | GUCG |
| TCT1.13 | 47 | GGCTCCGTGGCGCAATGGATAG | 147 | GGCUCCGUGGCGCAAUGGAUAGC |
| | | CGCATTGGACTTCAAATTCAAG | | GCAUUGGACUUCAAAUUCAAGGG |
| | | GGTTCCGGGTTCGAGTCCCGGC | | UUCCGGGUUCGAGUCCCGGCGGA |
| | | GGAGTCG | | GUCG |
| TCT1.14 | 48 | GGCTCCGTGGCGCAATGGATAG | 148 | GGCUCCGUGGCGCAAUGGAUAGC |
| | | CGCATTGGACTTCAAATTCAAA | | GCAUUGGACUUCAAAUUCAAAGA |
| | | GATTCCGGGTTCGAGTCCCGGC | | UUCCGGGUUCGAGUCCCGGCGGA |
| | | GGAGTCG | | GUCG |

TABLE 4
EXEMPLARY SUPPRESSOR TRNA SEQUENCE FOR OCHRE STOP
CODONS

| | SEQ | | SEQ | |
|---|---|---|---|---|
| | ID | | ID | |
| Name | NO: | 5′ to 3′ tRNA sequence (DNA) | NO: | 5′ to 3′ tRNA sequence (RNA) |
| CTG1-O | 49 | GGTTCCATGGTGTAATGGTTAG | 249 | GGUUCCAUGGUGUAAUGGUUAGC |
| | | CACTCTGGACTTTAAATCCAGC | | ACUCUGGACUUUAAAUCCAGCGA |
| | | GATCCGAGTTCAAATCTCGGTG | | UCCGAGUUCAAAUCUCGGUGGAA |
| | | GAACCT | | CCU |
| CTG2-O | 50 | GGTTCCATGGTGTAATGGTTAG | 250 | GGUUCCAUGGUGUAAUGGUUAGC |
| | | CACTCTGGACTTTAAATCCAGC | | ACUCUGGACUUUAAAUCCAGCGA |
| | | GATCCGAGTTCAAGTCTCGGTG | | UCCGAGUUCAAGUCUCGGUGGAA |
| | | GAACCT | | CCU |
| CTG3-O | 51 | GGTTCCATGGTGTAATGGTGAG | 251 | GGUUCCAUGGUGUAAUGGUGAGC |
| | | CACTCTGGACTTTAAATCCAGC | | ACUCUGGACUUUAAAUCCAGCGA |
| | | GATCCGAGTTCGAGTCTCGGTG | | UCCGAGUUCGAGUCUCGGUGGAA |
| | | GAACCT | | CCU |
| CTG4-O | 52 | GGTTCCATGGTGTAATGGTGAG | 252 | GGUUCCAUGGUGUAAUGGUGAGC |
| | | CACTCTGGACTTTAAATCCAGC | | ACUCUGGACUUUAAAUCCAGCGA |
| | | GATCCGAGTTCGAGTCTCGGTG | | UCCGAGUUCGAGUCUCGGUGGAA |
| | | GAACCT | | CCU |
| CTG5-O | 53 | GGTTCCATGGTGTAATGGTTAG | 253 | GGUUCCAUGGUGUAAUGGUUAGC |
| | | CACTCTGGACTTTAAATCCGGT | | ACUCUGGACUUUAAAUCCGGUAA |
| | | AATCCGAGTTCAAATCTCGGTG | | UCCGAGUUCAAAUCUCGGUGGAA |
| | | GAACCT | | CCU |
| CTG6-O | 54 | GGCCCCATGGTGTAATGGTCAG | 254 | GGCCCCAUGGUGUAAUGGUCAGC |
| | | CACTCTGGACTTTAAATCCAGC | | ACUCUGGACUUUAAAUCCAGCGA |
| | | GATCCGAGTTCAAATCTCGGTG | | UCCGAGUUCAAAUCUCGGUGGGA |
| | | GGACCC | | CCC |
| CTG7-O | 55 | GGTTCCATGGTGTAATGGTAAG | 255 | GGUUCCAUGGUGUAAUGGUAAGC |
| | | CACTCTGGACTTTAAATCCAGC | | ACUCUGGACUUUAAAUCCAGCCA |
| | | CATCTGAGTTCGAGTCTCTGTG | | UCUGAGUUCGAGUCUCUGUGGAA |
| | | GAACCT | | CCU |
| CTG8-O | 56 | GGTTCCATGGTGTAATGGTGAG | 256 | GGUUCCAUGGUGUAAUGGUGAGC |
| | | CACTTTGGACTTTAAATACAGT | | ACUUUGGACUUUAAAUACAGUGA |
| | | GATCAGAGTTCAAGTCTCACTG | | UCAGAGUUCAAGUCUCACUGGGA |
| | | GGACCT | | CCU |
| CTG9-O | 57 | GGTTCCATGGGTTAATGGTGAG | 257 | GGUUCCAUGGGUUAAUGGUGAGC |
| | | CACCCTGGACTTTAAATCAAGC | | ACCCUGGACUUUAAAUCAAGCGA |
| | | GATCCGAGTTCAAATCTCGGTG | | UCCGAGUUCAAAUCUCGGUGGUA |
| | | GTACCT | | CCU |
| CTG10-O | 58 | GGTTCCATGGTGTAATGGTGAC | 258 | GGUUCCAUGGUGUAAUGGUGACC |
| | | CACTTTGGACTTTAAATACAGT | | ACUUUGGACUUUAAAUACAGUGA |
| | | GATCAGAGTTCAAGTCTCACTG | | UCAGAGUUCAAGUCUCACUGGAA |
| | | GAACCT | | CCU |
| CTG11-O | 59 | GGTTCCATGGTGTAATGGTGAG | 259 | GGUUCCAUGGUGUAAUGGUGAGG |
| | | GGCTTTGGACTTTAACTACAGT | | GCUUUGGACUUUAACUACAGUGA |
| | | GATCAGAGTTCAAGTCTCAGTG | | UCAGAGUUCAAGUCUCAGUGGGA |
| | | GGACCT | | CCU |
| CTG12-O | 60 | GGTTCCATGGTGTAATGGTAAG | 260 | GGUUCCAUGGUGUAAUGGUAAGC |
| | | CACCCTGGACTTTAAATCCAGC | | ACCCUGGACUUUAAAUCCAGCAA |
| | | AACCAGAGTTCCAGTCTCAGCG | | CCAGAGUUCCAGUCUCAGCGUGG |
| | | TGGACCT | | ACCU |
| CTG13-O | 61 | GGTAGTGTAGTCTACTGGTTAA | 261 | GGUAGUGUAGUCUACUGGUUAAA |
| | | ACGCTTGGGCTTTAACATTAAC | | CGCUUGGGCUUUAACAUUAACGU |
| | | GTCCTGGGTTCAAATCCCAGCT | | CCUGGGUUCAAAUCCCAGCUUUG |
| | | TTGTCA | | UCA |
| TTG1-O | 62 | GGTCCCATGGTGTAATGGTTAG | 262 | GGUCCCAUGGUGUAAUGGUUAGC |
| | | CACTCTGGACTTTAAATCCAGC | | ACUCUGGACUUUAAAUCCAGCGA |
| | | GATCCGAGTTCAAATCTCGGTG | | UCCGAGUUCAAAUCUCGGUGGGA |
| | | GGACCT | | CCU |
| TTG2-O | 63 | GGTCCCATGGTGTAATGGTTAG | 263 | GGUCCCAUGGUGUAAUGGUUAGC |
| | | CACTCTGGACTTTAAATCCAGC | | ACUCUGGACUUUAAAUCCAGCAA |
| | | AATCCGAGTTCGAATCTCGGTG | | UCCGAGUUCGAAUCUCGGUGGGA |
| | | GGACCT | | CCU |
| TTG3-O | 64 | GGCCCCATGGTGTAATGGTTAG | 264 | GGCCCCAUGGUGUAAUGGUUAGC |
| | | CACTCTGGACTTTAAATCCAGC | | ACUCUGGACUUUAAAUCCAGCGA |
| | | GATCCGAGTTCAAATCTCGGTG | | UCCGAGUUCAAAUCUCGGUGGGA |
| | | GGACCT | | CCU |
| TTG4-O | 65 | GGTCCCATGGTGTAATGGTTAG | 265 | GGUCCCAUGGUGUAAUGGUUAGC |
| | | CACTCTGGGCTTTAAATCCAGC | | ACUCUGGGCUUUAAAUCCAGCAA |
| | | AATCCGAGTTCGAATCTTGGTG | | UCCGAGUUCGAAUCUUGGUGGGA |
| | | GGACCT | | CCU |
| TTG5-O | 66 | GGCTGTGTACCTCAGTGGGCAA | 266 | GGCUGUGUACCUCAGUGGGCAAG |
| | | GGGTATGGACTTTAAAGCCAGA | | GGUAUGGACUUUAAAGCCAGACU |
| | | CTATTTGGGTTCAAATCCCAGC | | AUUUGGGUUCAAAUCCCAGCUUG |
| | | TTGGCCT | | GCCU |
| TTG6-O | 67 | GACCATGTGGCCTAAGGGAAAA | 267 | GACCAUGUGGCCUAAGGGAAAAG |
| | | GACATCTCACTTTAGGTCAGAA | | ACAUCUCACUUUAGGUCAGAAGA |
| | | GATTGAGGGTTCAAGTCCTTTC | | UUGAGGGUUCAAGUCCUUUCAUG |
| | | ATGGTCA | | GUCA |
| tRX-Gln- | 68 | GGTACAGTGTTAAAGGGGAGAA | 268 | GGUACAGUGUUAAAGGGGAGAAA |
| NNN-1-O | | AAATTGCTGACTTTAAATACAG | | AAUUGCUGACUUUAAAUACAGUA |
| | | TAGACCTAGGTTTGAATCCTGG | | GACCUAGGUUUGAAUCCUGGCUU |
| | | CTTTACCA | | UACCA |

TABLE 5
EXEMPLARY SUPPRESSOR TRNA SEQUENCE FOR AMBER STOP
CODONS

| | SEQ | | SEQ | |
|---|---|---|---|---|
| | ID | | ID | |
| Name | NO: | 5′ to 3′ tRNA sequence (DNA) | NO: | 5′ to 3′ tRNA sequence (RNA) |
| CTG1-A | 69 | GGTTCCATGGTGTAATGGTTAG | 269 | GGUUCCAUGGUGUAAUGGUUAGC |
| | | CACTCTGGACTCTAAATCCAGC | | ACUCUGGACUCUAAAUCCAGCGA |
| | | GATCCGAGTTCAAATCTCGGTG | | UCCGAGUUCAAAUCUCGGUGGAA |
| | | GAACCT | | CCU |
| CTG2-A | 70 | GGTTCCATGGTGTAATGGTTAG | 270 | GGUUCCAUGGUGUAAUGGUUAGC |
| | | CACTCTGGACTCTAAATCCAGC | | ACUCUGGACUCUAAAUCCAGCGA |
| | | GATCCGAGTTCAAGTCTCGGTG | | UCCGAGUUCAAGUCUCGGUGGAA |
| | | GAACCT | | CCU |
| CTG3-A | 71 | GGTTCCATGGTGTAATGGTGAG | 271 | GGUUCCAUGGUGUAAUGGUGAGC |
| | | CACTCTGGACTCTAAATCCAGC | | ACUCUGGACUCUAAAUCCAGCGA |
| | | GATCCGAGTTCGAGTCTCGGTG | | UCCGAGUUCGAGUCUCGGUGGAA |
| | | GAACCT | | CCU |
| CTG4-A | 72 | GGTTCCATGGTGTAATGGTAAG | 272 | GGUUCCAUGGUGUAAUGGUAAGC |
| | | CACTCTGGACTCTAAATCCAGC | | ACUCUGGACUCUAAAUCCAGCGA |
| | | GATCCGAGTTCGAGTCTCGGTG | | UCCGAGUUCGAGUCUCGGUGGAA |
| | | GAACCT | | CCU |
| CTG5-A | 73 | GGTTCCATGGTGTAATGGTTAG | 273 | GGUUCCAUGGUGUAAUGGUUAGC |
| | | CACTCTGGACTCTAAATCCGGT | | ACUCUGGACUCUAAAUCCGGUAA |
| | | AATCCGAGTTCAAATCTCGGTG | | UCCGAGUUCAAAUCUCGGUGGAA |
| | | GAACCT | | CCU |
| CTG6-A | 74 | GGCCCCATGGTGTAATGGTCAG | 274 | GGCCCCAUGGUGUAAUGGUCAGC |
| | | CACTCTGGACTCTAAATCCAGC | | ACUCUGGACUCUAAAUCCAGCGA |
| | | GATCCGAGTTCAAATCTCGGTG | | UCCGAGUUCAAAUCUCGGUGGGA |
| | | GGACCC | | CCC |
| CTG7-A | 75 | GGTTCCATGGTGTAATGGTAAG | 275 | GGUUCCAUGGUGUAAUGGUAAGC |
| | | CACTCTGGACTCTAAATCCAGC | | ACUCUGGACUCUAAAUCCAGCCA |
| | | CATCTGAGTTCGAGTCTCTGTG | | UCUGAGUUCGAGUCUCUGUGGAA |
| | | GAACCT | | CCU |
| CTG8-A | 76 | GGTTCCATGGTGTAATGGTGAG | 276 | GGUUCCAUGGUGUAAUGGUGAGC |
| | | CACTTTGGACTCTAAATACAGT | | ACUUUGGACUCUAAAUACAGUGA |
| | | GATCAGAGTTCAAGTCTCACTG | | UCAGAGUUCAAGUCUCACUGGGA |
| | | GGACCT | | CCU |
| CTG9-A | 77 | GGTTCCATGGGTTAATGGTGAG | 277 | GGUUCCAUGGGUUAAUGGUGAGC |
| | | CACCCTGGACTCTAAATCAAGC | | ACCCUGGACUCUAAAUCAAGCGA |
| | | GATCCGAGTTCAAATCTCGGTG | | UCCGAGUUCAAAUCUCGGUGGUA |
| | | GTACCT | | CCU |
| CTG10-A | 78 | GGTTCCATGGTGTAATGGTGAC | 278 | GGUUCCAUGGUGUAAUGGUGACC |
| | | CACTTTGGACTCTAAATACAGT | | ACUUUGGACUCUAAAUACAGUGA |
| | | GATCAGAGTTCAAGTCTCACTG | | UCAGAGUUCAAGUCUCACUGGAA |
| | | GAACCT | | CCU |
| CTG11-A | 79 | GGTTCCATGGTGTAATGGTGAG | 279 | GGUUCCAUGGUGUAAUGGUGAGG |
| | | GGCTTTGGACTCTAACTACAGT | | GCUUUGGACUCUAACUACAGUGA |
| | | GATCAGAGTTCAAGTCTCAGTG | | UCAGAGUUCAAGUCUCAGUGGGA |
| | | GGACCT | | CCUU |
| CTG12-A | 80 | GGTTCCATGGTGTAATGGTAAG | 280 | GGUUCCAUGGUGUAAUGGUAAGC |
| | | CACCCTGGACTCTAAATCCAGC | | ACCCUGGACUCUAAAUCCAGCAA |
| | | AACCAGAGTTCCAGTCTCAGCG | | CCAGAGUUCCAGUCUCAGCGUGG |
| | | TGGACCT | | ACCU |
| CTG13-A | 81 | GGTAGTGTAGTCTACTGGTTAA | 281 | GGUAGUGUAGUCUACUGGUUAAA |
| | | ACGCTTGGGCTCTAACATTAAC | | CGCUUGGGCUCUAACAUUAACGU |
| | | GTCCTGGGTTCAAATCCCAGCT | | CCUGGGUUCAAAUCCCAGCUUUG |
| | | TTGTCA | | UCA |
| TTG1-A | 82 | GGTCCCATGGTGTAATGGTTAG | 282 | GGUCCCAUGGUGUAAUGGUUAGC |
| | | CACTCTGGACTCTAAATCCAGC | | ACUCUGGACUCUAAAUCCAGCGA |
| | | GATCCGAGTTCAAATCTCGGTG | | UCCGAGUUCAAAUCUCGGUGGGA |
| | | GGACCT | | CCUU |
| TTG2-A | 83 | GGTCCCATGGTGTAATGGTTAG | 283 | GGUCCCAUGGUGUAAUGGUUAGC |
| | | CACTCTGGACTCTAAATCCAGC | | ACUCUGGACUCUAAAUCCAGCAA |
| | | AATCCGAGTTCGAATCTCGGTG | | UCCGAGUUCGAAUCUCGGUGGGA |
| | | GGACCT | | CCUU |
| TTG3-A | 84 | GGCCCCATGGTGTAATGGTTAG | 284 | GGCCCCAUGGUGUAAUGGUUAGC |
| | | CACTCTGGACTCTAAATCCAGC | | ACUCUGGACUCUAAAUCCAGCGA |
| | | GATCCGAGTTCAAATCTCGGTG | | UCCGAGUUCAAAUCUCGGUGGGA |
| | | GGACCT | | CCUU |
| TTG4-A | 85 | GGTCCCATGGTGTAATGGTTAG | 285 | GGUCCCAUGGUGUAAUGGUUAGC |
| | | CACTCTGGGCTCTAAATCCAGC | | ACUCUGGGCUCUAAAUCCAGCAA |
| | | AATCCGAGTTCGAATCTTGGTG | | UCCGAGUUCGAAUCUUGGUGGGA |
| | | GGACCT | | CCUU |
| TTG5-A | 86 | GGCTGTGTACCTCAGTGGGCAA | 286 | GGCUGUGUACCUCAGUGGGCAAG |
| | | GGGTATGGACTCTAAAGCCAGA | | GGUAUGGACUCUAAAGCCAGACU |
| | | CTATTTGGGTTCAAATCCCAGC | | AUUUGGGUUCAAAUCCCAGCUUG |
| | | TTGGCCT | | GCCU |
| TTG6-A | 87 | GACCATGTGGCCTAAGGGAAAA | 287 | GACCAUGUGGCCUAAGGGAAAAG |
| | | GACATCTCACTCTAGGTCAGAA | | ACAUCUCACUCUAGGUCAGAAGA |
| | | GATTGAGGGTTCAAGTCCTTTC | | UUGAGGGUUCAAGUCCUUUCAUG |
| | | ATGGTCA | | GUCA |
| tRX-Gln- | 88 | GGTACAGTGTTAAAGGGGAGAA | 288 | GGUACAGUGUUAAAGGGGAGAAA |
| NNN-1-A | | AAATTGCTGACTCTAAATACAG | | AAUUGCUGACUCUAAAUACAGUA |
| | | TAGACCTAGGTTTGAATCCTGG | | GACCUAGGUUUGAAUCCUGGCUU |
| | | CTTTACCA | | UACCA |

Some embodiments refer to a DNA sequence (e.g., any of SEQ ID NOS: 1-48, 49-68, or 69-88). In some embodiments, the DNA sequence can be interchangeable with a similar RNA sequence (e.g., any of SEQ ID NOs: 101-148, 249-268, or 269-288). Some embodiments refer to an RNA sequence. In some embodiments, the RNA sequence can be interchangeable with a similar DNA sequence. In some embodiments, Us and Ts can be interchanged in a sequence provided herein. Some embodiments refer to a DNA sequence such as a DNA sequence provided in TABLE 3, TABLE 4, or TABLE 5. In some embodiments, the DNA sequence can be interchangeable with a similar RNA sequence provided herein, such as a corresponding RNA sequence in TABLE 3, TABLE 4, or TABLE 5. Some embodiments refer to an RNA sequence such as an RNA sequence provided in TABLE 3, TABLE 4, or TABLE 5. In some embodiments, the RNA sequence can be interchangeable with a similar DNA sequence provided herein, such as a corresponding DNA sequence in TABLE 3, TABLE 4, or TABLE 5. In some embodiments, a polynucleotide coding for a suppressor tRNA described herein can comprise the DNA sequence provided in TABLE 3, TABLE 4, or TABLE 5. In some embodiments, a polynucleotide coding for a suppressor tRNA described herein has the RNA sequence provided in TABLE 3, TABLE 4, or TABLE 5. In general, Us and Ts can be interchanged between the sequences, and still be useful for the methods and compositions described herein. Some embodiments describing a suppressor tRNA or suppressor tRNA sequence of a polynucleotide described herein can include a DNA or RNA sequence in TABLE 3, TABLE 4, or TABLE 5. Some embodiments describing a suppressor tRNA or suppressor tRNA sequence of a polynucleotide described herein can include a DNA sequence in TABLE 3. Some embodiments describing a suppressor tRNA or suppressor tRNA sequence of a polynucleotide described herein can include an RNA sequence in TABLE 3, TABLE 4, or TABLE 5. In some cases, a reference to any of SEQ ID NOS: 1-48 or 49-88 can include a reference to a corresponding sequence within SEQ ID NOS: 101-148 or 249-288, or vice versa. In some cases, a reference made herein to a suppressor tRNA comprising a DNA sequence of TABLE 3, TABLE 4, or TABLE 5 can include a tRNA comprising an RNA sequence that is encoded by a polynucleotide comprising the DNA sequence.

In some embodiments, the sequence of the opal stop codon anticodon loop (TCA) in a polynucleotide comprising any one of SEQ ID NOs: 3-48 is interchangeable with a sequence of the amber stop codon anticodon loop (CTA) for readthrough of an amber stop codon or with a sequence of the ochre stop codon anticodon (TTA) for readthrough of an ochre stop codon. In some embodiments, the sequence of the opal stop codon anticodon loop (UCA) in a polynucleotide comprising any one of SEQ ID NOs: 103-148 is interchangeable with a sequence of the amber stop codon anticodon loop (CUA) for readthrough of an amber stop codon or with a sequence of the ochre stop codon anticodon (UUA) for readthrough of an ochre stop codon.

In some embodiments, the sequence of the ochre stop codon anticodon loop (TTA) in a polynucleotide comprising any one of SEQ ID NOs: 49-68 is interchangeable with a sequence of the amber stop codon anticodon loop (CTA) for readthrough of an amber stop codon or with a sequence of the opal stop codon anticodon (TCA) for readthrough of an opal stop codon. In some embodiments, the sequence of the ochre stop codon anticodon loop (UUA) in a polynucleotide comprising any one of SEQ ID NOs: 249-268 is interchangeable with a sequence of the amber stop codon anticodon loop (CUA) for readthrough of an amber stop codon or with a sequence of the opal stop codon anticodon (UCA) for readthrough of an opal stop codon.

In some embodiments, the sequence of the amber stop codon anticodon loop (CTA) in a polynucleotide comprising any one of SEQ ID NOs: 69-88 is interchangeable with a sequence of the opal stop codon anticodon loop (TCA) for readthrough of an opal stop codon or with a sequence of the ochre stop codon anticodon (TTA) for readthrough of an ochre stop codon. In some embodiments, the sequence of the amber stop codon anticodon loop (CUA) in a polynucleotide comprising any one of SEQ ID NOs: 269-288 is interchangeable with a sequence of the opal stop codon anticodon loop (UCA) for readthrough of an opal stop codon or with a sequence of the ochre stop codon anticodon (UUA) for readthrough of an ochre stop codon.

In some embodiments, A can be a nucleobase comprising adenosine in a polynucleotide described herein. In some embodiments, A can be a nucleoside comprising a ribose or a deoxyribose, and adenosine. In some embodiments, A can be a nucleotide comprising a phosphate, a ribose or a deoxyribose, and adenosine. In some embodiments, T can be a nucleobase comprising thymine. In some embodiments, T can be a nucleoside comprising a ribose or a deoxyribose, and thymine. In some embodiments, T can be a nucleotide comprising a phosphate, a ribose or a deoxyribose, and thymine. In some embodiments, U can be a nucleobase comprising uracil. In some embodiments, U can be a nucleoside comprising a ribose or a deoxyribose, and uracil. In some embodiments, U can be a nucleotide comprising a phosphate, a ribose or a deoxyribose, and uracil. In some embodiments, C can be a nucleobase comprising cytosine. In some embodiments, C can be a nucleoside comprising a ribose or a deoxyribose, and cytosine. In some embodiments, C can be a nucleotide comprising a phosphate, a ribose or a deoxyribose, and cytosine. In some embodiments, G can be a nucleobase comprising guanine. In some embodiments, G can be a nucleoside comprising a ribose or a deoxyribose, and guanine. In some embodiments, G can be a nucleotide comprising a phosphate, a ribose or a deoxyribose, and guanine.

In some embodiments, the polynucleotide coding for a suppressor tRNA can comprise a sequence that has about 70% to about 99% identity to any of the sequences of SEQ ID NO: 3, 4, 5, 6, 7, 8, 9, 10, 11, 12, 13, 14, 15, 16, 17, 18, 19, 20, 21, or 22, as measured using BLAST. In some cases, the sequence identity between a polynucleotide coding for a suppressor tRNA and any of the sequences of SEQ ID NO: 3, 4, 5, 6, 7, 8, 9, 10, 11, 12, 13, 14, 15, 16, 17, 18, 19, 20, 21, or 22, can be from about 70% to about 80%, from 80% to about 90%, from about 85% to about 95%, from about 90% to about 95%, from about 95% to about 99%. In some cases, the sequence identity between a polynucleotide coding for a suppressor tRNA and any of the sequences of SEQ ID NO: 3, 4, 5, 6, 7, 8, 9, 10, 11, 12, 13, 14, 15, 16, 17, 18, 19, 20, 21, or 22, can be at least 70%, 71%, 72%, 73%, 74%, 75%, 76%, 77%, 78%, 79%, 80%, 81%, 82%, 83%, 84%, 85%, 86%, 87%, 88%, 90%, 91%, 92%, 93%, 94%, 95%, 96%, 97%, 98%, 99%, or more.

In some cases, the sequence identity between a polynucleotide coding for a suppressor tRNA and any of the sequences of SEQ ID NO: 3, 4, 5, 6, 7, 8, 9, 10, 11, 12, 13, 14, 15, 16, 17, 18, 19, 20, 21, or 22, can be at most about 99%, 98%, 97%, 69%, 59%, 94%, 93%, 92%, 91%, 89%, 88%, 87%, 86%, 85%, 84%, 83%, 82%, 81%, 80%, 79%, 78%, 77%, 76%, 75%, 74%, 73%, 72%, 71%, 70%, or less. For example, a polynucleotide coding for a suppressor tRNA comprising the sequence of SEQ ID NO: 4 can have a sequence identity of about 98.6% to SEQ ID NO: 3.

In some embodiments, the polynucleotide coding for a suppressor tRNA can comprise a sequence that has about 70% to about 99% identity to any of the sequences of SEQ ID NO: 103, 104, 105, 106, 107, 108, 109, 110, 111, 112, 113, 114, 115, 116, 117, 118, 119, 120, 121, or 122, as measured using BLAST. In some cases, the sequence identity between a polynucleotide coding for a suppressor tRNA and any of the sequences of SEQ ID NO: 103, 104, 105, 106, 107, 108, 109, 110, 111, 112, 113, 114, 115, 116, 117, 118, 119, 120, 121, or 122, can be from about 70% to about 80%, from 80% to about 90%, from about 85% to about 95%, from about 90% to about 95%, from about 95% to about 99%. In some cases, the sequence identity between a polynucleotide coding for a suppressor tRNA and any of the sequences of SEQ ID NO: 103, 104, 105, 106, 107, 108, 109, 110, 111, 112, 113, 114, 115, 116, 117, 118, 119, 120, 121, or 122, can be at least 70%, 71%, 72%, 73%, 74%, 75%, 76%, 77%, 78%, 79%, 80%, 81%, 82%, 83%, 84%, 85%, 86%, 87%, 88%, 90%, 91%, 92%, 93%, 94%, 95%, 96%, 97%, 98%, 99%, or more. In some cases, the sequence identity between a polynucleotide coding for a suppressor tRNA and any of the sequences of SEQ ID NO: 103, 104, 105, 106, 107, 108, 109, 110, 111, 112, 113, 114, 115, 116, 117, 118, 119, 120, 121, or 122, can be at most about 99%, 98%, 97%, 69%, 59%, 94%, 93%, 92%, 91%, 89%, 88%, 87%, 86%, 85%, 84%, 83%, 82%, 81%, 80%, 79%, 78%, 77%, 76%, 75%, 74%, 73%, 72%, 71%, 70%, or less. For example, a polynucleotide coding for a suppressor tRNA comprising the sequence of SEQ ID NO: 104 can have a sequence identity of about 98.6% to SEQ ID NO: 103.

In some cases, the polynucleotide coding for a suppressor tRNA can comprise a sequence that has about 70% to about 99% identity to any of the sequences of SEQ ID NOS: 3, 4, 5, or 6, as measured using BLAST. In some cases, the sequence identity between a polynucleotide coding for a suppressor tRNA and any of the sequences of SEQ ID NO: 3, 4, 5, or 6 can be from about 70% to about 80%, from 80% to about 90%, from about 85% to about 95%, from about 90% to about 95%, from about 95% to about 99%. In some cases, the sequence identity between a polynucleotide coding for a suppressor tRNA and any of the sequences of SEQ ID NO: 3, 4, 5, or 6 can be at least 70%, 71%, 72%, 73%, 74%, 75%, 76%, 77%, 78%, 79%, 80%, 81%, 82%, 83%, 84%, 85%, 86%, 87%, 88%, 90%, 91%, 92%, 93%, 94%, 95%, 96%, 97%, 98%, 99%, or more. In some cases, the sequence identity between a polynucleotide coding for a suppressor tRNA and any of the sequences of SEQ ID NO: 3, 4, 5, or 6 can be at most about 99%, 98%, 97%, 69%, 59%, 94%, 93%, 92%, 91%, 89%, 88%, 87%, 86%, 85%, 84%, 83%, 82%, 81%, 80%, 79%, 78%, 77%, 76%, 75%, 74%, 73%, 72%, 71%, 70%, or less. For example, a polynucleotide coding for a suppressor tRNA variant comprising the sequence of SEQ ID NO: 35 can comprise a sequence identity of about 97% to the sequence of SEQ ID NO: 6.

In some cases, a polynucleotide coding for a suppressor tRNA can comprise a sequence that has about 70% to about 99% identity to any of the sequences of SEQ ID NO: 3, 6, 7, 32, or 45. In some cases, the sequence identity between a polynucleotide coding for a suppressor tRNA and any of the sequences of SEQ ID NO: 3, 6, 7, 32, or 45 can be from about 70% to about 80%, from 80% to about 90%, from about 85% to about 95%, from about 90% to about 95%, from about 95% to about 99%.

In some cases, a polynucleotide coding for a suppressor tRNA can comprise a sequence that has about 70% to about 100% identity to a polynucleotide coding for a suppressor tRNA disclosed herein (e.g. comprising a sequence disclosed in Table 3). The polynucleotide coding for a suppressor tRNA sequence can be a sequence of Table 3. In some embodiments, the polynucleotide coding for a suppressor tRNA can comprise SEQ ID NO: 1. The polynucleotide coding for a suppressor tRNA can include SEQ ID NO: 1 or 101, and any of SEQ ID NOS: 3-48. The polynucleotide coding for a suppressor tRNA can include SEQ ID NO: 1 or 101, and any of SEQ ID NOs: 103-148. In some embodiments, the suppressor tRNA sequence of the polynucleotide described herein can comprise SEQ ID NO: 2. The polynucleotide coding for a suppressor tRNA can include any of SEQ ID NOS: 3-48, in addition to SEQ ID NO: 2. The polynucleotide coding for a suppressor tRNA can include any of SEQ ID NOs: 103-148, in addition to SEQ ID NO: 102. The polynucleotide coding for a suppressor tRNA can comprise or consist of SEQ ID NO: 1, any of SEQ ID NOS: 3-48, and SEQ ID NO: 2. The polynucleotide coding for a suppressor tRNA can comprise or consist of SEQ ID NO: 101, any of SEQ ID NOs: 103-148, and SEQ ID NO: 102. In some embodiments, the suppressor tRNA sequence of the polynucleotide described herein can comprise SEQ ID NO: 3. In some embodiments, the suppressor tRNA sequence of the polynucleotide described herein can comprise SEQ ID NO: 4. In some embodiments, the suppressor tRNA sequence can comprise SEQ ID NO: 5. In some embodiments, the suppressor tRNA sequence can comprise SEQ ID NO: 6. In some embodiments, the suppressor tRNA sequence can comprise SEQ ID NO: 7. In some embodiments, the suppressor tRNA sequence can comprise SEQ ID NO: 8. In some embodiments, the suppressor tRNA sequence can comprise SEQ ID NO: 9. In some embodiments, the suppressor tRNA sequence can comprise SEQ ID NO: 10. In some embodiments, the suppressor tRNA sequence can comprise SEQ ID NO: 11. In some embodiments, the suppressor tRNA sequence can comprise SEQ ID NO: 12. In some embodiments, the suppressor tRNA sequence can comprise SEQ ID NO: 13. In some embodiments, the suppressor tRNA sequence can comprise SEQ ID NO: 14. In some embodiments, the suppressor tRNA sequence can comprise SEQ ID NO: 15. In some embodiments, the suppressor tRNA sequence can comprise SEQ ID NO: 16. In some embodiments, the suppressor tRNA sequence can comprise SEQ ID NO: 17. In some embodiments, the suppressor tRNA sequence can comprise SEQ ID NO: 18. In some embodiments, the suppressor tRNA sequence can comprise SEQ ID NO: 19. In some embodiments, the suppressor tRNA sequence can comprise SEQ ID NO: 20. In some embodiments, the suppressor tRNA sequence can comprise SEQ ID NO: 21. In some embodiments, the suppressor tRNA sequence can comprise SEQ ID NO: 22. In some embodiments, the suppressor tRNA sequence can comprise SEQ ID NO: 23. In some embodiments, the suppressor tRNA sequence can comprise SEQ ID NO: 24. In some embodiments, the suppressor tRNA sequence can comprise SEQ ID NO: 25. In some embodiments, the suppressor tRNA sequence can comprise SEQ ID NO: 26. In some embodiments, the suppressor tRNA sequence can comprise SEQ ID NO: 27. In some embodiments, the suppressor tRNA sequence can comprise SEQ ID NO: 28. In some embodiments, the suppressor tRNA sequence can comprise SEQ ID NO: 29. In some embodiments, the suppressor tRNA sequence can comprise SEQ ID NO: 30. In some embodiments, the suppressor tRNA sequence can comprise SEQ ID NO: 31. In some embodiments, the suppressor tRNA sequence can comprise SEQ ID NO: 32. In some embodiments, the suppressor tRNA sequence can comprise SEQ ID NO: 33. In some embodiments, the suppressor tRNA sequence can comprise SEQ ID NO: 34. In some embodiments, the suppressor tRNA sequence can comprise SEQ ID NO: 35. In some embodiments, the suppressor tRNA sequence can comprise SEQ ID NO: 36. In some embodiments, the suppressor tRNA sequence can comprise SEQ ID NO: 37. In some embodiments, the suppressor tRNA sequence can comprise SEQ ID NO: 38. In some embodiments, the suppressor tRNA sequence can comprise SEQ ID NO: 39. In some embodiments, the suppressor tRNA sequence can comprise SEQ ID NO: 40. In some embodiments, the suppressor tRNA sequence can comprise SEQ ID NO: 41. In some embodiments, the suppressor tRNA sequence can comprise SEQ ID NO: 42. In some embodiments, the suppressor tRNA sequence can comprise SEQ ID NO: 43. In some embodiments, the suppressor tRNA sequence can comprise SEQ ID NO: 44. In some embodiments, the suppressor tRNA sequence can comprise SEQ ID NO: 45. In some embodiments, the suppressor tRNA sequence can comprise SEQ ID NO: 46. In some embodiments, the suppressor tRNA sequence can comprise SEQ ID NO: 47. In some embodiments, the suppressor tRNA sequence can comprise SEQ ID NO: 48. In some embodiments, the suppressor tRNA sequence can comprise SEQ ID NO: 101. In some embodiments, the suppressor tRNA sequence can comprise SEQ ID NO: 102. In some embodiments, the suppressor tRNA sequence can comprise SEQ ID NO: 103. In some embodiments, the suppressor tRNA sequence can comprise SEQ ID NO: 104. In some embodiments, the suppressor tRNA sequence can comprise SEQ ID NO: 105. In some embodiments, the suppressor tRNA sequence can comprise SEQ ID NO: 106. In some embodiments, the suppressor tRNA sequence can comprise SEQ ID NO: 107. In some embodiments, the suppressor tRNA sequence can comprise SEQ ID NO: 108. In some embodiments, the suppressor tRNA sequence can comprise SEQ ID NO: 109. In some embodiments, the suppressor tRNA sequence can comprise SEQ ID NO: 110. In some embodiments, the suppressor tRNA sequence can comprise SEQ ID NO: 111. In some embodiments, the suppressor tRNA sequence can comprise SEQ ID NO: 112. In some embodiments, the suppressor tRNA sequence can comprise SEQ ID NO: 113. In some embodiments, the suppressor tRNA sequence can comprise SEQ ID NO: 114. In some embodiments, the suppressor tRNA sequence can comprise SEQ ID NO: 115. In some embodiments, the suppressor tRNA sequence can comprise SEQ ID NO: 116. In some embodiments, the suppressor tRNA sequence can comprise SEQ ID NO: 117. In some embodiments, the suppressor tRNA sequence can comprise SEQ ID NO: 118. In some embodiments, the suppressor tRNA sequence can comprise SEQ ID NO: 119. In some embodiments, the suppressor tRNA sequence can comprise SEQ ID NO: 120. In some embodiments, the suppressor tRNA sequence can comprise SEQ ID NO: 121. In some embodiments, the suppressor tRNA sequence can comprise SEQ ID NO: 122. In some embodiments, the suppressor tRNA sequence can comprise SEQ ID NO: 123. In some embodiments, the suppressor tRNA sequence can comprise SEQ ID NO: 124. In some embodiments, the suppressor tRNA sequence can comprise SEQ ID NO: 125. In some embodiments, the suppressor tRNA sequence can comprise SEQ ID NO: 126. In some embodiments, the suppressor tRNA sequence can comprise SEQ ID NO: 127. In some embodiments, the suppressor tRNA sequence can comprise SEQ ID NO: 128. In some embodiments, the suppressor tRNA sequence can comprise SEQ ID NO: 129. In some embodiments, the suppressor tRNA sequence can comprise SEQ ID NO: 130. In some embodiments, the suppressor tRNA sequence can comprise SEQ ID NO: 131. In some embodiments, the suppressor tRNA sequence can comprise SEQ ID NO: 132. In some embodiments, the suppressor tRNA sequence can comprise SEQ ID NO: 133. In some embodiments, the suppressor tRNA sequence can comprise SEQ ID NO: 134. In some embodiments, the suppressor tRNA sequence can comprise SEQ ID NO: 135. In some embodiments, the suppressor tRNA sequence can comprise SEQ ID NO: 136. In some embodiments, the suppressor tRNA sequence can comprise SEQ ID NO: 137. In some embodiments, the suppressor tRNA sequence can comprise SEQ ID NO: 138. In some embodiments, the suppressor tRNA sequence can comprise SEQ ID NO: 139. In some embodiments, the suppressor tRNA sequence can comprise SEQ ID NO: 140. In some embodiments, the suppressor tRNA sequence can comprise SEQ ID NO: 141. In some embodiments, the suppressor tRNA sequence can comprise SEQ ID NO: 142. In some embodiments, the suppressor tRNA sequence can comprise SEQ ID NO: 143. In some embodiments, the suppressor tRNA sequence can comprise SEQ ID NO: 144. In some embodiments, the suppressor tRNA sequence can comprise SEQ ID NO: 145. In some embodiments, the suppressor tRNA sequence can comprise SEQ ID NO: 146. In some embodiments, the suppressor tRNA sequence can comprise SEQ ID NO: 147. In some embodiments, the suppressor tRNA sequence can comprise SEQ ID NO: 148. In some embodiments, the suppressor tRNA sequence can comprise any one of SEQ ID NOs: 49-88. In some embodiments, the suppressor tRNA sequence can comprise any one of SEQ ID NOs: 249-288.

In some cases, a suppressor tRNA sequence of a polynucleotide described herein can comprise a sequence that has about 70% to about 99% identity to the sequence of SEQ ID NO: 3. In some cases, a suppressor tRNA sequence can comprise a sequence that has about 70% to about 99% identity to the sequence of SEQ ID NO: 103. In some cases, a suppressor tRNA sequence can comprise a sequence that has at least 75% identity compared to the sequence of SEQ ID NO: 3 or 103. In some cases, a suppressor tRNA sequence can comprise a sequence that has at least 80% identity compared to the sequence of SEQ ID NO: 3 or 103. In some cases, a suppressor tRNA sequence can comprise a sequence that has at least 85% identity compared to the sequence of SEQ ID NO: 3 or 103. In some cases, a suppressor tRNA sequence can comprise a sequence that has at least 90% identity compared to the sequence of SEQ ID NO: 3 or 103. In some cases, a suppressor tRNA sequence can comprise a sequence that has at least 91% identity compared to the sequence of SEQ ID NO: 3 or 103. In some cases, a suppressor tRNA sequence can comprise a sequence that has at least 92% identity compared to the sequence of SEQ ID NO: 3 or 103. In some cases, a suppressor tRNA sequence can comprise a sequence that has at least 93% identity compared to the sequence of SEQ ID NO: 3 or 103. In some cases, a suppressor tRNA sequence can comprise a sequence that has at least 94% identity compared to the sequence of SEQ ID NO: 3 or 103. In some cases, a suppressor tRNA sequence can comprise a sequence that has at least 95% identity compared to the sequence of SEQ ID NO: 3 or 103. In some cases, a suppressor tRNA sequence can comprise a sequence that has at least 96% identity compared to the sequence of SEQ ID NO: 3 or 103. In some cases, a suppressor tRNA sequence can comprise a sequence that has at least 97% identity compared to the sequence of SEQ ID NO: 3 or 103. In some cases, a suppressor tRNA sequence can comprise a sequence that has at least 98% identity compared to the sequence of SEQ ID NO: 3 or 103. In some cases, a suppressor tRNA sequence can comprise a sequence that has at least 99% identity compared to the sequence of SEQ ID NO: 3 or 103. In some cases, a suppressor tRNA sequence can comprise a sequence that has 100% identity compared to the sequence of SEQ ID NO: 3 or 103. In some embodiments, the % identity is over a range (e.g. 70-100% of the length) of nucleotides of SEQ ID NO: 3 or 103. In some embodiments, the % identity can be over a range of 70% of the length of nucleotides of SEQ ID NO: 3 or 103. In some embodiments, the % identity can be over a range of 75% of the length of nucleotides of SEQ ID NO: 3 or 103. In some embodiments, the % identity can be over a range of 80% of the length of nucleotides of SEQ ID NO: 3 or 103. In some embodiments, the % identity can be over a range of 85% of the length of nucleotides of SEQ ID NO: 3 or 103. In some embodiments, the % identity is over a range of 90% of the length of nucleotides of SEQ ID NO: 3 or 103. In some embodiments, the % identity can be over a range of 95% of the length of nucleotides of SEQ ID NO: 3 or 103. In some embodiments, the % identity can be over a range of 100% of the length of nucleotides of SEQ ID NO: 3 or 103.

In some cases, a suppressor tRNA sequence of a polynucleotide described herein can comprise a sequence that has about 70% to about 99% identity to the sequence of SEQ ID NO: 32. In some cases, a suppressor tRNA sequence can comprise a sequence that has about 70% to about 99% identity to the sequence of SEQ ID NO: 132. In some cases, a suppressor tRNA sequence can comprise a sequence that has at least 75% identity compared to the sequence of SEQ ID NO: 32 or 132. In some cases, a suppressor tRNA sequence can comprise a sequence that has at least 80% identity compared to the sequence of SEQ ID NO: 32 or 132. In some cases, a suppressor tRNA sequence can comprise a sequence that has at least 85% identity compared to the sequence of SEQ ID NO: 32 or 132. In some cases, a suppressor tRNA sequence can comprise a sequence that has at least 90% identity compared to the sequence of SEQ ID NO: 32 or 132. In some cases, a suppressor tRNA sequence can comprise a sequence that has at least 91% identity compared to the sequence of SEQ ID NO: 32 or 132. In some cases, a suppressor tRNA sequence can comprise a sequence that has at least 92% identity compared to the sequence of SEQ ID NO: 32 or 132. In some cases, a suppressor tRNA sequence can comprise a sequence that has at least 93% identity compared to the sequence of SEQ ID NO: 32 or 132. In some cases, a suppressor tRNA sequence can comprise a sequence that has at least 94% identity compared to the sequence of SEQ ID NO: 32 or 132. In some cases, a suppressor tRNA sequence can comprise a sequence that has at least 95% identity compared to the sequence of SEQ ID NO: 32 or 132. In some cases, a suppressor tRNA sequence can comprise a sequence that has at least 96% identity compared to the sequence of SEQ ID NO: 32 or 132. In some cases, a suppressor tRNA sequence can comprise a sequence that has at least 97% identity compared to the sequence of SEQ ID NO: 32 or 132. In some cases, a suppressor tRNA sequence can comprise a sequence that has at least 98% identity compared to the sequence of SEQ ID NO: 32 or 132. In some cases, a suppressor tRNA sequence can comprise a sequence that has at least 99% identity compared to the sequence of SEQ ID NO: 32 or 132. In some cases, a suppressor tRNA sequence can comprise a sequence that has 100% identity compared to the sequence of SEQ ID NO: 32 or 132. In some embodiments, the % identity can be over a range (e.g. 70-100% of the length) of nucleotides of SEQ ID NO: 32 or 132. In some embodiments, the % identity can be over a range of 70% of the length of nucleotides of SEQ ID NO: 32 or 132. In some embodiments, the % identity can be over a range of 75% of the length of nucleotides of SEQ ID NO: 32 or 132. In some embodiments, the % identity can be over a range of 80% of the length of nucleotides of SEQ ID NO: 32 or 132. In some embodiments, the % identity can be over a range of 85% of the length of nucleotides of SEQ ID NO: 32 or 132. In some embodiments, the % identity can be over a range of 90% of the length of nucleotides of SEQ ID NO: 32 or 132. In some embodiments, the % identity can be over a range of 95% of the length of nucleotides of SEQ ID NO: 32 or 132. In some embodiments, the % identity can be over a range of 100% of the length of nucleotides of SEQ ID NO: 32 or 132.

In some cases, a suppressor tRNA sequence of a polynucleotide described herein can comprise a sequence that has about 70% to about 99% identity to the sequence of SEQ ID NO: 6. In some cases, a suppressor tRNA sequence can comprise a sequence that has about 70% to about 99% identity to the sequence of SEQ ID NO: 106. In some cases, a suppressor tRNA sequence can comprise a sequence that has at least 75% identity compared to the sequence of SEQ ID NO: 6 or 106. In some cases, a suppressor tRNA sequence can comprise a sequence that has at least 80% identity compared to the sequence of SEQ ID NO: 6 or 106. In some cases, a suppressor tRNA sequence can comprise a sequence that has at least 85% identity compared to the sequence of SEQ ID NO: 6 or 106. In some cases, a suppressor tRNA sequence can comprise a sequence that has at least 90% identity compared to the sequence of SEQ ID NO: 6 or 106. In some cases, a suppressor tRNA sequence can comprise a sequence that has at least 91% identity compared to the sequence of SEQ ID NO: 6 or 106. In some cases, a suppressor tRNA sequence can comprise a sequence that has at least 92% identity compared to the sequence of SEQ ID NO: 6 or 106. In some cases, a suppressor tRNA sequence can comprise a sequence that has at least 93% identity compared to the sequence of SEQ ID NO: 6 or 106. In some cases, a suppressor tRNA sequence can comprise a sequence that has at least 94% identity compared to the sequence of SEQ ID NO: 6 or 106. In some cases, a suppressor tRNA sequence can comprise a sequence that has at least 95% identity compared to the sequence of SEQ ID NO: 6 or 106. In some cases, a suppressor tRNA sequence can comprise a sequence that has at least 96% identity compared to the sequence of SEQ ID NO: 6 or 106. In some cases, a suppressor tRNA sequence can comprise a sequence that has at least 97% identity compared to the sequence of SEQ ID NO: 6 or 106. In some cases, a suppressor tRNA sequence can comprise a sequence that has at least 98% identity compared to the sequence of SEQ ID NO: 6 or 106. In some cases, a suppressor tRNA sequence can comprise a sequence that has at least 99% identity compared to the sequence of SEQ ID NO: 6 or 106. In some cases, a suppressor tRNA sequence can comprise a sequence that has 100% identity compared to the sequence of SEQ ID NO: 6 or 106. In some embodiments, the % identity can be over a range (e.g. 70-100% of the length) of nucleotides of SEQ ID NO: 6 or 106. In some embodiments, the % identity can be over a range of 70% of the length of nucleotides of SEQ ID NO: 6 or 106. In some embodiments, the % identity can be over a range of 75% of the length of nucleotides of SEQ ID NO: 6 or 106. In some embodiments, the % identity can be over a range of 80% of the length of nucleotides of SEQ ID NO: 6 or 106. In some embodiments, the % identity can be over a range of 85% of the length of nucleotides of SEQ ID NO: 6 or 106. In some embodiments, the % identity can be over a range of 90% of the length of nucleotides of SEQ ID NO: 6 or 106. In some embodiments, the % identity can be over a range of 95% of the length of nucleotides of SEQ ID NO: 6 or 106. In some embodiments, the % identity can be over a range of 100% of the length of nucleotides of SEQ ID NO: 6 or 106.

In some cases, a suppressor tRNA sequence of a polynucleotide described herein can comprise a sequence that has about 70% to about 99% identity to the sequence of SEQ ID NO: 45. In some cases, a suppressor tRNA sequence can comprise a sequence that has about 70% to about 99% identity to the sequence of SEQ ID NO: 145. In some cases, a suppressor tRNA sequence can comprise a sequence that has at least 75% identity compared to the sequence of SEQ ID NO: 45 or 145. In some cases, a suppressor tRNA sequence can comprise a sequence that has at least 80% identity compared to the sequence of SEQ ID NO: 45 or 145. In some cases, a suppressor tRNA sequence can comprise a sequence that has at least 85% identity compared to the sequence of SEQ ID NO: 45 or 145. In some cases, a suppressor tRNA sequence can comprise a sequence that has at least 90% identity compared to the sequence of SEQ ID NO: 45 or 145. In some cases, a suppressor tRNA sequence can comprise a sequence that has at least 91% identity compared to the sequence of SEQ ID NO: 45 or 145. In some cases, a suppressor tRNA sequence can comprise a sequence that has at least 92% identity compared to the sequence of SEQ ID NO: 45 or 145. In some cases, a suppressor tRNA sequence can comprise a sequence that has at least 93% identity compared to the sequence of SEQ ID NO: 45 or 145. In some cases, a suppressor tRNA can comprise a sequence that has at least 94% identity compared to the sequence of SEQ ID NO: 45 or 145. In some cases, a suppressor tRNA sequence can comprise a sequence that has at least 95% identity compared to the sequence of SEQ ID NO: 45 or 145. In some cases, a suppressor tRNA sequence can comprise a sequence that has at least 96% identity compared to the sequence of SEQ ID NO: 45 or 145. In some cases, a suppressor tRNA sequence can comprise a sequence that has at least 97% identity compared to the sequence of SEQ ID NO: 45 or 145. In some cases, a suppressor tRNA sequence can comprise a sequence that has at least 98% identity compared to the sequence of SEQ ID NO: 45 or 145. In some cases, a suppressor tRNA sequence can comprise a sequence that has at least 99% identity compared to the sequence of SEQ ID NO: 45 or 145. In some cases, a suppressor tRNA sequence can comprise a sequence that has 100% identity compared to the sequence of SEQ ID NO: 45 or 145. In some embodiments, the % identity can be over a range (e.g. 70-100% of the length) of nucleotides of SEQ ID NO: 45 or 145. In some embodiments, the % identity can be over a range of 70% of the length of nucleotides of SEQ ID NO: 45 or 145. In some embodiments, the % identity can be over a range of 75% of the length of nucleotides of SEQ ID NO: 45 or 145. In some embodiments, the % identity can be over a range of 80% of the length of nucleotides of SEQ ID NO: 45 or 145. In some embodiments, the % identity can be over a range of 85% of the length of nucleotides of SEQ ID NO: 45 or 145. In some embodiments, the % identity can be over a range of 90% of the length of nucleotides of SEQ ID NO: 45 or 145. In some embodiments, the % identity can be over a range of 95% of the length of nucleotides of SEQ ID NO: 45 or 145. In some embodiments, the % identity can be over a range of 100% of the length of nucleotides of SEQ ID NO: 45 or 145.

In some cases, a suppressor tRNA sequence of a polynucleotide described herein can comprise a sequence that has about 70% to about 99% identity to the sequence of SEQ ID NO: 7. In some cases, a suppressor tRNA sequence can comprise a sequence that has about 70% to about 99% identity to the sequence of SEQ ID NO: 107. In some cases, a suppressor tRNA sequence can comprise a sequence that has at least 75% identity compared to the sequence of SEQ ID NO: 7 or 107. In some cases, a suppressor tRNA sequence can comprise a sequence that has at least 80% identity compared to the sequence of SEQ ID NO: 7 or 107. In some cases, a suppressor tRNA sequence can comprise a sequence that has at least 85% identity compared to the sequence of SEQ ID NO: 7 or 107. In some cases, a suppressor tRNA sequence can comprise a sequence that has at least 90% identity compared to the sequence of SEQ ID NO: 7 or 107. In some cases, a suppressor tRNA sequence can comprise a sequence that has at least 91% identity compared to the sequence of SEQ ID NO: 7 or 107. In some cases, a suppressor tRNA sequence can comprise a sequence that has at least 92% identity compared to the sequence of SEQ ID NO: 7 or 107. In some cases, a suppressor tRNA sequence can comprise a sequence that has at least 93% identity compared to the sequence of SEQ ID NO: 7 or 107. In some cases, a suppressor tRNA sequence can comprise a sequence that has at least 94% identity compared to the sequence of SEQ ID NO: 7 or 107. In some cases, a suppressor tRNA sequence can comprise a sequence that has at least 95% identity compared to the sequence of SEQ ID NO: 7 or 107. In some cases, a suppressor tRNA sequence can comprise a sequence that has at least 96% identity compared to the sequence of SEQ ID NO: 7 or 107. In some cases, a suppressor tRNA sequence can comprise a sequence that has at least 97% identity compared to the sequence of SEQ ID NO: 7 or 107. In some cases, a suppressor tRNA sequence can comprise a sequence that has at least 98% identity compared to the sequence of SEQ ID NO: 7 or 107. In some cases, a suppressor tRNA sequence can comprise a sequence that has at least 99% identity compared to the sequence of SEQ ID NO: 7 or 107. In some cases, a suppressor tRNA sequence can comprise a sequence that has 100% identity compared to the sequence of SEQ ID NO: 7 or 107. In some embodiments, the % identity can be over a range (e.g. 70-100% of the length) of nucleotides of SEQ ID NO: 7 or 107. In some embodiments, the % identity can be over a range of 70% of the length of nucleotides of SEQ ID NO: 7 or 107. In some embodiments, the % identity can be over a range of 75% of the length of nucleotides of SEQ ID NO: 7 or 107. In some embodiments, the % identity can be over a range of 80% of the length of nucleotides of SEQ ID NO: 7 or 107. In some embodiments, the % identity can be over a range of 85% of the length of nucleotides of SEQ ID NO: 7 or 107. In some embodiments, the % identity can be over a range of 90% of the length of nucleotides of SEQ ID NO: 7 or 107. In some embodiments, the % identity can be over a range of 95% of the length of nucleotides of SEQ ID NO: 7 or 107. In some embodiments, the % identity can be over a range of 100% of the length of nucleotides of SEQ ID NO: 7 or 107.

Some embodiments a composition includes a suppressor tRNA sequence comprising one or more mutations with reference to a sequence provided in any of SEQ ID NOS: 3-22. In some embodiments, the suppressor tRNA sequence of a polynucleotide described herein can comprise at least 70%, at least 80%, at least 85%, at least 90%, at least 92%, at least 95%, at least 97%, at least 99%, or 100% sequence identity to any one of SEQ ID NO: 23-SEQ ID NO: 48.

In some embodiments, the suppressor tRNA sequence of a polynucleotide described herein can comprise a sequence that can be at least 70% identical to SEQ ID NO: 6. In some embodiments, the suppressor tRNA sequence can comprise a sequence that can be at least 75% identical to SEQ ID NO: 6. In some embodiments, the suppressor tRNA sequence can comprise a sequence that can be at least 80% identical to SEQ ID NO: 6. In some embodiments, the suppressor tRNA sequence can comprise a sequence that can be at least 85% identical to SEQ ID NO: 6. In some embodiments, the suppressor tRNA sequence can comprise a sequence that can be at least 90% identical to SEQ ID NO: 6. In some embodiments, the suppressor tRNA sequence can comprise a sequence that can be at least 95% identical to SEQ ID NO: 6.

In some embodiments, the suppressor tRNA sequence of a polynucleotide described herein can comprise a substitution at position 2, 4, 6, 12, 23, 27, 28, 31, 39, 40, 42, 43, 44, 46, 49, 50, 64, 65, 67, 69, or 71, of the sequence of SEQ ID NO: 6. In some embodiments, the suppressor tRNA sequence can comprise a substitution at position 2, 4, 6, 12, 23, 27, 28, 31, 39, 40, 42, 43, 44, 46, 49, 50, 64, 65, 67, 69, or 71, of the sequence of SEQ ID NO: 106. In some embodiments, the suppressor tRNA sequence can comprise a substitution at position 2 of SEQ ID NO: 6 or 106. In some embodiments, the suppressor tRNA sequence can comprise a substitution at position 4 of SEQ ID NO: 6 or 106. In some embodiments, the suppressor tRNA sequence can comprise a substitution at position 6 of SEQ ID NO: 6 or 106. In some embodiments, the suppressor tRNA sequence can comprise a substitution at position 12 of SEQ ID NO: 6 or 106. In some embodiments, the suppressor tRNA sequence can comprise a substitution at position 23 of SEQ ID NO: 6 or 106. In some embodiments, the suppressor tRNA sequence can comprise a substitution at position 27 of SEQ ID NO: 6 or 106. In some embodiments, the suppressor tRNA sequence can comprise a substitution at position 28 of SEQ ID NO: 6 or 106. In some embodiments, the suppressor tRNA sequence can comprise a substitution at position 31 of SEQ ID NO: 6 or 106. In some embodiments, the suppressor tRNA sequence can comprise a substitution at position 39 of SEQ ID NO: 6 or 106. In some embodiments, the suppressor tRNA sequence can comprise a substitution at position 40 of SEQ ID NO: 6 or 106. In some embodiments, the suppressor tRNA sequence can comprise a substitution at position 42 of SEQ ID NO: 6 or 106. In some embodiments, the suppressor tRNA sequence can comprise a substitution at position 43 of SEQ ID NO: 6 or 106. In some embodiments, the suppressor tRNA sequence can comprise a substitution at position 44 of SEQ ID NO: 6 or 106. In some embodiments, the suppressor tRNA sequence can comprise a substitution at position 46 of SEQ ID NO: 6 or 106. In some embodiments, the suppressor tRNA sequence can comprise a substitution at position 49 of SEQ ID NO: 6 or 106. In some embodiments, the suppressor tRNA sequence can comprise a substitution at position 50 of SEQ ID NO: 6 or 106. In some embodiments, the suppressor tRNA sequence can comprise a substitution at position 64 of SEQ ID NO: 6 or 106. In some embodiments, the suppressor tRNA sequence can comprise a substitution at position 65 of SEQ ID NO: 6 or 106. In some embodiments, the suppressor tRNA sequence can comprise a substitution at position 67 of SEQ ID NO: 6 or 106. In some embodiments, the suppressor tRNA sequence can comprise a substitution at position 69 of SEQ ID NO: 6 or 106. In some embodiments, the suppressor tRNA sequence can comprise a substitution at position 71 of SEQ ID NO: 6 or 106. In some embodiments. In some embodiments, the substitution at position 2 can be to a C. In some embodiments. In some embodiments, the substitution at position 4 can be to a C. In some embodiments, the substitution at position 6 can be to a T. In some embodiments, the substitution at position 6 can be to an A. In some embodiments, the substitution at position 12 can be to a C. In some embodiments, the substitution at position 23 can be to a G. In some embodiments, the substitution at position 27 can be to a C. In some embodiments, the substitution at position 28 can be to a C. In some embodiments, the substitution at position 31 can be to a C. In some embodiments, the substitution at position 39 can be to a G. In some embodiments, the substitution at position 40 can be to a C. In some embodiments, the substitution at position 42 can be to a G. In some embodiments, the substitution at position 43 can be to a G. In some embodiments, the substitution at position 44 can be to a G. In some embodiments, the substitution at position 46 can be to an A. In some embodiments, the substitution at position 49 can be to a G. In some embodiments, the substitution at position 50 can be to a T. In some embodiments, the substitution at position 64 can be to an A. In some embodiments, the substitution at position 65 can be to a C. In some embodiments, the substitution at position 67 can be to an A. In some embodiments, the substitution at position 67 can be to a T. In some embodiments, the substitution at position 69 can be to a G. In some embodiments, the substitution at position 71 can be to a C. In some embodiments, the substitution at position 71 can be to a G. In some embodiments, the sequence of the suppressor tRNA can include multiple substitutions. In some embodiments, the sequence of the suppressor tRNA can be identical to SEQ ID NO: 6 or 106, except for the substitution(s). In some embodiments, the suppressor tRNA can exhibit an increased stability in vivo, as compared with a comparable tRNA comprising the sequence provided in SEQ ID NO: 6, as determined by a proxy measurement, a half-life measurement, an amino acid charging efficiency measurement, or a measurement of binding to a synthetase or ribosomal machinery.

In some embodiments, the suppressor tRNA sequence of a polynucleotide described herein can comprise a sequence that can be at least 70% identical to SEQ ID NO: 3. In some embodiments, the suppressor tRNA sequence can comprise a sequence that can be at least 75% identical to SEQ ID NO: 3. In some embodiments, the suppressor tRNA sequence can comprise a sequence that can be at least 80% identical to SEQ ID NO: 3. In some embodiments, the suppressor tRNA sequence can comprise a sequence that can be at least 85% identical to SEQ ID NO: 3. In some embodiments, the suppressor tRNA sequence can comprise a sequence that can be at least 90% identical to SEQ ID NO: 3. In some embodiments, the suppressor tRNA sequence can comprise a sequence that can be at least 95% identical to SEQ ID NO: 3.

In some embodiments, the suppressor tRNA sequence of a polynucleotide described herein can comprise a substitution at position 2, 6, 13, 15, 22, 28, 31, 37, 39, 42, 44, 50, 64, 67, 71, or 72, of SEQ ID NO: 3 or 103. In some embodiments, the suppressor tRNA sequence can comprise a substitution at position 2 of SEQ ID NO: 3 or 103. In some embodiments, the suppressor tRNA sequence can comprise a substitution at position 6 of SEQ ID NO: 3 or 103. In some embodiments, the suppressor tRNA sequence can comprise a substitution at position 13 of SEQ ID NO: 3 or 103. In some embodiments, the suppressor tRNA sequence can comprise a substitution at position 15 of SEQ ID NO: 3 or 103. In some embodiments, the suppressor tRNA sequence can comprise a substitution at position 22 of SEQ ID NO: 3 or 103. In some embodiments, the suppressor tRNA sequence can comprise a substitution at position 28 of SEQ ID NO: 3 or 103. In some embodiments, the suppressor tRNA sequence can comprise a substitution at position 31 of SEQ ID NO: 3 or 103. In some embodiments, the suppressor tRNA sequence can comprise a substitution at position 37 of SEQ ID NO: 3 or 103. In some embodiments, the suppressor tRNA sequence can comprise a substitution at position 39 of SEQ ID NO: 3 or 103. In some embodiments, the suppressor tRNA sequence can comprise a substitution at position 42 of SEQ ID NO: 3 or 103. In some embodiments, the suppressor tRNA sequence can comprise a substitution at position 44 of SEQ ID NO: 3 or 103. In some embodiments, the suppressor tRNA sequence can comprise a substitution at position 50 of SEQ ID NO: 3 or 103. In some embodiments, the suppressor tRNA sequence can comprise a substitution at position 64 of SEQ ID NO: 3 or 103. In some embodiments, the suppressor tRNA sequence can comprise a substitution at position 67 of SEQ ID NO: 3 or 103. In some embodiments, the suppressor tRNA sequence can comprise a substitution at position 71 of SEQ ID NO: 3 or 103. In some embodiments, the suppressor tRNA sequence can comprise a substitution at position 72 of SEQ ID NO: 3 or 103. In some embodiments, the substitution at position 2 can be to a G. In some embodiments, the substitution at position 6 can be to a G. In some embodiments, the substitution at position 13 can be to a C. In some embodiments, the substitution at position 15 can be to a G. In some embodiments, the substitution at position 22 can be to a G. In some embodiments, the substitution at position 28 can be to a C. In some embodiments, the substitution at position 31 can be to an A. In some embodiments, the substitution at position 37 can be to a G. In some embodiments, the substitution at position 39 can be to a T. In some embodiments, the substitution at position 42 can be to a G. In some embodiments, the substitution at position 44 can be to an A. In some embodiments, the substitution at position 50 can be to a C. In some embodiments, the substitution at position 64 can be to a G. In some embodiments, the substitution at position 67 can be to a C. In some embodiments, the substitution at position 71 can be to a C. In some embodiments, the substitution at position 72 can be to a C. In some embodiments, the sequence of the suppressor tRNA includes multiple substitutions. In some embodiments, the sequence of the suppressor tRNA can be identical to SEQ ID NO: 3 or SEQ ID NO: 103, except for the substitution(s). In some embodiments, the suppressor tRNA exhibits an increased stability in vivo, as compared with a comparable tRNA comprising the sequence provided in SEQ ID NO: 3 or SEQ ID NO: 103, as determined by a proxy measurement, a half-life measurement, an amino acid charging efficiency measurement, or a measurement of binding to a synthetase or ribosomal machinery.

In some embodiments, the suppressor tRNA sequence of a polynucleotide described herein can comprise a sequence that can be at least 70% identical to SEQ ID NO: 5, and can comprise a substitution at position 73 of SEQ ID NO: 5. In some embodiments, the substitution at position 73 can be to a G. In some embodiments, the sequence of the suppressor tRNA can include multiple substitutions. In some embodiments, the sequence of the suppressor tRNA can be identical to SEQ ID NO: 5, except for the substitutions. In some embodiments, the suppressor tRNA can exhibit an increased stability in vivo, as compared with a comparable tRNA comprising the sequence provided in SEQ ID NO: 5, as determined by a proxy measurement, a half-life measurement, an amino acid charging efficiency measurement, or a measurement of binding to a synthetase or ribosomal machinery.

Disclosed herein, in some embodiments, are compositions comprising an suppressor tRNA exhibits an increased stability. In some embodiments, the increased stability can comprise increased thermodynamic stability.

### Compositions and Methods for Silencing Suppressor TRNA

Provided herein are compositions and methods for silencing of suppressor tRNA(s), e.g., during the production of packaging of virions comprising the same. In some cases, the silencing comprises pre-transcriptional gene repression. In some cases, the silencing comprises tRNA degradation and/or interference. In some cases, silencing comprises both pre-transcriptional gene repression and tRNA degradation and/or interference. In some cases, the silencing comprises a reduction in the amount or activity of suppressor tRNA(s) relative to a control.

In some cases, silencing comprises a reduction of at least 40%, e.g., at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% in the amount or activity of suppressor tRNA(s) relative to a control. In some cases, silencing comprises a reduction of at least 40% in the amount or activity of suppressor tRNA(s) relative to a control. In some cases, silencing comprises a reduction of at least 45% in the amount or activity of suppressor tRNA(s) relative to a control. In some cases, silencing comprises a reduction of at least 50% in the amount or activity of suppressor tRNA(s) relative to a control. In some cases, silencing comprises a reduction of at least 55% in the amount or activity of suppressor tRNA(s) relative to a control. In some cases, silencing comprises a reduction of at least 60% in the amount or activity of suppressor tRNA(s) relative to a control. In some cases, silencing comprises a reduction of at least 65% in the amount or activity of suppressor tRNA(s) relative to a control. In some cases, silencing comprises a reduction of at least 70% in the amount or activity of suppressor tRNA(s) relative to a control. In some cases, silencing comprises a reduction of at least 75% in the amount or activity of suppressor tRNA(s) relative to a control. In some cases, silencing comprises a reduction of at least 80% in the amount or activity of suppressor tRNA(s) relative to a control. In some cases, silencing comprises a reduction of at least 85% in the amount or activity of suppressor tRNA(s) relative to a control. In some cases, silencing comprises a reduction of at least 90% in the amount or activity of suppressor tRNA(s) relative to a control. In some cases, silencing comprises a reduction of at least 95% in the amount or activity of suppressor tRNA(s) relative to a control. In some cases, silencing comprises a reduction of at least 100% in the amount or activity of suppressor tRNA(s) relative to a control.

In some cases, e.g., in the case of a repressor element-suppressor tRNA construct, the control is an equivalent construct with the suppressor tRNA, but without the repressor element. In some cases, the control is a) expression of the suppressor tRNA in the cell(s) when the repressor is inhibited from binding to the repressor element, optionally wherein the repressor is inhibited from binding to the repressor element by binding of a switching agent to the repressor; or b) expression of the suppressor tRNA from reference cell(s) comprising a vector comprising the conditionally repressible suppressor tRNA sequence, wherein the reference cell(s) do not express the repressor. For example, the repressor is a Tet repressor protein and the switching agent is doxycycline.

Various gene repression systems are known and described in the art, and a person of ordinary skill in the art will understand which systems are suitable for repression based on context. See, e.g., Kallunki et al., “How to Choose the Right Inducible Gene Expression System for Mammalian Studies?” Cells 8:796:doi: 10.3390/cells8080796 (2019).

In some cases, silencing includes the use of an operator system, e.g., an inducible operator system. In some cases, the operator system is a Lac-controlled operator system. In some cases, the operator system is a Tetracycline-controlled operator system. In some cases, the operator system is a cumate-controlled operator system.

In some cases, silencing includes repression. In some cases, silencing includes repression of expression of a suppressor tRNA from a polynucleotide described herein.

In some cases, silencing includes the use of artificial transcription factors (ATFs) where a programmable DNA-binding domain (e.g., zinc fingers, transcription activator-like effectors, CRISPR-Cas) is coupled with an effector domain (e.g., KRAB) to alter gene transcription. In some cases, silencing includes targeted protein degradation (TPD), targeted RNA degradation, or RNA interference (RNAi).

Compositions for silencing suppressor tRNA are disclosed herein. A composition can comprise a conditionally repressible suppressor tRNA coding sequence. The composition can comprise a polynucleotide coding for a conditionally repressible suppressor tRNA coding sequence. In some embodiments, the conditionally repressible suppressor tRNA coding sequence comprises a sequence coding for a repressor element and a sequence coding for a suppressor tRNA. In some embodiments, the conditionally repressible suppressor tRNA coding sequence comprises sequence coding for a repressor element upstream of a sequence coding for a suppressor tRNA. In some embodiments, the conditionally repressible suppressor tRNA coding sequence is between inverted terminal repeat (ITR) sequences in a polynucleotide. In some embodiments, a composition for silencing a suppressor tRNA comprises moieties that target the suppressor tRNA for degradation.

In some embodiments, the conditionally repressible suppressor tRNA coding sequence comprises a sequence coding for a repressor element and a sequence coding for a suppressor tRNA. In some embodiments, the conditionally repressible suppressor tRNA coding sequence comprises 100%, at least 99%, at least 98%, at least 95%, or at least 90% sequence identity to any one of SEQ ID NOs: SEQ ID NOs: 158-162, 164-168, 171-175, 177-181, 182-194, or 199. In some embodiments, the conditionally repressible suppressor tRNA coding sequence is flanked by inverted terminal repeat (ITR) sequences in a polynucleotide, e.g., by a 5′ ITR and a 3′ ITR. In some embodiments, the 5′ ITR comprises at least 80%, 85%, 90%, 95%, 97%, 99%, or 100% sequence identity to any one of SEQ ID NOs: 225-226. In some embodiments, the 3′ ITR comprises at least 80%, 85%, 90%, 95%, 97%, 99%, or 100% sequence identity to any one of SEQ ID NOs: 227-228. In some embodiments, one or more conditionally repressible suppressor tRNA coding sequence(s) are flanked by ITR sequences in a polynucleotide. In some embodiments, a region comprising multiple copies of one or more conditionally repressible suppressor tRNA coding sequence(s) flanked by ITRs (e.g., by a 5′ ITR and 3′ ITR) comprises one or more sequences having at least 80%, 85%, 90%, 95%, 97%, 99%, or 100% sequence identity to any one of SEQ ID NOs: 158-162, 164-168, 171-175, 177-181, 182-194, or 199. In some embodiments, a region comprising one, two, three, four, five, six, seven, eight, nine, or ten of the one or more conditionally repressible suppressor tRNA coding sequence(s) flanked by ITRs (e.g., by a 5′ ITR and 3′ ITR) comprises one, two, three, four, five, six, seven, eight, nine, or ten of the one or more sequences having comprising at least 80%, 85%, 90%, 95%, 97%, 99%, or 100% sequence identity to any one of SEQ ID NOs: 158-162, 164-168, 171-175, 177-181, 182-194, or 199. In some embodiments, a polynucleotide as described herein comprises at least 80%, 85%, 90%, 95%, 97%, 99%, or 100% sequence identity to any one of SEQ ID NOs: 203-206 or 208-211. In some embodiments, a polynucleotide as described herein comprises at least 80%, 85%, 90%, 95%, 97%, 99%, or 100% sequence identity to any one of SEQ ID NOs: 203-206 or 208-211 without the sequence coding for transduction marker, wherein the sequence coding for the transduction marker comprises at least 80%, 85%, 90%, 95%, 97%, 99%, or 100% sequence identity to SEQ ID NO: 224.

In some embodiments, the sequence coding for a suppressor tRNA of a polynucleotide disclosed herein is for a suppressor tRNA capable of suppressing an opal stop codon, an ochre stop codon, or an amber stop codon. In some embodiments, the sequence coding for a suppressor tRNA comprises 100%, at least 99%, at least 98%, at least 95%, or at least 90% sequence identity to any one of SEQ ID NO: 3-SEQ ID NO: 48, SEQ ID NO: 103-SEQ ID NO: 148, SEQ ID NO: 49-68, or SEQ ID NO: 249-268. In some embodiments, the sequence of the opal stop codon anticodon loop (TCA) of any one of SEQ ID NOs: 3-48 is interchangeable with a sequence of the amber stop codon anticodon loop (CTA) for readthrough of an amber stop codon or with a sequence of the ochre stop codon anticodon (TTA) for readthrough of an ochre stop codon. In some embodiments, the sequence of the opal stop codon anticodon loop (UCA) of any one of SEQ ID NOs: 103-148 is interchangeable with a sequence of the amber stop codon anticodon loop (CUA) for readthrough of an amber stop codon or with a sequence of the ochre stop codon anticodon (UUA) for readthrough of an ochre stop codon. In some embodiments, the sequence coding for a suppressor tRNA comprises 100%, at least 99%, at least 98%, at least 95%, or at least 90% sequence identity to any one of SEQ ID NO: 3-SEQ ID NO: 48 or SEQ ID NO: 103-SEQ ID NO: 148, wherein the anticodon is engineered to bind to an ochre stop codon or an amber stop codon. In some embodiments, the sequence of the ochre stop codon anticodon loop (TTA) of any one of SEQ ID NOs: 49-68 is interchangeable with a sequence of the amber stop codon anticodon loop (CTA) for readthrough of an amber stop codon or with a sequence of the opal stop codon anticodon (TCA) for readthrough of an opal stop codon. In some embodiments, the sequence of the ochre stop codon anticodon loop (UUA) of any one of SEQ ID NOs: 249-268 is interchangeable with a sequence of the amber stop codon anticodon loop (CUA) for readthrough of an amber stop codon or with a sequence of the opal stop codon anticodon (UCA) for readthrough of an ochre stop codon. In some embodiments, the sequence coding for a suppressor tRNA comprises 100%, at least 99%, at least 98%, at least 95%, or at least 90% sequence identity to any one of SEQ ID NO: 49-SEQ ID NO: 68 or SEQ ID NO: 249-SEQ ID NO: 268, wherein the anticodon is engineered to bind to an opal stop codon or an amber stop codon. In some embodiments, the sequence of the amber stop codon anticodon loop (CTA) of any one of SEQ ID NOs: 69-88 is interchangeable with a sequence of the opal stop codon anticodon loop (TCA) for readthrough of an opal stop codon or with a sequence of the ochre stop codon anticodon (TTA) for readthrough of an ochre stop codon. In some embodiments, the sequence of the amber stop codon anticodon loop (CUA) of any one of SEQ ID NOs: 249-268 is interchangeable with a sequence of the opal stop codon anticodon loop (UCA) for readthrough of an opal stop codon or with a sequence of the ochre stop codon anticodon (UUA) for readthrough of an ochre stop codon. In some embodiments, the sequence coding for a suppressor tRNA comprises 100%, at least 99%, at least 98%, at least 95%, or at least 90% sequence identity to any one of SEQ ID NO: 69-SEQ ID NO: 88 or SEQ ID NO: 269-SEQ ID NO: 288, wherein the anticodon is engineered to bind to an ochre stop codon or an opal stop codon.

Also provided herein are methods of silencing suppressor tRNA. In some embodiments, the silencing utilizes the compositions as described herein. For example, a repressor and a repressor element. In some embodiments, a composition of siRNA, shRNA, DsRNA, or any combination thereof is used in the method of silencing suppressor tRNA.

A method of silencing a suppressor tRNA can comprise: a) providing a cell comprising a repressor; and b) transfecting the cell with a composition comprising a conditionally repressible suppressor tRNA coding sequence, wherein a repressor binds to the repressor element of the conditionally repressible suppressor tRNA coding sequence, thereby silencing expression of the suppressor tRNA. A method of silencing a suppressor tRNA can comprise: a) providing a cell comprising a repressor; and b) transfecting the cell with a composition comprising a conditionally repressible suppressor tRNA coding sequence is flanked by ITRs, wherein a repressor binds to the repressor element of the conditionally repressible suppressor tRNA coding sequence, thereby silencing expression of the suppressor tRNA.

In some aspects, a method of silencing a suppressor tRNA comprises: a) providing a cell comprising the suppressor tRNA; and b) transfecting the cell with a composition that comprises siRNA, shRNA, DsRNA or any combination; wherein the siRNA, shRNA, DsRNA or any combination thereof binds to the suppressor tRNA and targets the suppressor tRNA for degradation, thereby silencing the tRNA. In some aspects, a method of silencing a suppressor tRNA comprises: a) providing a cell for producing AAV encapsidating a polynucleotide coding for the suppressor tRNA, wherein the cell comprises a polynucleotide coding for the suppressor tRNA flanked by ITRs; and b) transfecting the cell with a composition that comprises siRNA, shRNA, DsRNA or any combination; wherein the siRNA, shRNA, DsRNA or any combination thereof binds to the suppressor tRNA and targets the suppressor tRNA for degradation, thereby silencing the tRNA.

### Repressor Element and Repressors

The compositions and methods described herein are useful, for example, for the conditional repression of suppressor tRNA coding sequence(s). Therefore, in some cases, provided herein are conditionally repressible suppressor tRNA coding sequence(s). In some cases, the conditionally repressible suppressor tRNA coding sequence(s) each comprise sequence(s) encoding repressor element(s) and sequence(s) encoding suppressor tRNA(s).

Provided herein are repressor elements and repressors for silencing suppressor tRNAs. In some embodiments, binding of a repressor to the repressor element represses expression of the suppressor tRNA. In some embodiments, the repressor element is flanked by ITRs. In some embodiments, binding of a repressor to the repressor element represses expression of the suppressor tRNA and enables production of AAV encapsidating a polynucleotide coding for the suppressor tRNA.

A repressor element can be any sequence capable of being bound by a repressor, wherein the binding by the repressor inhibits expression of a suppressor tRNA. The sequence of the suppressor tRNA that is inhibited can be upstream of the repressor element or downstream of the repressor element. The repressor can be any moiety capable of binding the repressor element, wherein the binding by the repressor to the repressor element inhibits expression of a suppressor tRNA. A repressor, for example, can be a protein, nucleotide, or hormone. In some embodiments, the repressor elements and repressors are from a Tet repressor system. In some embodiments, the repressor elements and repressors are from a LacZ repressor system. In some embodiments, the repressor elements and repressors are from an ecdysone repressor system. In some embodiments, the repressor elements and repressors are from an artificial transcription factor system. In some embodiments, the repressor elements and repressor are from a CRISPRi system. In some embodiments, the repressor element is flanked ITRs.

### Tet Repressor System

A Tet repressor system can be used to repress the expression of a suppressor tRNA. The Tet system comprises a repressor that binds to a repressor element. The Tet system comprises a repressor that binds to a repressor element that is flanked by ITRs. In some embodiments, the repressor is a Tet repressor protein (e.g., TetR, e.g., SEQ ID NO: 223, or expressed by a sequence comprising SEQ ID NO: 222). In some embodiments, the sequence coding for the repressor element is positioned at −7 of the sequence coding for the suppressor tRNA, wherein the −7 position is seven nucleotides upstream of the start site of the suppressor tRNA. In some embodiments, the sequence coding for the repressor element is positioned at +1 of the sequence coding for the suppressor tRNA, wherein the +1 position is one nucleotide before the start site of the suppressor tRNA. In some embodiments, the repressor element comprises an operon, optionally, wherein the operon comprises a Tet operator (TetO), optionally, wherein the TetO comprises TCCCTATCAGTGATAGAGA (SEQ ID NO: 150). In some embodiments, the repressor element comprises a tetracycline response element (TRE), wherein TRE is optionally a concatemer of TetO sequences; optionally, wherein the concatemer is 2, 3, 4, 5, or 6 TetO sequences. In some cases, the TRE further comprises a promoter. In some embodiments, the TRE is a fusion of the TetO sequence and a promoter. In some embodiments, the TRE is a fusion of the concatemer of TetO sequence and a promoter. In some embodiments, the promoter is a tRNA promoter or a fragment thereof. In some embodiments, the promoter is downstream of the TetO sequence or the concatemer of TetO sequences. In some embodiments, the promoter is upstream of the TetO sequence or the concatemer of TetO sequences.

In some embodiments, the sequence coding for the repressor element is positioned upstream of the the sequence coding for suppressor tRNA, e.g., at a position +1 or at a position from −1 to −45 from the sequence of coding for the suppressor tRNA. In some embodiments, the sequence coding for the repressor element is positioned upstream of the sequence coding for the suppressor tRNA, e.g., at a position +1 or at a position from −1 to −45 from the sequence coding for the suppressor tRNA (e.g., −1, −2, −3, −4, −5, −6 −7, −8, −9, −10, −11, −12, −13, −14, −15, −16, −17, −18, −19, −20, −21, −22, −23, −24, −25, −26, −27, −28, −29, −30, −31, −32, −33, −34, −35, −36, −37, −38, −39, −40, −41, −42, −43, −44, or −45), wherein the sequence coding for the repressor element positioned upstream of the the sequence coding for suppressor tRNA and the sequence coding for the suppressor tRNA are flanked by ITRs. In some embodiments, the sequence coding for the repressor element is positioned at +1 of the suppressor tRNA. Position +1 as used herein indicates the sequence coding for the repressor element is positioned one nucleotide before the start site of the suppressor tRNA, e.g., there is no nucleotide spacer between the end of the repressor element sequence and the suppressor tRNA start site. In some embodiments, the sequence coding for the repressor element is positioned at −7 of the suppressor tRNA, wherein −7 indicates the sequence coding for the repressor element is positioned 7 nucleotides before the start site of the suppressor tRNA, e.g., there is a seven nucleotide spacer between the end of the repressor element sequence and the suppressor tRNA start site. In some embodiments, the sequence coding for the repressor element is positioned at −12 of the suppressor tRNA, wherein −12 indicates the sequence coding for the repressor element is positioned 12 nucleotides before the start site of the suppressor tRNA, e.g., there is a twelve nucleotide spacer between the end of the repressor element sequence and the suppressor tRNA start site. In some embodiments, the sequence coding for the repressor element is positioned at −20 of the suppressor tRNA, wherein −20 indicates the sequence coding for the repressor element is positioned 20 nucleotides before the start site of the suppressor tRNA, e.g., there is a twenty nucleotide spacer between the end of the repressor element sequence and the suppressor tRNA start site. In some embodiments, the sequence coding for the repressor element is positioned at −30 of the suppressor tRNA, wherein −30 indicates the sequence coding for the repressor element is positioned 30 nucleotides before the start site of the suppressor tRNA, e.g., there is a 30 nucleotide spacer between the end of the repressor element sequence and the suppressor tRNA start site. In some embodiments, the sequence coding for the repressor element is positioned at −45 of the suppressor tRNA, wherein −45 indicates the sequence coding for the repressor element is positioned 45 nucleotides before the start site of the suppressor tRNA, e.g., there is a forty-five nucleotide spacer between the end of the repressor element sequence and the suppressor tRNA start site. As used herein, the sequence coding for the repressor element is positioned at −X of the suppressor tRNA indicates the sequence coding for the repressor element is positioned X nucleotides before the start site of the sequence coding for the suppressor tRNA, e.g., there is a X nucleotide spacer between the end of the repressor element sequence and the sequence coding for the suppressor tRNA start site.

In some embodiments, two or more sequences coding for two or more repressor elements are positioned upstream of the suppressor tRNA, e.g., a sequence coding for a repressor element of the two or more sequences coding for two or more repressor elements that is closest to the sequence encoding the suppressor tRNA is at a position +1 or at a position from −1 to −45 from the sequence coding for the suppressor tRNA. In some embodiments, two or more sequences coding for two or more repressor elements are positioned upstream of the sequence coding for the suppressor tRNA, e.g., a sequence coding for a repressor element of the two or more sequences coding for two or more repressor elements that is closest to the sequence encoding the suppressor tRNA is at a position +1 or at a position from −1 to −45 from the sequence coding for the suppressor tRNA, wherein the two or more sequences coding for two or more repressor elements positioned upstream of the sequence coding for the suppressor tRNA and the sequence coding for the suppressor tRNA are flanked by ITRs. In some embodiments, the two or more sequences coding for two or more repressor elements are positioned at +1 of the sequence encoding the suppressor tRNA, wherein the +1 position is of a sequence coding for a repressor element of the two or more sequences coding for two or more repressor elements that is closest to the sequence encoding the suppressor tRNA. In some embodiments, the two or more sequences coding for two or more repressor elements are positioned at −7 of the sequence encoding the suppressor tRNA, wherein the −7 position is of a sequence coding for a repressor element of the two or more sequences coding for two or more repressor elements that is closest to the sequence encoding the suppressor tRNA. In some embodiments, the two or more sequences coding for two or more repressor elements are positioned at −12 of the sequence encoding the suppressor tRNA, wherein the −12 position is of a sequence coding for a repressor element of the two or more sequences coding for two or more repressor elements that is closest to the sequence encoding the suppressor tRNA. In some embodiments, the two or more sequences coding for two or more repressor elements are positioned at −20 of the sequence encoding the suppressor tRNA, wherein the −20 position is of a sequence coding for a repressor element of the two or more sequences coding for two or more repressor elements that is closest to the sequence encoding the suppressor tRNA. In some embodiments, the two or more sequences coding for two or more repressor elements are positioned at −30 of the sequence encoding the suppressor tRNA, wherein the −30 position is of a sequence coding for a repressor element of the two or more sequences coding for two or more repressor elements that is closest to the sequence encoding the suppressor tRNA. In some embodiments, the two or more sequences coding for two or more repressor elements are positioned at −45 of the sequence encoding the suppressor tRNA, wherein the −45 position is of a sequence coding for a repressor element of the two or more sequences coding for two or more repressor elements that is closest to the sequence encoding the suppressor tRNA.

In some embodiments, the sequence coding for the repressor element is positioned downstream of the sequence encoding the suppressor tRNA, e.g., at a position +2 to +45 from the sequence encoding the suppressor tRNA (e.g., +2, +3, +4, +5, +6+7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, +38, +39, +40, +41, +42, +43, +44, or +45). In some embodiments, the sequence coding for the repressor element is positioned downstream of the sequence coding for the suppressor tRNA, e.g., at a position +2 to +45 from the sequence coding for the suppressor tRNA, wherein the sequence coding for the repressor element positioned downstream of the sequence encoding the suppressor tRNA and the sequence encoding the suppressor tRNA are flanked by ITRs. In some embodiments, the sequence coding for the repressor element is positioned at +2 of the sequence encoding the suppressor tRNA. In some embodiments, the sequence coding for the repressor element is positioned at +3 of the sequence encoding the suppressor tRNA. In some embodiments, the sequence coding for the repressor element is positioned at +7 of the sequence encoding the suppressor tRNA. In some embodiments, the sequence coding for the repressor element is positioned at +12 of the sequence encoding the suppressor tRNA. In some embodiments, the sequence coding for the repressor element is positioned at +20 of the sequence encoding the suppressor tRNA. In some embodiments, the sequence coding for the repressor element is positioned at +30 of the sequence encoding the suppressor tRNA. In some embodiments, the sequence coding for the sequence encoding the repressor element is positioned at +45 of the suppressor tRNA. As used herein, the sequence coding for the repressor element is positioned at +X of the suppressor tRNA indicates the sequence coding for the repressor element is positioned X nucleotides after the last nucleotide of the sequence coding for the suppressor tRNA, e.g., there is a X nucleotide spacer between the end of the sequence coding for the suppressor tRNA and the repressor element sequence start site, except for position +1, which is defined as described above.

In some embodiments, two or more sequences coding for two or more repressor elements are positioned downstream of the sequence coding for the suppressor tRNA, e.g., a sequence coding for a repressor element of the two or more sequences coding for two or more repressor elements that is closest to the sequence encoding the suppressor tRNA is at a position +2 to +45 from the sequence coding for the suppressor tRNA. In some embodiments, two or more sequences coding for two or more repressor elements are positioned downstream of the sequence coding for the suppressor tRNA, e.g., a sequence coding for a repressor element of the two or more sequences coding for two or more repressor elements that is closest to the sequence encoding the suppressor tRNA is at a position +2 to +45 from the sequence coding for the suppressor tRNA, wherein two or more sequences coding for two or more repressor elements positioned downstream of the sequence coding for the suppressor tRNA and the the sequence coding for the suppressor tRNA are flanked by ITRs. In some embodiments, the two or more sequences coding for two or more repressor elements are positioned at +7 of the sequence encoding the suppressor tRNA, wherein the +7 position is of a sequence coding for a repressor element of the two or more sequences coding for two or more repressor elements that is closest to the sequence encoding the suppressor tRNA. In some embodiments, the two or more sequences coding for two or more repressor elements are positioned at +12 of the sequence encoding the suppressor tRNA, wherein the +12 position is of a sequence coding for a repressor element of the two or more sequences coding for two or more repressor elements that is closest to the sequence encoding the suppressor tRNA. In some embodiments, the two or more sequences coding for two or more repressor elements are positioned at +20 of the sequence encoding the suppressor tRNA, wherein the +20 position is of a sequence coding for a repressor element of the two or more sequences coding for two or more repressor elements that is closest to the sequence encoding the suppressor tRNA. In some embodiments, the two or more sequences coding for two or more repressor elements are positioned at +30 of the sequence encoding the suppressor tRNA, wherein the +30 position is of a sequence coding for a repressor element of the two or more sequences coding for two or more repressor elements that is closest to the sequence encoding the suppressor tRNA. In some embodiments, the two or more sequences coding for two or more repressor elements are positioned at +45 of the sequence encoding the suppressor tRNA, wherein the +45 position is of a sequence coding for a repressor element of the two or more sequences coding for two or more repressor elements that is closest to the sequence encoding the suppressor tRNA.

In some embodiments, the conditionally repressible suppressor tRNA coding sequence is flanked by ITRs, e.g., by a 5′ ITR and a 3′ ITR. In some embodiments, the composition comprises one or more conditionally repressible suppressor tRNA coding sequences. In some embodiments, the composition comprises one or more conditionally repressible suppressor tRNA coding sequences flanked by ITRs. In some embodiments, the composition comprises two conditionally repressible suppressor tRNA coding sequences. In some embodiments, the composition comprises two conditionally repressible suppressor tRNA coding sequences flanked by ITRs. In some embodiments, the composition comprises three conditionally repressible suppressor tRNA coding sequences. In some embodiments, the composition comprises three conditionally repressible suppressor tRNA coding sequences flanked by ITRs. In some embodiments, the composition comprises six conditionally repressible suppressor tRNA coding sequences. In some embodiments, the composition comprises six conditionally repressible suppressor tRNA coding sequences flanked by ITRs. In some embodiments, the one or more conditionally repressible suppressor tRNA coding sequence are flanked by inverted terminal repeats (ITRs). In some embodiments, the two conditionally repressible suppressor tRNA coding sequences, the three conditionally repressible suppressor tRNA coding sequences, or the six conditionally repressible suppressor tRNA coding sequences are flanked by ITRs.

In some embodiments, the 5′ ITR comprises a polynucleotide having at least 80%, 85%, 90%, 95%, 97%, 99%, or 100% sequence identity to any one of SEQ ID NOs: 225-226. In some embodiments, the 3′ ITR comprises a polynucleotide having at least 80%, 85%, 90%, 95%, 97%, 99%, or 100% sequence identity to any one of SEQ ID NOs: 227-228.

### Artificial Transcription Factors

An artificial transcription factor system can be used to repress the expression of a suppressor tRNA. See, e.g., Heiderscheit et al., “Reprogramming Cell Fate with Artificial Transcription Factors,” FEBS Letters 592:888-900 (2018).

In some cases, the artificial transcription factor system is a CRISPRi system. The CRISPRi comprises a repressor that binds to a repressor element. In some embodiments, the conditionally repressible suppressor tRNA coding sequence comprises a guide RNA binding sequence and encodes a suppressor tRNA. In some embodiments, the guide RNA binding sequence comprises a PAM sequence. In some embodiments, the guide RNA binding sequence is from 15 to 30 nucleotides in length. In some embodiments, the guide RNA binding sequence is positioned at +1 or at −1 to −45 (e.g., −1, −2, −3, −4, −5, −6 −7, −8, −9, −10, −11, −12, −13, −14, −15, −16, −17, −18, −19, −20, −21, −22, −23, −24, −25, −26, −27, −28, −29, −30, −31, −32, −33, −34, −35, −36, −37, −38, −39, −40, −41, −42, −43, −44, or −45) of the sequence coding for the suppressor tRNA. In some embodiments, the guide RNA binding sequence is positioned at +2 to +45 from the sequence encoding the suppressor tRNA (e.g., +2, +3, +4, +5, +6+7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, +38, +39, +40, +41, +42, +43, +44, or +45). In some embodiments, binding of the guide RNA binding sequence to a guide RNA complexed to a Cas protein that is linked or fused to a repressor domain, represses expression of the suppressor tRNA. In some embodiments, the repressor is a guide RNA complexed to a Cas protein, wherein the Cas protein is linked or fused to a repressor domain. In some embodiments, Cas protein is a catalytically dead Cas protein. In some embodiments, the repressor domain is a krab domain. In some embodiments, the conditionally repressible suppressor tRNA coding sequence is flanked by ITRs. In some embodiments, the composition comprises one or more conditionally repressible suppressor tRNA coding sequences. In some embodiments, the composition comprises one or more conditionally repressible suppressor tRNA coding sequences flanked ITRs. In some embodiments, the composition comprises two conditionally repressible suppressor tRNA coding sequences. In some embodiments, the composition comprises two conditionally repressible suppressor tRNA coding sequences flanked by ITRs. In some embodiments, the composition comprises three conditionally repressible suppressor tRNA coding sequences. In some embodiments, the composition comprises three conditionally repressible suppressor tRNA coding sequences flanked by ITRs. In some embodiments, the composition comprises six conditionally repressible suppressor tRNA coding sequences. In some embodiments, the composition comprises six conditionally repressible suppressor tRNA coding sequences flanked by ITRs. In some embodiments, the one or more conditionally repressible suppressor tRNA coding sequence are flanked by ITRs. In some embodiments, the two conditionally repressible suppressor tRNA coding sequences, the three conditionally repressible suppressor tRNA coding sequences, or the six conditionally repressible suppressor tRNA coding sequences are flanked by ITRs.

Suppressor tRNA Degradation and Interference

Provided here are compositions for silencing a suppressor tRNA that comprise moieties that interference with the structure and/or function of the tRNA and/or target the suppressor tRNA for degradation. Provided here are compositions for silencing a suppressor tRNA that comprise moieties that interference with the structure and/or function of the tRNA and/or target the suppressor tRNA for degradation during AAV production. In some aspects, a composition comprises one or more short interfering RNA (siRNA), one or more short hairpin RNA (shRNA), one or more dicer-substrate RNAs (DsRNA), or any combination thereof, wherein the one or more siRNA, the one or more shRNA, or the one or more DsRNA bind to a suppressor tRNA. In some embodiments, upon binding of the one or more siRNA, the one or more shRNA, or the one or more DsRNA, the suppressor tRNA is targeted for degradation. In some embodiments, upon binding of the one or more siRNA, the one or more shRNA, or the one or more DsRNA, the suppressor tRNA is degraded by a RNA induced silencing complex. In some embodiments, the one or more short interfering RNA (siRNA), one or more short hairpin RNA (shRNA), one or more dicer-substrate RNAs (DsRNA), or any combination thereof, wherein the one or more siRNA, the one or more shRNA, or the one or more DsRNA bind to an anticodon region of the suppressor tRNA. In some embodiments, the one or more siRNA are 15 nucleotides to 25 nucleotides in length. In some embodiments, the one or more siRNA are 19 nucleotides in length. In some embodiments, the one or more DsRNA are 25 to 35 nucleotides in length. In some embodiments, the one or more DsRNA are 27 nucleotides in length. In some embodiments, the composition further comprises a suppressor tRNA.

Compositions and Methods for Producing Virion Encapsidating Suppressor tRNA

Provided herein are compositions and methods for producing virion encapsidating a polynucleotide coding for a suppressor tRNA. In some embodiments, during the methods of producing the virion, the suppressor tRNA is silenced. In some embodiments, the silencing utilizes the compositions as described herein (e.g., a composition comprising conditionally repressible suppressor coding sequence(s)). For example, the silencing utilizes a repressor and a repressor element for silencing expression of the suppressor tRNA from the polynucleotide coding for the suppressor tRNA. In some embodiments, a composition of siRNA, shRNA, DsRNA, or any combination thereof is used in the method of silencing suppressor tRNA. In some embodiments, the compositions and methods for producing virion encapsidating a polynucleotide coding for a suppressor tRNA comprise stop codon engineered AAV nucleotide sequence(s) in which the AAV nucleotide sequence(s) code for a protein or nucleotide involved in or required for the production of AAV virion and uses thereof during AAV production.

### Nucleic Acid Vectors and Cells

The compositions and methods described herein are useful, for example, for the production of virion encapsidating a polynucleotide coding for a suppressor tRNA. Therefore, among other things, provided herein are nucleic acid vectors(s) and cell(s) comprising polynucleotide sequence(s) comprising suppressor tRNA(s) (e.g., one or more conditionally repressible suppressor tRNAs). In some cases, e.g., for production of AAV virion, the nucleic acid vector comprises a polynucleotide comprising, from 5′ to 3′: a 5′ AAV inverted terminal repeat (ITR) sequence; a polynucleotide sequence comprising one or more suppressor tRNA coding sequence(s); and a 3′ AAV inverted terminal repeat (ITR) sequence. In some cases, e.g., for production of AAV virion, the nucleic acid vector comprises a polynucleotide comprising, from 5′ to 3′: a 5′ AAV inverted terminal repeat (ITR) sequence; a polynucleotide sequence comprising one or more conditionally repressible suppressor tRNA coding sequence(s); and a 3′ AAV inverted terminal repeat (ITR) sequence. In some embodiments, the 5′ ITR comprises a polynucleotide having at least 80%, 85%, 90%, 95%, 97%, 99%, or 100% sequence identity to any one of SEQ ID NOs: 225-226. In some embodiments, the 3′ ITR comprises a polynucleotide having at least 80%, 85%, 90%, 95%, 97%, 99%, or 100% sequence identity to any one of SEQ ID NOs: 227-228.

Provided herein are nucleic acid vector(s) comprising nucleic acid sequence(s) encoding suppressor tRNA(s) (e.g., polynucleotide(s) encoding suppressor tRNA(s)) and AAV polynucleotides encoding AAV nucleotide sequence(s) encoding element(s) for producing AAV virion. Provided herein are nucleic acid vector(s) comprising nucleic acid sequence(s) encoding suppressor tRNA(s) (e.g., polynucleotide(s) encoding suppressor tRNA(s)), wherein the nucleic acid sequence(s) encoding suppressor tRNA(s) (e.g., polynucleotide(s) encoding suppressor tRNA(s)) further comprise sequences encoding ITRs that flank the suppressor tRNA(s) sequence(s), and AAV nucleotide sequence(s) encoding element(s) for producing AAV virion. In some cases, the nucleic acid sequence(s) encoding suppressor tRNA(s) (e.g., polynucleotide(s) encoding suppressor tRNA(s)) and AAV nucleotide sequence(s) encoding element(s) for producing AAV virion are on the same vector. In some cases, the nucleic acid sequence(s) encoding suppressor tRNA(s) (e.g., polynucleotide(s) encoding suppressor tRNA(s)) and AAV nucleotide sequence(s) encoding element(s) for producing AAV virion are on different vectors. In some cases, the nucleic acid sequence(s) encoding suppressor tRNA(s) (e.g., polynucleotide(s) encoding suppressor tRNA(s)) are in a vector and AAV nucleotide sequence(s) encoding element(s) for producing AAV virion are integrated into the genome of a cell or a cell line. In some cases, the nucleic acid sequence(s) encoding suppressor tRNA(s) (e.g., polynucleotide(s) encoding suppressor tRNA(s)) are integrated into the genome of a cell or a cell line and AAV nucleotide sequence(s) encoding element(s) for producing AAV virion are in a vector. In some cases, the nucleic acid sequence(s) encoding suppressor tRNA(s) (e.g., polynucleotide(s) encoding suppressor tRNA(s)) and AAV nucleotide sequence(s) encoding element(s) for producing AAV virion are integrated into the genome of a cell or a cell line. In some cases, integration of the nucleic acid sequence(s) encoding suppressor tRNA(s) (e.g., polynucleotide(s) encoding suppressor tRNA(s)) and/or AAV nucleotide sequence(s) encoding element(s) for producing AAV virion into the genome of the cell or cell line is stable integration into the genome of the cell. In some embodiments, the polynucleotide comprising sequence(s) encoding suppressor tRNA(s) is a conditionally repressible suppressor tRNA coding sequence(s). In some embodiments, the polynucleotide comprising sequence(s) encoding suppressor tRNA(s) is not a conditionally repressible suppressor tRNA coding sequence(s). In some embodiments, the polynucleotide comprising sequence(s) encoding suppressor tRNA(s) lack repressor element(s). In some embodiments, the AAV nucleotide sequence(s) encoding element(s) for producing AAV virion is a stop codon engineered AAV nucleotide sequence(s). In some embodiments, the AAV nucleotide sequence(s) encoding for one or more element(s) for producing AAV virion as described herein comprise stop codon engineered AAV nucleotide sequence(s) wherein the engineered stop codon is engineered not to be the stop codon recognized or that is bound by the suppressor tRNA expressed by the nucleic acid sequence(s) encoding one or more suppressor tRNA(s) (e.g., polynucleotide(s) encoding suppressor tRNA(s)).

In some cases, the nucleic acid vectors and constructs described herein comprise one or more tags (e.g., quantification tags and/or transduction tags). A person of skill in the art will appreciate that these components are useful, for example, for measurements, but not for therapeutic efficacy. Therefore, in some cases, the nucleic acid vectors and constructs described herein include such tags and, in other cases, the nucleic acid vectors and constructs described herein exclude such tags.

In some embodiments, the nucleotide sequence(s) encoding suppressor tRNA(s) (e.g., polynucleotide(s) encoding suppressor tRNA(s)) and comprises 100%, at least 99%, at least 98%, at least 95%, or at least 90% sequence identity to any one of SEQ ID NOs: 203-206 or 208-212, with or without the transduction marker. In some embodiments, a polynucleotide coding for a transduction marker comprises a CMV promoter sequence that drives expression of a Thy 1.1 sequence. In some embodiments, the polynucleotide coding for the transduction marker comprises 100%, at least 99%, at least 98%, at least 95%, or at least 90% sequence identity to SEQ ID NO: 224. The transduction markers of SEQ ID NOs 203-212 are as follows:

| SEQ ID NO: | Transduction Marker Location |
|---|---|
| 203 | 347-1298 |
| 204 | 739-1690 |
| 205 | 302-1253 |
| 206 | 604-1555 |
| 207 | 604-1555 |
| 208 | 323-1274 |
| 209 | 667-1618 |
| 210 | 849-1800 |
| 211 | 714-1665 |
| 212 | 714-1665 |

Thus, provided herein is a composition comprising a nucleic acid vector system comprising one or more nucleic acid vector(s) comprising: nucleic acid sequence(s) encoding one or more suppressor tRNA(s) (e.g., polynucleotide(s) encoding suppressor tRNA(s)), e.g., suppressor tRNA(s) described herein, or conditionally repressible suppressor tRNA coding sequence(s) described herein, wherein the nucleic acid sequence(s) encoding one or more suppressor tRNA(s), e.g., suppressor tRNA(s) described herein, or conditionally repressible suppressor tRNA coding sequence(s) described herein are flanked by ITRs; and AAV nucleotide sequence(s) encoding for one or more element(s) for producing AAV virion as described herein, e.g., stop codon engineered AAV nucleotide sequence(s). Further provided herein is a composition comprising a cell or cell line comprising one or more nucleic acid sequences(s) comprising: nucleic acid sequence(s) encoding one or more suppressor tRNA(s), e.g., suppressor tRNA(s) described herein, or conditionally repressible suppressor tRNA coding sequence(s) described herein, wherein the nucleic acid sequence(s) encoding one or more suppressor tRNA(s), e.g., suppressor tRNA(s) described herein, or conditionally repressible suppressor tRNA coding sequence(s) described herein are flanked by ITRs; and one or more AAV nucleotide sequence(s) encoding for element(s) for producing AAV virion as described herein, e.g., stop codon engineered AAV nucleotide sequence(s). Further provided herein is a composition comprising a cell or cell line comprising one or more polynucleotide(s) having nucleic acid sequences(s) comprising: nucleic acid sequence(s) encoding one or more suppressor tRNA(s), e.g., suppressor tRNA(s) described herein, or conditionally repressible suppressor tRNA coding sequence(s) described herein, wherein the nucleic acid sequence(s) encoding one or more suppressor tRNA(s), e.g., suppressor tRNA(s) described herein, or conditionally repressible suppressor tRNA coding sequence(s) described herein are flanked by ITRs; and one or more AAV nucleotide sequence(s) encoding for element(s) for producing AAV virion as described herein, e.g., stop codon engineered AAV nucleotide sequence(s). In some embodiments, the AAV nucleotide sequence(s) encoding for one or more element(s) for producing AAV virion as described herein comprise stop codon engineered AAV nucleotide sequence(s) wherein the engineered stop codon is engineered not to be the stop codon recognized or that is bound by the suppressor tRNA expressed by the nucleic acid sequence(s) encoding one or more suppressor tRNA(s) (e.g., polynucleotide(s) encoding suppressor tRNA(s)). In some embodiments, when the AAV nucleotide sequence(s) encoding for one or more element(s) for producing AAV virion as described herein comprise stop codon engineered AAV nucleotide sequence(s) wherein the engineered stop codon is engineered not to be the stop codon recognized or that is bound by the suppressor tRNA expressed by the nucleic acid sequence(s) encoding one or more suppressor tRNA(s) (e.g., polynucleotide(s) encoding suppressor tRNA(s)), the polynucleotide(s) encoding the suppressor tRNA(s) are not encoding conditionally repressible suppressor tRNA coding sequence(s). In some embodiments, when the AAV nucleotide sequence(s) encoding for one or more element(s) for producing AAV virion as described herein comprise stop codon engineered AAV nucleotide sequence(s) wherein the engineered stop codon is engineered not to be the stop codon recognized or that is bound by the suppressor tRNA expressed by the nucleic acid sequence(s) encoding one or more suppressor tRNA(s) (e.g., polynucleotide(s) encoding suppressor tRNA(s)), the polynucleotide(s) encoding the suppressor tRNA(s) lack repressor element(s).

In some cases, the nucleic acid sequence(s) encoding one or more suppressor tRNA(s) (e.g., the polynucleotide(s) encoding suppressor tRNA(s)) are flanked by inverted terminal repeats (ITR) sequences, e.g., AAV ITRs (see, e.g., Earley et al., “Adeno-Associated Virus Serotype-Specific Inverted Terminal Repeat Sequence Role in Vector Transgene Expression,” Human Gene Therapy 31:10.1089/human.2019.274 (2020)), e.g., AAV2 ITRs. In some cases, a polynucleotide coding for a suppressor tRNA as disclosed herein further comprises ITR sequences flanking the suppressor tRNA sequence. In some cases, the nucleic acid sequence(s) encoding one or more suppressor tRNA(s) are flanked by ITR sequences and further comprise one or more repressor elements and/or a binding sites for an artificial transcription factor. In some cases, the polynucleotide coding for a suppressor tRNA comprises ITRs flanking the nucleic acid sequence(s) encoding one or more suppressor tRNA(s) and comprises one or more repressor elements and/or a binding sites for an artificial transcription factor. In some cases, the nucleic acid sequence(s) encoding one or more conditionally repressible suppressor tRNA(s) are flanked by ITR sequences. In some cases, the polynucleotide coding for a suppressor tRNA comprises the nucleic acid sequence(s) encoding one or more conditionally repressible suppressor tRNA(s) that are flanked by ITR sequences. The flanking ITR sequences can be a 5′ ITR sequence and 3′ ITR sequence. In some embodiments, the 5′ ITR comprises a polynucleotide having at least 80%, 85%, 90%, 95%, 97%, 99%, or 100% sequence identity to any one of SEQ ID NOs: 225-226. In some embodiments, the 3′ ITR comprises a polynucleotide having at least 80%, 85%, 90%, 95%, 97%, 99%, or 100% sequence identity to any one of SEQ ID NOs: 227-228.

In some cases, the nucleic acid sequence encoding one or more suppressor tRNA(s) or conditionally repressible suppressor tRNA coding sequence(s) comprises, from 5′ to 3′: a 5′ ITR sequence, a suppressor tRNA domain, and a 3′ ITR sequence. In some cases, the suppressor tRNA domain comprises, from 5′ to 3′, a repressor element and/or a binding site for an artificial transcription factor, and a nucleic acid sequence encoding a suppressor tRNA (e.g., a polynucleotide encoding a suppressor tRNA). In some cases, the suppressor tRNA domain comprises, from 5′ to 3′, one or more sequences, each comprising, from 5′ to 3′, a repressor element and/or a binding site for an artificial transcription factor, and a nucleic acid sequence encoding a suppressor tRNA (e.g., a polynucleotide encoding a suppressor tRNA).

Suitable repressor elements and artificial transcription factor binding suites are described above with respect to compositions and methods for silencing suppressor tRNAs.

In some cases, the nucleic acid sequence encoding one or more suppressor RNA(s) or conditionally repressible suppressor tRNA coding sequence(s) comprises a promoter sequence. In some cases, the promoter sequence is an RNA Polymerase type III promoter sequence or portion thereof. In some cases, the RNA polymerase type III promoter sequence is selected from a U6 promoter sequence, optionally a hU6 promoter sequence or mU6 promoter sequence. In some cases, the hU6 promoter sequence comprises one or more promoter sequence element(s) selected from the group consisting of a SPH element, an Oct-1 element, a proximal sequence element (PSE), and a TATA element. In some cases, the promoter sequence element(s) are interspersed between repressor element sequence(s) and suppressor tRNA sequence(s). In some cases, the repressor element sequence(s) are downstream of the SPH and Oct-1 element(s). In some cases, the repressor element sequence(s) are before the PSE element, between the PSE and TATA elements and/or after the TATA element.

In some cases, the promoter sequence comprises a tRNA promoter sequence or fragment thereof.

In some cases, the promoter sequence is upstream of the one or more suppressor tRNA(s) or conditionally repressible suppressor tRNA coding sequence(s). In some cases, the promoter sequence is downstream of the one or more suppressor tRNA(s) or conditionally repressible suppressor tRNA coding sequence(s). In some cases, the promoter sequence is interspersed within the one or more suppressor tRNA(s) or conditionally repressible suppressor tRNA coding sequence(s).

In some embodiments, the conditionally repressible suppressor tRNA coding sequence flanked by ITRs (e.g., by a 5′ ITR and 3′ ITR) comprises at least 80%, 85%, 90%, 95%, 97%, 99%, or 100% sequence identity to any one of SEQ ID NOs: 158-162, 164-168, 171-175, 177-181, 182-194, or 199. In some embodiments, a region comprising multiple copies of one or more conditionally repressible suppressor tRNA coding sequence(s) flanked by ITRs (e.g., by a 5′ ITR and 3′ ITR) comprises one or more sequences having at least 80%, 85%, 90%, 95%, 97%, 99%, or 100% sequence identity to any one of SEQ ID NOs: 158-162, 164-168, 171-175, 177-181, 182-194, or 199. In some embodiments, a region comprising one, two, three, four, five, six, seven, eight, nine, or ten of the one or more conditionally repressible suppressor tRNA coding sequence(s) flanked by ITRs (e.g., by a 5′ ITR and 3′ ITR) comprises one, two, three, four, five, six, seven, eight, nine, or ten of the one or more sequences having comprising at least 80%, 85%, 90%, 95%, 97%, 99%, or 100% sequence identity to any one of SEQ ID NOs: 158-162, 164-168, 171-175, 177-181, 182-194, or 199. In some embodiments, a polynucleotide as described herein comprises at least 80%, 85%, 90%, 95%, 97%, 99%, or 100% sequence identity to any one of SEQ ID NOs: 203-206 or 208-211. In some embodiments, a polynucleotide as described herein comprises at least 80%, 85%, 90%, 95%, 97%, 99%, or 100% sequence identity to any one of SEQ ID NOs: 203-206 or 208-211 without the sequence coding for transduction marker, wherein the sequence coding for the transduction marker comprises at least 80%, 85%, 90%, 95%, 97%, 99%, or 100% sequence identity to SEQ ID NO: 224. In some embodiments, the 5′ ITR comprises a polynucleotide having at least 80%, 85%, 90%, 95%, 97%, 99%, or 100% sequence identity to any one of SEQ ID NOs: 225-226. In some embodiments, the 3′ ITR comprises a polynucleotide having at least 80%, 85%, 90%, 95%, 97%, 99%, or 100% sequence identity to any one of SEQ ID NOs: 227-228.

In some cases, the element(s) for producing AAV virion of an AAV nucleotide sequence are any proteins or nucleotides involved in or required for the production of AAV virion in a cell. In some cases, the element(s) for producing AAV virion are stop codon engineered (e.g., is an AAV polynucleotide comprising a stop codon engineered AAV nucleotide sequence). In some cases, the stop codon engineered element (e.g., an AAV polynucleotide comprising a stop codon engineered AAV nucleotide sequence) comprises one or more changes, relative to a reference sequence (e.g., a wild-type sequence) selected from the group consisting of: ochre to amber, ochre to opal, opal to ochre, opal to amber, amber to ochre, amber to opal, and combinations thereof. Preferably, the stop codon engineered element (e.g., an AAV polynucleotide comprising a stop codon engineered AAV nucleotide sequence) comprises one or more changes, relative to a reference sequence (e.g., a wild-type sequence) selected from the group consisting of: ochre to amber or ochre to opal when the suppressor tRNA expressed from the polynucleotide coding for the suppressor tRNA binds to ochre stop codons; opal to amber or opal to ochre when the suppressor tRNA expressed from the polynucleotide coding for the suppressor tRNA binds to opal stop codons; or amber to opal or amber to ochre when the suppressor tRNA expressed from the polynucleotide coding for the suppressor tRNA binds to amber stop codons. In some cases, the element(s) for producing AAV virion using a polynucleotide coding for an AAV nucleotide sequence are selected from the group consisting of polynucleotide coding for: a Rep protein, a Cap protein, a Helper protein, VA RNA, an AAP protein, an MAAP protein, a Protein X protein, and combinations thereof. In some cases, the element(s) for producing AAV virion using a polynucleotide coding for an AAV nucleotide sequence are selected from the group consisting of polynucleotide coding for: a Rep protein, a Cap protein, a Helper protein, VA RNA, an AAP protein, an MAAP protein, a Protein X protein, a Hexon Assembly Protein, a Hexon-associated Precursor protein, a 23K endoprotease, and combinations thereof. In some cases, the element(s) for producing AAV virion using a polynucleotide coding for a stop codon engineered AAV nucleotide sequence are selected from the group consisting of polynucleotide coding for: a Rep protein, a Cap protein, a Helper protein, VA RNA, an AAP protein, an MAAP protein, a Protein X protein, and combinations thereof, wherein one or more stop codons of the AAV nucleotide sequence are engineered to be a different stop codon. In some cases, the element(s) for producing AAV virion using a polynucleotide coding for a stop codon engineered AAV nucleotide sequence are selected from the group consisting of polynucleotide coding for: a Rep protein, a Cap protein, a Helper protein, VA RNA, an AAP protein, an MAAP protein, a Protein X protein, a Hexon Assembly Protein, a Hexon-associated Precursor protein, a 23K endoprotease, and combinations thereof, wherein one or more stop codons of the AAV nucleotide sequence are engineered to be a different stop codon. In some embodiments, the Rep protein is a Rep2 protein. In some embodiments, the stop codon engineered AAV nucleotide sequence comprises an ochre stop codon of the sequence coding for Rep changed to an opal stop codon or an opal stop codon. In some embodiments, the Rep protein is Rep78, Rep52, Rep68, Rep40, or any combination thereof. In some embodiments, an opal stop codon of the sequence coding for the Rep68 is changed to an amber stop codon or an ochre stop codon. In some embodiments, the stop codon engineered AAV nucleotide sequence comprises an opal stop codon of the sequence coding for the Rep68 is engineered to an amber stop codon or an ochre stop codon. In some embodiments, an opal stop codon of the sequence coding for the Rep40 is changed to an amber stop codon or an ochre stop codon. In some embodiments, the stop codon engineered AAV nucleotide sequence comprises an opal stop codon of the sequence coding for the Rep40 is engineered to an amber stop codon or an ochre stop codon. In some embodiments, an ochre stop codon of the sequence coding for the Rep78 is changed to an amber stop codon or an opal stop codon. In some embodiments, the stop codon engineered AAV nucleotide sequence comprises an ochre stop codon of the sequence coding for the Rep78 is engineered to an amber stop codon or an opal stop codon. In some embodiments, an ochre stop codon of the sequence coding for the Rep52 is changed to an amber stop codon or an opal stop codon. In some embodiments, the stop codon engineered AAV nucleotide sequence comprises an ochre stop codon of the sequence coding for the Rep52 is engineered to an amber stop codon or an opal stop codon. In some embodiments, the Cap protein is VP1, VP2, VP3, or any combination thereof. In some embodiments, an ochre stop codon of the sequence coding for VP1 is changed to an amber stop codon or an opal stop codon. In some embodiments, the stop codon engineered AAV nucleotide sequence comprises an ochre stop codon of the sequence coding for the VP1 is engineered to an amber stop codon or an opal stop codon. In some embodiments, an ochre stop codon of the sequence coding for VP2 is changed to an amber stop codon or an opal stop codon. In some embodiments, the stop codon engineered AAV nucleotide sequence comprises an ochre stop codon of the sequence coding for the VP2 is engineered to an amber stop codon or an opal stop codon. In some embodiments, an ochre stop codon of the sequence coding for VP3 is changed to an amber stop codon or an opal stop codon. In some embodiments, the stop codon engineered AAV nucleotide sequence comprises an ochre stop codon of the sequence coding for the VP3 is engineered to an amber stop codon or an opal stop codon. In some embodiments, the Cap protein is a Cap5 protein. In some embodiments, an ochre stop codon of the sequence coding for Cap5 is changed to an amber stop codon or an opal stop codon. In some embodiments, the stop codon engineered AAV nucleotide sequence comprises an ochre stop codon of the sequence coding for the Cap5 is engineered to an amber stop codon or an opal stop codon. In some embodiments, the Helper protein is E1a, E1b, E4, E2a, or any combination thereof. In some embodiments, a stop codon of the sequence coding for E1a is changed to a different stop codon. In some embodiments, the stop codon engineered AAV nucleotide sequence comprises a stop codon of the sequence coding for the E1a is engineered to a different stop codon. In some embodiments, the stop codon engineered AAV nucleotide sequence comprises an ochre stop codon of the sequence coding for the E1a is engineered to an amber stop codon or an opal stop codon. In some embodiments, a stop codon of the sequence coding for E1b is changed to a different stop codon. In some embodiments, the stop codon engineered AAV nucleotide sequence comprises a stop codon of the sequence coding for the E1b is engineered to a different stop codon. In some embodiments, the E1b protein is a 55k E1b protein, a 19K E1b protein, or both. In some embodiments, a stop codon of the sequence coding for 55k E1b protein is changed to a different stop codon. In some embodiments, a stop codon of the sequence coding for 55k E1b protein is changed from an opal stop codon to an ochre stop codon or an amber stop codon. In some embodiments, a stop codon of the sequence coding for 19k E1b protein is changed to a different stop codon. In some embodiments, a stop codon of the sequence coding for 19k E1b protein is changed from an opal stop codon to an ochre stop codon or an amber stop codon. In some embodiments, a stop codon of the sequence coding for E4 is changed to a different stop codon. In some embodiments, the stop codon engineered AAV nucleotide sequence comprises a stop codon of the sequence coding for the E4 is engineered to a different stop codon. In some embodiments, E4 is E4orf1. In some embodiments, E4 is E4orf2. In some embodiments, E4 is E4orf3. In some embodiments, E4 is E4orf4. In some embodiments, E4 is E4orf6. In some embodiments, the stop codon engineered AAV nucleotide sequence comprises a stop codon of the sequence coding for the E4orf1 is engineered from an ochre stop codon to an opal stop codon or an amber stop codon. In some embodiments, the stop codon engineered AAV nucleotide sequence comprises a stop codon of the sequence coding for the E4orf2 is engineered from an opal stop codon to an ochre stop codon or an amber stop codon. In some embodiments, the stop codon engineered AAV nucleotide sequence comprises a stop codon of the sequence coding for the E4orf3 is engineered from an ochre stop codon to an opal stop codon or an amber stop codon. In some embodiments, the stop codon engineered AAV nucleotide sequence comprises a stop codon of the sequence coding for the E4orf4 is engineered from an amber stop codon to an opal stop codon or an ochre stop codon. In some embodiments, the stop codon engineered AAV nucleotide sequence comprises a stop codon of the sequence coding for the E4orf6 is engineered from an amber stop codon to an opal stop codon or an ochre stop codon. In some embodiments, a stop codon of the sequence coding for E2a is changed to a different stop codon. In some embodiments, the stop codon engineered AAV nucleotide sequence comprises a stop codon of the sequence coding for the E2a is engineered to a different stop codon. In some embodiments, the stop codon engineered AAV nucleotide sequence comprises a stop codon of the sequence coding for the E2a is engineered from an ochre stop codon to an opal stop codon or an amber stop codon. In some embodiments, a stop codon of the sequence coding for VA RNA is changed to a different stop codon. In some embodiments, the stop codon engineered AAV nucleotide sequence comprises a stop codon of the sequence coding for the VA RNA is engineered to a different stop codon. In some embodiments, an opal stop codon of the sequence coding for AAP is changed to an amber stop codon or an ochre stop codon. In some embodiments, the stop codon engineered AAV nucleotide sequence comprises an opal stop codon of the sequence coding for the AAP is engineered to an amber stop codon or an ochre stop codon. In some embodiments, an amber stop codon of the sequence coding for MAAP is changed to an opal stop codon or an ochre stop codon. In some embodiments, the stop codon engineered AAV nucleotide sequence comprises an amber stop codon of the sequence coding for the MAAP is engineered to an opal stop codon or an ochre stop codon. In some embodiments, an opal stop codon of the sequence coding for Protein X is changed to an amber stop codon or an ochre stop codon. In some embodiments, the stop codon engineered AAV nucleotide sequence comprises an opal stop codon of the sequence coding for the Protein X is engineered to an amber stop codon or an ochre stop codon. In some embodiments, the stop codon engineered AAV nucleotide sequence comprises an ochre stop codon of the sequence coding for the Fiber is engineered to an amber stop codon or an opal stop codon. In some embodiments, the stop codon engineered AAV nucleotide sequence comprises an amber stop codon of the sequence coding for the Hexon Assembly protein that is engineered to an ochre stop codon or an opal stop codon. In some embodiments, the stop codon engineered AAV nucleotide sequence comprises an opal stop codon of the sequence coding for the Hexon-associated Precursor protein that is engineered to an ochre stop codon or an amber stop codon. In some embodiments, the stop codon engineered AAV nucleotide sequence comprises an ochre stop codon of the sequence coding for the 23k Endoprotease that is engineered to an opal stop codon or an amber stop codon. In some embodiments, a protein associated with the expression of the Hexon assembly protein comprises an opal stop codon, and the stop codon engineered AAV nucleotide sequence comprises an opal stop codon of the protein associated with the expression of the Hexon assembly protein that is engineered to an ochre stop codon or an amber stop codon. In some embodiments, a protein associated with the expression of the 23k Endoprotease comprises an opal stop codon, and the stop codon engineered AAV nucleotide sequence comprises an opal stop codon of the protein associated with the expression of the 23k Endoprotease that is engineered to an ochre stop codon or an amber stop codon. In some embodiments, a protein associated with the expression of the VP1 protein comprises an opal stop codon, and the stop codon engineered AAV nucleotide sequence comprises an opal stop codon of the protein associated with the expression of the VP1 protein that is engineered to an ochre stop codon or an amber stop codon. In some embodiments, a protein associated with the expression of the E2a protein comprises an opal stop codon, and the stop codon engineered AAV nucleotide sequence comprises an opal stop codon of the protein associated with the expression of the E2a protein that is engineered to an ochre stop codon or an amber stop codon. In some embodiments, a protein associated with the expression of the Rep2 protein comprises an opal stop codon, and the stop codon engineered AAV nucleotide sequence comprises an opal stop codon of the protein associated with the expression of the Rep2 protein that is engineered to an ochre stop codon or an amber stop codon. In some embodiments, the one or more stop codon engineered AAV nucleotide sequence(s) comprises one or more sequences comprising 100%, at least 99%, at least 98%, at least 95%, or at least 90% sequence identity to SEQ ID NOs: 214-221, with one or more stop codons engineered to a different stop codon. In some embodiments, the sequence is in plasmid. In some embodiments, the AAV polynucleotide coding for a AAV nucleotide sequence is stably integrated into the nuclear genome of the cell. In some embodiments, the AAV polynucleotide coding for a stop codon engineered AAV nucleotide sequence is stably integrated into the nuclear genome of the cell.

Also provided herein are cells suitable for use in the compositions and methods described herein. For example, a cell comprises the compositions for silencing suppressor tRNA. The cell can comprise a polynucleotide coding for a suppressor tRNA. The cell can comprise a polynucleotide coding for a suppressor tRNA, wherein the suppressor tRNA sequence is flanked by ITRs. The cell can comprise a composition comprising one or more suppressor tRNA coding sequences, wherein the one or more conditionally repressible suppressor tRNA coding sequence are flanked by a pair of ITRs (e.g., from 5′ to 3′: 5′ITR, a first suppressor tRNA coding sequence, a second suppressor tRNA coding sequence, a third suppressor tRNA coding sequence, and a 3′ITR). The cell can comprise a composition comprising a conditionally repressible suppressor tRNA coding sequence. The cell can comprise a composition comprising a conditionally repressible suppressor tRNA coding sequence, wherein the conditionally repressible suppressor tRNA coding sequence is flanked by ITRs. In some embodiments, the conditionally repressible suppressor tRNA coding sequence comprises a sequence coding for a repressor element and a sequence coding for a suppressor tRNA. In some embodiments, the conditionally repressible suppressor tRNA coding sequence comprises sequence coding for a repressor element upstream of a sequence coding for a suppressor tRNA. In some embodiments, the conditionally repressible suppressor tRNA coding sequence comprises sequence coding for a repressor element downstream of a sequence coding for a suppressor tRNA. The cell can comprise a composition comprising one or more conditionally repressible suppressor tRNA coding sequences, wherein the one or more conditionally repressible suppressor tRNA coding sequence are flanked by a pair of ITRs (e.g., from 5′ to 3′: 5′ITR, a first conditionally repressible suppressor tRNA coding sequence, a second conditionally repressible suppressor tRNA coding sequence, a third conditionally repressible suppressor tRNA coding sequence, and a 3′ITR). In some embodiments, the 5′ ITR comprises a polynucleotide having at least 80%, 85%, 90%, 95%, 97%, 99%, or 100% sequence identity to any one of SEQ ID NOs: 225-226. In some embodiments, the 3′ ITR comprises a polynucleotide having at least 80%, 85%, 90%, 95%, 97%, 99%, or 100% sequence identity to any one of SEQ ID NOs: 227-228. In some embodiments, a cell comprises the conditionally repressible suppressor tRNA coding sequence flanked by ITRs (e.g., by a 5′ ITR and 3′ ITR) comprising at least 80%, 85%, 90%, 95%, 97%, 99%, or 100% sequence identity to any one of SEQ ID NOs: 158-162, 164-168, 171-175, 177-181, 182-194, or 199. In some embodiments, a cell comprises a region comprising multiple copies of one or more conditionally repressible suppressor tRNA coding sequence(s) flanked by ITRs (e.g., by a 5′ ITR and 3′ ITR) comprises one or more sequences having at least 80%, 85%, 90%, 95%, 97%, 99%, or 100% sequence identity to any one of SEQ ID NOs: 158-162, 164-168, 171-175, 177-181, 182-194, or 199. In some embodiments, a cell comprises a region comprising one, two, three, four, five, six, seven, eight, nine, or ten of the one or more conditionally repressible suppressor tRNA coding sequence(s) flanked by ITRs (e.g., by a 5′ ITR and 3′ ITR) comprises one, two, three, four, five, six, seven, eight, nine, or ten of the one or more sequences having comprising at least 80%, 85%, 90%, 95%, 97%, 99%, or 100% sequence identity to any one of SEQ ID NOs: 158-162, 164-168, 171-175, 177-181, 182-194, or 199. In some embodiments, a cell comprises a polynucleotide as described herein comprises at least 80%, 85%, 90%, 95%, 97%, 99%, or 100% sequence identity to any one of SEQ ID NOs: 203-206 or 208-211. In some embodiments, a cell comprises a polynucleotide as described herein comprises at least 80%, 85%, 90%, 95%, 97%, 99%, or 100% sequence identity to any one of SEQ ID NOs: 203-206 or 208-211 without the sequence coding for transduction marker, wherein the sequence coding for the transduction marker comprises at least 80%, 85%, 90%, 95%, 97%, 99%, or 100% sequence identity to SEQ ID NO: 224. In some embodiments, the cell comprises a composition comprising moieties that target the suppressor tRNA for degradation. In some embodiments, the cell comprises a composition comprising moieties that target the suppressor tRNA for degradation and a polynucleotide coding for the suppressor tRNA, wherein the suppressor tRNA sequence is flanked by ITRs. The moieties that target the suppressor tRNA degradation upon binding can be siRNA, shRNA, DsRNA, or any combination thereof. As another example, a cell comprises the compositions for encapsidating a suppressor tRNA. The cell can comprise a composition comprising a suppressor tRNA coding sequence and a stop codon engineered AAV nucleotide sequence. The cell can comprise a composition comprising a polynucleotide comprising a suppressor tRNA coding sequence and a polynucleotide comprising a stop codon engineered AAV nucleotide sequence. In some embodiments, the stop codon engineered AAV nucleotide sequence is engineered from a stop codon capable of being readthrough by the suppressor tRNA to a different stop codon that is not capable of being readthrough by the suppressor tRNA. For example, if the suppressor tRNA is capable of reading through opal stop codons, the stop codon engineered AAV nucleotide sequence comprises engineering any opal stop codons present in the AAV nucleotide sequence to an ochre stop codon or amber stop codon. In some embodiments, if the suppressor tRNA is capable of reading through ochre stop codons, the stop codon engineered AAV nucleotide sequence comprises engineering any ochre stop codons present in the AAV nucleotide sequence to an opal stop codon or amber stop codon.

In some embodiments, a polynucleotide comprising the sequence coding for a suppressor tRNA in the cell is for a suppressor tRNA capable of suppressing an opal stop codon, an ochre stop codon, or an amber stop codon. In some embodiments, the sequence coding for a suppressor tRNA in the cell is for a suppressor tRNA capable of suppressing an opal stop codon, an ochre stop codon, or an amber stop codon. In some embodiments, the sequence coding for a suppressor tRNA in the cell comprises 100%, at least 99%, at least 98%, at least 95%, or at least 90% sequence identity to any one of SEQ ID NO: 3-SEQ ID NO: 48 or SEQ ID NO: 103-SEQ ID NO: 148. In some embodiments, the sequence of the opal stop codon anticodon loop (TCA) of any one of SEQ ID NOs: 3-48 is interchangeable with a sequence of the amber stop codon anticodon loop (CTA) for readthrough of an amber stop codon or with a sequence of the ochre stop codon anticodon (TTA) for readthrough of an ochre stop codon. In some embodiments, the sequence of the opal stop codon anticodon loop (UCA) of any one of SEQ ID NOs: 103-148 is interchangeable with a sequence of the amber stop codon anticodon loop (CUA) for readthrough of an amber stop codon or with a sequence of the ochre stop codon anticodon (UUA) for readthrough of an ochre stop codon. In some embodiments, the sequence coding for a suppressor tRNA comprises 100%, at least 99%, at least 98%, at least 95%, or at least 90% sequence identity to any one of SEQ ID NO: 3-SEQ ID NO: 48 or SEQ ID NO: 103-SEQ ID NO: 148, wherein the anticodon is engineered to bind to an ochre stop codon or an amber stop codon. In some embodiments, the sequence of the ochre stop codon anticodon loop (TTA) of any one of SEQ ID NOs: 49-68 is interchangeable with a sequence of the amber stop codon anticodon loop (CTA) for readthrough of an amber stop codon or with a sequence of the opal stop codon anticodon (TCA) for readthrough of an opal stop codon. In some embodiments, the sequence of the ochre stop codon anticodon loop (UUA) of any one of SEQ ID NOs: 249-268 is interchangeable with a sequence of the amber stop codon anticodon loop (CUA) for readthrough of an amber stop codon or with a sequence of the opal stop codon anticodon (UCA) for readthrough of an ochre stop codon. In some embodiments, the sequence coding for a suppressor tRNA comprises 100%, at least 99%, at least 98%, at least 95%, or at least 90% sequence identity to any one of SEQ ID NO: 49-SEQ ID NO: 68 or SEQ ID NO: 249-SEQ ID NO: 268, wherein the anticodon is engineered to bind to an opal stop codon or an amber stop codon. In some embodiments, the sequence of the amber stop codon anticodon loop (CTA) of any one of SEQ ID NOs: 69-88 is interchangeable with a sequence of the opal stop codon anticodon loop (TCA) for readthrough of an opal stop codon or with a sequence of the ochre stop codon anticodon (TTA) for readthrough of an ochre stop codon. In some embodiments, the sequence of the amber stop codon anticodon loop (CUA) of any one of SEQ ID NOs: 249-268 is interchangeable with a sequence of the opal stop codon anticodon loop (UCA) for readthrough of an opal stop codon or with a sequence of the ochre stop codon anticodon (UUA) for readthrough of an ochre stop codon. In some embodiments, the sequence coding for a suppressor tRNA comprises 100%, at least 99%, at least 98%, at least 95%, or at least 90% sequence identity to any one of SEQ ID NO: 69-SEQ ID NO: 88 or SEQ ID NO: 269-SEQ ID NO: 288, wherein the anticodon is engineered to bind to an ochre stop codon or an opal stop codon.

A cell as provided herein can further comprise a polynucleotide comprising an AAV nucleotide sequence for producing AAV virions, wherein the suppressor tRNA is encapsidated in the produced virion. A cell as provided herein can further comprise an AAV nucleotide sequence for producing AAV virions, wherein the suppressor tRNA is encapsidated in the produced virion. In some embodiments, a cell further comprises a polynucleotide comprising a stop codon engineered AAV nucleotide sequence for producing AAV virions, wherein the suppressor tRNA is encapsidated in the produced virion. In some embodiments, a cell further comprises a stop codon engineered AAV nucleotide sequence for producing AAV virions, wherein the suppressor tRNA is encapsidated in the produced virion. In some cases, the AAV nucleotide sequence encodes for element(s) for producing AAV virion are any proteins or nucleotides involved in or required for the production of AAV virion in a cell. In some cases, the stop codon engineered AAV nucleotide sequence encodes the element(s) for producing AAV virion that are stop codon engineered. In some cases, the stop codon engineered AAV nucleotide sequence comprises one or more changes, relative to a reference sequence (e.g., a wild-type sequence) selected from the group consisting of: ochre to amber, ochre to opal, opal to ochre, opal to amber, amber to ochre, amber to opal, and combinations thereof. In some cases, the element(s) for producing AAV virion of a stop codon engineered AAV nucleotide sequence comprises one or more changes, relative to a reference sequence (e.g., a wild-type sequence) selected from the group consisting of: ochre to amber, ochre to opal, opal to ochre, opal to amber, amber to ochre, amber to opal, and combinations thereof. In some cases, the element(s) for producing AAV virion of an AAV nucleotide sequence in the cell are selected from the group consisting of: a Rep protein, a Cap protein, a Helper protein, VA RNA, an AAP protein, an MAAP protein, a Protein X protein, and combinations thereof. In some cases, the element(s) for producing AAV virion using a polynucleotide coding for an AAV nucleotide sequence are selected from the group consisting of polynucleotide coding for: a Rep protein, a Cap protein, a Helper protein, VA RNA, an AAP protein, an MAAP protein, a Protein X protein, a Hexon Assembly Protein, a Hexon-associated Precursor protein, a 23K endoprotease, and combinations thereof. In some cases, the element(s) for producing AAV virion using a polynucleotide coding for a stop codon engineered AAV nucleotide sequence are selected from the group consisting of polynucleotide coding for: a Rep protein, a Cap protein, a Helper protein, VA RNA, an AAP protein, an MAAP protein, a Protein X protein, and combinations thereof, wherein one or more stop codons of the AAV nucleotide sequence are engineered to be a different stop codon. In some cases, the element(s) for producing AAV virion using a polynucleotide coding for a stop codon engineered AAV nucleotide sequence are selected from the group consisting of polynucleotide coding for: a Rep protein, a Cap protein, a Helper protein, VA RNA, an AAP protein, an MAAP protein, a Protein X protein, a Hexon Assembly Protein, a Hexon-associated Precursor protein, a 23K endoprotease, and combinations thereof, wherein one or more stop codons of the AAV nucleotide sequence are engineered to be a different stop codon. In some embodiments, the Rep protein is a Rep2 protein. In some embodiments, the stop codon engineered AAV nucleotide sequence comprises an ochre stop codon of the sequence coding for Rep changed to an opal stop codon or an opal stop codon. In some embodiments, the Rep protein is Rep78, Rep52, Rep68, Rep40, or any combination thereof. In some embodiments, an opal stop codon of the sequence coding for the Rep68 is changed to an amber stop codon or an ochre stop codon. In some embodiments, the stop codon engineered AAV nucleotide sequence comprises an opal stop codon of the sequence coding for the Rep68 is engineered to an amber stop codon or an ochre stop codon. In some embodiments, an opal stop codon of the sequence coding for the Rep40 is changed to an amber stop codon or an ochre stop codon. In some embodiments, the stop codon engineered AAV nucleotide sequence comprises an opal stop codon of the sequence coding for the Rep40 is engineered to an amber stop codon or an ochre stop codon. In some embodiments, an ochre stop codon of the sequence coding for the Rep78 is changed to an amber stop codon or an opal stop codon. In some embodiments, the stop codon engineered AAV nucleotide sequence comprises an ochre stop codon of the sequence coding for the Rep78 is engineered to an amber stop codon or an opal stop codon. In some embodiments, an ochre stop codon of the sequence coding for the Rep52 is changed to an amber stop codon or an opal stop codon. In some embodiments, the stop codon engineered AAV nucleotide sequence comprises an ochre stop codon of the sequence coding for the Rep52 is engineered to an amber stop codon or an opal stop codon. In some embodiments, the Cap protein is VP1, VP2, VP3, or any combination thereof. In some embodiments, an ochre stop codon of the sequence coding for VP1 is changed to an amber stop codon or an opal stop codon. In some embodiments, the stop codon engineered AAV nucleotide sequence comprises an ochre stop codon of the sequence coding for the VP1 is engineered to an amber stop codon or an opal stop codon. In some embodiments, an ochre stop codon of the sequence coding for VP2 is changed to an amber stop codon or an opal stop codon. In some embodiments, the stop codon engineered AAV nucleotide sequence comprises an ochre stop codon of the sequence coding for the VP2 is engineered to an amber stop codon or an opal stop codon. In some embodiments, an ochre stop codon of the sequence coding for VP3 is changed to an amber stop codon or an opal stop codon. In some embodiments, the stop codon engineered AAV nucleotide sequence comprises an ochre stop codon of the sequence coding for the VP3 is engineered to an amber stop codon or an opal stop codon. In some embodiments, the Cap protein is a Cap5 protein. In some embodiments, an ochre stop codon of the sequence coding for Cap5 is changed to an amber stop codon or an opal stop codon. In some embodiments, the stop codon engineered AAV nucleotide sequence comprises an ochre stop codon of the sequence coding for the Cap5 is engineered to an amber stop codon or an opal stop codon. In some embodiments, the Helper protein is E1a, E1b, E4, E2a, or any combination thereof. In some embodiments, a stop codon of the sequence coding for E1a is changed to a different stop codon. In some embodiments, the stop codon engineered AAV nucleotide sequence comprises a stop codon of the sequence coding for the E1a is engineered to a different stop codon. In some embodiments, the stop codon engineered AAV nucleotide sequence comprises an ochre stop codon of the sequence coding for the E1a is engineered to an amber stop codon or an opal stop codon. In some embodiments, a stop codon of the sequence coding for E1b is changed to a different stop codon. In some embodiments, the stop codon engineered AAV nucleotide sequence comprises a stop codon of the sequence coding for the E1b is engineered to a different stop codon. In some embodiments, the E1b protein is a 55k E1b protein, a 19K E1b protein, or both. In some embodiments, a stop codon of the sequence coding for 55k E1b protein is changed to a different stop codon. In some embodiments, a stop codon of the sequence coding for 55k E1b protein is changed from an opal stop codon to an ochre stop codon or an amber stop codon. In some embodiments, a stop codon of the sequence coding for 19k E1b protein is changed to a different stop codon. In some embodiments, a stop codon of the sequence coding for 19k E1b protein is changed from an opal stop codon to an ochre stop codon or an amber stop codon. In some embodiments, a stop codon of the sequence coding for E4 is changed to a different stop codon. In some embodiments, the stop codon engineered AAV nucleotide sequence comprises a stop codon of the sequence coding for the E4 is engineered to a different stop codon. In some embodiments, E4 is E4orf1. In some embodiments, E4 is E4orf2. In some embodiments, E4 is E4orf3. In some embodiments, E4 is E4orf4. In some embodiments, E4 is E4orf6. In some embodiments, the stop codon engineered AAV nucleotide sequence comprises a stop codon of the sequence coding for the E4orf1 is engineered from an ochre stop codon to an opal stop codon or an amber stop codon. In some embodiments, the stop codon engineered AAV nucleotide sequence comprises a stop codon of the sequence coding for the E4orf2 is engineered from an opal stop codon to an ochre stop codon or an amber stop codon. In some embodiments, the stop codon engineered AAV nucleotide sequence comprises a stop codon of the sequence coding for the E4orf3 is engineered from an ochre stop codon to an opal stop codon or an amber stop codon. In some embodiments, the stop codon engineered AAV nucleotide sequence comprises a stop codon of the sequence coding for the E4orf4 is engineered from an amber stop codon to an opal stop codon or an ochre stop codon. In some embodiments, the stop codon engineered AAV nucleotide sequence comprises a stop codon of the sequence coding for the E4orf6 is engineered from an amber stop codon to an opal stop codon or an ochre stop codon. In some embodiments, a stop codon of the sequence coding for E2a is changed to a different stop codon. In some embodiments, the stop codon engineered AAV nucleotide sequence comprises a stop codon of the sequence coding for the E2a is engineered to a different stop codon. In some embodiments, the stop codon engineered AAV nucleotide sequence comprises a stop codon of the sequence coding for the E2a is engineered from an ochre stop codon to an opal stop codon or an amber stop codon. In some embodiments, a stop codon of the sequence coding for VA RNA is changed to a different stop codon. In some embodiments, the stop codon engineered AAV nucleotide sequence comprises a stop codon of the sequence coding for the VA RNA is engineered to a different stop codon. In some embodiments, an opal stop codon of the sequence coding for AAP is changed to an amber stop codon or an ochre stop codon. In some embodiments, the stop codon engineered AAV nucleotide sequence comprises an opal stop codon of the sequence coding for the AAP is engineered to an amber stop codon or an ochre stop codon. In some embodiments, an amber stop codon of the sequence coding for MAAP is changed to an opal stop codon or an ochre stop codon. In some embodiments, the stop codon engineered AAV nucleotide sequence comprises an amber stop codon of the sequence coding for the MAAP is engineered to an opal stop codon or an ochre stop codon. In some embodiments, an opal stop codon of the sequence coding for Protein X is changed to an amber stop codon or an ochre stop codon. In some embodiments, the stop codon engineered AAV nucleotide sequence comprises an opal stop codon of the sequence coding for the Protein X is engineered to an amber stop codon or an ochre stop codon. In some embodiments, the stop codon engineered AAV nucleotide sequence comprises an ochre stop codon of the sequence coding for the Fiber is engineered to an amber stop codon or an opal stop codon. In some embodiments, the stop codon engineered AAV nucleotide sequence comprises an amber stop codon of the sequence coding for the Hexon Assembly protein that is engineered to an ochre stop codon or an opal stop codon. In some embodiments, the stop codon engineered AAV nucleotide sequence comprises an opal stop codon of the sequence coding for the Hexon-associated Precursor protein that is engineered to an ochre stop codon or an amber stop codon. In some embodiments, the stop codon engineered AAV nucleotide sequence comprises an ochre stop codon of the sequence coding for the 23k Endoprotease that is engineered to an opal stop codon or an amber stop codon. In some embodiments, a protein associated with the expression of the Hexon assembly protein comprises an opal stop codon, and the stop codon engineered AAV nucleotide sequence comprises an opal stop codon of the protein associated with the expression of the Hexon assembly protein that is engineered to an ochre stop codon or an amber stop codon. In some embodiments, a protein associated with the expression of the 23k Endoprotease comprises an opal stop codon, and the stop codon engineered AAV nucleotide sequence comprises an opal stop codon of the protein associated with the expression of the 23k Endoprotease that is engineered to an ochre stop codon or an amber stop codon. In some embodiments, a protein associated with the expression of the VP1 protein comprises an opal stop codon, and the stop codon engineered AAV nucleotide sequence comprises an opal stop codon of the protein associated with the expression of the VP1 protein that is engineered to an ochre stop codon or an amber stop codon. In some embodiments, a protein associated with the expression of the E2a protein comprises an opal stop codon, and the stop codon engineered AAV nucleotide sequence comprises an opal stop codon of the protein associated with the expression of the E2a protein that is engineered to an ochre stop codon or an amber stop codon. In some embodiments, a protein associated with the expression of the Rep2 protein comprises an opal stop codon, and the stop codon engineered AAV nucleotide sequence comprises an opal stop codon of the protein associated with the expression of the Rep2 protein that is engineered to an ochre stop codon or an amber stop codon. In some embodiments, the one or more stop codon engineered AAV nucleotide sequence(s) comprises one or more sequences comprising 100%, at least 99%, at least 98%, at least 95%, or at least 90% sequence identity to SEQ ID NOs: 214-221, with one or more stop codons engineered to a different stop codon. In some embodiments, the sequence is in plasmid. In some embodiments, the AAV polynucleotide coding for a AAV nucleotide sequence is stably integrated into the nuclear genome of the cell. In some embodiments, the AAV polynucleotide coding for a stop codon engineered AAV nucleotide sequence is stably integrated into the nuclear genome of the cell. In some embodiments, the cell is a mammalian cell. In some embodiments, the cell for producing AAV is a mammalian cell for producing AAV. In some embodiments, the cell is an HEK293 cell or a CHO cell. In some embodiments, the cell for producing AAV is an HEK293 cell or a CHO cell. In some embodiments, the cell is an insect cell. In some embodiments, the cell is an Sf9 cell. In some embodiments, the cell for producing AAV is an insect cell. In some embodiments, the cell for producing AAV is an Sf9 cell. In some embodiments, the cell is capable of producing a virion encapsidating the suppressor tRNA. In some embodiments, the cell is capable of producing a virion encapsidating the conditionally repressible suppressor tRNA coding sequence.

In some embodiments, the cell further comprises a repressor. In some embodiments, the cell for producing AAV further comprises a repressor. The repressor can be any moiety capable of binding the repressor element, wherein the binding by the repressor to the repressor element inhibits expression of a suppressor tRNA. A repressor, for example, can be a protein, nucleotide, or hormone. In some embodiments, the repressor is a Tet repressor protein or a guide RNA complexed to a catalytically dead Cas protein that is linked or fused to a repressor domain. In some embodiments, the repressor domain is a krab domain.

In some embodiments, a Tet repressor protein is a TetR protein. In some embodiments, the Tet repressor has at at least 80%, 85%, 90%, 95%, 97%, 99%, or 100% sequence identity SEQ ID NO: 223. In some embodiments, the cell further comprises a polynucleotide comprising a sequence coding for a repressor. In some embodiments, the cell further comprises a polynucleotide comprising a sequence coding for a repressor having least 80%, 85%, 90%, 95%, 97%, 99%, or 100% sequence identity SEQ ID NO: 222.

In some embodiments, during the methods of producing the virion, the suppressor tRNA is silenced, but when the produced virion is delivered to a subject (e.g., a patient), the suppressor tRNA is not silenced. For example, the silencing in the cells for producing AAV utilizes a repressor and a repressor element for silencing expression of the suppressor tRNA from the polynucleotide coding for the suppressor tRNA that is silenced in the cells for producing AAV that express a repressor, but when the produced virion is delivered to a subject (e.g., a patient), the repressor is absent in the subject's cells and suppressor tRNA is therefore not silenced. In some embodiments, a composition of siRNA, shRNA, DsRNA, or any combination thereof is used in the method of silencing suppressor tRNA in cells for producing AAV, but when the produced virion is delivered to a subject (e.g., a patient), the composition of siRNA, shRNA, DsRNA, or any combination thereof is absent in the subject's cells and suppressor tRNA is therefore not silenced.

### Methods

A method of silencing suppressor tRNA for virion production can comprise: a) providing a cell comprising a repressor; b) transfecting the cell with a sequence coding for a helper protein, a sequence coding for a Rep protein, and a sequence coding for a Cap protein; c) transfecting the cell with the composition comprising a conditionally repressible suppressor tRNA coding sequence, wherein the conditionally repressible suppressor tRNA coding sequence is flanked by ITRs; and d) producing virions encapsidating the conditionally repressible suppressor tRNA coding sequence, wherein the repressor binds to a repressor element of the conditionally repressible suppressor tRNA coding sequence, thereby silencing expression of the suppressor tRNA for virion production.

A method of silencing suppressor tRNA for virion production can comprise: a) providing a cell comprising a repressor; b) transfecting the cell with a polynucleotide comprising sequence coding for a helper protein, a polynucleotide comprising a sequence coding for a Rep protein, and a polynucleotide comprising a sequence coding for a Cap protein; c) transfecting the cell with a composition comprising a polynucleotide having a conditionally repressible suppressor tRNA coding sequence, wherein the conditionally repressible suppressor tRNA coding sequence is flanked by ITRs; and d) producing virions encapsidating the conditionally repressible suppressor tRNA coding sequence, wherein the repressor binds to a repressor element of the conditionally repressible suppressor tRNA coding sequence, thereby silencing expression of the suppressor tRNA for virion production.

A method of silencing suppressor tRNA for virion production can comprise: a) providing a cell comprising a suppressor tRNA flanked by ITRs; b) transfecting the cell with a sequence coding for a helper protein, a sequence coding for a Rep protein, and a sequence coding for a Cap protein; c) transfecting the cell with a composition that comprises siRNA, shRNA, DsRNA or any combination; and d) producing virions encapsidating the conditionally repressible suppressor tRNA coding sequence, wherein the siRNA, shRNA, DsRNA or any combination thereof binds to the suppressor tRNA and targets the suppressor tRNA for degradation, thereby silencing the suppressor tRNA for virion production.

A method of silencing suppressor tRNA for virion production can comprise: a) providing a cell comprising a polynucleotide having sequence coding for a suppressor tRNA flanked by ITRs; b) transfecting the cell with a polynucleotide comprising sequence coding for a helper protein, a polynucleotide comprising a sequence coding for a Rep protein, and a polynucleotide comprising a sequence coding for a Cap protein; and d) producing virions encapsidating the polynucleotide comprising conditionally repressible suppressor tRNA coding sequence, wherein the siRNA, shRNA, DsRNA or any combination thereof binds to the suppressor tRNA and targets the suppressor tRNA for degradation, thereby silencing the suppressor tRNA for virion production.

In some cases, silencing comprises a reduction of at least 40%, e.g., at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% in the amount or activity of suppressor tRNA(s) relative to a control. In some cases, silencing comprises a reduction of at least 40% in the amount or activity of suppressor tRNA(s) relative to a control. In some cases, silencing comprises a reduction of at least 45% in the amount or activity of suppressor tRNA(s) relative to a control. In some cases, silencing comprises a reduction of at least 50% in the amount or activity of suppressor tRNA(s) relative to a control. In some cases, silencing comprises a reduction of at least 55% in the amount or activity of suppressor tRNA(s) relative to a control. In some cases, silencing comprises a reduction of at least 60% in the amount or activity of suppressor tRNA(s) relative to a control. In some cases, silencing comprises a reduction of at least 65% in the amount or activity of suppressor tRNA(s) relative to a control. In some cases, silencing comprises a reduction of at least 70% in the amount or activity of suppressor tRNA(s) relative to a control. In some cases, silencing comprises a reduction of at least 75% in the amount or activity of suppressor tRNA(s) relative to a control. In some cases, silencing comprises a reduction of at least 80% in the amount or activity of suppressor tRNA(s) relative to a control. In some cases, silencing comprises a reduction of at least 85% in the amount or activity of suppressor tRNA(s) relative to a control. In some cases, silencing comprises a reduction of at least 90% in the amount or activity of suppressor tRNA(s) relative to a control. In some cases, silencing comprises a reduction of at least 95% in the amount or activity of suppressor tRNA(s) relative to a control. In some cases, silencing comprises a reduction of at least 100% in the amount or activity of suppressor tRNA(s) relative to a control.

In some cases, e.g., in the case of a repressor element-suppressor tRNA construct, the control is an equivalent construct with the suppressor tRNA, but without the repressor element. In some cases, the control is a) expression of the suppressor tRNA in the cell(s) when the repressor is inhibited from binding to the repressor element, optionally wherein the repressor is inhibited from binding to the repressor element by binding of a switching agent to the repressor; or b) expression of the suppressor tRNA from reference cell(s) comprising the vector, wherein the reference cell(s) do not express the repressor. For example, the repressor is a Tet repressor protein and the switching agent is doxycycline.

A method of producing virion encapsidating a sequence coding for a suppressor tRNA can comprise a) providing a cell comprising a repressor; b) transfecting the cell with a sequence coding for a helper protein, a sequence coding for a Rep protein, and a sequence coding for a Cap protein; c) transfecting the cell with the composition comprising a conditionally repressible suppressor tRNA coding sequence, wherein the conditionally repressible suppressor tRNA coding sequence is flanked by ITRs; and d) producing virions encapsidating the sequence coding for a suppressor tRNA, wherein the repressor binds to the repressor element of the conditionally repressible suppressor tRNA coding sequence, thereby silencing expression of the suppressor tRNA.

A method of producing virion encapsidating a polynucleotide comprising a sequence coding for a suppressor tRNA, can comprise a) providing a cell comprising a repressor; b) transfecting the cell with a polynucleotide comprising sequence coding for a helper protein, a polynucleotide comprising a sequence coding for a Rep protein, and a polynucleotide comprising a sequence coding for a Cap protein; c) transfecting the cell with the composition comprising a polynucleotide having a conditionally repressible suppressor tRNA coding sequence that comprises the sequence coding for the suppressor tRNA and a repressor element, wherein the conditionally repressible suppressor tRNA coding sequence is flanked by ITRs; and d) producing virions encapsidating the polynucleotide comprising the sequence coding for the suppressor tRNA, wherein the repressor binds to the repressor element of the conditionally repressible suppressor tRNA coding sequence, thereby silencing expression of the suppressor tRNA.

A method of producing virion encapsidating a polynucleotide comprising a sequence coding for a suppressor tRNA, can comprise a) providing a cell comprising a repressor; b) transfecting the cell with a polynucleotide comprising sequence coding for a helper protein, a polynucleotide comprising a sequence coding for a Rep protein, and a polynucleotide comprising a sequence coding for a Cap protein; c) transfecting the cell with the composition comprising a polynucleotide having a conditionally repressible suppressor tRNA coding sequence that comprises the sequence coding for the suppressor tRNA and a repressor element, wherein the conditionally repressible suppressor tRNA coding sequence is flanked by ITRs; and d) producing virions encapsidating the polynucleotide having a conditionally repressible suppressor tRNA coding sequence that comprises the sequence coding for the suppressor tRNA, wherein the repressor binds to a repressor element of the conditionally repressible suppressor tRNA coding sequence, thereby silencing expression of the suppressor tRNA.

A method of producing virion encapsidating a sequence coding for a suppressor tRNA can comprise a) providing a cell comprising a suppressor tRNA flanked by ITRs; b) transfecting the cell with a sequence coding for a helper protein, a sequence coding for a Rep protein, and a sequence coding for a Cap protein; c) transfecting the cell with a composition that comprises siRNA, shRNA, DsRNA or any combination; and d) producing virions encapsidating the suppressor tRNA coding sequence, wherein the siRNA, shRNA, DsRNA or any combination thereof binds to the suppressor tRNA and targets the suppressor tRNA for degradation, thereby silencing the suppressor tRNA.

A method of producing virion encapsidating a polynucleotide comprising a sequence coding for a suppressor tRNA can comprise a) providing a cell comprising the polynucleotide comprising the suppressor tRNA flanked by ITRs; b) transfecting the cell with a polynucleotide comprising sequence coding for a helper protein, a polynucleotide comprising a sequence coding for a Rep protein, and a polynucleotide comprising a sequence coding for a Cap protein; c) transfecting the cell with a composition that comprises siRNA, shRNA, DsRNA or any combination; and d) producing virions encapsidating the polynucleotide comprising a sequence coding for a suppressor tRNA, wherein the siRNA, shRNA, DsRNA or any combination thereof binds to the suppressor tRNA and targets the suppressor tRNA for degradation, thereby silencing the suppressor tRNA.

A method of producing virion encapsidating a polynucleotide comprising a sequence coding for a suppressor tRNA can comprise a) providing a cell comprising the polynucleotide comprising the suppressor tRNA flanked by ITRs; b) transfecting the cell with a polynucleotide comprising sequence coding for a helper protein, a polynucleotide comprising a sequence coding for a Rep protein, and a polynucleotide comprising a sequence coding for a Cap protein, wherein the polynucleotide comprising sequence coding for the helper protein, the polynucleotide comprising the sequence coding for the Rep protein, and the polynucleotide comprising the sequence coding for the Cap protein, or any combination thereof, comprise one or more engineered stop codons; and c) producing virions encapsidating the polynucleotide comprising a sequence coding for a suppressor tRNA.

A method of producing virion encapsidating a sequence coding for a suppressor tRNA can comprise: a) providing a cell comprising: a sequence encoding a suppressor tRNA flanked by ITRs, a sequence encoding a helper protein, a sequence encoding a VA RNA, a sequence encoding a Rep protein, a sequence encoding a Cap protein, and optionally a sequence coding for AAP, a sequence coding for MAAP, and/or a sequence coding for Protein X, wherein one or more of the sequence encoding the helper protein, the sequence encoding the VA RNA, the sequence encoding the Rep protein, the sequence coding for the Cap protein, the sequence coding for AAP, the sequence coding for MAAP, and/or the sequence coding for Protein X is stop codon engineered; and b) culturing the cell under conditions suitable for expression and encapsidation of the suppressor tRNA.

In some embodiments, the repressor is a Tet repressor protein or a guide RNA complexed to a catalytically dead Cas protein that is linked or fused to a repressor domain. In some embodiments, the repressor domain is a krab domain. In some embodiments, the Rep protein is Rep78, Rep52, Rep68, Rep40, or any combination thereof. In some embodiments, an opal stop codon of the sequence coding for the Rep68 is changed to an amber stop codon or an ochre stop codon. In some embodiments, an opal stop codon of the sequence coding for the Rep40 is changed to an amber stop codon or an ochre stop codon. In some embodiments, an ochre stop codon of the sequence coding for the Rep78 is changed to an amber stop codon or an opal stop codon. In some embodiments, an ochre stop codon of the sequence coding for the Rep52 is changed to an amber stop codon or an opal stop codon. In some embodiments, the Cap protein is VP1, VP2, VP3, or any combination thereof. In some embodiments, an ochre stop codon of the sequence coding for VP1 is changed to an amber stop codon or an opal stop codon. In some embodiments, an ochre stop codon of the sequence coding for VP2 is changed to an amber stop codon or an opal stop codon. In some embodiments, an ochre stop codon of the sequence coding for VP3 is changed to an amber stop codon or an opal stop codon. In some embodiments, the helper protein is E1a, E1b, E4, E2a, VA, or any combination thereof. In some embodiments, the cell is a mammalian cell. In some embodiments, the cell is an HEK293 cell or a CHO cell. In some embodiments, the cell is an insect cell. In some embodiments, the cell is an Sf9 cell. In some embodiments, the virion is an AAV virion. In some embodiments, a capsid of the AAV virion is selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combinations thereof. In some embodiments, the AAV virion is comprises a hybrid capsid. In some embodiments, the hybrid capsid comprises any combination of capsids selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combinations thereof. In some embodiments, the hybrid capsid is AAV2/5 or AAV-DJ.

Provided herein are methods for increasing the titer of virions encapsidating a suppressor tRNA. Provided herein are methods for increasing the titer of virions encapsidating a polynucleotide coding for a suppressor tRNA. In some embodiments, during the packaging of suppressor tRNA sequence into the virion, the suppressor tRNA is silenced, producing an increase in titer of encapsidated suppressor tRNAs compared to methods of producing the virion wherein the suppressor tRNA is not silenced during packaging. In some embodiments, during the packaging of a polynucleotide comprising the suppressor tRNA sequence into the virion, the suppressor tRNA is silenced, producing an increase in titer of encapsidated polynucleotide comprising the suppressor tRNA sequence compared to methods of producing the virion wherein the suppressor tRNA is not silenced during packaging. In some embodiments, the silencing utilizes the compositions as described herein. For example, a repressor and a repressor element. In some embodiments, a composition of siRNA, shRNA, DsRNA, or any combination thereof is used in the method of silencing suppressor tRNA.

Provided herein are methods for increasing the titer of virions encapsidating a polynucleotide coding for a suppressor tRNA. In some embodiments, during the replication of viral molecules (e.g., viral protein and/or viral RNA) for virion production, the suppressor tRNA is silenced, producing an increase in titer of encapsidated polynucleotide coding for a suppressor tRNA compared to methods of producing the virion wherein the suppressor tRNA is not silenced during replication of viral molecules. In some embodiments, the silencing utilizes the compositions as described herein. For example, a repressor and a repressor element. In some embodiments, a composition of siRNA, shRNA, DsRNA, or any combination thereof is used in the method of silencing suppressor tRNA.

Provided herein are methods for increasing the titer of virions encapsidating a polynucleotide coding for a suppressor tRNA. In some embodiments, during replication of viral molecules (e.g., viral protein and/or viral RNA) for virion production and packaging of polynucleotide coding for a suppressor tRNA sequence into the virion, the suppressor tRNA is silenced, producing an increase in titer of encapsidated polynucleotide coding for a suppressor tRNAs compared to methods of producing the virion wherein the suppressor tRNA is not silenced during replication of viral molecules (e.g., viral protein and/or viral RNA) for virion production and packaging of polynucleotide coding for a suppressor tRNA sequence into the virion. In some embodiments, the silencing utilizes the compositions as described herein. For example, a repressor and a repressor element. In some embodiments, a composition of siRNA, shRNA, DsRNA, or any combination thereof is used in the method of silencing suppressor tRNA.

A method of increasing titer of virions encapsidating a sequence coding for a suppressor tRNA by silencing the suppressor tRNA can comprise a) providing a cell comprising a repressor; b) transfecting the cell with a sequence coding for a helper protein, a sequence coding for a Rep protein, and a sequence coding for a Cap protein; c) transfecting the cell with a composition comprising a conditionally repressible suppressor tRNA coding sequence, wherein the conditionally repressible suppressor tRNA coding sequence is flanked by ITRs; and d) producing virions encapsidating the sequence coding for a suppressor tRNA, wherein the repressor binds to a repressor element of the conditionally repressible suppressor tRNA coding sequence, thereby silencing expression of the suppressor tRNA for virion production, and wherein the titer of virions encapsidating the sequence coding for the suppressor tRNA is increased compared to titer of virions encapsidating the sequence for the suppressor tRNA produced by steps b)-d) or produced by transfecting the cell with a sequence coding for a suppressor tRNA and transfecting the cell with a sequence coding for a helper protein, a sequence coding for a Rep protein, and a sequence coding for a Cap protein.

A method of increasing titer of virions encapsidating a polynucleotide comprising a sequence coding for a suppressor tRNA by silencing the suppressor tRNA can comprise a) providing a cell comprising a repressor; b) transfecting the cell with a polynucleotide comprising sequence coding for a helper protein, a polynucleotide comprising a sequence coding for a Rep protein, and a polynucleotide comprising a sequence coding for a Cap protein; c) transfecting the cell with a composition comprising a polynucleotide having a conditionally repressible suppressor tRNA coding sequence that comprises the sequence coding for the suppressor tRNA and a repressor element, wherein the conditionally repressible suppressor tRNA coding sequence is flanked by ITRs; and d) producing virions encapsidating the polynucleotide comprising the sequence for the suppressor tRNA, wherein the repressor binds to the repressor element of the conditionally repressible suppressor tRNA coding sequence, thereby silencing expression of the suppressor tRNA for virion production, and wherein the titer of virions encapsidating the sequence coding for the suppressor tRNA is increased compared to titer of virions encapsidating the polynucleotide comprising the sequence for the suppressor tRNA produced by steps b)-d) and not step a) or produced by transfecting the cell with the polynucleotide sequence coding for the suppressor tRNA instead of the polynucleotide having the conditionally repressible suppressor tRNA coding sequence that comprises the sequence coding for the suppressor tRNA and the repressor element and transfecting the cell with transfecting the cell with a polynucleotide comprising sequence coding for a helper protein, a polynucleotide comprising a sequence coding for a Rep protein, and a polynucleotide comprising a sequence coding for a Cap protein.

A method of increasing titer of virions encapsidating a polynucleotide comprising a sequence coding for a suppressor tRNA by silencing the suppressor tRNA can comprise a) providing a cell comprising a repressor; b) transfecting the cell with a polynucleotide comprising sequence coding for a helper protein, a polynucleotide comprising a sequence coding for a Rep protein, and a polynucleotide comprising a sequence coding for a Cap protein; c) transfecting the cell with a composition comprising a polynucleotide having a conditionally repressible suppressor tRNA coding sequence that comprises the sequence coding for the suppressor tRNA and the repressor element, wherein the conditionally repressible suppressor tRNA coding sequence is flanked by ITRs; and d) producing virions encapsidating the polynucleotide comprising the sequence for the suppressor tRNA, wherein the repressor binds to the repressor element of the conditionally repressible suppressor tRNA coding sequence, thereby silencing expression of the suppressor tRNA for virion production, and wherein the titer of virions encapsidating the polynucleotide comprising the sequence coding for the suppressor tRNA is increased compared to titer of virions encapsidating the polynucleotide comprising the sequence for the suppressor tRNA produced by steps b)-d) and not a) wherein the transfected cell lacks a repressor or produced by transfecting the cell with the polynucleotide comprising the polynucleotide having the sequence coding for the suppressor tRNA and transfecting the cell with the polynucleotide comprising the sequence coding for the helper protein, the polynucleotide comprising the sequence coding for the Rep protein, and the polynucleotide comprising the sequence coding for the Cap protein.

A method of increasing titer of virions encapsidating a polynucleotide comprising a sequence coding for a suppressor tRNA by silencing the suppressor tRNA can comprise a) providing a cell comprising a repressor; b) transfecting the cell comprising the repressor with a polynucleotide comprising sequence coding for a helper protein, a polynucleotide comprising a sequence coding for a Rep protein, and a polynucleotide comprising a sequence coding for a Cap protein; c) transfecting the cell comprising the repressor with a composition comprising a polynucleotide having a conditionally repressible suppressor tRNA coding sequence that comprises the sequence coding for the suppressor tRNA and the repressor element, wherein the conditionally repressible suppressor tRNA coding sequence is flanked by ITRs; and d) producing virions encapsidating the polynucleotide comprising the sequence for the suppressor tRNA, wherein the repressor binds to the repressor element of the conditionally repressible suppressor tRNA coding sequence, thereby silencing expression of the suppressor tRNA for virion production, and wherein the titer of virions encapsidating the polynucleotide comprising the sequence coding for the suppressor tRNA is increased compared to titer of virions encapsidating the polynucleotide comprising the sequence for the suppressor tRNA produced by a) providing a cell comprising lacking the repressor; b) transfecting the cell lacking the repressor with the polynucleotide comprising the sequence coding for the helper protein, the polynucleotide comprising the sequence coding for the Rep protein, and the polynucleotide comprising the sequence coding for the Cap protein; c) transfecting the cell lacking the repressor with a composition comprising a polynucleotide having a conditionally repressible suppressor tRNA coding sequence that comprises the sequence coding for the suppressor tRNA and the repressor element, wherein the conditionally repressible suppressor tRNA coding sequence is flanked by ITRs; and d) producing virions encapsidating the polynucleotide comprising the sequence for the suppressor tRNA.

A method of increasing titer of virions encapsidating a polynucleotide comprising a sequence coding for a suppressor tRNA by silencing the suppressor tRNA can comprise a) providing a cell comprising a repressor; b) transfecting the cell with a polynucleotide comprising sequence coding for a helper protein, a polynucleotide comprising a sequence coding for a Rep protein, and a polynucleotide comprising a sequence coding for a Cap protein; c) transfecting the cell with a composition comprising a polynucleotide having a conditionally repressible suppressor tRNA coding sequence that comprises the sequence coding for the suppressor tRNA and the repressor element, wherein the conditionally repressible suppressor tRNA coding sequence is flanked by ITRs; and d) producing virions encapsidating the polynucleotide comprising the sequence for the suppressor tRNA, wherein the repressor binds to the repressor element of the conditionally repressible suppressor tRNA coding sequence, thereby silencing expression of the suppressor tRNA for virion production, and wherein the titer of virions encapsidating the polynucleotide comprising the polynucleotide comprising the sequence coding for the suppressor tRNA is increased compared to titer of virions encapsidating the polynucleotide comprising the sequence for the suppressor tRNA produced by transfecting the cell with the polynucleotide comprising the sequence coding for a suppressor tRNA and transfecting the cell with a sequence coding for a helper protein, a sequence coding for a Rep protein, and a sequence coding for a Cap protein.

A method of increasing titer of virions encapsidating a sequence coding for a suppressor tRNA by silencing the suppressor tRNA can comprise a) transfecting a cell with the sequence coding for the suppressor tRNA flanked by ITRs; b) transfecting the cell with a sequence coding for a helper protein, a sequence coding for a Rep protein, and a sequence coding for a Cap protein; c) transfecting the cell with a composition that comprises siRNA, shRNA, DsRNA or any combination; and d) producing virions encapsidating the sequence coding for the suppressor tRNA, wherein the siRNA, shRNA, DsRNA or any combination thereof binds to the suppressor tRNA and targets the suppressor tRNA for degradation, thereby silencing the suppressor tRNA, and wherein the titer of virions encapsidating the sequence coding for the suppressor tRNA is increased compared to titer of virions encapsidating the sequence for the suppressor tRNA produced by steps a), b), and d).

A method of increasing titer of virions encapsidating a polynucleotide comprising a sequence coding for a suppressor tRNA by silencing the suppressor tRNA can comprise a) transfecting a cell comprising the polynucleotide comprising the sequence coding for the suppressor tRNA flanked by ITRs; b) transfecting the cell with a polynucleotide comprising sequence coding for a helper protein, a polynucleotide comprising a sequence coding for a Rep protein, and a polynucleotide comprising a sequence coding for a Cap protein; c) transfecting the cell with a composition that comprises siRNA, shRNA, DsRNA or any combination; and d) producing virions encapsidating the polynucleotide comprising the sequence coding for the suppressor tRNA, wherein the siRNA, shRNA, DsRNA or any combination thereof binds to the suppressor tRNA and targets the suppressor tRNA for degradation, thereby silencing the suppressor tRNA, and wherein the titer of virions encapsidating the polynucleotide comprising the the sequence coding for the suppressor tRNA is increased compared to titer of virions encapsidating the sequence for the suppressor tRNA produced by steps a), b), and d).

A method of increasing titer of virions encapsidating a polynucleotide comprising a sequence coding for a suppressor tRNA by silencing the suppressor tRNA can comprise a) transfecting a cell comprising the polynucleotide comprising the sequence coding for the suppressor tRNA flanked by ITRs; b) transfecting the cell with a polynucleotide comprising sequence coding for a helper protein, a polynucleotide comprising a sequence coding for a Rep protein, and a polynucleotide comprising a sequence coding for a Cap protein; c) transfecting the cell with a composition that comprises siRNA, shRNA, DsRNA or any combination; and d) producing virions encapsidating the polynucleotide comprising the sequence coding for the suppressor tRNA, wherein the siRNA, shRNA, DsRNA or any combination thereof binds to the suppressor tRNA and targets the suppressor tRNA for degradation, thereby silencing the suppressor tRNA, and wherein the titer of virions encapsidating the polynucleotide comprising the the sequence coding for the suppressor tRNA is increased compared to titer of virions encapsidating the sequence for the suppressor tRNA produced without step c) transfecting the cell with a composition that comprises siRNA, shRNA, DsRNA or any combination.

A method of increasing titer of virions encapsidating a polynucleotide comprising a sequence coding for a suppressor tRNA by silencing the suppressor tRNA can comprise a) transfecting a cell comprising the polynucleotide comprising the sequence coding for the suppressor tRNA flanked by ITRs; b) transfecting the cell with a polynucleotide comprising sequence coding for a helper protein, a polynucleotide comprising a sequence coding for a Rep protein, and a polynucleotide comprising a sequence coding for a Cap protein; c) transfecting the cell with a composition that comprises siRNA, shRNA, DsRNA or any combination; and d) producing virions encapsidating the polynucleotide comprising the sequence coding for the suppressor tRNA, wherein the siRNA, shRNA, DsRNA or any combination thereof binds to the suppressor tRNA and targets the suppressor tRNA for degradation, thereby silencing the suppressor tRNA, and wherein the titer of virions encapsidating the polynucleotide comprising the the sequence coding for the suppressor tRNA is increased compared to titer of virions encapsidating the sequence for the suppressor tRNA produced in the absenc of the composition comprising siRNA, shRNA, DsRNA or any combination.

A method of increasing titer of virions encapsidating a sequence coding for a suppressor tRNA can comprise: a) providing a cell comprising: a sequence encoding a suppressor tRNA flanked by ITRs, a sequence encoding a helper protein, a sequence encoding a VA RNA, a sequence encoding a Rep protein, a sequence encoding a Cap protein, and optionally a sequence coding for AAP, a sequence coding for MAAP, and/or a sequence coding for Protein X, wherein one or more of the sequence encoding the helper protein, the sequence encoding the VA RNA, the sequence encoding the Rep protein, the sequence coding for the Cap protein, the sequence coding for AAP, the sequence coding for MAAP, and/or the sequence coding for Protein X is stop codon engineered; and b) culturing the cell under conditions suitable for expression and encapsidation of the suppressor tRNA. In some cases, the Rep protein is Rep78, Rep52, Rep68, Rep40, or any combination thereof.

In some embodiments, the repressor is a Tet repressor protein or a guide RNA complexed to a catalytically dead Cas protein that is linked or fused to a repressor domain. In some embodiments, the repressor domain is a krab domain. In some embodiments, the Rep protein is Rep78, Rep52, Rep68, Rep40, or any combination thereof. In some embodiments, an opal stop codon of the sequence coding for the Rep68 is changed to an amber stop codon or an ochre stop codon. In some embodiments, an opal stop codon of the sequence coding for the Rep40 is changed to an amber stop codon or an ochre stop codon. In some embodiments, an ochre stop codon of the sequence coding for the Rep78 is changed to an amber stop codon or an opal stop codon. In some embodiments, an ochre stop codon of the sequence coding for the Rep52 is changed to an amber stop codon or an opal stop codon. In some embodiments, the Cap protein is VP1, VP2, VP3, or any combination thereof. In some embodiments, an ochre stop codon of the sequence coding for VP1 is changed to an amber stop codon or an opal stop codon. In some embodiments, an ochre stop codon of the sequence coding for VP2 is changed to an amber stop codon or an opal stop codon. In some embodiments, an ochre stop codon of the sequence coding for VP3 is changed to an amber stop codon or an opal stop codon. In some embodiments, the helper protein is E1a, E1b, E4, E2a, VA, or any combination thereof. In some embodiments, the cell is a mammalian cell. In some embodiments, the cell is an HEK293 cell or a CHO cell. In some embodiments, the cell is an insect cell. In some embodiments, the cell is an Sf9 cell. In some embodiments, the virion is an AAV virion. In some embodiments, a capsid of the AAV virion is selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combinations thereof. In some embodiments, the AAV virion is comprises a hybrid capsid. In some embodiments, the hybrid capsid comprises any combination of capsids selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combinations thereof. In some embodiments, the hybrid capsid is AAV2/5 or AAV-DJ. In some embodiments, the conditionally repressible suppressor tRNA coding sequence flanked by ITRs (e.g., by a 5′ ITR and 3′ ITR) comprises at least 80%, 85%, 90%, 95%, 97%, 99%, or 100% sequence identity to any one of SEQ ID NOs: 158-162, 164-168, 171-175, 177-181, 182-194, or 199. In some embodiments, a region comprising multiple copies of one or more conditionally repressible suppressor tRNA coding sequence(s) flanked by ITRs (e.g., by a 5′ ITR and 3′ ITR) comprises one or more sequences having at least 80%, 85%, 90%, 95%, 97%, 99%, or 100% sequence identity to any one of SEQ ID NOs: 158-162, 164-168, 171-175, 177-181, 182-194, or 199. In some embodiments, a region comprising one, two, three, four, five, six, seven, eight, nine, or ten of the one or more conditionally repressible suppressor tRNA coding sequence(s) flanked by ITRs (e.g., by a 5′ ITR and 3′ ITR) comprises one, two, three, four, five, six, seven, eight, nine, or ten of the one or more sequences having comprising at least 80%, 85%, 90%, 95%, 97%, 99%, or 100% sequence identity to any one of SEQ ID NOs: 158-162, 164-168, 171-175, 177-181, 182-194, or 199. In some embodiments, a polynucleotide as described herein comprises at least 80%, 85%, 90%, 95%, 97%, 99%, or 100% sequence identity to any one of SEQ ID NOs: 203-206 or 208-211. In some embodiments, a polynucleotide as described herein comprises at least 80%, 85%, 90%, 95%, 97%, 99%, or 100% sequence identity to any one of SEQ ID NOs: 203-206 or 208-211 without the sequence coding for transduction marker, wherein the sequence coding for the transduction marker comprises at least 80%, 85%, 90%, 95%, 97%, 99%, or 100% sequence identity to SEQ ID NO: 224. In some embodiments, the 5′ ITR comprises a polynucleotide having at least 80%, 85%, 90%, 95%, 97%, 99%, or 100% sequence identity to any one of SEQ ID NOs: 225-226. In some embodiments, the 3′ ITR comprises a polynucleotide having at least 80%, 85%, 90%, 95%, 97%, 99%, or 100% sequence identity to any one of SEQ ID NOs: 227-228.

### Virions

Provided herein are virions encapsidating a suppressor tRNA. Provided herein are virions encapsidating a polynucleotide comprising a sequence coding for suppressor tRNA. The virion can encapsidate a conditionally repressible suppressor tRNA coding sequence. The virion can encapsidate a polynucleotide comprising a conditionally repressible suppressor tRNA coding sequence. In some embodiments, the conditionally repressible suppressor tRNA coding sequence comprises a sequence coding for a repressor element and a sequence coding for a suppressor tRNA, as previously described herein. In some embodiments, the conditionally repressible suppressor tRNA coding sequence comprises sequence coding for a repressor element upstream of a sequence coding for a suppressor tRNA. A repressor element can be any sequence capable of being bound by a repressor, wherein the binding by the repressor inhibits expression of a suppressor tRNA. The sequence of the suppressor tRNA that is inhibited can be upstream of the repressor element or downstream of the repressor element. In some embodiments, the conditionally repressible suppressor tRNA coding sequence comprises at least 80%, 85%, 90%, 95%, 97%, 99%, or 100% sequence identity to any one of SEQ ID NOs: 158-162, 164-168, 171-175, 177-181, 182-194, or 199. In some embodiments, multiple copies of one or more conditionally repressible suppressor tRNA coding sequence(s) encaspidated by the virion comprises one or more sequences having at least 80%, 85%, 90%, 95%, 97%, 99%, or 100% sequence identity to any one of SEQ ID NOs: 158-162, 164-168, 171-175, 177-181, 182-194, or 199. In some embodiments, one, two, three, four, five, six, seven, eight, nine, or ten of the one or more conditionally repressible suppressor tRNA coding sequence(s) encapsidated by the virion comprises one, two, three, four, five, six, seven, eight, nine, or ten of the one or more sequences having comprising at least 80%, 85%, 90%, 95%, 97%, 99%, or 100% sequence identity to any one of SEQ ID NOs: 158-162, 164-168, 171-175, 177-181, 182-194, or 199. In some embodiments, the virion encapsidates a polynucleotide having of at least 80%, 85%, 90%, 95%, 97%, 99%, or 100% sequence identity to any one of SEQ ID NOs: 203-206 or 208-211. In some embodiments, the virion encapsidates a polynucleotide having of at least 80%, 85%, 90%, 95%, 97%, 99%, or 100% sequence identity to any one of SEQ ID NOs: 203-206 or 208-211 without the sequence coding for transduction marker, wherein the sequence coding for the transduction marker comprises at least 80%, 85%, 90%, 95%, 97%, 99%, or 100% sequence identity to SEQ ID NO: 224. In some embodiments, the sequence coding for a suppressor tRNA encapsidated by the virion is for a suppressor tRNA capable of suppressing an opal stop codon, an ochre stop codon, or an amber stop codon. In some embodiments, the sequence coding for a suppressor tRNA encapsidated by the virion comprises 100%, at least 99%, at least 98%, at least 95%, or at least 90% sequence identity to any one of SEQ ID NO: 3-SEQ ID NO: 48 or SEQ ID NO: 103-SEQ ID NO: 148. In some embodiments, the sequence of the opal stop codon anticodon loop (TCA) of any one of SEQ ID NOs: 3-48 is interchangeable with a sequence of the amber stop codon anticodon loop (CTA) for readthrough of an amber stop codon or with a sequence of the ochre stop codon anticodon (TTA) for readthrough of an ochre stop codon. In some embodiments, the sequence of the opal stop codon anticodon loop (UCA) of any one of SEQ ID NOs: 103-148 is interchangeable with a sequence of the amber stop codon anticodon loop (CUA) for readthrough of an amber stop codon or with a sequence of the ochre stop codon anticodon (UUA) for readthrough of an ochre stop codon. In some embodiments, the sequence coding for a suppressor tRNA comprises 100%, at least 99%, at least 98%, at least 95%, or at least 90% sequence identity to any one of SEQ ID NO: 3-SEQ ID NO: 48 or SEQ ID NO: 103-SEQ ID NO: 148, wherein the anticodon is engineered to bind to an ochre stop codon or an amber stop codon.

Provided herein is a virion encapsidating a suppressor tRNA produced by the any of the methods as described herein. Provided herein is a virion encapsidating polynucleotide comprising a sequence coding for a suppressor tRNA produced by the any of the methods as described herein. In some embodiments, the method comprises silencing the suppressor tRNA during virion packaging to produce a virion encapsidating a suppressor tRNA. In some embodiments, the method comprises silencing the suppressor tRNA during replication of viral molecules (e.g., viral protein and/or viral RNA) for production of a virion encapsidating a suppressor tRNA. In some embodiments, the method comprises silencing the suppressor tRNA during viral molecule replication and virion packaging to produce a virion encapsidating a suppressor tRNA. In some embodiments, the silencing of the suppressor tRNA utilizes a conditionally repressible suppressor tRNA coding sequence, wherein the repressor binds to the repressor element during viral molecule replication and/or virion packaging to produce a virion encapsidating a suppressor tRNA. In some embodiments, the silencing of the suppressor tRNA utilizes siRNA, shRNA, DsRNA, or any combination thereof, wherein the siRNA, shRNA, and/or DsRNA binds to the suppressor tRNA and targets the suppressor tRNA for degradation during viral molecule replication and/or virion packaging to produce a virion encapsidating a polynucleotide comprising a sequence coding for a suppressor tRNA. In some cases, silencing comprises a reduction of at least 40%, e.g., at least 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% in the amount or activity of suppressor tRNA(s) relative to a control.

A virion can encapsidates the sequence coding for a conditionally repressible suppressor tRNA coding sequence as described herein. A virion can encapsidates the polynucleotide comprising the sequence coding for a conditionally repressible suppressor tRNA coding sequence as described herein. In some embodiments, the conditionally repressible suppressor tRNA coding sequence comprises a sequence coding for a repressor element and a sequence coding for a suppressor tRNA. In some embodiments, the conditionally repressible suppressor tRNA coding sequence comprises sequence coding for a repressor element upstream of a sequence coding for a suppressor tRNA. In some embodiments, the conditionally repressible suppressor tRNA coding sequence is flanked by ITR sequences, e.g, by a 5′ ITR sequence and a 3′ ITR sequence. In some embodiments, the 5′ ITR sequence comprises at least 80%, 85%, 90%, 95%, 97%, 99%, or 100% sequence identity to any one of SEQ ID NOs: 225-226. In some embodiments, the 3′ ITR sequence comprises at least 80%, 85%, 90%, 95%, 97%, 99%, or 100% sequence identity to any one of SEQ ID NOs: 227-228.

In some embodiments, the virion is an AAV virion. In some embodiments, a capsid of the AAV virion is selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combinations thereof. In some embodiments, the AAV virion is comprises a hybrid capsid. In some embodiments, the hybrid capsid comprises any combination of capsids selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combinations thereof. In some embodiments, the hybrid capsid is AAV2/5 or AAV-DJ.

### Methods of Treating a Subject

Premature stop codons leading to mutations in proteins have been implicated in many severe diseases and disorders, such as Rett Syndrome, Dravet Syndrome, and muscular dystrophies such as Duchenne Muscular Dystrophy. Translation of a mRNA molecule that contains a premature stop codon can cause premature termination of the translation process to produce a truncated polypeptide or protein. As used herein, “premature stop codon” can be used interchangeably with “premature termination codon” (PTC). The unintended truncation of proteins vital to neurodevelopment, particularly in children, can cause severe and life-altering diseases or disorders.

In some cases, the stop codon can be opal (TGA; UGA), ochre (TAA; UAA), or an amber (TAG; UAG) stop codon. In some cases, the disease-causing mutation in the mRNA sequence can comprise an opal stop codon (TGA; UGA) in the place of a codon encoding Arg (e.g., CGU, CGC, CGA, CGG, AGA, or AAG). In some cases, the disease-causing mutation in the mRNA sequence can comprise an ochre (TAA; UAA) or an ochre (TAG; UAG) stop codon in the place of a codon encoding Glutamine (e.g., CCA, CAG). In some cases, the premature stop codon results in a truncated version of the polypeptide or protein. In some cases, the disease, disorder, or condition can be caused by an increased level of a truncated version of the polypeptide, or a decreased level of substantially full-length polypeptide.

In some aspects, a method of treating a subject having a disease associated with a premature stop codon comprises administering the virion comprising a sequence of a suppressor tRNA or a conditionally repressible suppressor tRNA sequence to the subject. In some aspects, a method of treating a subject having a disease associated with a premature stop codon comprises administering the virion comprising the polynucleotide having a sequence of a suppressor tRNA or a conditionally repressible suppressor tRNA sequence to the subject. In some embodiments, the disease associated with a premature stop codon is Rett syndrome, autism, West syndrome, Lennox-Gastaut syndrome, epileptic encephalopathy (EEP), Pitt-Hopkins syndrome, or any combination thereof. In some cases, a disease or condition can comprise cystic fibrosis, deafness (e.g. autosomal dominant 17 deafness, autosomal dominant 13 deafness, autosomal dominant 11 deafness) retinitis pigmentosa or any combination thereof. In some cases, a disease or condition can comprise Tay-Sachs, Parkinson's, Cystic Fibrosis, Usher syndrome, Wolman disease, a liver disease (Alpha-1 antitrypsin (AAT) deficiency), or any combination thereof. A disease or condition can comprise a neurodegenerative disease, a muscular disorder, a metabolic disorder, an ocular disorder (e.g. an ocular disease), a cancer, or any combination thereof. The the disease associated with a premature stop codon comprise cystic fibrosis, albinism, Alzheimer disease, Amyotrophic lateral sclerosis, Asthma, B-thalassemia, Cadasil syndrome, Charcot-Marie-Tooth disease, Chronic Obstructive Pulmonary Disease (COPD), dementia, Distal Spinal Muscular Atrophy (DSMA), Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Myotonic dystrophy types I and II, neurofibromatosis, Niemann-Pick disease type A, B and C, NY-esol related cancer, Parkinson's disease, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, X-linked immunodeficiency, various forms of cancer (e.g., BRCA1 and 2 linked breast cancer and ovarian cancer). The the disease associated with a premature stop codon can be a muscular dystrophy, an ornithine transcarbamylase deficiency, a breast cancer, an ovarian cancer, a prostate cancer, a lung cancer, a skin cancer, Stargardt macular dystrophy, Charcot-Marie-Tooth disease, or any combination thereof. A disease associated with a premature stop codon can be a muscular dystrophy. A muscular dystrophy can include myotonic, Duchenne, Becker, Limb-girdle, facioscapulohumeral, congenital, oculopharyngeal, distal, Emery-Dreifuss, or any combination thereof, the disease associated with a premature stop codon can comprise pain, such as chronic pain. Pain can include neuropathic pain, nociceptive pain, or a combination thereof. Nociceptive pain can include visceral pain, somatic pain, or a combination thereof.

In some embodiments, the disease associated with a premature stop codon is Rett syndrome. For example, Rett Syndrome can be caused by an Arg-to-stop mutation in a polypeptide sequence of MeCP2. In a healthy individual, the MeCP2 protein contains an Arginine (Arg) at amino acid positions 168, 255, 270, 294, 198, 186 and 453 with reference to SEQ ID NO: 89 (MAAAAAAAPSGGGGGGEEERLEEKSEDQDLQGLKDKPLKFKKVKKDKKEEKEGKHE PVQPSAHHSAEPAEAGKAETSEGSGSAPAVPEASASPKQRRSIIRDRGPMYDDPTLPEGW TRKLKQRKSGRSAGKYDVYLINPQGKAFRSKVELIAYFEKVGDTSLDPNDFDFTVTGRG SPSRREQKPPKKPKSPKAPGTGRGRGRPKGSGTTRPKAATSEGVQVKRVLEKSPGKLLV KMPFQTSPGGKAEGGGATTSTQVMVIKRPGRKRKAEADPQAIPKKRGRKPGSVVAAAA AEAKKKAVKESSIRSVQETVLPIKKRKTRETVSIEVKEVVKPLLVSTLGEKSGKGLKTCK SPGRKSKESSPKGRSSSASSPPKKEHHHHHHHSESPKAPVPLLPPLPPPPPEPESSEDPTSPP EPQDLSSSVCKEEKMPRGGSLESDGCPKEPAKTQPAVATAATAAEKYKHRGEGERKDI VSSSMPRPNREEPVDSRTPVTERVS), or with reference to SEQ ID NO: 90 (MVAGMLGLREEKSEDQDLQGLKDKPLKFKKVKKDKKEEKEGKHEPVQPSAHHSAEP AEAGKAETSEGSGSAPAVPEASASPKQRRSIIRDRGPMYDDPTLPEGWTRKLKQRKSGR SAGKYDVYLINPQGKAFRSKVELIAYFEKVGDTSLDPNDFDFTVTGRGSPSRREQKPPK KPKSPKAPGTGRGRGRPKGSGTTRPKAATSEGVQVKRVLEKSPGKLLVKMPFQTSPGG KAEGGGATTSTQVMVIKRPGRKRKAEADPQAIPKKRGRKPGSVVAAAAAEAKKKAVK ESSIRSVQETVLPIKKRKTRETVSIEVKEVVKPLLVSTLGEKSGKGLKTCKSPGRKSKESS PKGRSSSASSPPKKEHHHHHHHSESPKAPVPLLPPLPPPPPEPESSEDPTSPPEPQDLSSSV CKEEKMPRGGSLESDGCPKEPAKTQPAVATAATAAEKYKHRGEGERKDIVSSSMPRPN REEPVDSRTPVTERVS). In Rett Syndrome patients, a mutation in the MeCP2 protein that causes premature termination of translation of the MeCP2 protein at amino acid position 168, 255, 270, 294, 198, 186 and 453 causes loss-of-function of MeCP2, and collectively account for more than 25% of Rett-causing mutations. Arginine (Arg) in MECP2 can also be found at positions R8 (for isoform 2 only), R9 (for isoform 1 only), R84, R85, R89, R91, R106, R111, R115, R133, R162, R167, R188, R190, R211, R250, R253, R268, R306, R309, R344, R354, R420, R458, R468, R471, R478, and R484. The present disclosure provides engineered tRNAs and engineered tRNA variants that are capable of premature stop codon read-through of a premature stop codon that can be present at any position in the MeCP2 protein, including any one of: R168X, R255X, R270X, R294X, R198X, R186X, R453X, R8X (in isoform 2), R9X (in isoform 1), R84X, R85X, R89X, R91X, R106X, R111X, R115X, R133X, R162X, R167X, R188 X, R190X, R211X, R250X, R253X, R268X, R306X, R309X, R344X, R354X, R420X, R458X, R468X, R471X, R478X, R484X, or any combination thereof. In some cases, Rett Syndrome in a subject can be treated by administering the compositions described herein to the subject, wherein the composition can comprise an engineered tRNA or variant thereof with an anticodon sequence that base pairs with the disease-causing premature stop codon (e.g., UGA). During translation, the engineered tRNA or variant thereof can be charged with an Arg, which can be transferred to the growing MeCP2 polypeptide, thereby at least substantially restoring MeCP2 expression in the subject. In some cases, the substantially restored MeCP2 polypeptide can be functional as compared with a WT MeCP2 protein.

In some aspects, a method of treating a subject having Rett syndrome comprises administering the virion of as disclosed herein comprising a sequence of a suppressor tRNA or a conditionally repressible suppressor tRNA sequence as disclosed herein to the subject. In some embodiments, the subject is a human.

Also provided herein is a method for readthrough of a premature stop codon in a subject having a disease associated with a premature stop codon, the method comprising: producing encapsidated virion by a method comprising: culturing any of the cell(s) described herein under conditions effective (a) to replicate the polynucleotide comprising one or more conditionally repressible suppressor tRNA coding sequence(s) and express the Cap protein; wherein expression of the suppressor tRNA is repressed compared to a control; optionally wherein the expression of the suppressor tRNA is repressed at least 40%, at least 45%, at least 50%, at least 55%, at least 60%, at least 65%, at least 70%, at least 75%, at least 80%, at least 85%, or at least 90% relative to a control; optionally wherein the control is: a) expression of the suppressor tRNA in the cell(s) when the repressor is inhibited from binding to the repressor element, optionally wherein the repressor is inhibited from binding to the repressor element by binding of a switching agent to the repressor; or b) expression of the suppressor tRNA from reference cell(s) comprising a vector comprising the conditionally repressible suppressor tRNA coding sequence, wherein the reference cell(s) do not express the repressor; and administering the virion to a subject having a disease associated with a premature stop codon under conditions effective for expression of the suppressor tRNA and readthrough of the premature stop codon. In some cases, the disease associated with a premature stop codon is Rett syndrome, Dravet syndrome, or Duchenne Muscular Dystrophy. In some cases, the disease associated with a premature stop codin is Rett syndrome. In some cases, the subject is a human.

### EXAMPLES

The following examples are included for illustrative purposes only and are not intended to limit the scope of the invention.

### Example 1: Silencing Suppressor tRNAs Using a Tet Repressor System

This example describes using a Tet repressor system to silence suppressor tRNAs during AAV virion production.

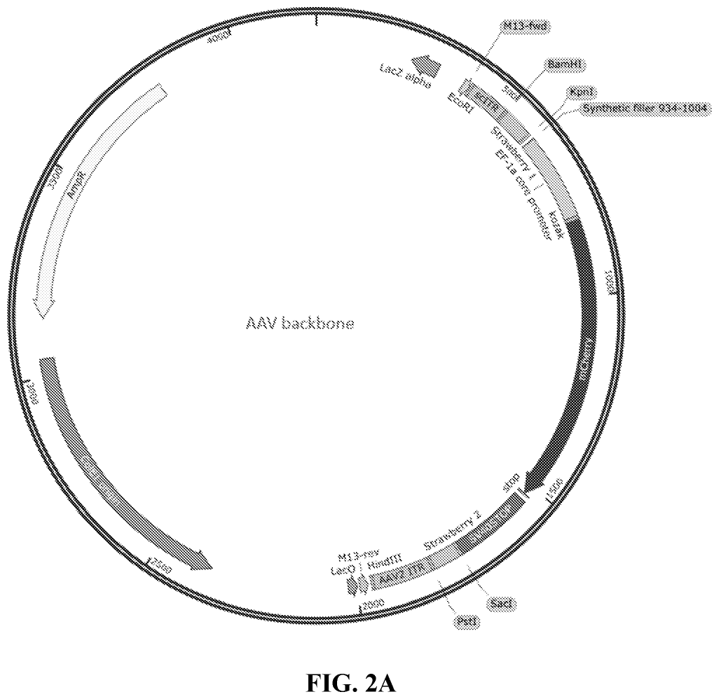

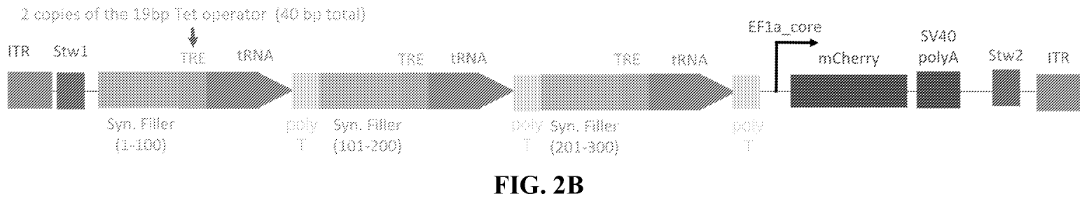

An AAV backbone plasmid as shown in FIG. 2A, but having the sequence between ITRs as shown in FIG. 2B, was generated. The ITR-ITR sequence comprised three conditionally repressible suppressor tRNA coding sequences (3 copies of a sequence comprising a sequence of the repressor element (TRE sequence) and the sequence coding for the suppressor RNA (tRNA)), synthetic filler sequences (100 bps), an EF1a_core-mCherry sequence (transduction marker), and strawberry sequences (quantification tags for ddPCR quantification of episomes/AAV transcripts). The TRE sequence comprised two repeats of the Tet operator (19 bp) with a TC spacer: TCCCTATCAGTGATAGAGATCTCCCTATCAGTGATAGAGA (SEQ ID NO: 149). Three different plasmids encoding different suppressor tRNAs (CCT4, CCT2.10, or CCT5 (control tRNA; non-functional)) were generated.

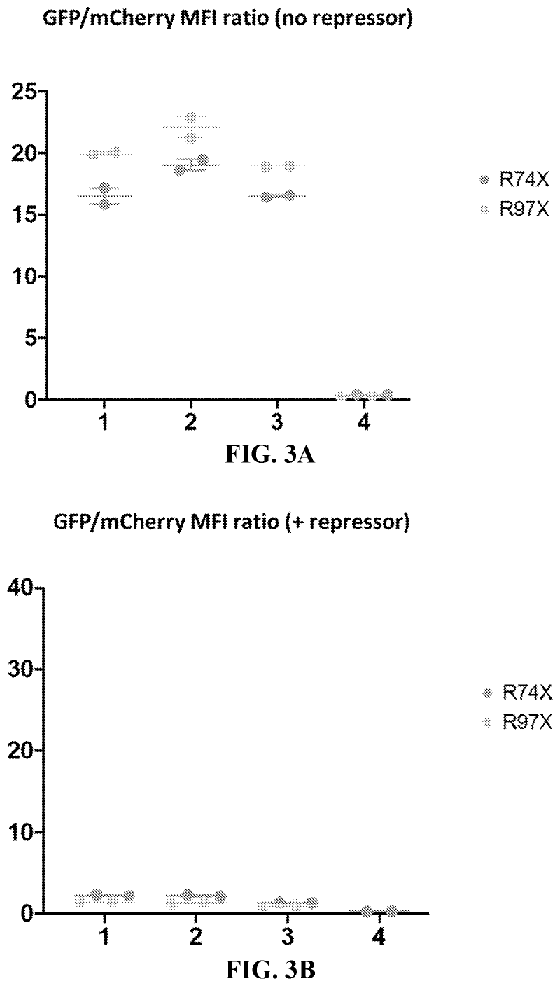

Cells not expressing a Tet repressor (FIG. 3A; “no repressor”) or cells stably expressing a Tet repressor (FIG. 3B; “+repressor”) were transfected with a first plasmid comprising either a R74X premature stop codon or a R97X premature stop codon inserted into a GFP sequence (broken GFP) and a second plasmid comprising the ITR-ITR sequence of FIG. 2B having conditionally repressible suppressor tRNA coding sequences as described above. The x-axis of the graphs in FIG. 3A and FIG. 3B are as follows: 1: 3×CCT4-TRE+1; 2: 3×CCT4-TRE-7; 3: 3×CCT2.10-TRE+1; or 4: 3×CCT5-TRE+1 (control) (3× indicates the plasmid had three copies of the repressor element and sequence coding for the tRNA (suppressor tRNA for construct 1-3 and non-functional tRNA for construct 4), TRE indicates the repressor element was a TRE sequence comprising two TetO sequences separated by a TC spacer (SEQ ID NO: 149); +1 or −7 indicates the position of the repressor element with respect to the start site of the tRNA sequence, wherein +1 indicates no spacer nucleotides between the end of the TRE sequence and the start site of the tRNA sequence and −7 indicates a spacer of seven nucleotides between the end of the TRE sequence and the start site of the tRNA sequence). Detection of a GFP signal indicates the suppressor tRNA was expressed and there was subsequent readthrough of the premature stop codon to produce detectable GFP. Detection of mCherry indicates successful transfection of the plasmid comprising repressor element upstream of the sequence coding for the suppressor tRNA. The transfected cells not expressing a Tet repressor had a high ratio of GFP/mCherry MFI, indicating readthrough of the broken GFP and therefore, expression of the suppressor tRNAs (FIG. 3A). Transfected cells expressing a Tet repressor had a low ratio of GFP/mCherry MFI, indicating low to no readthrough of the broken GFP and therefore, indicating silencing of expression of the suppressor tRNAs (FIG. 3B).

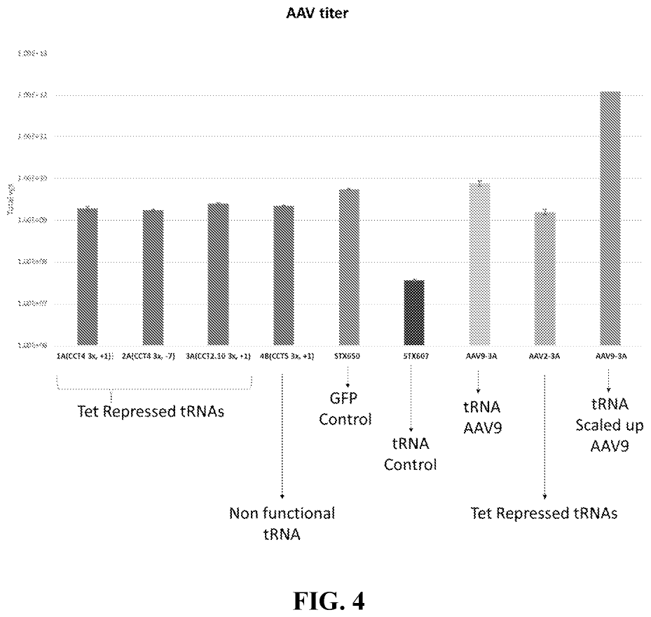

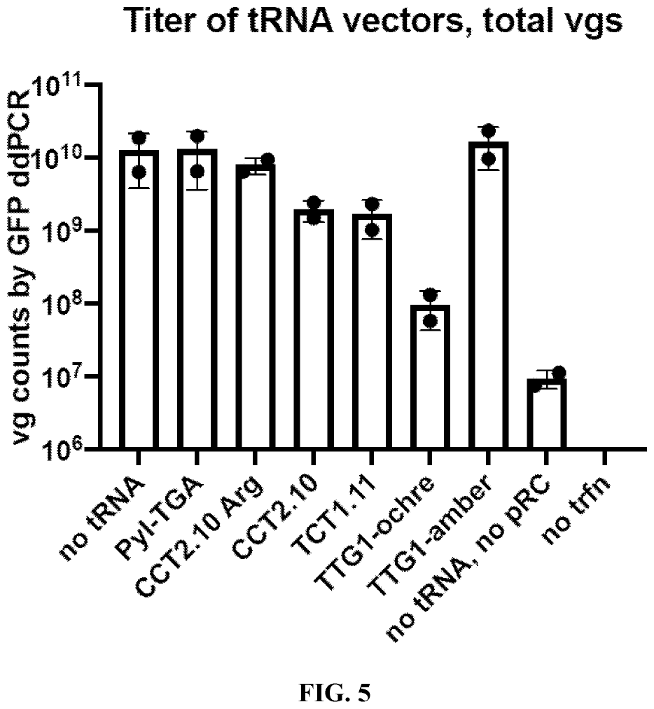

Triple transfection was performed in cells expressing the Tet repressor, with an AAV helper plasmid, an AAV Rep/Cap plasmid, and a tRNA suppressor plasmid as follows: 1A: AAV2, CCT4 3×, +1; 2A: AAV2, CCT4 3×, −7; 3A: AAV2, CCT2.10 3×, +1; 4B: AAV2, CCT5 3×, +1; STX650: AAV2, CMV-GFP control; STX0607: AAV2, CCT2.10 3×, no repressor element; AAV9-3A: AAV9, CCT2.10 3×, +1; AAV2-3A: AAV2, CCT2.10 3×, +1 (3× indicates the plasmid had three copies of the repressor element and suppressor tRNA sequence, +1 or −7 indicates the position of the repressor element, which was a TRE sequence comprising two TetO sequences separated by a TC spacer (SEQ ID NO: 149), which were located between two ITR sequences in the plasmid) for the production of AAV virion encapsidating the polynucleotide comprising the sequence coding for the suppressor tRNA. The AAV serotypes tested were AAV2 or AAV9 as indicated. Production was scaled up (pooling multiple lots of produced AAV) for AAV9-3A as shown for the right most bar on the graph in FIG. 4. During the production of AAV virion encapsidating the polynucleotide comprising the sequence coding for the suppressor tRNA, the Tet repressor bound to the polynucleotides encoding the TRE sequence to silence the expression of the suppressor tRNAs from the polynucleotides comprising the sequence coding for the suppressor tRNAs. Cells transfected with a plasmid comprising a TRE sequence upstream of the suppressor tRNA sequence produced increased AAV titer compared to cells transfected with a plasmid lacking a TRE sequence upstream of the suppressor tRNA sequence (FIG. 4).

### Example 2: Silencing Suppressor tRNAs Using a CRISPRi System

This example describes using a CRISPRi system to silence suppressor tRNAs during AAV virion production.

Three different suppressor tRNA plasmids (each encoding for different suppressor tRNAs (CCT4, CCT2.10, or CCT5 (control tRNA; non-functional))) are generated, in which a plasmid comprises an AAV backbone plasmid comprising a guide RNA binding sequence (repressor element) upstream of a sequence coding for a suppressor tRNA, which is between two ITRs.

Cells not expressing a dCas9 linked to a krab domain or cells expressing a dCas9 linked to a krab domain are quadruple transfected with the guide RNAs that bind to the guide RNA binding sequence and are capable of being complexed with the dCas9 linked to a krab domain; with an AAV helper plasmid; with an AAV Rep/Cap plasmid; and independently, with each suppressor tRNA plasmid as described above, for the production of AAV virion encapsidating the sequence coding for the suppressor tRNA. During the production of AAV virion in the transfected cells, the guide RNA is complexed with the dCas9 linked to the krab domain in the cells that express the dCas9 linked to the krab domain, and the guide RNA of this complex is then bound to the guide RNA binding sequence of the suppressor tRNA plasmid to silence the expression of the suppressor tRNAs. The suppressor tRNA of cells not expressing the dCas9 linked to the krab domain are not silenced. The AAV virion are produced and are tested for titer, genome encapsidation, and infectivity of AAV virion produced by the cells not expressing the dCas9 linked to the krab domain compared to by the cells expressing the dCas9 linked to the krab domain.

### Example 3: Silencing Suppressor tRNAs Using a siRNA, shRNA, and DsRNA

This example describes using siRNA, shRNA, and DsRNA to silence suppressor tRNAs during AAV virion production.

Three different suppressor tRNA plasmids (each encoding different suppressor tRNAs (CCT4, CCT2.10, or CCT5 (control tRNA; non-functional))) are generated, in which the plasmids comprise an AAV backbone plasmid comprising a sequence between two ITRs coding for the suppressor tRNA.

siRNA, shRNA, and DsRNA that bind to the anti-codon region of the suppressor tRNAs are generated.

Cells are either quadruple transfected with the above generated siRNA, shRNA, and DsRNA, with an AAV helper plasmid, with an AAV Rep/Cap plasmid, and independently, with each suppressor tRNA plasmid as described above; or are triple transfected with an AAV helper plasmid, with an AAV Rep/Cap plasmid, and independently, with each suppressor tRNA plasmid as described above, for the production of AAV virion encapsidating the sequence coding for the suppressor tRNA. During the production of AAV virion, the siRNA, shRNA, and DsRNA of the quadruple transfected cells are bound to the expressed suppressor tRNA (e.g., see FIG. 1D), and the expressed suppressor tRNA are therefore targeted for degradation and are subsequently degraded by RISC, resulting in silencing of the suppressor tRNAs. The suppressor tRNAs of the triple transfected cell are not silenced. The AAV virion are produced and are tested for titer, genome encapsidation, and infectivity of AAV virion that is produced by the quadruple transfected cells versus that is produced by the triple transfected cells.

### Example 4: Impact of tRNA on AAV Titer

This example describes assessing the impact of suppressor tRNA expression on AAV titer.

Low AAV titer (75% up to 2 logs lower than control vectors) was observed for virion encapsidating polynucleotides comprising opal suppressor tRNA coding sequences.

The following constructs between the ITR-ITR sequence of a tRNA plasmid were used to assess tRNA impact on AAV titer: (1) a sequence encoding no tRNA control (eF1alpha-driven GFP only); (2) a sequence encoding hU6-driven tRNA-Pyl-TGA, mU6-driven tRNA-Pyl-TGA, and GFP; (3) a sequence encoding hU6-driven CCT2.10 arginine tRNA, mU6-driven CCT2.10 arginine tRNA, and eF1alpha-driven GFP; (4) a sequence encoding hU6-driven CCT2.10 opal suppressor tRNA, mU6-driven CCT2.10 opal suppressor tRNA, and eF1 alpha-driven GFP; (5) a sequence encoding hU6-driven TCT1.11 opal suppressor tRNA, mU6-driven TCT1.11 opal suppressor tRNA, and eFlalpha-driven GFP; (5) a sequence encoding hU6-driven TTG1-ochre suppressor tRNA, mU6-driven TTG1-ochre suppressor tRNA, and eF1 alpha-driven GFP; or (6) a sequence encoding hU6-driven TTG1-amber suppressor tRNA, mU6-driven TTG1-amber suppressor tRNA, and eF1alpha-driven GFP. In these plasmids, CCT2.10 arginine tRNA comprises GCCCCAGTGGCCTAATGGATAAGGCACTGGCCTCCTAAGCCAGGGATTGCGGGTTC GAGTCCCGCCTGGGGTG (SEQ ID NO: 151); TTG1-amber comprises GGTCCCATGGTGTAATGGTTAGCACTCTGGACTCTAAATCCAGCGATCCGAGTTCAA ATCTCGGTGGGACC (SEQ ID NO: 152); TTG1-ochre comprises GGTCCCATGGTGTAATGGTTAGCACTCTGGACTTTAAATCCAGCGATCCGAGTTCAA ATCTCGGTGGGACC (SEQ ID NO: 153); CCT2.10 opal suppressor tRNA comprises SEQ ID NO: 32; and TCT1.11 opal suppressor tRNA comprises SEQ ID NO: 45.

HEK293 cells were transfected with plasmids encoding AAV helper proteins (pHelper), plasmids encoding Rep/Cap proteins (pRC), and independently, each of a tRNA plasmid described above (1-6), with two replicates for each tRNA plasmid. A control was transfected with the no tRNA control plasmid and the pHelper, but not the pRC. Three days after transfection, the cells and media were harvested, and AAV virion was released by freeze/thaw. Cells and media from a no transfection control (no trfn) were also harvested and freeze/thawed.

AAV Titer was measured by ddPCR using a GFP primer/probe set. As shown in FIG. 5, the tRNA Pyl-TGA was produced without a loss of AAV titer. There was loss of AAV titer observed for both the opal suppressor tRNAs (CCT2.10 and TCT.11) and ochre suppressor tRNAs (TTG1-ochre), which was most pronounced for the ochre suppressor tRNAs. There was not, in contrast, a low in titer for the amber suppressor tRNAs (TTG1-amber).

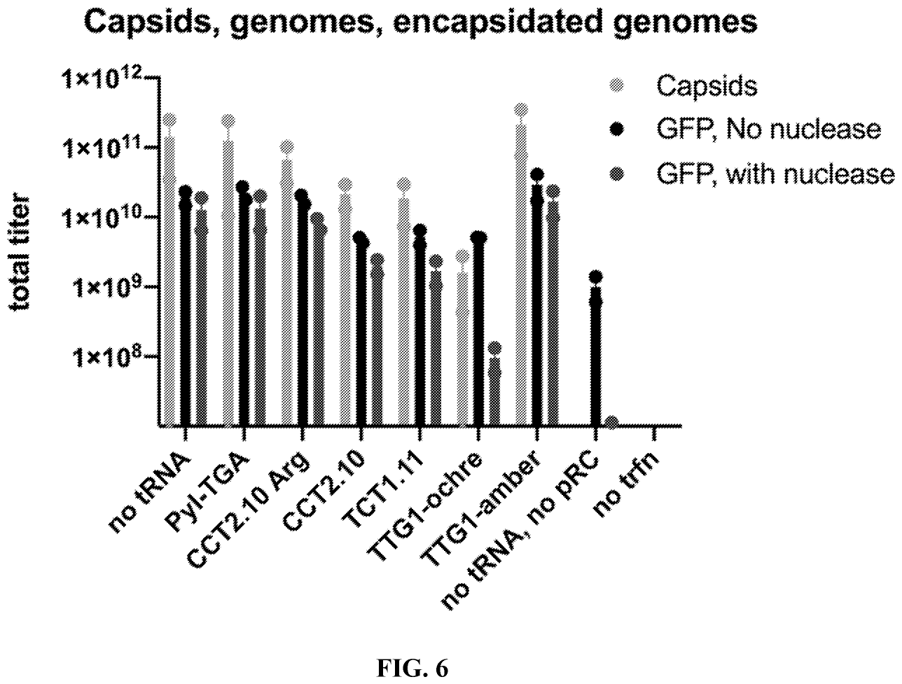

The AAV virion was treated without benzonase treatment (“No nuclease”) or with benzonase treatment (“with nuclease”), and AAV titer was measured by ddPCR using a GFP primer/probe set. ELISA was performed to measure capsid titer (“Capsids”). Titer produced from cells transfected with plasmids as described above showed decreased titer after nuclease (benzonase) treatments, as shown in FIG. 6 (compare middle bars to right bars for each transfection tested). Cells transfected with the tRNA plasmid encoding for TTG1-ochre suppressor tRNA showed a 99% loss of titer after nuclease (benzonase) treatment compared to cells transfected with the no tRNA control plasmid. Additionally, cells that were transfected with TTG1-ochre produced more replicated genomes (middle bar of TTG1-ochre in FIG. 6) than capsids (left bar of TTG1-ochre in FIG. 6) and produced ˜1% of capsids compared to the capsids produced by cells transfected with the no tRNA control (left bar of no tRNA).

### Example 5: Stop Codon Engineered AAV Nucleotides for Encapsidation of Suppressors tRNAS

This example describes assessing the impact of plasmids encoding stop codon engineered AAV nucleotides on AAV titer.

### Opal Stop Codon Engineering

The following Rep/Cap plasmids encoding stop codon engineered AAV nucleotides were used to assess their impact on AAV titer: (1) a Rep/Cap plasmid (pRC2, SEQ ID NO: 154); (2) a Rep/Cap plasmid encoding TGA stop codons engineered to TAA stop codons (Opal; pRC2_Opal, SEQ ID NO: 155); (3) a Rep/Cap plasmid encoding TGA stop codons engineered to arginine codons (Arg); and (4) a Rep/Cap plasmid encoding the TGA stop codon of AAP engineered to a TAA stop codon (AAP-TAA).

HEK293 cells were transfected with plasmids encoding AAV helper proteins (pHelper); pRC2, Opal, Arg, or AAP-TAA, independently; and a payload plasmid comprising a sequence between two ITRs encoding mCherry (614) or encoding three TCT suppressor tRNAs and mCherry (606). Three days after transfection, the cells and media were harvested, and AAV virion was released by freeze/thaw.

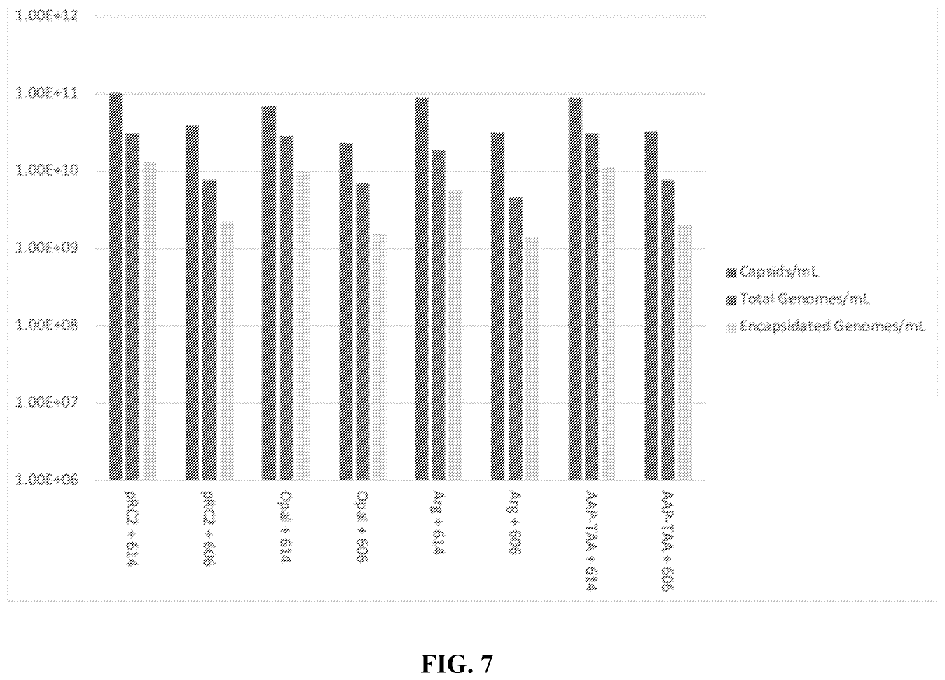

The AAV virion was treated without (middle bar for each transfection; “Total Genomes/mL”) or with (right bar for each transfection; “Encapsidated Genomes/mL”) a nuclease (benzonase) treatment, and then AAV titer was measured by ddPCR using a GFP primer/probe set. ELISA was performed to measure capsid titer (left bar for each transfection; “Capsids/mL”). Titer produced from cells transfected with plasmids as described above showed decreased after nuclease (benzonase) treatments, as shown in FIG. 7 (compare middle bars to right bars for each transfection). The titer without or with nuclease (benzonase) treatment and the capsid titer did not vary significantly between the transfections with different plasmids.

### Ochre Stop Codon Engineering

The following Rep/Cap plasmids encoding stop codon engineered AAV nucleotides were used to assess their impact on AAV titer: (1) a plasmid encoding Rep and Cap proteins (pRC2; SEQ ID NO: 154); and (2) a plasmid encoding Rep and Cap proteins with any TAA stop codons changed to TAG stop codons (pRC2-Ochre, SEQ ID NO: 156).

HEK293 cells were transfected with plasmids encoding AAV helper proteins (pHelper), plasmids encoding Rep/Cap proteins (pRC2; or pRC2-Ochre), and a payload plasmid comprising either a sequence between two ITRs encoding TTG1-ochre suppressor tRNA (STX894) or a sequence between two ITRs encoding a CCT5 tRNAs (control tRNA that is non-functional; STX956, SEQ ID NO:157). Three days after transfection, the cells and media were harvested, and AAV virion was released by freeze/thaw.

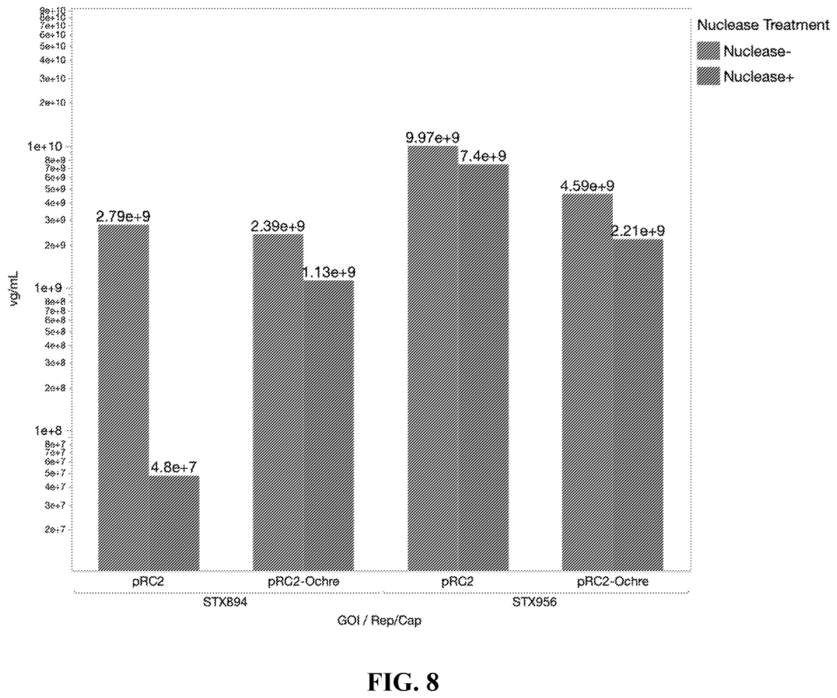

The AAV virion was treated without (left bar for each transfection; “Nuclease−”) or with (right bar for each transfection; “Nuclease+”) a nuclease (benzonase) treatment, and AAV titer was measured by ddPCR. Titer produced from cells transfected with plasmids as described above showed decreased after nuclease (benzonase) treatments, as shown in FIG. 8 (compare left bars to right bars for each transfection). The encapsidated virion titer from the cells transfected with the pRC2 plasmid and the STX894 plasmid after nuclease treatment is decreased by about 2 logs compared to the encapsidated virion titer from the cells transfected with the pRC2 plasmid and the STX956 plasmid after nuclease treatment. Additionally, the encapsidated virion titer from the cells transfected with the pRC2-Ochre plasmid and the STX894 plasmid after nuclease treatment is about comparable to the encapsidated virion titer from the transfection with the pRC2-ochre plasmid and the STX956 plasmid after nuclease treatment.

TABLE 6
EXAMPLE 5 SEQUENCES

| SEQ ID | | |
|---|---|---|
| NO: | Description | Sequence |
| 154 | pRC2 | gcgcgccgatatcgttaacgccccgcgccggccgctctagaacta |
| | | gtggatcccccggaagatcagaagttcctattccgaagttcctat |
| | | tctctagaaagtataggaacttctgatctgcgcagccgccatgcc |
| | | ggggttttacgagattgtgattaaggtccccagcgaccttgacga |
| | | gcatctgcccggcatttctgacagctttgtgaactgggtggccga |
| | | gaaggaatgggagttgccgccagattctgacatggatctgaatct |
| | | gattgagcaggcacccctgaccgtggccgagaagctgcagcgcga |
| | | ctttctgacggaatggcgccgtgtgagtaaggccccggaggccct |
| | | tttctttgtgcaatttgagaagggagagagctacttccacatgca |
| | | cgtgctcgtggaaaccaccggggtgaaatccatggttttgggacg |
| | | tttcctgagtcagattcgcgaaaaactgattcagagaatttaccg |
| | | cgggatcgagccgactttgccaaactggttcgcggtcacaaagac |
| | | cagaaatggcgccggaggcgggaacaaggtggtggatgagtgcta |
| | | catccccaattacttgctccccaaaacccagcctgagctccagtg |
| | | ggcgtggactaatatggaacagtatttaagcgcctgtttgaatct |
| | | cacggagcgtaaacggttggtggcgcagcatctgacgcacgtgtc |
| | | gcagacgcaggagcagaacaaagagaatcagaatcccaattctga |
| | | tgcgccggtgatcagatcaaaaacttcagccaggtacatggagct |
| | | ggtcgggtggctcgtggacaaggggattacctcggagaagcagtg |
| | | gatccaggaggaccaggcctcatacatctccttcaatgcggcctc |
| | | caactcgcggtcccaaatcaaggctgccttggacaatgcgggaaa |
| | | gattatgagcctgactaaaaccgcccccgactacctggtgggcca |
| | | gcagcccgtggaggacatttccagcaatcggatttataaaatttt |
| | | ggaactaaacgggtacgatccccaatatgcggcttccgtctttct |
| | | gggatgggccacgaaaaagttcggcaagaggaacaccatctggct |
| | | gtttgggcctgcaactaccgggaagaccaacatcgcggaggccat |
| | | agcccacactgtgcccttctacgggtgcgtaaactggaccaatga |
| | | gaactttcccttcaacgactgtgtcgacaagatggtgatctggtg |
| | | ggaggaggggaagatgaccgccaaggtcgtggagtcggccaaagc |
| | | cattctcggaggaagcaaggtgcgcgtggaccagaaatgcaagtc |
| | | ctcggcccagatagacccgactcccgtgatcgtcacctccaacac |
| | | caacatgtgcgccgtgattgacgggaactcaacgaccttcgaaca |
| | | ccagcagccgttgcaagaccggatgttcaaatttgaactcacccg |
| | | ccgtctggatcatgactttgggaaggtcaccaagcaggaagtcaa |
| | | agactttttccggtgggcaaaggatcacgtggttgaggtggagca |
| | | tgaattctacgtcaaaaagggtggagccaagaaaagacccgcccc |
| | | cagtgacgcagatataagtgagcccaaacgggtgcgcgagtcagt |
| | | tgcgcagccatcgacgtcagacgcggaagcttcgatcaactacgc |
| | | agacaggtaccaaaacaaatgttctcgtcacgtgggcatgaatct |
| | | gatgctgtttccctgcagacaatgcgagagaatgaatcagaattc |
| | | aaatatctgcttcactcacggacagaaagactgtttagagtgctt |
| | | tcccgtgtcagaatctcaacccgtttctgtcgtcaaaaaggcgta |
| | | tcagaaactgtgctacattcatcatatcatgggaaaggtgccaga |
| | | cgcttgcactgcctgcgatctggtcaatgtggatttggatgactg |
| | | catctttgaacaataaatgatttaaatcaggtatggctgccgatg |
| | | gttatcttccagattggctcgaggacactctctctgaaggaataa |
| | | gacagtggtggaagctcaaacctggcccaccaccaccaaagcccg |
| | | cagagcggcataaggacgacagcaggggtcttgtgcttcctgggt |
| | | acaagtacctcggacccttcaacggactcgacaagggagagccgg |
| | | tcaacgaggcagacgccgcggccctcgagcacgacaaagcctacg |
| | | accggcagctcgacagcggagacaacccgtacctcaagtacaacc |
| | | acgccgacgcggagtttcaggagcgccttaaagaagatacgtctt |
| | | ttgggggcaacctcggacgagcagtcttccaggcgaaaaagaggg |
| | | ttcttgaacctctgggcctggttgaggaacctgttaagacggctc |
| | | cgggaaaaaagaggccggtagagcactctcctgtggagccagact |
| | | cctcctcgggaaccggaaaggcgggccagcagcctgcaagaaaaa |
| | | gattgaattttggtcagactggagacgcagactcagtacctgacc |
| | | cccagcctctcggacagccaccagcagccccctctggtctgggaa |
| | | ctaatacgatggctacaggcagtggcgcaccaatggcagacaata |
| | | acgagggcgccgacggagtgggtaattcctcgggaaattggcatt |
| | | gcgattccacatggatgggcgacagagtcatcaccaccagcaccc |
| | | gaacctgggccctgcccacctacaacaaccacctctacaaacaaa |
| | | tttccagccaatcaggagcctcgaacgacaatcactactttggct |
| | | acagcaccccttgggggtattttgacttcaacagattccactgcc |
| | | acttttcaccacgtgactggcaaagactcatcaacaacaactggg |
| | | gattccgacccaagagactcaacttcaagctctttaacattcaag |
| | | tcaaagaggtcacgcagaatgacggtacgacgacgattgccaata |
| | | accttaccagcacggttcaggtgtttactgactcggagtaccagc |
| | | tcccgtacgtcctcggctcggcgcatcaaggatgcctcccgccgt |
| | | tcccagcagacgtcttcatggtgccacagtatggatacctcaccc |
| | | tgaacaacgggagtcaggcagtaggacgctåcttcattttactgc |
| | | ctggagtactttccttctcagatgctgcgtaccggaaacaacttt |
| | | accttcagctacacttttgaggacgttcctttccacagcagctac |
| | | gctcacagccagagtctggaccgtctcatgaatcctctcatcgac |
| | | cagtacctgtattacttgagcagaacaaacactccaagtggaacc |
| | | accacgcagtcaaggcttcagttttctcaggccggagcgagtgac |
| | | attcgggaccagtctaggaactggcttcctggaccctgttaccgc |
| | | cagcagcgagtatcaaagacatctgcggataacaacaacagtgaa |
| | | tactcgtggactggagctaccaagtaccacctcaatggcagagac |
| | | tctctggtgaatccgggcccggccatggcaagccacaaggacgat |
| | | gaagaaaagttttttcctcagagcggggttctcatctttgggaag |
| | | caaggctcagagaaaacaaatgtggacattgaaaaggtcatgatt |
| | | acagacgaagaggaaatcaggacaaccaatcccgtggctacggag |
| | | cagtatggttctgtatctaccaacctccagagaggcaacagacaa |
| | | gcagctaccgcagatgtcaacacacaaggcgttcttccaggcatg |
| | | gtctggcaggacagagatgtgtaccttcaggggcccatctgggca |
| | | aagattccacacacggacggacattttcacccctctcccctcatg |
| | | ggtggattcggacttaaacaccctcctccacagattctcatcaag |
| | | aacaccccggtacctgcgaatccttcgaccaccttcagtgcggca |
| | | aagtttgcttccttcatcacacagtactccacgggacaggtcagc |
| | | gtggagatcgagtgggagctgcagaaggaaaacagcaaacgctgg |
| | | aatcccgaaattcagtacacttccaactacaacaagtctgttaat |
| | | gtggactttactgtggacactaatggcgtgtattcagagcctcgc |
| | | cccattggcaccagatacctgactcgtaatctgtaattgcttgtt |
| | | aatcaataaaccgtttaattcgtttcagttgaactttggtctctg |
| | | cgtatttctttcttatctagtttccatggctacgtagataagtag |
| | | catggcgggttaatcattaactacagcccgggcgtttaaacagcg |
| | | ggcggaggggtggagtcgtgacgtgaattacgtcatagggttagg |
| | | gaggtcctgtattagaggtcacgtgagtgttttgcgacattttgc |
| | | gacaccatgtggtctcgctgggggggggggcccgagtgagcacgc |
| | | agggtctccattttgaagcgggaggtttgaacgagcgctggcgcg |
| | | ctcactggccgtcgttttacaacgtcgtgactgggaaaaccctgg |
| | | cgttacccaacttaatcgccttgcagcacatccccctttcgccag |
| | | ctggcgtaatagcgaagaggcccgcaccgatcgcccttcccaaca |
| | | gttgcgcagcctgaatggcgaatggaaattgtaagcgttaatatt |
| | | ttgttaaaattcgcgttaaatttttgttaaatcagctcatttttt |
| | | taaccaataggccgaaatcggcaaaatcccttataaatcaaaaga |
| | | atagaccgagatagggttgagtgttgttccagtttggaacaagag |
| | | tccactattaagaacgtggactccaacgtcaaagggcgaaaaacc |
| | | gtctatcagggcgatggcccactacgtgaaccatcaccctaatca |
| | | agttttttggggtcgaggtgccgtaaagcactaaatcggaaccct |
| | | aaagggagcccccgatttagagcttgacggggaaagccggcgaac |
| | | gtggcgagaaaggaagggaagaaagcgaaaggagcgggcgctagg |
| | | gcgctggcaagtgtagcggtcacgctgcgcgtaaccaccacaccc |
| | | gccgcgcttaatgcgccgctacagggcgcgtcaggtggcactttt |
| | | cggggaaatgtgcgcggaacccctatttgtttatttttctaaata |
| | | cattcaaatatgtatccgctcatgagacaataaccctgataaatg |
| | | cttcaataatattgaaaaaggaagagtatgagtattcaacatttc |
| | | cgtgtcgcccttattcccttttttgcggcattttgccttcctgtt |
| | | tttgctcacccagaaacgctggtgaaagtaaaagatgctgaagat |
| | | cagttgggtgcacgagtgggttacatcgaactggatctcaacagc |
| | | ggtaagatccttgagagttttcgccccgaagaacgttttccaatg |
| | | atgagcacttttaaagttctgctatgtggcgcggtattatcccgt |
| | | attgacgccgggcaagagcaactcggtcgccgcatacactattct |
| | | cagaatgacttggttgagtactcaccagtcacagaaaagcatctt |
| | | acggatggcatgacagtaagagaattatgcagtgctgccataacc |
| | | atgagtgataacactgcggccaacttacttctgacaacgatcgga |
| | | ggaccgaaggagctaaccgcttttttgcacaacatgggggatcat |
| | | gtaactcgccttgatcgttgggaaccggagctgaatgaagccata |
| | | ccaaacgacgagcgtgacaccacgatgcctgtagcaatggcaaca |
| | | acgttgcgcaaactattaactggcgaactacttactctagcttcc |
| | | cggcaacaattaatagactggatggaggcggataaagttgcagga |
| | | ccacttctgcgctcggcccttccggctggctggtttattgctgat |
| | | aaatctggagccggtgagcgtgggtctcgcggtatcattgcagca |
| | | ctggggccagatggtaagccctcccgtatcgtagttatctacacg |
| | | acggggagtcaggcaactatggatgaacgaaatagacagatcgct |
| | | gagataggtgcctcactgattaagcattggtaactgtcagaccaa |
| | | gtttactcatatatactttagattgatttaaaacttcatttttaa |
| | | tttaaaaggatctaggtgaagatcctttttgataatctcatgacc |
| | | aaaatcccttaacgtgagttttcgttccactgagcgtcagacccc |
| | | gtagaaaagatcaaaggatcttcttgagatcctttttttctgcgc |
| | | gtaatctgctgcttgcaaacaaaaaaaccaccgctaccagcggtg |
| | | gtttgtttgccggatcaagagctaccaactctttttccgaaggta |
| | | actggcttcagcagagcgcagataccaaatactgttcttctagtg |
| | | tagccgtagttaggccaccacttcaagaactctgtagcaccgcct |
| | | acatacctcgctctgctaatcctgttaccagtggctgctgccagt |
| | | ggcgataagtcgtgtcttaccgggttggactcaagacgatagtta |
| | | ccggataaggcgcagcggtcgggctgaacggggggttcgtgcaca |
| | | cagcccagcttggagcgaacgacctacaccgaactgagataccta |
| | | cagcgtgagctatgagaaagcgccacgcttcccgaagggagaaag |
| | | gcggacaggtatccggtaagcggcagggtcggaacaggagagcgc |
| | | acgagggagcttccagggggaaacgcctggtatctttatagtcct |
| | | gtcgggtttcgccacctctgacttgagcgtcgatttttgtgatgc |
| | | tcgtcaggggggcggagcctatggaaaaacgccagcaacgcggcc |
| | | tttttacggttcctggccttttgctggccttttgctcacatgttc |
| | | tttcctgcgttatcccctgattctgtggataaccgtattaccgcc |
| | | tttgagtgagctgataccgctcgccgcagccgaacgaccgagcgc |
| | | agcgagtcagtgagcgaggaagcggaagagcgcccaatacgcaaa |
| | | ccgcctctccccgcgcgttggccgattcattaatgcagctggcac |
| | | gacaggtttcccgactggaaagcgggcagtgagcgcaacgcaatt |
| | | aatgtgagttagctcactcattaggcaccccaggctttacacttt |
| | | atgcttccggctcgtatgttgtgtggaattgtgagcggataacaa |
| | | tttcacacaggaaacagctatgaccatgattacgccaa |
| 155 | pRC2_Opal | gcgcgccgatatcgttaacgccccgcgccggccgctctagaacta |
| | | gtggatcccccggaagatcagaagttcctattccgaagttcctat |
| | | tctctagaaagtataggaacttctgatctgcgcagccgccatgcc |
| | | ggggttttacgagattgtgattaaggtccccagcgaccttgacga |
| | | gcatctgcccggcatttctgacagctttgtgaactgggtggccga |
| | | gaaggaatgggagttgccgccagattctgacatggatctgaatct |
| | | gattgagcaggcacccctgaccgtggccgagaagctgcagcgcga |
| | | ctttctgacggaatggcgccgtgtgagtaaggccccggaggccct |
| | | tttctttgtgcaatttgagaagggagagagctacttccacatgca |
| | | cgtgctcgtggaaaccaccggggtgaaatccatggttttgggacg |
| | | tttcctgagtcagattcgcgaaaaactgattcagagaatttaccg |
| | | cgggatcgagccgactttgccaaactggttcgcggtcacaaagac |
| | | cagaaatggcgccggaggcgggaacaaggtggtggatgagtgcta |
| | | catccccaattacttgctccccaaaacccagcctgagctccagtg |
| | | ggcgtggactaatatggaacagtatttaagcgcctgtttgaatct |
| | | cacggagcgtaaacggttggtggcgcagcatctgacgcacgtgtc |
| | | gcagacgcaggagcagaacaaagagaatcagaatcccaattctga |
| | | tgcgccggtgatcagatcaaaaacttcagccaggtacatggagct |
| | | ggtcgggtggctcgtggacaaggggattacctcggagaagcagtg |
| | | gatccaggaggaccaggcctcatacatctccttcaatgcggcctc |
| | | caactcgcggtcccaaatcaaggctgccttggacaatgcgggaaa |
| | | gattatgagcctgactaaaaccgcccccgactacctggtgggcca |
| | | gcagcccgtggaggacatttccagcaatcggatttataaaatttt |
| | | ggaactaaacgggtacgatccccaatatgcggcttccgtctttct |
| | | gggatgggccacgaaaaagttcggcaagaggaacaccatctggct |
| | | gtttgggcctgcaactaccgggaagaccaacatcgcggaggccat |
| | | agcccacactgtgcccttctacgggtgcgtaaactggaccaatga |
| | | gaactttcccttcaacgactgtgtcgacaagatggtgatctggtg |
| | | ggaggaggggaagatgaccgccaaggtcgtggagtcggccaaagc |
| | | cattctcggaggaagcaaggtgcgcgtggaccagaaatgcaagtc |
| | | ctcggcccagatagacccgactcccgtgatcgtcacctccaacac |
| | | caacatgtgcgccgtgattgacgggaactcaacgaccttcgaaca |
| | | ccagcagccgttgcaagaccggatgttcaaatttgaactcacccg |
| | | ccgtctggatcatgactttgggaaggtcaccaagcaggaagtcaa |
| | | agactttttccggtgggcaaaggatcacgtggttgaggtggagca |
| | | tgaattctacgtcaaaaagggtggagccaagaaaagacccgcccc |
| | | cagtgacgcagatataagtgagcccaaacgggtgcgcgagtcagt |
| | | tgcgcagccatcgacgtcagacgcggaagcttcgatcaactacgc |
| | | agacaggtaccaaaacaaatgttctcgtcacgtgggcatgaatct |
| | | gatgctgtttccctgcagacaatgcgagagaatgaatcagaattc |
| | | aaatatctgcttcactcacggacagaaagactgtttagagtgctt |
| | | tcccgtgtcagaatctcaacccgtttctgtcgtcaaaaaggcgta |
| | | tcagaaactgtgctacattcatcatatcatgggaaaggtgccaga |
| | | cgcttgcactgcctgcgatctggtcaatgtggatttggatgactg |
| | | catctttgaacaataaatgatttaaatcaggtatggctgccgatg |
| | | gttatcttccagattggctcgaggacactctctctaaaggaataa |
| | | gacagtggtggaagctcaaacctggcccaccaccaccaaagcccg |
| | | cagagcggcataaggacgacagcaggggtcttgtgcttcctgggt |
| | | acaagtacctcggacccttcaacggactcgacaagggagagccgg |
| | | tcaacgaggcagacgccgcggccctcgagcacgacaaagcctacg |
| | | accggcagctcgacagcggagacaacccgtacctcaagtacaacc |
| | | acgccgacgcggagtttcaggagcgccttaaagaagatacgtctt |
| | | ttgggggcaacctcggacgagcagtcttccaggcgaaaaagaggg |
| | | ttcttgaacctctgggcctggttgaggaacctgttaagacggctc |
| | | cgggaaaaaagaggccggtagagcactctcctgtggagccagact |
| | | cctcctcgggaaccggaaaggcgggccagcagcctgcaagaaaaa |
| | | gattgaattttggtcagactggagacgcagactcagtacctgacc |
| | | cccagcctctcggacagccaccagcagccccctctggtctgggaa |
| | | ctaatacgatggctacaggcagtggcgcaccaatggcagacaata |
| | | acgagggcgccgacggagtgggtaattcctcgggaaattggcatt |
| | | gcgattccacatggatgggcgacagagtcatcaccaccagcaccc |
| | | gaacctgggccctgcccacctacaacaaccacctctacaaacaaa |
| | | tttccagccaatcaggagcctcgaacgacaatcactactttggct |
| | | acagcaccccttgggggtattttgacttcaacagattccactgcc |
| | | acttttcaccacgtgactggcaaagactcatcaacaacaactggg |
| | | gattccgacccaagagactcaacttcaagctctttaacattcaag |
| | | tcaaagaggtcacgcagaatgacggtacgacgacgattgccaata |
| | | accttaccagcacggttcaggtgtttactgactcggagtaccagc |
| | | tcccgtacgtcctcggctcggcgcatcaaggatgcctcccgccgt |
| | | tcccagcagacgtcttcatggtgccacagtatggatacctcaccc |
| | | taaacaacgggagtcaggcagtaggacgctcttcattttactgcc |
| | | tggagtactttccttctcagatgctgcgtaccggaaacaacttta |
| | | ccttcagctacacttttgaggacgttcctttccacagcagctacg |
| | | ctcacagccagagtctggaccgtctcatgaatcctctcatcgacc |
| | | agtacctgtattacttgagcagaacaaacactccaagtggaacca |
| | | ccacgcagtcaaggcttcagttttctcaggccggagcgagtgaca |
| | | ttcgggaccagtctaggaactggcttcctggaccctgttaccgcc |
| | | agcagcgagtatcaaagacatctgcggataacaacaacagtgaat |
| | | actcgtggactggagctaccaagtaccacctcaatggcagagact |
| | | ctctggtgaatccgggcccggccatggcaagccacaaggacgatg |
| | | aagaaaagttttttcctcagagcggggttctcatctttgggaagc |
| | | aaggctcagagaaaacaaatgtggacattgaaaaggtcatgatta |
| | | cagacgaagaggaaatcaggacaaccaatcccgtggctacggagc |
| | | agtatggttctgtatctaccaacctccagagaggcaacagacaag |
| | | cagctaccgcagatgtcaacacacaaggcgttcttccaggcatgg |
| | | tctggcaggacagagatgtgtaccttcaggggcccatctgggcaa |
| | | agattccacacacggacggacattttcacccctctcccctcatgg |
| | | gtggattcggacttaaacaccctcctccacagattctcatcaaga |
| | | acaccccggtacctgcgaatccttcgaccaccttcagtgcggcaa |
| | | agtttgcttccttcatcacacagtactccacgggacaggtcagcg |
| | | tggagatcgagtgggagctgcagaaggaaaacagcaaacgctgga |
| | | atcccgaaattcagtacacttccaactacaacaagtctgttaatg |
| | | tggactttactgtggacactaatggcgtgtattcagagcctcgcc |
| | | ccattggcaccagatacctgactcgtaatctgtaattgcttgtta |
| | | atcaataaaccgtttaattcgtttcagttgaactttggtctctgc |
| | | gtatttctttcttatctagtttccatggctacgtagataagtagc |
| | | atggcgggttaatcattaactacagcccgggcgtttaaacagcgg |
| | | gcggaggggtggagtcgtgacgtgaattacgtcatagggttaggg |
| | | aggtcctgtattagaggtcacgtgagtgttttgcgacattttgcg |
| | | acaccatgtggtctcgctgggggggggggcccgagtgagcacgca |
| | | gggtctccattttgaagcgggaggtttgaacgagcgctggcgcgc |
| | | tcactggccgtcgttttacaacgtcgtgactgggaaaaccctggc |
| | | gttacccaacttaatcgccttgcagcacatccccctttcgccagc |
| | | tggcgtaatagcgaagaggcccgcaccgatcgcccttcccaacag |
| | | ttgcgcagcctgaatggcgaatggaaattgtaagcgttaatattt |
| | | tgttaaaattcgcgttaaatttttgttaaatcagctcattttttt |
| | | aaccaataggccgaaatcggcaaaatcccttataaatcaaaagaa |
| | | tagaccgagatagggttgagtgttgttccagtttggaacaagagt |
| | | ccactattaagaacgtggactccaacgtcaaagggcgaaaaaccg |
| | | tctatcagggcgatggcccactacgtgaaccatcaccctaatcaa |
| | | gttttttggggtcgaggtgccgtaaagcactaaatcggaacccta |
| | | aagggagcccccgatttagagcttgacggggaaagccggcgaacg |
| | | tggcgagaaaggaagggaagaaagcgaaaggagcgggcgctaggg |
| | | cgctggcaagtgtagcggtcacgctgcgcgtaaccaccacacccg |
| | | ccgcgcttaatgcgccgctacagggcgcgtcaggtggcacttttc |
| | | ggggaaatgtgcgcggaacccctatttgtttatttttctaaatac |
| | | attcaaatatgtatccgctcatgagacaataaccctgataaatgc |
| | | ttcaataatattgaaaaaggaagagtatgagtattcaacatttcc |
| | | gtgtcgcccttattcccttttttgcggcattttgccttcctgttt |
| | | ttgctcacccagaaacgctggtgaaagtaaaagatgctgaagatc |
| | | agttgggtgcacgagtgggttacatcgaactggatctcaacagcg |
| | | gtaagatccttgagagttttcgccccgaagaacgttttccaatga |
| | | tgagcacttttaaagttctgctatgtggcgcggtattatcccgta |
| | | ttgacgccgggcaagagcaactcggtcgccgcatacactattctc |
| | | agaatgacttggttgagtactcaccagtcacagaaaagcatctta |
| | | cggatggcatgacagtaagagaattatgcagtgctgccataacca |
| | | tgagtgataacactgcggccaacttacttctgacaacgatcggag |
| | | gaccgaaggagctaaccgcttttttgcacaacatgggggatcatg |
| | | taactcgccttgatcgttgggaaccggagctgaatgaagccatac |
| | | caaacgacgagcgtgacaccacgatgcctgtagcaatggcaacaa |
| | | cgttgcgcaaactattaactggcgaactacttactctagcttccc |
| | | ggcaacaattaatagactggatggaggcggataaagttgcaggac |
| | | cacttctgcgctcggcccttccggctggctggtttattgctgata |
| | | aatctggagccggtgagcgtgggtctcgcggtatcattgcagcac |
| | | tggggccagatggtaagccctcccgtatcgtagttatctacacga |
| | | cggggagtcaggcaactatggatgaacgaaatagacagatcgctg |
| | | agataggtgcctcactgattaagcattggtaactgtcagaccaag |
| | | tttactcatatatactttagattgatttaaaacttcatttttaat |
| | | ttaaaaggatctaggtgaagatcctttttgataatctcatgacca |
| | | aaatcccttaacgtgagttttcgttccactgagcgtcagaccccg |
| | | tagaaaagatcaaaggatcttcttgagatcctttttttctgcgcg |
| | | taatctgctgcttgcaaacaaaaaaaccaccgctaccagcggtgg |
| | | tttgtttgccggatcaagagctaccaactctttttccgaaggtaa |
| | | ctggcttcagcagagcgcagataccaaatactgttcttctagtgt |
| | | agccgtagttaggccaccacttcaagaactctgtagcaccgccta |
| | | catacctcgctctgctaatcctgttaccagtggctgctgccagtg |
| | | gcgataagtcgtgtcttaccgggttggactcaagacgatagttac |
| | | cggataaggcgcagcggtcgggctgaacggggggttcgtgcacac |
| | | agcccagcttggagcgaacgacctacaccgaactgagatacctac |
| | | agcgtgagctatgagaaagcgccacgcttcccgaagggagaaagg |
| | | cggacaggtatccggtaagcggcagggtcggaacaggagagcgca |
| | | cgagggagcttccagggggaaacgcctggtatctttatagtcctg |
| | | tcgggtttcgccacctctgacttgagcgtcgatttttgtgatgct |
| | | cgtcaggggggcggagcctatggaaaaacgccagcaacgcggcct |
| | | ttttacggttcctggccttttgctggccttttgctcacatgttct |
| | | ttcctgcgttatcccctgattctgtggataaccgtattaccgcct |
| | | ttgagtgagctgataccgctcgccgcagccgaacgaccgagcgca |
| | | gcgagtcagtgagcgaggaagcggaagagcgcccaatacgcaaac |
| | | cgcctctccccgcgcgttggccgattcattaatgcagctggcacg |
| | | acaggtttcccgactggaaagcgggcagtgagcgcaacgcaatta |
| | | atgtgagttagctcactcattaggcaccccaggctttacacttta |
| | | tgcttccggctcgtatgttgtgtggaattgtgagcggataacaat |
| | | ttcacacaggaaacagctatgaccatgattacgccaa |
| 156 | pRC2_Ochre | gcgcgccgatatcgttaacgccccgcgccggccgctctagaacta |
| | | gtggatcccccggaagatcagaagttcctattccgaagttcctat |
| | | tctctagaaagtataggaacttctgatctgcgcagccgccatgcc |
| | | ggggttttacgagattgtgattaaggtccccagcgaccttgacga |
| | | gcatctgcccggcatttctgacagctttgtgaactgggtggccga |
| | | gaaggaatgggagttgccgccagattctgacatggatctgaatct |
| | | gattgagcaggcacccctgaccgtggccgagaagctgcagcgcga |
| | | ctttctgacggaatggcgccgtgtgagtaaggccccggaggccct |
| | | tttctttgtgcaatttgagaagggagagagctacttccacatgca |
| | | cgtgctcgtggaaaccaccggggtgaaatccatggttttgggacg |
| | | tttcctgagtcagattcgcgaaaaactgattcagagaatttaccg |
| | | cgggatcgagccgactttgccaaactggttcgcggtcacaaagac |
| | | cagaaatggcgccggaggcgggaacaaggtggtggatgagtgcta |
| | | catccccaattacttgctccccaaaacccagcctgagctccagtg |
| | | ggcgtggactaatatggaacagtatttaagcgcctgtttgaatct |
| | | cacggagcgtaaacggttggtggcgcagcatctgacgcacgtgtc |
| | | gcagacgcaggagcagaacaaagagaatcagaatcccaattctga |
| | | tgcgccggtgatcagatcaaaaacttcagccaggtacatggagct |
| | | ggtcgggtggctcgtggacaaggggattacctcggagaagcagtg |
| | | gatccaggaggaccaggcctcatacatctccttcaatgcggcctc |
| | | caactcgcggtcccaaatcaaggctgccttggacaatgcgggaaa |
| | | gattatgagcctgactaaaaccgcccccgactacctggtgggcca |
| | | gcagcccgtggaggacatttccagcaatcggatttataaaatttt |
| | | ggaactaaacgggtacgatccccaatatgcggcttccgtctttct |
| | | gggatgggccacgaaaaagttcggcaagaggaacaccatctggct |
| | | gtttgggcctgcaactaccgggaagaccaacatcgcggaggccat |
| | | agcccacactgtgcccttctacgggtgcgtaaactggaccaatga |
| | | gaactttcccttcaacgactgtgtcgacaagatggtgatctggtg |
| | | ggaggaggggaagatgaccgccaaggtcgtggagtcggccaaagc |
| | | cattctcggaggaagcaaggtgcgcgtggaccagaaatgcaagtc |
| | | ctcggcccagatagacccgactcccgtgatcgtcacctccaacac |
| | | caacatgtgcgccgtgattgacgggaactcaacgaccttcgaaca |
| | | ccagcagccgttgcaagaccggatgttcaaatttgaactcacccg |
| | | ccgtctggatcatgactttgggaaggtcaccaagcaggaagtcaa |
| | | agactttttccggtgggcaaaggatcacgtggttgaggtggagca |
| | | tgaattctacgtcaaaaagggtggagccaagaaaagacccgcccc |
| | | cagtgacgcagatataagtgagcccaaacgggtgcgcgagtcagt |
| | | tgcgcagccatcgacgtcagacgcggaagcttcgatcaactacgc |
| | | agacaggtaccaaaacaaatgttctcgtcacgtgggcatgaatct |
| | | gatgctgtttccctgcagacaatgcgagagaatgaatcagaattc |
| | | aaatatctgcttcactcacggacagaaagactgtttagagtgctt |
| | | tcccgtgtcagaatctcaacccgtttctgtcgtcaaaaaggcgta |
| | | tcagaaactgtgctacattcatcatatcatgggaaaggtgccaga |
| | | cgcttgcactgcctgcgatctggtcaatgtggatttggatgactg |
| | | catctttgaacaataaatgatttaaatcaggtatggctgccgatg |
| | | gttatcttccagattggctcgaggacactctctctgaaggaataa |
| | | gacagtggtggaagctcaaacctggcccacgggtcttgtgcttcc |
| | | tgggtacaagtacctcggacccttcaacggactcgacaagggaga |
| | | gccggtcaacgaggcagacgccgcggccctcgagcacgacaaagc |
| | | ctacgaccggcagctcgacagcggagacaacccgtacctcaagta |
| | | caaccacgccgacgcggagtttcaggagcgccttaaagaagatac |
| | | gtcttttgggggcaacctcggacgagcagtcttccaggcgaaaaa |
| | | gagggttcttgaacctctgggcctggttgaggaacctgttaagac |
| | | ggctccgggaaaaaagaggccggtagagcactctcctgtggagcc |
| | | agactcctcctcgggaaccggaaaggcgggccagcagcctgcaag |
| | | aaaaagattgaattttggtcagactggagacgcagactcagtacc |
| | | tgacccccagcctctcggacagccaccagcagccccctctggtct |
| | | gggaactaatacgatggctacaggcagtggcgcaccaatggcaga |
| | | caataacgagggcgccgacggagtgggtaattcctcgggaaattg |
| | | gcattgcgattccacatggatgggcgacagagtcatcaccaccag |
| | | cacccgaacctgggccctgcccacctacaacaaccacctctacaa |
| | | acaaatttccagccaatcaggagcctcgaacgacaatcactactt |
| | | tggctacagcaccccttgggggtattttgacttcaacagattcca |
| | | ctgccacttttcaccacgtgactggcaaagactcatcaacaacaa |
| | | ctggggattccgacccaagagactcaacttcaagctctttaacat |
| | | tcaagtcaaagaggtcacgcagaatgacggtacgacgacgattgc |
| | | caataaccttaccagcacggttcaggtgtttactgactcggagta |
| | | ccagctcccgtacgtcctcggctcggcgcatcaaggatgcctccc |
| | | gccgttcccagcagacgtcttcatggtgccacagtatggatacct |
| | | caccctgaacaacgggagtcaggcagtaggacgctcttcatttta |
| | | ctgcctggagtactttccttctcagatgctgcgtaccggaaacaa |
| | | ctttaccttcagctacacttttgaggacgttcctttccacagcag |
| | | ctacgctcacagccagagtctggaccgtctcatgaatcctctcat |
| | | cgaccagtacctgtattacttgagcagaacaaacactccaagtgg |
| | | aaccaccacgcagtcaaggcttcagttttctcaggccggagcgag |
| | | tgacattcgggaccagtctaggaactggcttcctggaccctgtta |
| | | ccgccagcagcgagtatcaaagacatctgcggataacaacaacag |
| | | tgaatactcgtggactggagctaccaagtaccacctcaatggcag |
| | | agactctctggtgaatccgggcccggccatggcaagccacaagga |
| | | cgatgaagaaaagttttttcctcagagcggggttctcatctttgg |
| | | gaagcaaggctcagagaaaacaaatgtggacattgaaaaggtcat |
| | | gattacagacgaagaggaaatcaggacaaccaatcccgtggctac |
| | | ggagcagtatggttctgtatctaccaacctccagagaggcaacag |
| | | acaagcagctaccgcagatgtcaacacacaaggcgttcttccagg |
| | | catggtctggcaggacagagatgtgtaccttcaggggcccatctg |
| | | ggcaaagattccacacacggacggacattttcacccctctcccct |
| | | catgggtggattcggacttaaacaccctcctccacagattctcat |
| | | caagaacaccccggtacctgcgaatccttcgaccaccttcagtgc |
| | | ggcaaagtttgcttccttcatcacacagtactccacgggacaggt |
| | | cagcgtggagatcgagtgggagctgcagaaggaaaacagcaaacg |
| | | ctggaatcccgaaattcagtacacttccaactacaacaagtctgt |
| | | taatgtggactttactgtggacactaatggcgtgtattcagagcc |
| | | tcgccccattggcaccagatacctgactcgtaatctgtagttgct |
| | | tgttaatcaatagaccgtttagttcgtttcagttgaactttggtc |
| | | tctgcgtatttctttcttatctagtttccatggctacgtagataa |
| | | gtagcatggcgggttaatcattaactacagcccgggcgtttaaac |
| | | agcgggcggaggggtggagtcgtgacgtgaattacgtcatagggt |
| | | tagggaggtcctgtattagaggtcacgtgagtgttttgcgacatt |
| | | ttgcgacaccatgtggtctcgctgggggggggggcccgagtgagc |
| | | acgcagggtctccattttgaagcgggaggtttgaacgagcgctgg |
| | | cgcgctcactggccgtcgttttacaacgtcgtgactgggaaaacc |
| | | ctggcgttacccaacttaatcgccttgcagcacatccccctttcg |
| | | ccagctggcgtaatagcgaagaggcccgcaccgatcgcccttccc |
| | | aacagttgcgcagcctgaatggcgaatggaaattgtaagcgttaa |
| | | tattttgttaaaattcgcgttaaatttttgttaaatcagctcatt |
| | | tttttaaccaataggccgaaatcggcaaaatcccttataaatcaa |
| | | aagaatagaccgagatagggttgagtgttgttccagtttggaaca |
| | | agagtccactattaagaacgtggactccaacgtcaaagggcgaaa |
| | | aaccgtctatcagggcgatggcccactacgtgaaccatcacccta |
| | | atcaagttttttggggtcgaggtgccgtaaagcactaaatcggaa |
| | | ccctaaagggagcccccgatttagagcttgacggggaaagccggc |
| | | gaacgtggcgagaaaggaagggaagaaagcgaaaggagcgggcgc |
| | | tagggcgctggcaagtgtagcggtcacgctgcgcgtaaccaccac |
| | | acccgccgcgcttaatgcgccgctacagggcgcgtcaggtggcac |
| | | ttttcggggaaatgtgcgcggaacccctatttgtttatttttcta |
| | | aatacattcaaatatgtatccgctcatgagacaataaccctgata |
| | | aatgcttcaataatattgaaaaaggaagagtatgagtattcaaca |
| | | tttccgtgtcgcccttattcccttttttgcggcattttgccttcc |
| | | tgtttttgctcacccagaaacgctggtgaaagtaaaagatgctga |
| | | agatcagttgggtgcacgagtgggttacatcgaactggatctcaa |
| | | cagcggtaagatccttgagagttttcgccccgaagaacgttttcc |
| | | aatgatgagcacttttaaagttctgctatgtggcgcggtattatc |
| | | ccgtattgacgccgggcaagagcaactcggtcgccgcatacacta |
| | | ttctcagaatgacttggttgagtactcaccagtcacagaaaagca |
| | | tcttacggatggcatgacagtaagagaattatgcagtgctgccat |
| | | aaccatgagtgataacactgcggccaacttacttctgacaacgat |
| | | cggaggaccgaaggagctaaccgcttttttgcacaacatggggga |
| | | tcatgtaactcgccttgatcgttgggaaccggagctgaatgaagc |
| | | cataccaaacgacgagcgtgacaccacgatgcctgtagcaatggc |
| | | aacaacgttgcgcaaactattaactggcgaactacttactctagc |
| | | ttcccggcaacaattaatagactggatggaggcggataaagttgc |
| | | aggaccacttctgcgctcggcccttccggctggctggtttattgc |
| | | tgataaatctggagccggtgagcgtgggtctcgcggtatcattgc |
| | | agcactggggccagatggtaagccctcccgtatcgtagttatcta |
| | | cacgacggggagtcaggcaactatggatgaacgaaatagacagat |
| | | cgctgagataggtgcctcactgattaagcattggtaactgtcaga |
| | | ccaagtttactcatatatactttagattgatttaaaacttcattt |
| | | ttaatttaaaaggatctaggtgaagatcctttttgataatctcat |
| | | gaccaaaatcccttaacgtgagttttcgttccactgagcgtcaga |
| | | ccccgtagaaaagatcaaaggatcttcttgagatcctttttttct |
| | | gcgcgtaatctgctgcttgcaaacaaaaaaaccaccgctaccagc |
| | | ggtggtttgtttgccggatcaagagctaccaactctttttccgaa |
| | | ggtaactggcttcagcagagcgcagataccaaatactgttcttct |
| | | agtgtagccgtagttaggccaccacttcaagaactctgtagcacc |
| | | gcctacatacctcgctctgctaatcctgttaccagtggctgctgc |
| | | cagtggcgataagtcgtgtcttaccgggttggactcaagacgata |
| | | gttaccggataaggcgcagcggtcgggctgaacggggggttcgtg |
| | | cacacagcccagcttggagcgaacgacctacaccgaactgagata |
| | | cctacagcgtgagctatgagaaagcgccacgcttcccgaagggag |
| | | aaaggcggacaggtatccggtaagcggcagggtcggaacaggaga |
| | | gcgcacgagggagcttccagggggaaacgcctggtatctttatag |
| | | tcctgtcgggtttcgccacctctgacttgagcgtcgatttttgtg |
| | | atgctcgtcaggggggcggagcctatggaaaaacgccagcaacgc |
| | | ggcctttttacggttcctggccttttgctggccttttgctcacat |
| | | gttctttcctgcgttatcccctgattctgtggataaccgtattac |
| | | cgcctttgagtgagctgataccgctcgccgcagccgaacgaccga |
| | | gcgcagcgagtcagtgagcgaggaagcggaagagcgcccaatacg |
| | | caaaccgcctctccccgcgcgttggccgattcattaatgcagctg |
| | | gcacgacaggtttcccgactggaaagcgggcagtgagcgcaacgc |
| | | aattaatgtgagttagctcactcattaggcaccccaggctttaca |
| | | ctttatgcttccggctcgtatgttgtgtggaattgtgagcggata |
| | | acaatttcacacaggaaacagctatgaccatgattacgccaa |
| 157 | CCT5 | GCCCCAGTGGCCTGATGGATAAGGTACTGGCCTTCAAAGCCAGGG |
| | | ATTGTGGGTTCGAGTTCCACCTGGGGTA |

### Example 6: Silencing Suppressor tRNAs Increases AAV Titer

This example demonstrates that silencing suppressor tRNAs during AAV virion production increases AAV titer compared to AAV virion production without silencing suppressor tRNAs.

A 3A plasmid was generated as follows. An AAV backbone plasmid as shown in FIG. 2A, but having the sequence between ITRs as shown in FIG. 2B, was generated. The ITR-ITR sequence comprised three conditionally repressible CCT2.10 suppressor tRNA coding sequences (3× a sequence comprising a sequence of the repressor element (TRE sequence) at +1 position immediately upstream of the sequence coding for the suppressor tRNA), synthetic filler sequences (100 bps), an EF1a_core-mCherry sequence (transduction marker), and strawberry sequences (quantification tags for ddPCR quantification of episomes/AAV transcripts). The TRE sequence comprised two repeats of the Tet operator (19 bp) with a TC spacer: TCCCTATCAGTGATAGAGATCTCCCTATCAGTGATAGAGA (SEQ ID NO: 149). The AAV serotype was AAV2. A control STX650 plasmid comprising CMV-GFP was also generated.

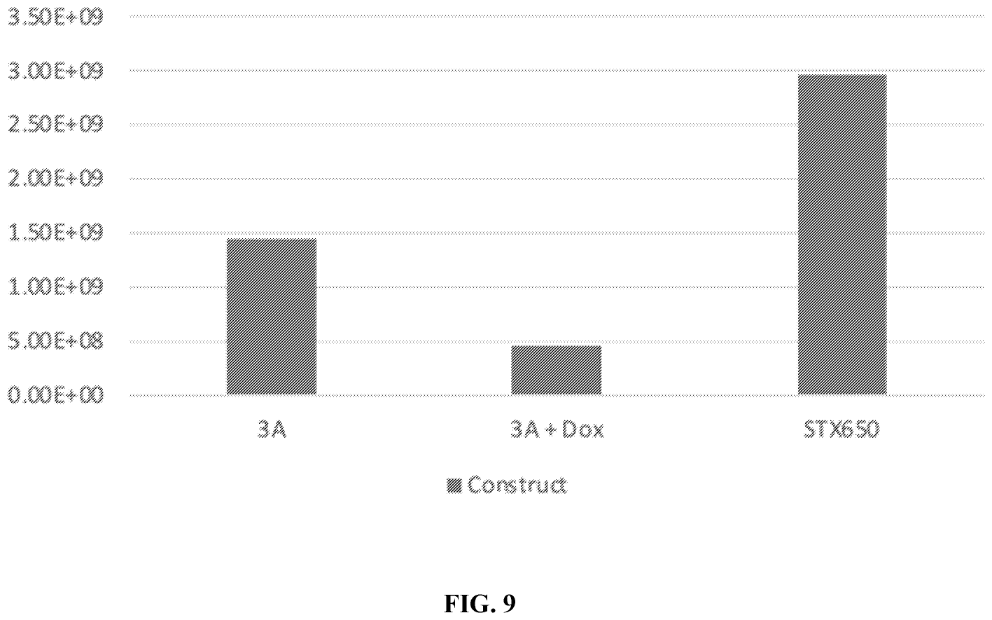

Triple transfection was performed in cells stably expressing the Tet repressor, with an AAV helper plasmid, an AAV Rep/Cap plasmid, and the 3A plasmid in the absence of doxycycline, the 3A plasmid in the presence of doxycycline, or the control STX650 plasmid, for the production of AAV virion encapsidating the polynucleotide comprising the sequence coding for the suppressor tRNA. During the production of AAV virion encapsidating the polynucleotide comprising the sequence coding for the suppressor tRNA from the 3A plasmid in the absence of doxycycline, the Tet repressor bound to the polynucleotide encoding the TRE sequence to silence the expression of the suppressor tRNAs. During the production of AAV virion encapsidating the polynucleotide comprising the sequence coding for the suppressor tRNA from the 3A plasmid in the presence of doxycycline, the Tet repressor bound to doxycycline instead of the polynucleotide encoding the TRE sequence, allowing the expression of the suppressor tRNAs. FIG. 9 shows increased AAV titer produced by cells transfected with a plasmid comprising a TRE sequence upstream of the suppressor tRNA sequence in the absence of doxycycline compared to cells transfected with a plasmid comprising a TRE sequence upstream of the suppressor tRNA sequence in the presence of doxycycline.

### Example 7: Assessment of Repressor Elements of a Repressor Element-Suppressor tRNA Construct in a Plasmid

This example assesses the impact of the repressor element and of repressor element placement in repressor element-suppressor tRNA constructs. A Repressor+ context (HEK 293 cells stably expressing a Tet repressor) and a Repressor-context (HEK 293 cells (lacking expression of a Tet repressor)) were used to assess this impact. The Repressor+ context provides a means for identifying how the number of TetO sequences in a repressor element and placement of the repressor element affect repression of the suppressor tRNA expression resulting in suppressor tRNA silencing, e.g., during AAV production, and therefore affecting AAV titer. The Repressor-context provides a means for identifying the functional influence of the number of TetO sequences in a repressor element and placement of the repressor element in the absence of the repressor, e.g., to assess the impact of the repressor element on the expression and function of suppressor tRNA in a therapeutically relevant context.

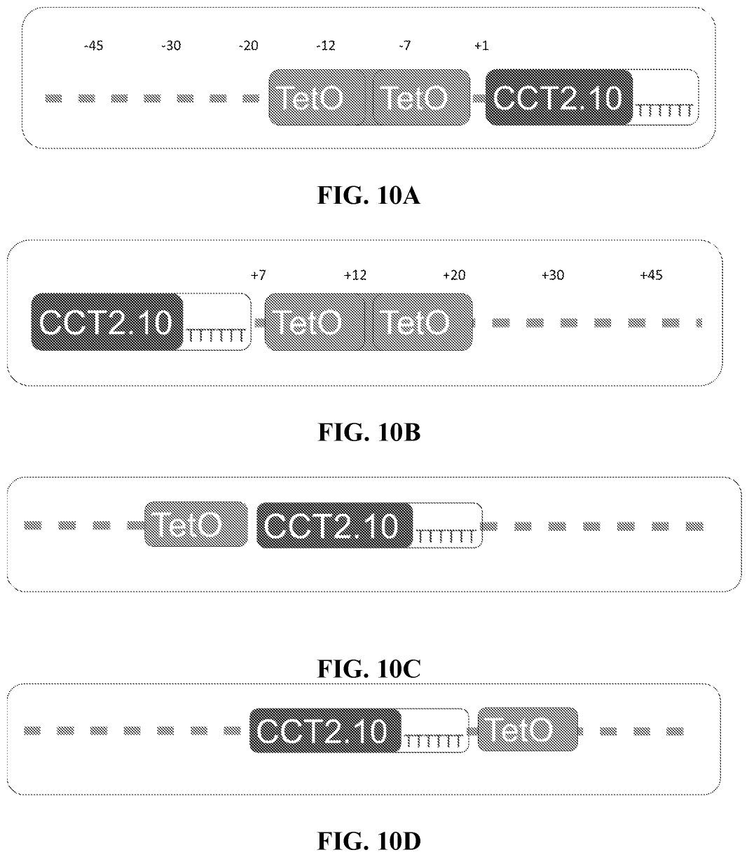

A series of plasmids comprising a polynucleotide encoding for one or two TetO sequences of the repressor element at various positions upstream or downstream of the suppressor tRNA sequence were generated by cloning the polynucleotide comprising the sequence of the repressor element into ITR free plasmids in which the suppressor tRNA sequence was a single suppressor tRNA sequence coding for CCT2.10, with co-expression of Thy1.1 as a transfection marker, driven by CMV-350. Schematic depictions of example repressor element-suppressor tRNA constructs are shown in FIGS. 10A-10D.

FIG. 10A shows a schematic depiction of an example repressor element-suppressor tRNA construct comprising the repressor element (two TetO sequences (TetO), e.g., two TetO sequences separated by a TC spacer (e.g., SEQ ID NO: 149)) placed upstream of the suppressor tRNA sequence (CCT2.10). FIG. 10B shows a schematic depiction of an example repressor element-suppressor tRNA construct comprising the repressor element (two TetO sequences (TetO), e.g., two TetO sequences separated by a TC spacer (e.g., SEQ ID NO: 149)) placed downstream of the suppressor tRNA sequence (CCT2.10). The numbers +1, −7, −12, −20, −30 and −45 indicate the different positions of the repressor element (two TetO sequences (TetO), e.g., two TetO sequences separated by a TC spacer (e.g., SEQ ID NO: 149)) relative to the suppressor tRNA sequence in FIG. 10A and FIG. 10B. FIG. 10C shows a schematic depiction of an example repressor element-suppressor tRNA construct comprising the repressor element (one TetO sequence (TetO); e.g., SEQ ID NO: 150) placed upstream of the tRNA sequence (CCT2.10). FIG. 10D shows a schematic depiction of an example repressor element-suppressor tRNA construct comprising the repressor element (one TetO sequence (TetO); e.g., SEQ ID NO: 150) placed downstream of the tRNA sequence (CCT2.10).

The repressor element and the placement of repressor element in each plasmid tested is described in TABLE 7 below. The position of the TetO sequence that is closest to the sequence of the suppressor tRNA is indicated for plasmids in which two copies of the TetO sequence of the repressor element are present. For controls, a plasmid without the repressor element and having a single copy of the sequence coding for the suppressor tRNA (noTET_CCT2.10) and a plasmid without the repressor element and having a single copy of the same suppressor tRNA but having a wild-type anticodon (noTET_CCT2.10-WT) were generated. Constructs shown in TABLE 7 were cloned into the backbone of SEQ ID NO: 213. The sequence for the active part of the plasmid for each construct is provided below as SEQ ID NOs: 158-181.

TABLE 7
REPRESSOR ELEMENT-SUPPRESSOR TRNA CONSTRUCTS
AND CORRESPONDING CONTROLS

| | | | | Number | |
|---|---|---|---|---|---|
| | | Repressor | | of TetO | |
| | | Element Position | | sequences | |
| | | compared to | Repressor | in | Suppressor |
| | SEQ ID | Suppressor | Element | Repressor | tRNA |
| Construct | NO: | tRNA sequence | Spacing | Element | Sequence |
| 1TETp1-CCT2.10 | 163 | Upstream | +1 (no spacing, | 1 | CCT2.10 |
| | | | Repressor | | |
| | | | Element right | | |
| | | | before the | | |
| | | | suppressor | | |
| | | | tRNA sequence | | |
| | | | start site) | | |
| 1TETm7-CCT2.10 | 158 | Upstream | −7 | 1 | CCT2.10 |
| 1TETm12-CCT2.10 | 159 | Upstream | −12 | 1 | CCT2.10 |
| 1TETm20-CCT2.10 | 160 | Upstream | −20 | 1 | CCT2.10 |
| 1TETm30-CCT2.10 | 161 | Upstream | −30 | 1 | CCT2.10 |
| 1TETm45-CCT2.10 | 162 | Upstream | −45 | 1 | CCT2.10 |
| 1TETp7-CCT2.10 | 164 | Downstream | +7 | 1 | CCT2.10 |
| 1TETp12-CCT2.10 | 165 | Downstream | +12 | 1 | CCT2.10 |
| 1TETp20-CCT2.10 | 166 | Downstream | +20 | 1 | CCT2.10 |
| 1TETp30-CCT2.10 | 167 | Downstream | +30 | 1 | CCT2.10 |
| 1TETp45-CCT2.10 | 168 | Downstream | +45 | 1 | CCT2.10 |
| TETp1-CCT2.10 | 176 | Upstream | +1 (no spacing, | 2 | CCT2.10 |
| | | | Repressor | | |
| | | | Element right | | |
| | | | before the | | |
| | | | suppressor | | |
| | | | tRNA sequence | | |
| | | | start site) | | |
| TETm7-CCT2.10 | 171 | Upstream | −7 | 2 | CCT2.10 |
| TETm12-CCT2.10 | 172 | Upstream | −12 | 2 | CCT2.10 |
| TETm20-CCT2.10 | 173 | Upstream | −20 | 2 | CCT2.10 |
| TETm30-CCT2.10 | 174 | Upstream | −30 | 2 | CCT2.10 |
| TETm45-CCT2.10 | 175 | Upstream | −45 | 2 | CCT2.10 |
| TETp7-CCT2.10 | 177 | Downstream | +7 | 2 | CCT2.10 |
| TETp12-CCT2.10 | 178 | Downstream | +12 | 2 | CCT2.10 |
| TETp20-CCT2.10 | 179 | Downstream | +20 | 2 | CCT2.10 |
| TETp30-CCT2.10 | 180 | Downstream | +30 | 2 | CCT2.10 |
| TETp45-CCT2.10 | 181 | Downstream | +45 | 2 | CCT2.10 |
| noTET_CCT2.10-WT | 170 | None | N/A | 0 | CCT2.10 |
| noTET_CCT2.10 | 169 | None | N/A | 0 | CCT2.10 WT |
| | | | | | anticodon |

Repressor− cells or Repressor+ cells stably expressing broken GFP (a R74X premature stop codon or a R97X premature stop codon inserted into a GFP sequence) were transfected with the plasmids comprising the constructs of TABLE 7. Detection of a GFP signal indicates the suppressor tRNA was expressed and that there was subsequent readthrough of the premature stop codon to produce detectable GFP. Detection and quantification of the GFP signal was performed using flow cytometry.

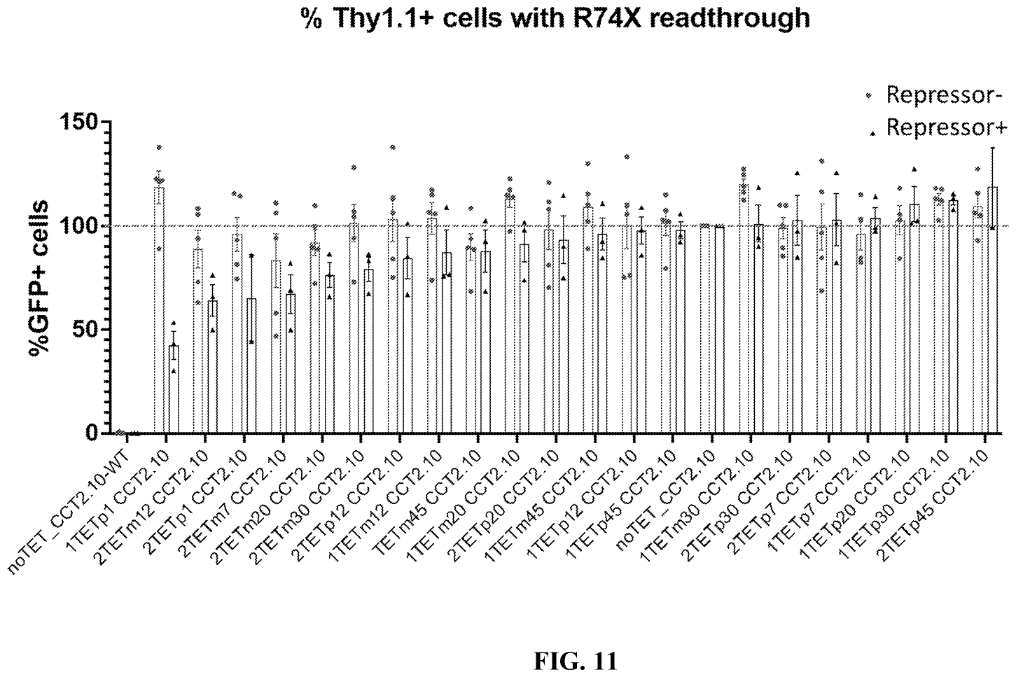

FIG. 11 shows a graph of % GFP+ cells of transfected cells (Thy 1.1+ cells) after independent transfection of either HEK293 cells (Repressor-) stably expressing a GFP reporter comprising a R74X premature stop codon (left bars) or HEK293 cells expressing a Tet repressor (Repressor+) that stably express a GFP reporter comprising a R74X premature stop codon (right bars) with plasmids comprising the repressor element-suppressor tRNA constructs or control constructs of TABLE 7 to assess the number of the TetO sequences of the repressor element and the placement of repressor element relative to the sequence coding for the suppressor tRNA. The % GFP+ cells were normalized to the % GFP+ cells of the control noTET_CCT2.10-WT.

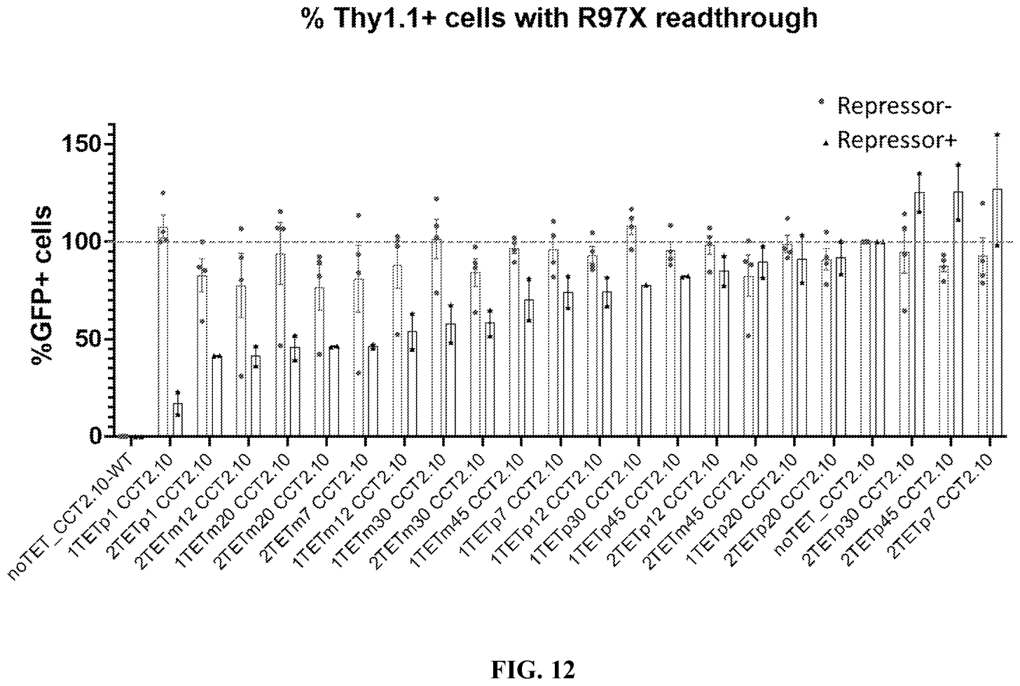

FIG. 12 shows a graph of % GFP+ cells of transfected cells (Thy 1.1+ cells) after independent transfection of either HEK293 cells (Repressor-) stably expressing a GFP reporter comprising a R97X premature stop codon or HEK293 cells expressing a Tet repressor (Repressor+) that stably express a GFP reporter comprising a R97X premature stop codon with plasmids comprising the repressor element-suppressor tRNA constructs or control constructs of TABLE 7 to assess the number of the TetO sequences of the repressor element and the placement of repressor element relative to the sequence coding for the suppressor tRNA. The % GFP+ cells were normalized to the % GFP+ cells of the control noTET_CCT2.10-WT.

Importantly, FIG. 11 and FIG. 12 demonstrated both silencing of suppressor tRNA expression in the presence of a repressor and suppressor tRNA expression from constructs comprising the repressor element in the absence of the corresponding repressor. More specifically, these results demonstrated that, in the presence of the repressor (e.g., in Repressor+ cells): 1) placement of the repressor element at the +1 position provided greater repression than placement of the repressor element at other positions, 2) placement of the repressor element upstream of the suppressor tRNA sequence provided greater suppression than placement downstream of the suppressor tRNA sequence, 3) two copies of the TetO sequence in a repressor element provided greater repression than one copy of a TetO sequence in a repressor element, with the primary exception of the +1 position where one copy of a TetO sequence in a repressor element performed better. These results also demonstrated that, in the absence of the repressor (e.g., in Repressor-cells), the number of copies of the TetO sequence in the repressor element and the placement of the repressor element did not prevent expression of the suppressor tRNA.

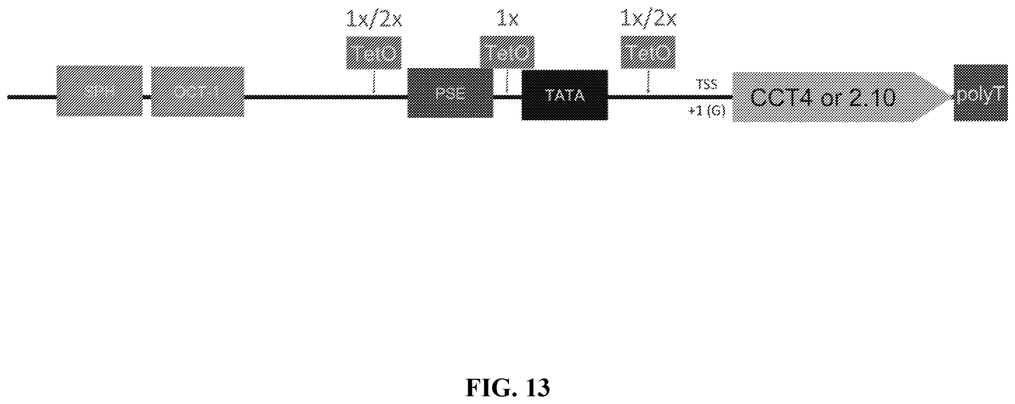

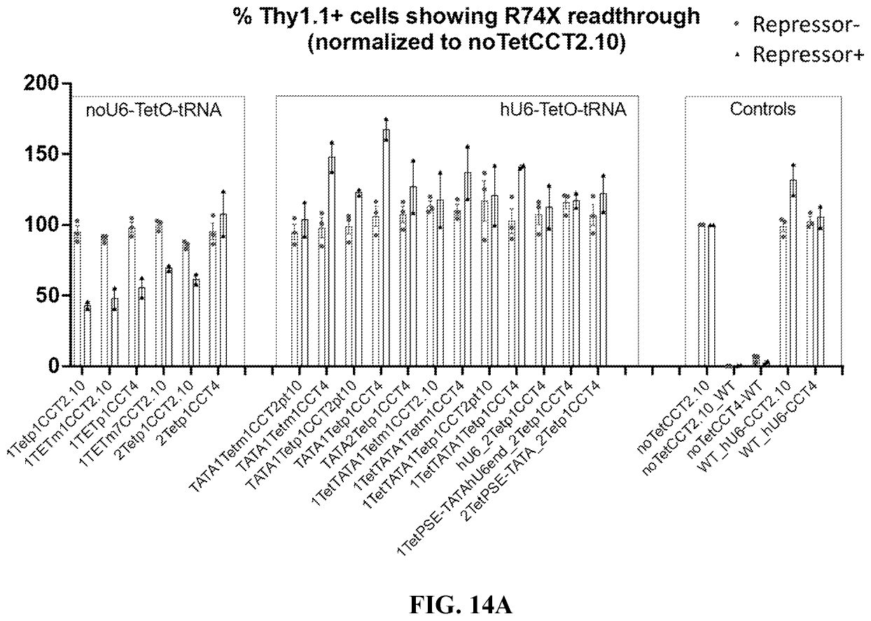

Additionally, a set of repressor element-suppressor tRNA constructs with the repressor element placed at different positons within or at the end of an hU6 promoter (hU6-repressor element-suppressor tRNA constructs) were tested. FIG. 13 shows a schematic depiction of exemplary variations of repressor element-suppressor tRNA constructs comprising a hU6 promoter as described herein. SPH, OCT-1, PSE and TATA are elements of the hU6 promoter. Variations of the placement of the repressor elements with the elements of the hU6 promoter are shown, with the repressor element (“TetO”) placed in one or more of the three positions downstream of the SPH and OCT-1 elements—either before the PSE element, between the PSE and TATA box, or after the TATA box, as shown on FIG. 13. As indicated, the repressor element may be present as one TetO sequence (as indicated by 1×; e.g., SEQ ID NO: 150) or two TetO sequences (as indicated by 2×; e.g., SEQ ID NO: 149). TSS indicates the transcriptional start site of the sequence coding for a suppressor tRNA (e.g., CCT4 or CCT2.10). More specifically, the constructs that were tested are described in TABLE 8. All constructs were cloned into an ITR-free a plasmid of SEQ ID NO: 213. The active payload sequence (tRNA with or without the upstream tetO and/or hU6) for each construct are provided below as SEQ ID NOs: 182-202.

TABLE 8
HU6-REPRESSOR ELEMENT-SUPPRESSOR TRNA
CONSTRUCTS AND CORRESPONDING CONTROLS

| | | | | | Number | |
|---|---|---|---|---|---|---|
| | | | Repressor | | of | |
| | | | Element Position | | Repressor | |
| | | | (that is the | Repressor | Elements | |
| | | | closest to the | Element(s) | (copies of | |
| | | | suppressor | (Tet(s)) | TetO | |
| | | | tRNA sequence) | Spacing | sequence | |
| hU6-Repressor Element- | SEQ | | relative to the | Relative to | in | Suppressor |
| Suppressor tRNA | ID | hU6 | suppressor | PSE and | Repressor | tRNA |
| Construct | NO: | Promoter | tRNA sequence | TATA | Element) | sequence |
| noU6-TetO-tRNA control #1 | 163 | N/A | +1 (immediately | N/A | 1 (1 TetO | CCT2.10 |
| (1Tetp1CCT2.10) | | | upstream of the | | sequence) | |
| | | | suppressor tRNA | | | |
| | | | sequence) | | | |
| noU6-TetO-tRNA control #2 | 195 | N/A | −1 (2 bases | N/A | 1 (1 TetO | CCT2.10 |
| (1TETm1CCT2.10) | | | upstream of the | | sequence) | |
| | | | suppressor tRNA | | | |
| | | | sequence) | | | |
| noU6-TetO-tRNA control #3 | 197 | N/A | +1 | N/A | 1 (1 TetO | CCT4 |
| (1TETp1CCT4) | | | | | sequence) | |
| noU6-TetO-tRNA control #4 | 196 | N/A | −7 (8 bases | N/A | 1 (1 TetO | CCT2.10 |
| (1TETm7CCT2.10) | | | upstream of the | | sequence) | |
| | | | suppressor tRNA | | | |
| | | | sequence) | | | |
| noU6-TetO-tRNA control #5 | 176 | N/A | +1 | N/A | 1 (2 TetO | CCT2.10 |
| (2Tetp1CCT2.10) | | | | | sequences) | |
| noU6-TetO-tRNA control #6 | 198 | N/A | +1 | N/A | 1 (2 TetO | CCT4 |
| (2Tetp1CCT4) | | | | | sequences) | |
| hU6-tetO-tRNA construct #1 | 186 | hU6 | −1 | PSE- | 1 (1 TetO | CCT2.10 |
| (TATAITetm1CCT2pt10) | | | | TATA-Tet | sequence) | |
| hU6-tetO-tRNA construct #2 | 187 | hU6 | −1 | PSE- | 1 (1 TetO | CCT4 |
| (TATA1Tetm1CCT4) | | | | TATA-Tet | sequence) | |
| hU6-tetO-tRNA construct #3 | 188 | hU6 | +1 | PSE- | 1 (1 TetO | CCT2.10 |
| (TATAITetp1CCT2pt10) | | | | TATA-Tet | sequence) | |
| hU6-tetO-tRNA construct #4 | 189 | hU6 | +1 | PSE- | 1 (2 TetO | CCT4 |
| (TATA1Tetp1CCT4) | | | | TATA-Tet | sequences) | |
| hU6-tetO-tRNA construct #5 | 190 | hU6 | +1 | PSE- | 1 (2 TetO | CCT4 |
| (TATA2Tetp1CCT4) | | | | TATA- | sequences) | |
| | | | | Tet-Tet | | |
| hU6-tetO-tRNA construct #6 | 182 | hU6 | +1 | PSE-Tet- | 2 (1 TetO | CCT2.10 |
| (1TetTATA1Tetm1CCT2.10) | | | | TATA-Tet | sequence | |
| | | | | | each) | |
| hU6-tetO-tRNA construct #7 | 183 | hU6 | −1 | PSE-Tet- | 2 (1 TetO | CCT4 |
| (1TetTATA1Tetm1CCT4) | | | | TATA-Tet | sequence | |
| | | | | | each) | |
| hU6-tetO-tRNA construct #8 | 184 | hU6 | +1 | PSE-Tet- | 2 (1 TetO | CCT2.10 |
| (1TetTATA1Tetp1CCT2.10) | | | | TATA-Tet | sequence | |
| | | | | | each) | |
| hU6-tetO-tRNA construct #9 | 185 | hU6 | −1 | PSE-Tet- | 2 (1 TetO | CCT4 |
| (1TetTATA1Tetp1CCT4) | | | | TATA-Tet | sequence | |
| | | | | | each) | |
| hU6-tetO-tRNA construct | 193 | hU6 | +1 | PSE- | 1 (2 TetO | CCT4 |
| #10 | | | | TATA-Tet | sequences) | |
| (hU6_2Tetp1CCT4) | | | | | | |
| hU6-tetO-tRNA construct | 192 | hU6 | +1 | Tet-PSE- | 1 (1 TetO | CCT4 |
| #11 | | | | TATA- | sequence; | |
| (1TetPSE- | | | | hU6end- | before | |
| TATAhU6end_2Tetp1CCT4) | | | | Tet | PSE) | |
| | | | | | 1 (2 TetO | |
| | | | | | sequences; | |
| | | | | | after hU6 | |
| | | | | | end) | |
| hU6-tetO-tRNA construct | 191 | hU6 | +1 | Tet-PSE- | 2 (2 TetO | CCT4 |
| #12 | | | | TATA-Tet | sequences | |
| (2TetPSE- | | | | | each) | |
| TATA_2Tetp1CCT4) | | | | | | |
| Control Construct #1 | 169 | N/A | N/A | N/A | N/A | CCT2.10 |
| (noTetCCT2.10) | | | | | | |
| Control Construct #2 | 170 | N/A | N/A | N/A | N/A | CCT2.10 |
| (noTetCCT2.10_WT) | | | | | | WT |
| Control Construct #3 | 194 | N/A | N/A | N/A | N/A | CCT4 WT |
| (noTetCCT4-WT) | | | | | | |
| Control Construct #4 | 199 | hU6 | N/A | N/A | N/A | CCT2.10 |
| (WT_hU6-CCT2.10) | | | | | | |
| Control Construct #5 | 201 | hU6 | N/A | N/A | N/A | CCT4 |
| WT_hU6-CCT4 | | | | | | |

Repressor− cells or Repressor+ cells stably expressing broken GFP (a R74X premature stop codon or a R97X premature stop codon inserted into a GFP sequence) were transfected with the plasmids comprising the constructs of TABLE 8. Detection of a GFP signal indicates the suppressor tRNA was expressed and that there was subsequent readthrough of the premature stop codon to produce detectable GFP. Detection and quantification of the GFP signal was performed using flow cytometry.

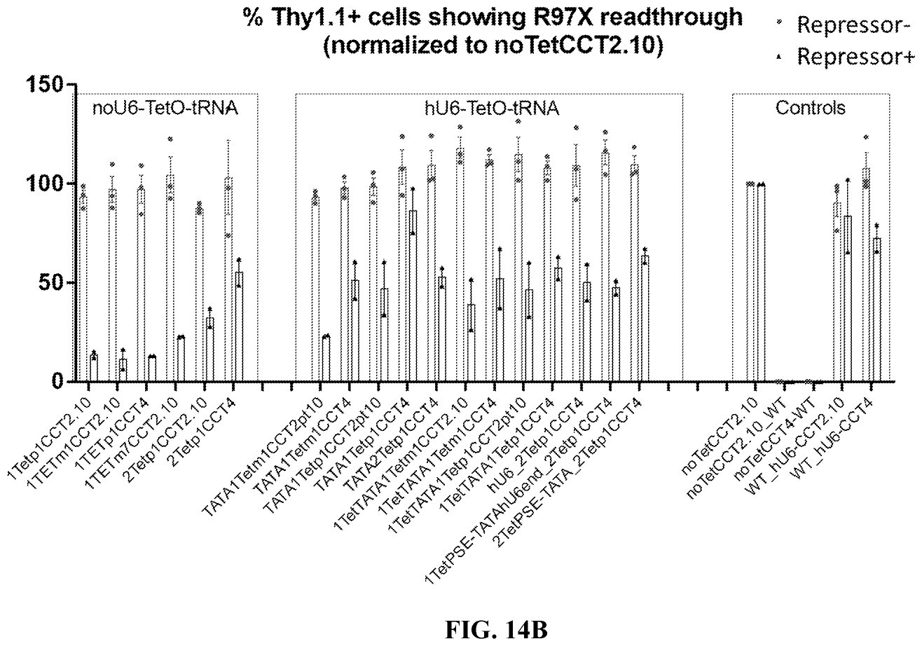

FIG. 14A shows a graph of % GFP+ cells of transfected cells (Thy 1.1+ cells) after independent transfection of either HEK293 cells (Repressor-) stably expressing a GFP reporter comprising a R74X premature stop codon (left bars) or HEK293 cells expressing a Tet repressor (Repressor+) that stably express a GFP reporter comprising a R74X premature stop codon (right bars) with plasmids comprising the hU6-repressor element-suppressor tRNA constructs or control constructs of TABLE 8 to assess the number of the TetO sequences of the repressor element and the placement of repressor element relative to elements of the hU6 promoter and the sequence coding for the suppressor tRNA. The % GFP+ cells were normalized to the % GFP+ cells of the control noTetCCT2.10.

FIG. 14B shows a graph of % GFP+ cells of transfected cells (Thy 1.1+ cells) after independent transfection of either HEK293 cells (Repressor−) stably expressing a GFP reporter comprising a R97X premature stop codon or HEK293 cells expressing a Tet repressor (Repressor+) that stably express a GFP reporter comprising a R97X premature stop codon with plasmids comprising the hU6-repressor element-suppressor tRNA constructs or control constructs of TABLE 8 to assess the number of the TetO sequences of the repressor element and the placement of repressor element relative to elements of the hU6 promoter and the sequence coding for the suppressor tRNA. The % GFP+ cells were normalized to the % GFP+ cells of the control noTetCCT2.10.

### Example 8: Assessment of Various Repressor Element-Suppressor tRNA AAV Constructs

This example assessed the impact of the repressor element, repressor element placement in repressor element-suppressor tRNA constructs, and the number of repressor element-suppressor tRNA constructs between ITRs (repressor element-suppressor tRNA AAV constructs). The impact of self-complementary ITRs and single-stranded AAV ITRs in repressor element-suppressor tRNA AAV constructs for producing self-complementary AAV (scAAV) or single-stranded AAV (ssAAV) was also assessed. A Repressor+context (HEK 293 cells stably expressing a Tet repressor) and a Repressor− context (HEK 293 cells (lacking expression of a Tet repressor)) were used to assess this impact. The Repressor+context provides a means for identifying how the various designs of the repressor element-suppressor tRNA AAV constructs affect repression of the suppressor tRNA expression resulting in suppressor tRNA silencing, e.g., during AAV production, and therefore affecting AAV titer. The Repressor− context provides a means for identifying the functional influence of the various designs of the repressor element-suppressor tRNA AAV constructs in the absence of the repressor, e.g., to assess the impact of the various designs of the repressor element-suppressor tRNA AAV constructs on the expression and function of suppressor tRNA in a therapeutically relevant context.

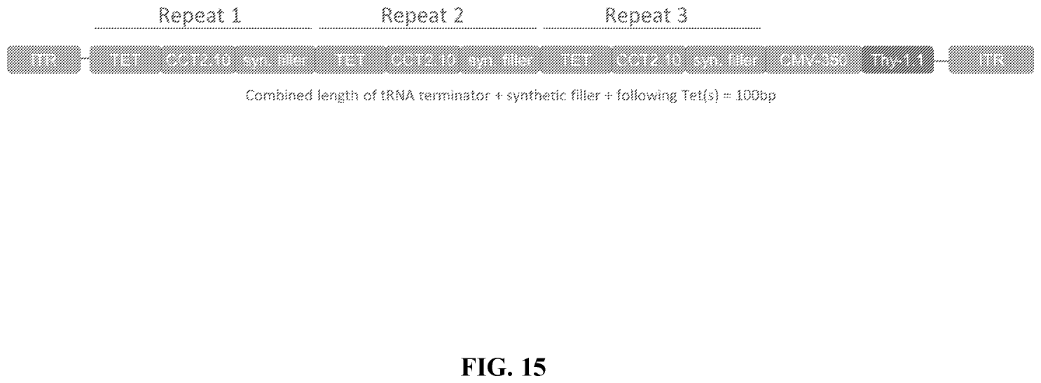

Six of the repressor element-suppressor tRNA constructs identified in EXAMPLE 8 were used to produced repressor element-suppressor tRNA AAV constructs. More specifically, the repressor element, repressor element placement in repressor element-suppressor tRNA constructs, and the number of repressor element-suppressor tRNA constructs between ITRs to produce repressor element-suppressor tRNA AAV constructs in AAV plasmids for testing are described in TABLE 9 below. An exemplary schematic of a repressor element-suppressor tRNA AAV construct is shown in FIG. 15. Four control plasmids were also generated for testing: a positive control plasmid comprising a three copies of sequence coding for CCT2.10 suppressor tRNA between ITRs (STX0607), a control plasmid comprising a repressor element-tRNA AAV construct having a sequence coding for CCT2.10 WT anticodon between ITRs (STX1078 as described in TABLE 9), a control plasmid comprising a repressor element-tRNA AAV construct having a sequence coding for CCT2.10 WT anticodon between ITRs (STX1083 as described in TABLE 9), and a control plasmid comprising a sequence coding for Rab7 and Thy 1.1 between ITRs (STX1007) were also generated for testing. Sequences of repressor element-suppressor tRNA AAV constructs (STX1074-STX1077 and STX1079-STX1082) and of the repressor element-tRNA AAV constructs (STX1078 and STX1083) are provided as SEQ ID NOs: 203-212.

TABLE 9
REPRESSOR ELEMENT-SUPPRESSOR TRNA AAV CONSTRUCTS

| | | | | | Repeats of | |
|---|---|---|---|---|---|---|
| | | Repressor | | | Repressor | |
| | | Element | | | Element- | |
| | | Spacing | | | Suppressor | |
| | | Relative to | | | tRNA | |
| Repressor | | Suppressor | | | constructs | |
| Element- | | tRNA | | | in | |
| Suppressor | | Sequence in | Number | | Repressor | |
| tRNA AAV | | Repressor | of TetO | | Element- | |
| Construct or | | Element- | sequences | | Suppressor | SSAAV |
| Repressor | | Suppressor | in | Suppressor | tRNA | or |
| Element-tRNA | SEQ ID | tRNA | Repressor | tRNA | AAV | SCAAV |
| AAV Construct | NO: | construct | Element | sequence | construct | produced |
| STX1074: | 203 | −45 | 1 | CCT2.10 | 1 | scAAV |
| sc1Tetm45 1X | | | | | | |
| CCT2.10 | | | | | | |
| STX1075: | 204 | −45 | 1 | CCT2.10 | 3 | scAAV |
| sc 1Tetm45 3X | | | | | | |
| CCT2.10 | | | | | | |
| STX1076: | 205 | +1 | 1 | CCT2.10 | 1 | scAAV |
| sc 1Tetp1 1X | | | | | | |
| CCT2.10 | | | | | | |
| STX1077: | 206 | +1 | 1 | CCT2.10 | 3 | scAAV |
| sc 1Tetp1 3X | | | | | | |
| CCT2.10 | | | | | | |
| STX1079: | 208 | +1 | 2 | CCT2.10 | 1 | SCAAV |
| sc2Tetp1 1X | | | | | | |
| CCT2.10 | | | | | | |
| STX1080: | 209 | +1 | 2 | CCT2.10 | 3 | scAAV |
| sc2Tetp1 3X | | | | | | |
| CCT2.10 | | | | | | |
| STX1078: | 207 | +1 | 1 | CCT2.10 | 3 | scAAV |
| sc1Tetp1 3X | | | | WT | | |
| CCT2.10 WT | | | | anticodon | | |
| anticodon | | | | | | |
| STX1081: | 210 | −45 | 1 | CCT2.10 | 3 | SSAAV |
| ss1 Tetm45 3X | | | | | | |
| CCT2.10 | | | | | | |
| STX1082: | 211 | +1 | 1 | CCT2.10 | 3 | SSAAV |
| ss1Tetp1 3X | | | | | | |
| CCT2.10 | | | | | | |
| STX1083: | 212 | +1 | 1 | CCT2.10 | 3 | SSAAV |
| ss1Tetp1 3X | | | | WT | | |
| CCT2.10 WT | | | | anticodon | | |
| anticodon | | | | | | |

Repressor− cells or Repressor+ cells stably expressing broken GFP (a R74X premature stop codon or a R97X premature stop codon inserted into a GFP sequence) were transfected with the plasmids comprising the repressor element-suppressor tRNA AAV constructs (STX1074-STX1077 and STX1079-STX1082) of TABLE 9, the repressor element-tRNA AAV constructs of TABLE 9, or the control plasmids as described above. Detection of a GFP signal indicates the suppressor tRNA was expressed and that there was subsequent readthrough of the premature stop codon to produce detectable GFP. Detection and quantification of the GFP signal was performed using flow cytometry.

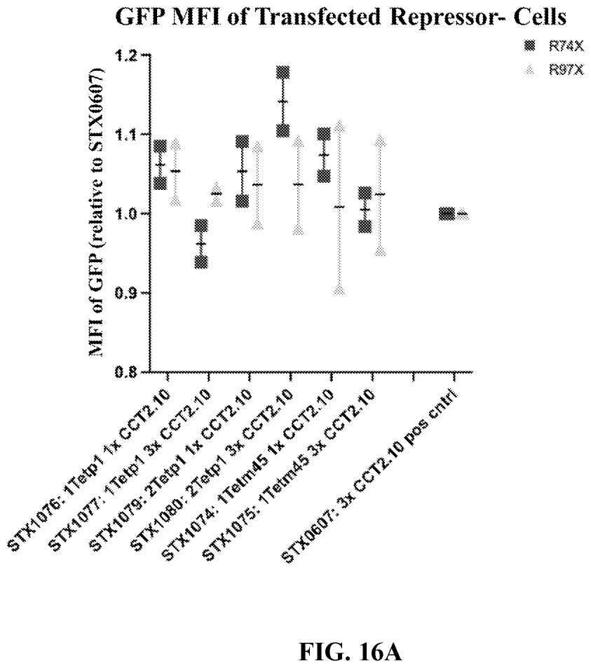

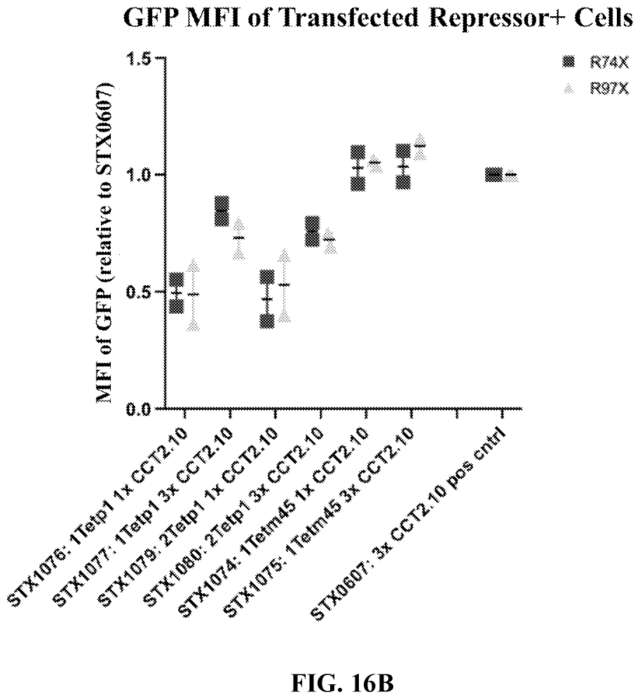

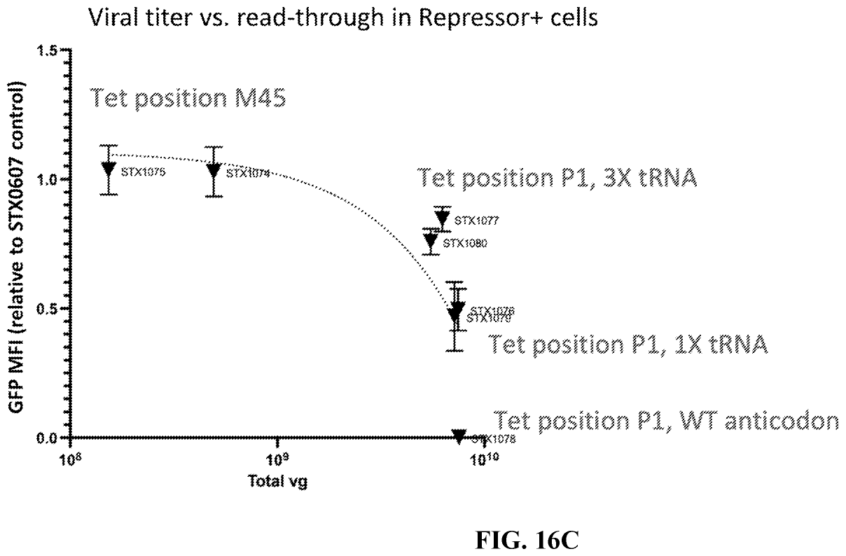

More specifically, FIG. 16A shows the GFP mean fluorescence intensity (MFI) relative to positive control plasmid comprising STX0607 detected in HEK 293 cells (Repressor−) stably expressing a broken GFP reporter (having a R74X premature stop codon or R97X premature stop codon) after tranfection with the plasmids comprising the repressor element-suppressor tRNA AAV constructs (STX1074-STX1077 and STX1079-STX1082) of TABLE 9 and the positive control plasmid comprising STX0607. Percent GFP+ cells was similar for all repressor element-suppressor tRNA AAV constructs and compared to the positive control, indicating minimal impact of the various repressor element-suppressor tRNA AAV construct designs on the expression and function of suppressor tRNA in a therapeutically relevant context. FIG. 16B shows the GFP mean fluorescence intensity (MFI) relative to positive control plasmid comprising STX0607 detected in HEK293 cells expressing a Tet repressor (Repressor+) that also stably express a broken GFP reporter (having a R74X premature stop codon or R97X premature stop codon) after tranfection with the plasmids comprising the repressor element-suppressor tRNA AAV constructs (STX1074-STX1077 and STX1079-STX1082) of TABLE 9 and the positive control plasmid comprising STX0607. Percent GFP+ cells differed between the repressor element-suppressor tRNA AAV constructs and differed compared to the positive control, indicating different repressor element-suppressor tRNA AAV construct design can impact repression of the suppressor tRNA expression that results in suppressor tRNA silencing, e.g., during AAV production, and therefore likely affect AAV titer.

Thus, AAV titer produced using repressor element-suppressor tRNA AAV constructs was also assessed. Plasmids comprising the repressor element-suppressor tRNA AAV constructs (STX1074-STX1077 and STX1079-STX1080) of TABLE 9, the control plasmid comprising a repressor element-tRNA AAV construct (STX1078) of TABLE 9, the control plasmid comprising STX605 (CMV-GFP between ITRs), or the control plasmid comprising ST0607 (a three copies of sequence coding for CCT2.10 suppressor tRNA between ITRs) were transfected into HEK293 cells with an AAV Rep/Cap plasmid and an AAV helper plasmid. The produced AAV titer was assessed for each triple transfection is shown in TABLE 10.

TABLE 10
AAV TITER PRODUCED USING REPRESSOR
ELEMENT-SUPPRESSOR TRNA AAV
CONSTRUCTS AND OF CONTROLS

| Construct | Titer |
|---|---|
| STX0607 | 3.9E+08 |
| STX650 | 1.4E+10 |
| STX1078 | 7.6E+09 |
| STX1074 | 4.9E+08 |
| STX1075 | 1.5E+08 |
| STX1076 | 7.5E+09 |
| STX1077 | 6.3E+09 |
| STX1079 | 7.2E+09 |
| STX1080 | 5.5E+09 |

The impact of the various designs of the the repressor element-suppressor tRNA AAV constructs on suppressor tRNA silencing (as shown in FIG. 16B) and on AAV titer (as shown in TABLE 10) is plotted in FIG. 16C, which plots of the GFP mean fluorescence intensity (MFI) relative to positive control plasmid comprising STX0607 detected in HEK293 cells expressing a Tet repressor (Repressor+) that also stably express a broken GFP reporter (having a R74X premature stop codon) after tranfection with the plasmids comprising the repressor element-suppressor tRNA AAV constructs (STX1074-STX1077 and STX1079-STX1082) of TABLE 10 vs. viral titer (total vgs) produced from HEK293 cells expressing a Tet repressor (Repressor+) that were triple transfected with an AAV helper plasmid, an AAV Rep/Cap plasmid, and a plasmid comprising a repressor element-suppressor tRNA AAV construct (STX1074-STX1077 and STX1079-STX1082) of TABLE 9 or a repressor element-tRNA AAV construct (STX1078) of TABLE 9. Notably, complete suppression of suppressor tRNA expression is not required to increase viral titer. Based on this data, the preferrable repressor element-suppressor tRNA AAV constructs are constructs showing suppressor tRNA suppression and high viral titer, e.g., STX1077, STX1080, STX1076, and STX1079.

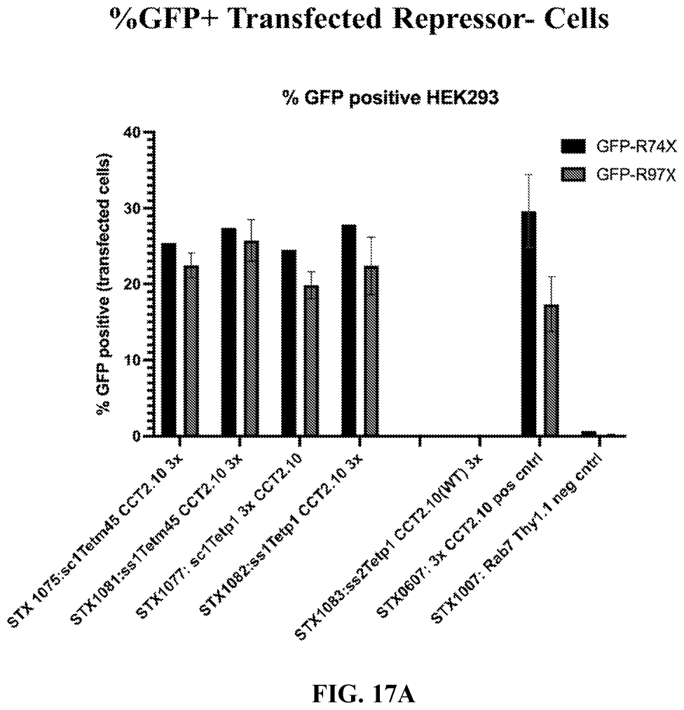

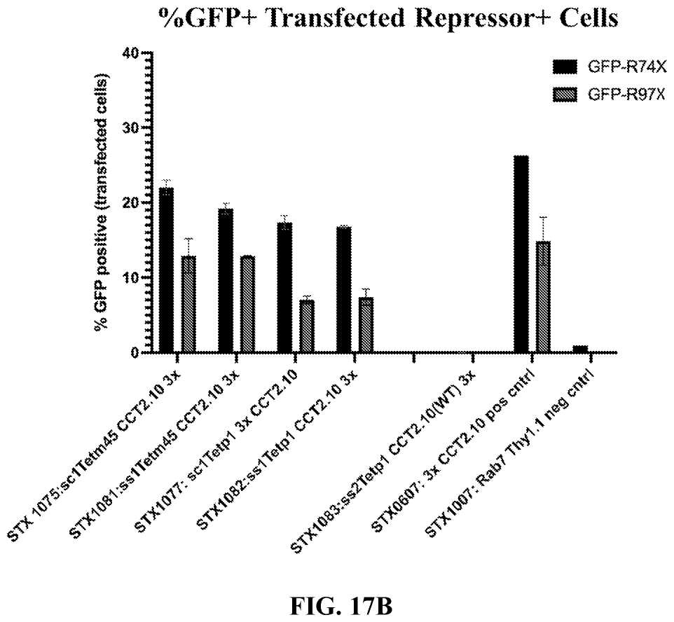

FIGS. 17A-17B show graphs of percent GFP positive cells detected in HEK 293 cells (Repressor-) stably expressing a broken GFP reporter (having a R74X premature stop codon or R97X premature stop codon) (FIG. 17A) or percent GFP positive cells detected in HEK293 cells expressing a Tet repressor (Repressor+) that also stably express a broken GFP reporter (having a R74X premature stop codon or R97X premature stop codon) (FIG. 17B) after independent transfection with the plasmids comprising the repressor element-suppressor tRNA AAV constructs (STX1074-STX1077 and STX1079-STX1082) of TABLE 9, the control plasmid comprising a repressor element-tRNA AAV construct (STX1083) of TABLE 9, the positive control plasmid comprising STX0607, or the negative control plasmid comprising STX1007, to compare the impact of repressor element-suppressor tRNA ssAAV constructs versus repressor element-suppressor tRNA scAAV constructs. Based on this data, both the repressor element-suppressor tRNA ssAAV constructs and repressor element-suppressor tRNA scAAV constructs performed similarly.

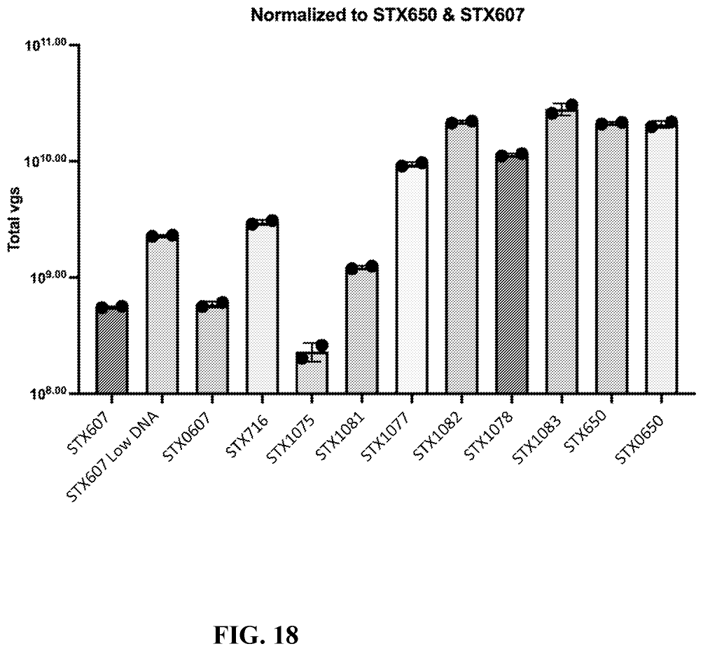

AAV titer produced using repressor element-suppressor tRNA ssAAV constructs compared to AAV titer produced using repressor element-suppressor tRNA scAAV constructs was also assessed. Plasmids comprising the repressor element-suppressor tRNA AAV constructs (STX1075; STX1077; STX1081; or STX1082) of TABLE 9, control plasmids comprising a repressor element-tRNA AAV construct (STX1078; or STX1083) of TABLE 9, the control plasmid comprising STX650 (CMV-GFP between ITRs), the control plasmid comprising ST0607 (a three copies of sequence coding for CCT2.10 suppressor tRNA between ITRs) or the control plasmid comprising STX716 (a three copies of sequence coding for CCT2.10-WT tRNA between ITRs) were transfected into HEK293 cells with an AAV Rep/Cap plasmid and an AAV helper plasmid. The produced AAV titer was assessed for each triple transfection, which is shown in FIG. 18 as total vgs. Notably, the repressor element-suppressor tRNA AAV constructs used to produce the ssAAV showed increased titer of 2-4X as compared to the repressor element-suppressor tRNA AAV constructs used to produce the scAAV (normalized to AAV titer produced using control plasmid comprising STX650 (CMV-GFP between ITRs) and the control plasmid comprising ST0607 (a three copies of sequence coding for CCT2.10 suppressor tRNA between ITRs).

### Example 9: Transduction of and Readthrough in HEK293 Cells with AAVs Encapsidating Repressor Element-Suppressor tRNA AAV Constructs

This example assessed whether AAV produced using repressor element-suppressor tRNA AAV constructs can successfully transduce cells and express functional suppressor tRNA. HEK 293 cells stably expressing broken GFP (a R74X premature stop codon or a R97X premature stop codon inserted into a GFP sequence) were transduced at an MOI of 1×10⁴ of AAV produced using the plasmid comprising the STX1080 or STX1082 repressor element-suppressor tRNA AAV constructs of TABLE 9, a control plasmid comprising the STX1078 repressor element-tRNA AAV construct of TABLE 9, or a control plasmid comprising STX650 (CMV-GFP between ITRs) construct. Additional controls included a no transduction control (“no infection”) and transfection of the HEK293 cells stably expressing broken GFP (a R74X premature stop codon or a R97X premature stop codon inserted into a GFP sequence) with the plasmid comprising the STX1080 repressor element-suppressor tRNA AAV constructs of TABLE 9. The constructs further comprised Thy 1.1, which was used as a marker of cell transduction. Cells expressing Thy 1.1 indicates the cells were transduced with the construct. Detection of a GFP signal indicates the suppressor tRNA was expressed and that there was subsequent readthrough of the premature stop codon to produce detectable GFP. Detection and quantification of the GFP signal was performed using flow cytometry.

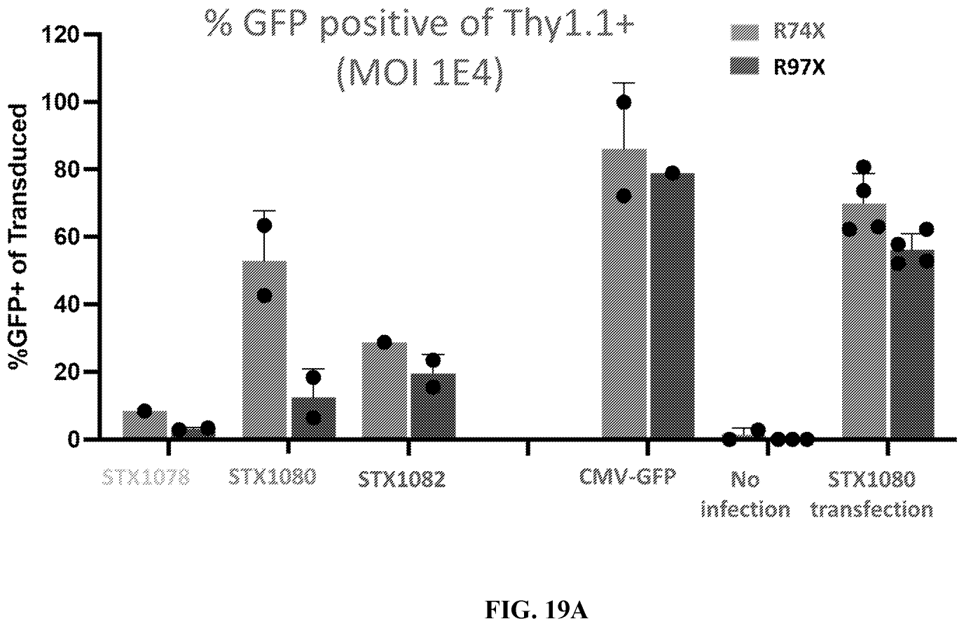

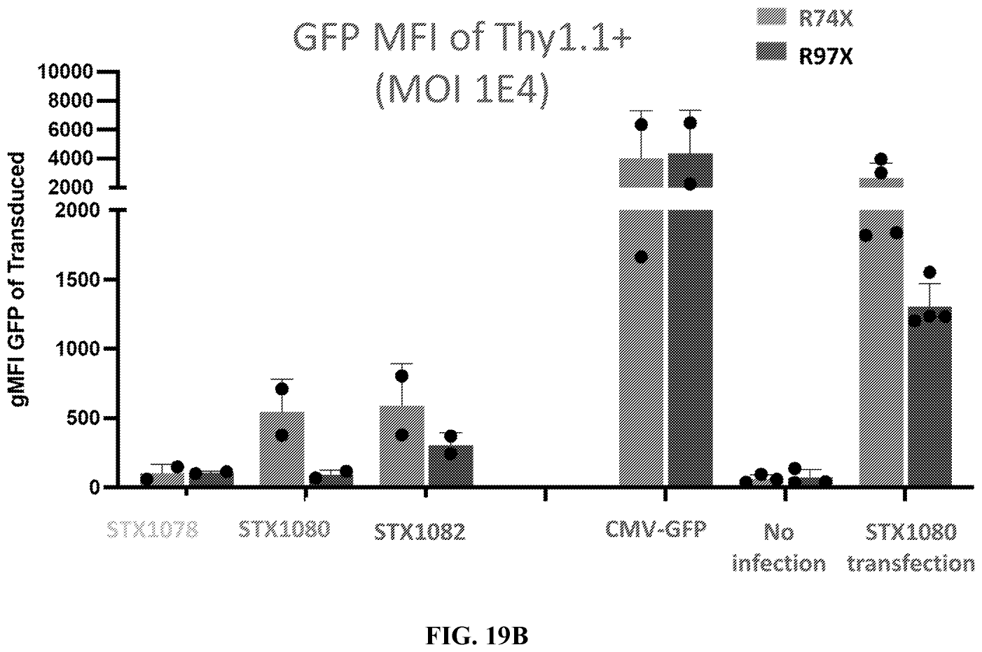

FIG. 19A shows a graph of the percent of transduced HEK 293 cells (Thy 1.1+ cells) expressing GFP after transduction and compared to controls. Positive GFP expression indicates readthrough. FIG. 19B shows a graph of the MFI of GFP positive cells of transduced HEK 293 cells (Thy 1.1+ cells) expressing GFP after transduction and compared to controls. Higher GFP MFI indicates increased readthrough per cell.

As shown in FIGS. 19A-19B, AAV produced using repressor element-suppressor tRNA AAV constructs can successfully transduce cells and express functional suppressor tRNA.

### Example 10: Transduction of and Readthrough in Rett Mouse Model Primary Neuronal Cells Using AAVs Encapsidating Repressor Element-Suppressor tRNA AAV Constructs

Having demonstrated that AAV produced using repressor element-suppressor tRNA AAV constructs can improve titer during AAV production and can produce AAV encapsidating repressor element-suppressor tRNA AAV constructs that also successfully transduce and express functional suppressor tRNA in HEK293 cells, preliminary experiments were carried out to demonstrate the therapeutic utility of these constructs for premature stop codon read-through of a clinically relevant mutation, e.g., a mutation in MECP2 (associated with Rett Syndrome), in a disease relevant cell, neurons.

For this experiment, mouse primary neuronal cells from a Rett Mouse Model (comprises a R168X mutation in MECP2) were used, in which these cells, like HEK293 cells, do not express the Tet repressor protein. The R168X mouse primary neuronal cells were transduced with AAV encapsidating repressor element-suppressor tRNA AAV constructs (STX1080 or STX1082) of TABLE 9 or AAV encapsidating a repressor element-tRNA AAV construct (STX1078) of TABLE 9 as a control. Two different AAV production lots were tested for STX1080. The suppressor tRNAs expressed from the repressor element-suppressor tRNA AAV constructs were engineered to read through the opal stop codons, such as the R168X premature stop codon in MECP2, which should result in expression of full-length MeCP2 protein after transduction with AAV encapsidating repressor element-suppressor tRNA AAV constructs. Mouse primary neuronal cells from a wild-type mouse and non-transduced R168X mouse primary neuronal cells were also assessed as controls.

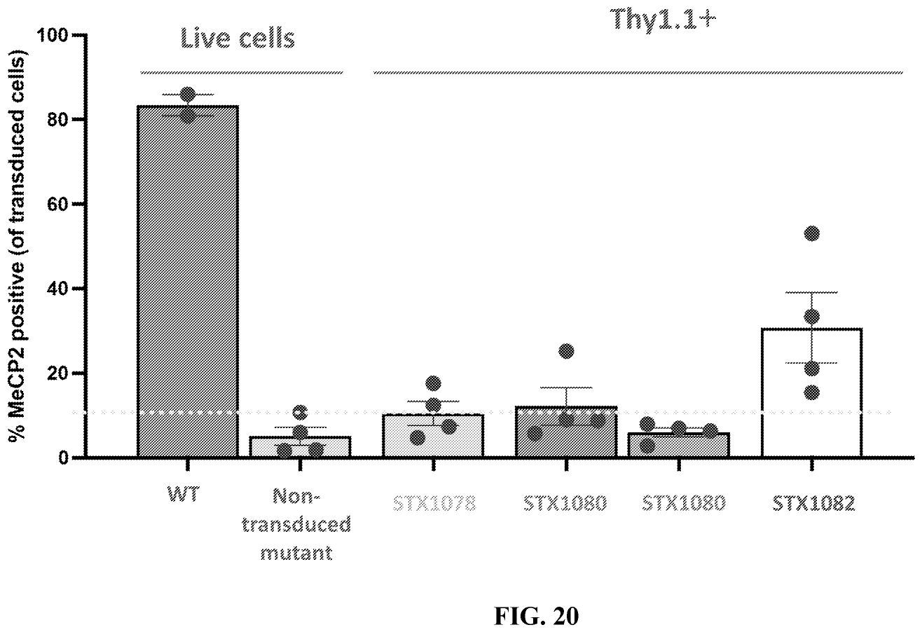

As shown in FIG. 20, at least one of the neuron transductions was successful in this first preliminary experiment, with the transduced cells having detectable full-length MeCP2 protein, demonstrating read-through of the R168X MeCP2 premature stop codon in primary neurons and therefore, demonstrating the therapeutic utility of these constructs for premature stop codon read-through of a clinically relevant mutation in a disease relevant cell.

Further experiments will be carried out to improve the potency of the AAVs and subsequent expression of suppressor tRNAs from AAV encapsidating repressor element-suppressor tRNA AAV constructs in this therapeutically relevant target.

### Example 11: Improved Production of AAV Vectors Using Ochre-Suppressor tRNAs

This example describes improving production of AAV vectors by decreasing suppressor-mediated readthrough of AAV capsid protein stop codons.

The VP reading frame of AAV2 ends with an Ochre stop, followed by two more in-frame ochre stops downstream. Readthrough at the C-terminus could disrupt capsid assembly at the 2-fold axis of symmetry by potentially adding up to 33 amino acids to the VP proteins.

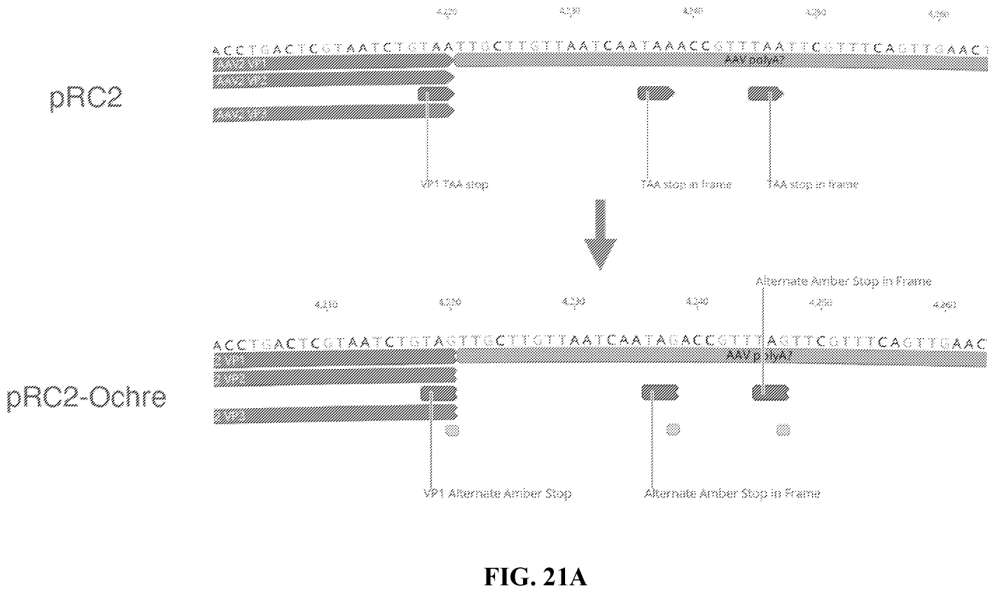

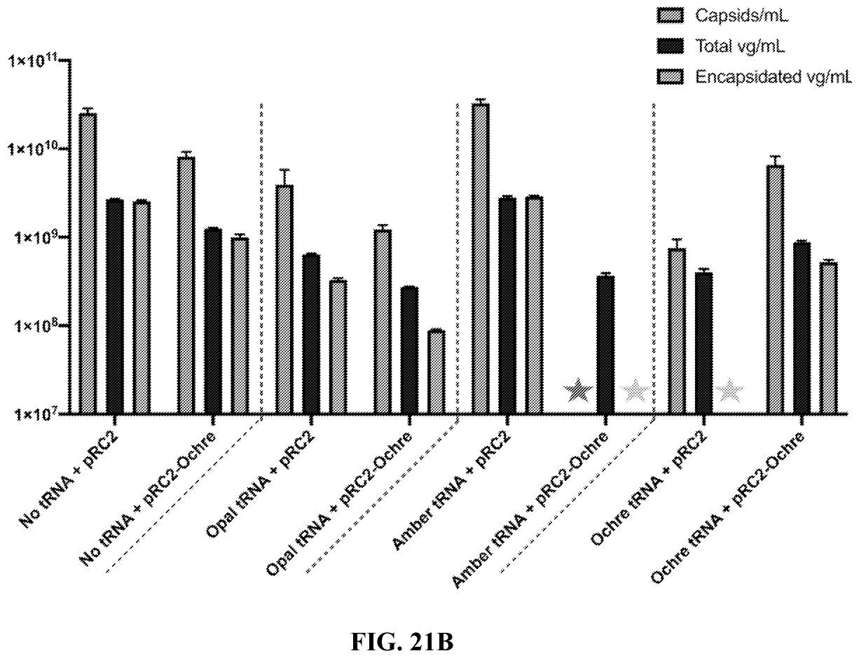

As shown in FIG. 21A, the ochre VP1 stop codon and the in-frame ochre stop codons in the Rep/Cap plasmid (pRC2; SEQ ID NO: 154) were engineered to amber stop codons to generate the pRC2-Ochre plasmid (SEQ ID NO: 156). AAV payload plasmids were triple transfected with AAV helper protein plasmids and either pRC2 plasmid or pRC2-Ochre plasmid. The AAV payload plasmids used in the triple transfection comprised, between ITRs, a sequence encoding an opal suppressor tRNA (Opal tRNA), a sequence encoding an amber suppressor tRNA (Amber tRNA), a sequence encoding an ochre suppressor tRNA (Ochre tRNA), or a sequence encoding a non-functional CCT5 tRNA (No tRNA), which produce AAV encapsidating the sequence encoding an opal suppressor tRNA (Opal AAV), AAV encapsidating the sequence encoding an amber suppressor tRNA (Amber AAV), AAV encapsidating the sequence encoding an ochre suppressor tRNA (Ochre AAV), or AAV encapsidating the sequence encoding a non-functional CCT5 tRNA (no tRNA AAV), respectively.

Three days after transfection, the cells and media were harvested, and AAV virion was released by freeze/thaw. As shown in FIG. 21B: 1) no tRNA AAV production using pRC2-Ochre has a reduced titer compared to using pRC2 (comparing “No tRNA+pRC2” and “No tRNA+pRC2-Ochre”); 2) Opal AAV production was low with both pRC2 and pRC2-Ochre (“Opal tRNA+pRC2” and “Opal tRNA+pRC2-Ochre” compared to “No tRNA+pRC2”); 3) Amber AAV production was undetectable using pRC2-Ochre (see “Amber tRNA+pRC2-Ochre”; compare to, e.g., “Amber tRNA+pRC2”); and 4) pRC2-Ochre restored capsids and encapsidated genomes when producing Ochre AAV (compare “Ochre tRNA+pRC2” to “Ochre tRNA+pRC2-Ochre”).

Taken together, these results demonstrate that ochre suppressor-mediated readthrough of the rAAV capsid protein stop codon severely affected AAV titer and that mutation of the ochre stop codon(s) on a protein critical to AAV production, such as VP1 can at least partially restore AAV titer.

### Example 12: Treating Rett Syndrome

This example describes using AAV virions comprising a polynucleotide having a sequence coding for suppressor tRNA to treat a subject having Rett Syndrome.

The AAV virion comprising a polynucleotide having one or more sequences encoding CCT4 that is produced by cells comprising the Tet repressor as described in EXAMPLE 1 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT2.10 that is produced by cells comprising the Tet repressor as described in EXAMPLE 1 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT4 that is produced by cells comprising the dCas9 linked to a krab domain as described in EXAMPLE 2 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT2.10 that is produced by cells comprising the dCas9 linked to a krab domain as described in EXAMPLE 2 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT4 that is produced by cells transfected with siRNA, shRNA, and DsRNA as described in EXAMPLE 3 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT2.10 that is produced by cells transfected with siRNA, shRNA, and DsRNA as described in EXAMPLE 3 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT2.10-opal that is produced by cells comprising the Tet repressor as described in EXAMPLE 4 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT2.10-TCT1.11-opal that is produced by cells comprising the Tet repressor as described in EXAMPLE 4 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT4 that is produced by cells transfected with at least one stop codon engineered viral molecule, such as a viral molecule as described in EXAMPLE 5, are administered to a subject having Rett Syndrome. The at least one stop codon is engineered from an opal stop codon to an ochre stop or an amber stop. Symptoms associated with Rett Syndrome are alleviated by the suppressor tRNA readthrough of the premature stop codon in MECP2.

The AAV virion comprising a polynucleotide having one or more sequences encoding CCT2.10 that is produced by cells transfected with at least one stop codon engineered viral molecule, such as a viral molecule as described in EXAMPLE 5, are administered to a subject having Rett Syndrome. The at least one stop codon is engineered from an opal stop codon to an ochre stop or an amber stop. Symptoms associated with Rett Syndrome are alleviated by the suppressor tRNA readthrough of the premature stop codon in MECP2.

The AAV virion comprising a polynucleotide having one or more sequences encoding CCT2.10 that is produced by cells comprising the Tet repressor as described in EXAMPLE 6 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT2.10 that is produced by cells comprising the Tet repressor as described in EXAMPLE 7 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT4 that is produced by cells comprising the Tet repressor as described in EXAMPLE 7 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT2.10 that is produced by cells comprising the Tet repressor as described in EXAMPLE 8 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT2.10 that is produced by cells comprising the Tet repressor as described in EXAMPLE 9 are administered to a subject having Rett Syndrome. Symptoms associated with Rett Syndrome are alleviated by the suppressor tRNA readthrough of the premature stop codon in MECP2.

The AAV virion comprising a polynucleotide having one or more sequences encoding CCT21.0 that is produced by cells comprising the Tet repressor as described in EXAMPLE 10 are administered to a subject having Rett Syndrome. Symptoms associated with Rett Syndrome are alleviated by the suppressor tRNA readthrough of the premature stop codon in MECP2.

SEQUENCES

| | | SEQ |
|---|---|---|
| | | ID |
| Description | Sequence | NO: |
| 1TETm7 | TCCCTATCAGTGATAGAGATGCAGAAGCCCCAGTGGCCTAATGGATAA | 158 |
| CCT2.10 | GGCACTGGCCTCCTAAGCCAGGGATTGCGGGTTCGAGTCCCGCCTGGG | |
| | GTGTTTTTT | |
| 1TETm12 | TCCCTATCAGTGATAGAGAAGAGTTGCAGAAGCCCCAGTGGCCTAATG | 159 |
| CCT2.10 | GATAAGGCACTGGCCTTCAAAGCCAGGGATTGCGGGTTCGAGTCCCGC | |
| | CTGGGGTGTTTTTT | |
| 1TETm20 | TCCCTATCAGTGATAGAGAATCAGATAAGAGTTGCAGAAGCCCCAGTG | 160 |
| CCT2.10 | GCCTAATGGATAAGGCACTGGCCTTCAAAGCCAGGGATTGCGGGTTCG | |
| | AGTCCCGCCTGGGGTGTTTTTT | |
| 1TETm30 | TCCCTATCAGTGATAGAGAATAGCCGTTGATCAGATAAGAGTTGCAGA | 161 |
| CCT2.10 | AGCCCCAGTGGCCTAATGGATAAGGCACTGGCCTTCAAAGCCAGGGAT | |
| | TGCGGGTTCGAGTCCCGCCTGGGGTGTTTTTT | |
| 1TETm45 | TCCCTATCAGTGATAGAGATGGCAATTCCACGTTATAGCCGTTGATCA | 162 |
| CCT2.10 | GATAAGAGTTGCAGAAGCCCCAGTGGCCTAATGGATAAGGCACTGGCC | |
| | TTCAAAGCCAGGGATTGCGGGTTCGAGTCCCGCCTGGGGTGTTTTTT | |
| 1TETp1 | TCCCTATCAGTGATAGAGAGCCCCAGTGGCCTAATGGATAAGGCACTG | 163 |
| CCT2.10 | GCCTTCAAAGCCAGGGATTGCGGGTTCGAGTCCCGCCTGGGGTGTTTT | |
| | TT | |
| 1TETp7 | TGCAGAAGCCCCAGTGGCCTAATGGATAAGGCACTGGCCTTCAAAGCC | 164 |
| CCT2.10 | AGGGATTGCGGGTTCGAGTCCCGCCTGGGGTGTTTTTTTTCCCTATCA | |
| | GTGATAGAGA | |
| 1TETp12 | TGCAGAAGCCCCAGTGGCCTAATGGATAAGGCACTGGCCTTCAAAGCC | 165 |
| CCT2.10 | AGGGATTGCGGGTTCGAGTCCCGCCTGGGGTGTTTTTTTATCTGTCCC | |
| | TATCAGTGATAGAGA | |
| 1TETp20 | TGCAGAAGCCCCAGTGGCCTAATGGATAAGGCACTGGCCTTCAAAGCC | 166 |
| CCT2.10 | AGGGATTGCGGGTTCGAGTCCCGCCTGGGGTGTTTTTTTATCTGCAAG | |
| | CCTGTCCCTATCAGTGATAGAGA | |
| 1TETp30 | TGCAGAAGCCCCAGTGGCCTAATGGATAAGGCACTGGCCTTCAAAGCC | 167 |
| CCT2.10 | AGGGATTGCGGGTTCGAGTCCCGCCTGGGGTGTTTTTTTATCTGCAAG | |
| | CCTGGCAATTCCACTCCCTATCAGTGATAGAGA | |
| 1TETp45 | TGCAGAAGCCCCAGTGGCCTAATGGATAAGGCACTGGCCTTCAAAGCC | 168 |
| CCT2.10 | AGGGATTGCGGGTTCGAGTCCCGCCTGGGGTGTTTTTTTATCTGCAAG | |
| | CCTGGCAATTCCACGTTATAGCCGTTGATTCCCTATCAGTGATAGAGA | |
| noTET | TGCAGAAGCCCCAGTGGCCTAATGGATAAGGCACTGGCCTTCAAAGCC | 169 |
| CCT2.10 | AGGGATTGCGGGTTCGAGTCCCGCCTGGGGTGTTTTTT | |
| noTET | TGCAGAAGCCCCAGTGGCCTAATGGATAAGGCACTGGCCTCCTAAGCC | 170 |
| CCT2.10- | AGGGATTGCGGGTTCGAGTCCCGCCTGGGGTGTTTTTT | |
| WT | | |
| TETm7 | TCCCTATCAGTGATAGAGATCTCCCTATCAGTGATAGAGATGCAGAAG | 171 |
| CCT2.10 | CCCCAGTGGCCTAATGGATAAGGCACTGGCCTTCAAAGCCAGGGATTG | |
| | CGGGTTCGAGTCCCGCCTGGGGTGTTTTTT | |
| TETm12 | TCCCTATCAGTGATAGAGATCTCCCTATCAGTGATAGAGAAGAGTTGC | 172 |
| CCT2.10 | AGAAGCCCCAGTGGCCTAATGGATAAGGCACTGGCCTTCAAAGCCAGG | |
| | GATTGCGGGTTCGAGTCCCGCCTGGGGTGTTTTTT | |
| TETm20 | TCCCTATCAGTGATAGAGATCTCCCTATCAGTGATAGAGAATCAGATA | 173 |
| CCT2.10 | AGAGTTGCAGAAGCCCCAGTGGCCTAATGGATAAGGCACTGGCCTTCA | |
| | AAGCCAGGGATTGCGGGTTCGAGTCCCGCCTGGGGTGTTTTTT | |
| TETm30 | TCCCTATCAGTGATAGAGATCTCCCTATCAGTGATAGAGAATAGCCGT | 174 |
| CCT2.10 | TGATCAGATAAGAGTTGCAGAAGCCCCAGTGGCCTAATGGATAAGGCA | |
| | CTGGCCTTCAAAGCCAGGGATTGCGGGTTCGAGTCCCGCCTGGGGTGT | |
| | TTTTT | |
| TETm45 | TCCCTATCAGTGATAGAGATCTCCCTATCAGTGATAGAGATGGCAATT | 175 |
| CCT2.10 | CCACGTTATAGCCGTTGATCAGATAAGAGTTGCAGAAGCCCCAGTGGC | |
| | CTAATGGATAAGGCACTGGCCTTCAAAGCCAGGGATTGCGGGTTCGAG | |
| | TCCCGCCTGGGGTGTTTTTT | |
| TETp1 | TCCCTATCAGTGATAGAGATCTCCCTATCAGTGATAGAGAGCCCCAGT | 176 |
| CCT2.10 | GGCCTAATGGATAAGGCACTGGCCTTCAAAGCCAGGGATTGCGGGTTC | |
| | GAGTCCCGCCTGGGGTGTTTTTT | |
| TETp7 | TGCAGAAGCCCCAGTGGCCTAATGGATAAGGCACTGGCCTTCAAAGCC | 177 |
| CCT2.10 | AGGGATTGCGGGTTCGAGTCCCGCCTGGGGTGTTTTTTTTCCCTATCA | |
| | GTGATAGAGATCTCCCTATCAGTGATAGAGA | |
| TETp12 | TGCAGAAGCCCCAGTGGCCTAATGGATAAGGCACTGGCCTTCAAAGCC | 178 |
| CCT2.10 | AGGGATTGCGGGTTCGAGTCCCGCCTGGGGTGTTTTTTTATCTGTCCC | |
| | TATCAGTGATAGAGATCTCCCTATCAGTGATAGAGA | |
| TETp20 | TGCAGAAGCCCCAGTGGCCTAATGGATAAGGCACTGGCCTTCAAAGCC | 179 |
| CCT2.10 | AGGGATTGCGGGTTCGAGTCCCGCCTGGGGTGTTTTTTTATCTGCAAG | |
| | CCTGTCCCTATCAGTGATAGAGATCTCCCTATCAGTGATAGAGA | |
| TETp30 | TGCAGAAGCCCCAGTGGCCTAATGGATAAGGCACTGGCCTTCAAAGCC | 180 |
| CCT2.10 | AGGGATTGCGGGTTCGAGTCCCGCCTGGGGTGTTTTTTTATCTGCAAG | |
| | CCTGGCAATTCCACTCCCTATCAGTGATAGAGATCTCCCTATCAGTGA | |
| | TAGAGA | |
| TETp45 | TGCAGAAGCCCCAGTGGCCTAATGGATAAGGCACTGGCCTTCAAAGCC | 181 |
| CCT2.10 | AGGGATTGCGGGTTCGAGTCCCGCCTGGGGTGTTTTTTTATCTGCAAG | |
| | CCTGGCAATTCCACGTTATAGCCGTTGATTCCCTATCAGTGATAGAGA | |
| | TCTCCCTATCAGTGATAGAGA | |
| 1TetTATA1 | GAGGGCCTATTTCCCATGATTCCTTCATATTTGCATATACGATACAAG | 182 |
| Tetm1CCT2.10 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTCCCTATCAGTGATAGAGATTATATATCTTCCCTATCAG | |
| | TGATAGAGACGCCCCAGTGGCCTAATGGATAAGGCACTGGCCTTCAAA | |
| | GCCAGGGATTGCGGGTTCGAGTCCCGCCTGGGGTGTTTTTT | |
| 1TetTATA1 | GAGGGCCTATTTCCCATGATTCCTTCATATTTGCATATACGATACAAG | 183 |
| Tetm1CCT4 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTCCCTATCAGTGATAGAGATTATATATCTTCCCTATCAG | |
| | TGATAGAGACGCCCCAGTGGCCTAATGGATAAGGCATTGGCCTTCAAA | |
| | GCCAGGGATTGTGGGTTCGAGTCCCATCTGGGGTGTTTTTT | |
| 1TetTATA1 | GAGGGCCTATTTCCCATGATTCCTTCATATTTGCATATACGATACAAG | 184 |
| Tetp1CCT2pt10 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTCCCTATCAGTGATAGAGATTATATATCTTTCCCTATCA | |
| | GTGATAGAGAGCCCCAGTGGCCTAATGGATAAGGCACTGGCCTTCAAA | |
| | GCCAGGGATTGCGGGTTCGAGTCCCGCCTGGGGTGTTTTTT | |
| 1TetTATA1 | GAGGGCCTATTTCCCATGATTCCTTCATATTTGCATATACGATACAAG | 185 |
| Tetp1CCT4 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTCCCTATCAGTGATAGAGATTATATATCTTTCCCTATCA | |
| | GTGATAGAGAGCCCCAGTGGCCTAATGGATAAGGCATTGGCCTTCAAA | |
| | GCCAGGGATTGTGGGTTCGAGTCCCATCTGGGGTGTTTTTT | |
| TATA1Tetm1 | GAGGGCCTATTTCCCATGATTCCTTCATATTTGCATATACGATACAAG | 186 |
| CCT2pt10 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTATTTCGATTTCTTGGCTTTATATATCTTCCCTATCAGT | |
| | GATAGAGACGCCCCAGTGGCCTAATGGATAAGGCACTGGCCTTCAAAG | |
| | CCAGGGATTGCGGGTTCGAGTCCCGCCTGGGGTGTTTTTT | |
| TATA1Tetm1 | GAGGGCCTATTTCCCATGATTCCTTCATATTTGCATATACGATACAAG | 187 |
| CCT4 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTATTTCGATTTCTTGGCTTTATATATCTTCCCTATCAGT | |
| | GATAGAGACGCCCCAGTGGCCTAATGGATAAGGCATTGGCCTTCAAAG | |
| | CCAGGGATTGTGGGTTCGAGTCCCATCTGGGGTGTTTTTT | |
| TATA1Tetp1 | GAGGGCCTATTTCCCATGATTCCTTCATATTTGCATATACGATACAAG | 188 |
| CCT2pt10 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTATTTCGATTTCTTGGCTTTATATATCTTTCCCTATCAG | |
| | TGATAGAGAGCCCCAGTGGCCTAATGGATAAGGCACTGGCCTTCAAAG | |
| | CCAGGGATTGCGGGTTCGAGTCCCGCCTGGGGTGTTTTTT | |
| TATA1Tetp1 | GAGGGCCTATTTCCCATGATTCCTTCATATTTGCATATACGATACAAG | 189 |
| CCT4 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTATTTCGATTTCTTGGCTTTATATATCTTTCCCTATCAG | |
| | TGATAGAGAGCCCCAGTGGCCTAATGGATAAGGCATTGGCCTTCAAAG | |
| | CCAGGGATTGTGGGTTCGAGTCCCATCTGGGGTGTTTTTT | |
| TATA2Tetp1 | GAGGGCCTATTTCCCATGATTCCTTCATATTTGCATATACGATACAAG | 190 |
| CCT4 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTATTTCGATTTCTTGGCTTTATATATCTCCCTATCAGTG | |
| | ATAGAGATCGTCCCTATCAGTGATAGAGAGCCCCAGTGGCCTAATGGA | |
| | TAAGGCATTGGCCTTCAAAGCCAGGGATTGTGGGTTCGAGTCCCATCT | |
| | GGGGTGTTTTTT | |
| 2TetPSEATA2 | GAGGGCCTATTTCCCATGATTCCTTCATATTTGCATATACGATACAAG | 191 |
| Tetp1CCT4 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | CCCTATCAGTGATAGAGATCTCCCTATCAGTGATAGAGACTTACCGTA | |
| | ACTTGAAAGTATTTCGATTTCTTGGCTTTATATATCTCCCTATCAGTG | |
| | ATAGAGATCGTCCCTATCAGTGATAGAGAGCCCCAGTGGCCTAATGGA | |
| | TAAGGCATTGGCCTTCAAAGCCAGGGATTGTGGGTTCGAGTCCCATCT | |
| | GGGGTGTTTTTT | |
| 1TetPSE- | GAGGGCCTATTTCCCATGATTCCTTCATATTTGCATATACGATACAAG | 192 |
| TATAhU6end_ | GCTGTTAGAGAGATAATTAGAATTAATTTGACTGTAAACACAAAGATA | |
| 2Tetp1CCT4 | TTAGTACAAAATACGTGACGTAGAAAGTAATAATTTCTTGGGTAGTTT | |
| | GCAGTTTTAAAATTATGTTTTCCCTATCAGTGATAGAGACTTACCGTA | |
| | ACTTGAAAGTATTTCGATTTCTTGGCTTTATATATCTTGTGGAAAGGA | |
| | CGAAACACCGTCCCTATCAGTGATAGAGATCTCCCTATCAGTGATAGA | |
| | GAGCCCCAGTGGCCTAATGGATAAGGCATTGGCCTTCAAAGCCAGGGA | |
| | TTGTGGGTTCGAGTCCCATCTGGGGTGTTTTTT | |
| hU6_2Tetp1 | GAGGGCCTATTTCCCATGATTCCTTCATATTTGCATATACGATACAAG | 193 |
| CCT4 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTATTTCGATTTCTTGGCTTTATATATCTTGTGGAAAGGA | |
| | CGAAACACCGTCCCTATCAGTGATAGAGATCTCCCTATCAGTGATAGA | |
| | GAGCCCCAGTGGCCTAATGGATAAGGCATTGGCCTTCAAAGCCAGGGA | |
| | TTGTGGGTTCGAGTCCCATCTGGGGTGTTTTTT | |
| noTetCCT4- | GCCCCAGTGGCCTAATGGATAAGGCATTGGCCTCCTAAGCCAGGGATT | 194 |
| WT | GTGGGTTCGAGTCCCATCTGGGGTGTTTTTT | |
| 1TETm1 | TCCCTATCAGTGATAGAGACGCCCCAGTGGCCTAATGGATAAGGCACT | 195 |
| CCT2.10 | GGCCTTCAAAGCCAGGGATTGCGGGTTCGAGTCCCGCCTGGGGTGTTT | |
| | TTT | |
| 1TETm7 | TCCCTATCAGTGATAGAGATGCAGAAGCCCCAGTGGCCTAATGGATAA | 196 |
| CCT2.10 | GGCACTGGCCTTCAAAGCCAGGGATTGCGGGTTCGAGTCCCGCCTGGG | |
| | GTGTTTTTT | |
| 1TETp1CCT4 | TCCCTATCAGTGATAGAGAGCCCCAGTGGCCTAATGGATAAGGCATTG | 197 |
| | GCCTTCAAAGCCAGGGATTGTGGGTTCGAGTCCCATCTGGGGTGTTTT | |
| | TT | |
| 2Tetp1CCT4 | TCCCTATCAGTGATAGAGATCTCCCTATCAGTGATAGAGAGCCCCAGT | 198 |
| | GGCCTAATGGATAAGGCATTGGCCTTCAAAGCCAGGGATTGTGGGTTC | |
| | GAGTCCCATCTGGGGTGTTTTTT | |
| WT_hU6- | GAGGGCCTATTTCCCATGATTCCTTCATATTTGCATATACGATACAAG | 199 |
| CCT2.10 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTATTTCGATTTCTTGGCTTTATATATCTTGTGGAAAGGA | |
| | CGAAACACCGCCCCAGTGGCCTAATGGATAAGGCACTGGCCTTCAAAG | |
| | CCAGGGATTGCGGGTTCGAGTCCCGCCTGGGGTGTTTTTT | |
| WT_hU6- | GAGGGCCTATTTCCCATGATTCCTTCATATTTGCATATACGATACAAG | 200 |
| CCT2.10_ | GCTGTTAGAGAGATAATTAGAATTAATTTGACTGTAAACACAAAGATA | |
| WT | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTATTTCGATTTCTTGGCTTTATATATCTTGTGGAAAGGA | |
| | CGAAACACCGCCCCAGTGGCCTAATGGATAAGGCACTGGCCTCCTAAG | |
| | CCAGGGATTGCGGGTTCGAGTCCCGCCTGGGGTGTTTTTT | |
| WT_hU6- | GAGGGCCTATTTCCCATGATTCCTTCATATTTGCATATACGATACAAG | 201 |
| CCT4 | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTATTTCGATTTCTTGGCTTTATATATCTTGTGGAAAGGA | |
| | CGAAACACCGCCCCAGTGGCCTAATGGATAAGGCATTGGCCTTCAAAG | |
| | CCAGGGATTGTGGGTTCGAGTCCCATCTGGGGTGTTTTTT | |
| WT_hU6- | GAGGGCCTATTTCCCATGATTCCTTCATATTTGCATATACGATACAAG | 202 |
| CCT4-WT | GCTGTTAGAGAGATAATTAGAATTAATTTGACTGTAAACACAAAGATA | |
| | TTAGTACAAAATACGTGACGTAGAAAGTAATAATTTCTTGGGTAGTTT | |
| | GCAGTTTTAAAATTATGTTTTAAAATGGACTATCATATGCTTACCGTA | |
| | ACTTGAAAGTATTTCGATTTCTTGGCTTTATATATCTTGTGGAAAGGA | |
| | CGAAACACCGCCCCAGTGGCCTAATGGATAAGGCATTGGCCTCCTAAG | |
| | CCAGGGATTGTGGGTTCGAGTCCCATCTGGGGTGTTTTTT | |
| 1Tetm45_1X_ | TCTGCGCGCTCGCTCGCTCACTGAGGCCGGGCGACCAAAGGTCGCCCG | 203 |
| CCT2.10_ | ACGCCCGGGCTTTGCCCGGGCGGCCTCAGTGAGCGAGCGAGCGCGCAG | |
| pscAAV_ | AGGATCCTATCAAGAACCAACCTAGAACAGGATACCACAGTGAATATC | |
| sCITR2_CMV- | ACTGTTAGATCCCTATCAGTGATAGAGATGGCAATTCCACGTTATAGC | |
| Thy1.1-pA | CGTTGATCAGATAAGAGTTGCAGAAGCCCCAGTGGCCTAATGGATAAG | |
| | GCACTGGCCTTCAAAGCCAGGGATTGCGGGTTCGAGTCCCGCCTGGGG | |
| | TGTTTTTTTTGGACCGTTATAACCGGTATTATCTTCGTGTTATTGCCG | |
| | TCGTAGCAGACCTGGCATTATGCCCAGTACATGACCTTATGGGACTTT | |
| | CCTACTTGGCAGTACATCTACGTATTAGTCATCGCTATTACCATGGTG | |
| | ATGCGGTTTTGGCAGTACATCAATGGGCGTGGATAGCGGTTTGACTCA | |
| | CGGGGATTTCCAAGTCTCCACCCCATTGACGTCAATGGGAGTTTGTTT | |
| | TGGCACCAAAATCAACGGGACTTTCCAAAATGTCGTAACAACTCCGCC | |
| | CCATTGACGCAAATGGGCGGTAGGCGTGTACGGTGGGAGGTCTATATA | |
| | AGCAGAGCTCGTTTAGTGAACCGTCAGATCGCCTGGAGACGCCATCCA | |
| | CGCTGTTTTGACCTCCATAGTAGAGCCGCCACCATGGCCACAACCATG | |
| | GGTCTTTTCTGCAATCACCGTCCTCGAGGCACCATGAACCCAGCCATC | |
| | AGCGTCGCTCTCCTGCTCTCAGTCTTGCGGGTGTCCCGAGGGCAGAAG | |
| | GTGACCAGCCTGACAGCCTGCCTGGTGAACCAAAACCTTCGCCTGGAC | |
| | TGCCGCCATGAGAATAACACCAAGGATAACTCCATCCAGCATGAGTTC | |
| | AGCCTGACCCGAGAGAAGAGGAAGCACGTGCTCTCAGGCACCCTTGGG | |
| | ATACCCGAGCACACGTACCGCTCCCGCGTCACCCTCTCCAACCAGCCC | |
| | TATATCAAGGTCCTTACCCTAGCCAACTTCACCACCAAGGATGAGGGC | |
| | GACTACTTTTGTGAGCTTCGCGTCTCGGGCGCGAATCCCATGAGCTCC | |
| | AATAAAAGTATCAGTGTGTATAGAGACAAGCTGGTCAAGTGTGGCGGC | |
| | ATAAGCCTGCTGGTTCAGAACACATCCTGGATGCTGCTGCTGCTGCTT | |
| | TCCCTCTCCCTCCTCCAAGCCCTGGACTTCATTTCTCTGTAAAATAAA | |
| | GGAAATTTATTTTCATTGCAATAGTGTGTTGGAATTTTTTGTGTCTCT | |
| | CACAAGCGGCGCTCACTGTAGACCTTGACACACCACTGATCGGCAGAT | |
| | TCCTTAACTGTAATCAAGAGTTCTTAGAGGAGACAATCCAACTCCTTG | |
| | GACTCCTCGAATAACTCTACTCGGTTCACAGTGGAACACTATTGATTG | |
| | TAGATTACGGTGAGGCCGTGCTCAGACTTCGAGATCGATCACAAGATT | |
| | GTATCCAAGCGGAAGACTTAGAACAATCTGCAAGCTAATACCACGGCA | |
| | GATCGTATTGGAGTATAGGATTCTACCTAATACGGTATTCAGTCAGGA | |
| | GATTAAGATTGCGCAGTGCAGATTCTGAACTTGTCAAGTTCCTTACTC | |
| | GCAGCGTACTGGCCTAGCTATCGTGCTCACACTGCTATCTTGGTGCGA | |
| | CTATTGTTCTGTTGCAGTCACCGTGTGTAGTGTATCTGCAGTCAGACT | |
| | ACTCTCTACGGAGGCTGCTTAAGTATCAGCGCTGAGTTGTCGCTTGAT | |
| | TGTGCTGGTGACTATAAGTCCTGAAGTCCGATTGTTGCCTCAATTGTT | |
| | ACTGTAGCGATACGATTAAGCGACGATATAGGCGACGGAAGATACAGC | |
| | TTGCTATTGAGTGCGCGTTCTATCGTGACAGATTATTAGAAGTGTAGA | |
| | CCGGAGGATCAGATAGATCGGCCTACCGCACACCTGAGATCAATACGT | |
| | CGGTAAGAGCGCTGTAAGGACTAATACAATAGTAATAAGACCTATTAT | |
| | AGAGGGTACTAAGATTGCGGCCTGGCCACTGACTAAGATAGAAGCTGT | |
| | AACTTGCCTCAGATTGTGGCCGCTGATCTGCAGAGGAACCCCTAGTGA | |
| | TGGAGTTGGCCACTCCCTCTCTGCGCGCTCGCTCGCTCACTGAGGCCG | |
| | CCCGGGCAAAGCCCGGGCGTCGGGCGACCTTTGGTCGCCCGGCCTCAG | |
| | TGAGCGAGCGAGCGCGCAGAGAGGGAGTGGCCAA | |
| 1Tetm45_3X_ | TCTGCGCGCTCGCTCGCTCACTGAGGCCGGGCGACCAAAGGTCGCCCG 204 | |
| CCT2.10_ | ACGCCCGGGCTTTGCCCGGGCGGCCTCAGTGAGCGAGCGAGCGCGCAG | |
| pscAAV_ | AGGATCCTATCAAGAACCAACCTAGAACAGGATACCACAGTGAATATC | |
| sCITR2_CMV- | ACTGTTAGATCCCTATCAGTGATAGAGATGGCAATTCCACGTTATAGC | |
| Thy1.1-pA | CGTTGATCAGATAAGAGTTGCAGAAGCCCCAGTGGCCTAATGGATAAG | |
| | GCACTGGCCTTCAAAGCCAGGGATTGCGGGTTCGAGTCCCGCCTGGGG | |
| | TGTTTTTTCGATAGGCTGTTGTAAGGCGATTGGTGAAGGAATACGCGG | |
| | TACAAGTACACTCTCCCTATCAGTGATAGAGAACTGTTAACAAGACAC | |
| | AGTGTGTTGTCTATTCGGTTGGTGCAGAAGCCCCAGTGGCCTAATGGA | |
| | TAAGGCACTGGCCTTCAAAGCCAGGGATTGCGGGTTCGAGTCCCGCCT | |
| | GGGGTGTTTTTTGAATAGTTACAGCGATTACCGCCTATTACCAACACG | |
| | GAAGCACTAGACTAATATCCCTATCAGTGATAGAGACTGACTGGTACA | |
| | GGAAGTATCTGATCTAGACGAATTGGTGCAGAAGCCCCAGTGGCCTAA | |
| | TGGATAAGGCACTGGCCTTCAAAGCCAGGGATTGCGGGTTCGAGTCCC | |
| | GCCTGGGGTGTTTTTTTTGGACCGTTATAACCGGTATTATCTTCGTGT | |
| | TATTGCCGTCGTAGCAGACCTGGCATTATGCCCAGTACATGACCTTAT | |
| | GGGACTTTCCTACTTGGCAGTACATCTACGTATTAGTCATCGCTATTA | |
| | CCATGGTGATGCGGTTTTGGCAGTACATCAATGGGCGTGGATAGCGGT | |
| | TTGACTCACGGGGATTTCCAAGTCTCCACCCCATTGACGTCAATGGGA | |
| | GTTTGTTTTGGCACCAAAATCAACGGGACTTTCCAAAATGTCGTAACA | |
| | ACTCCGCCCCATTGACGCAAATGGGCGGTAGGCGTGTACGGTGGGAGG | |
| | TCTATATAAGCAGAGCTCGTTTAGTGAACCGTCAGATCGCCTGGAGAC | |
| | GCCATCCACGCTGTTTTGACCTCCATAGTAGAGCCGCCACCATGGCCA | |
| | CAACCATGGGTCTTTTCTGCAATCACCGTCCTCGAGGCACCATGAACC | |
| | CAGCCATCAGCGTCGCTCTCCTGCTCTCAGTCTTGCGGGTGTCCCGAG | |
| | GGCAGAAGGTGACCAGCCTGACAGCCTGCCTGGTGAACCAAAACCTTC | |
| | GCCTGGACTGCCGCCATGAGAATAACACCAAGGATAACTCCATCCAGC | |
| | ATGAGTTCAGCCTGACCCGAGAGAAGAGGAAGCACGTGCTCTCAGGCA | |
| | CCCTTGGGATACCCGAGCACACGTACCGCTCCCGCGTCACCCTCTCCA | |
| | ACCAGCCCTATATCAAGGTCCTTACCCTAGCCAACTTCACCACCAAGG | |
| | ATGAGGGCGACTACTTTTGTGAGCTTCGCGTCTCGGGCGCGAATCCCA | |
| | TGAGCTCCAATAAAAGTATCAGTGTGTATAGAGACAAGCTGGTCAAGT | |
| | GTGGCGGCATAAGCCTGCTGGTTCAGAACACATCCTGGATGCTGCTGC | |
| | TGCTGCTTTCCCTCTCCCTCCTCCAAGCCCTGGACTTCATTTCTCTGT | |
| | AAAATAAAGGAAATTTATTTTCATTGCAATAGTGTGTTGGAATTTTTT | |
| | GTGTCTCTCACAAGCGGCGCTCACTGTAGACCTTGACACACCACTGAT | |
| | CGGCAGATTCCTTAACTGTAATCAAGAGTTCTTAGAGGAGACAATCCA | |
| | ACTCCTTGGACTCCTCGAATAACTCTACTCGGTTCACAGTGGAACACT | |
| | ATTGATTGTAGATTACGGTGAGGCCGTGCTCAGACTTCGAGATCGATC | |
| | ACAAGATTGTATCCAAGCGGAAGACTTAGAACAATCTGCAAGCTAATA | |
| | CCACGGCAGATCGTATTGGAGTATAGGATTCTACCTAATACGGTATTC | |
| | AGTCAGGAGATTAAGATTGCGCAGTGCAGATTCTGAACTTGTCAAGTT | |
| | CCTTAGTACTAAGATTGCGGCCTGGCCACTGACTAAGATAGAAGCTGT | |
| | AACTTGCCTCAGATTGTGGCCGCTGATCTGCAGAGGAACCCCTAGTGA | |
| | TGGAGTTGGCCACTCCCTCTCTGCGCGCTCGCTCGCTCACTGAGGCCG | |
| | CCCGGGCAAAGCCCGGGCGTCGGGCGACCTTTGGTCGCCCGGCCTCAG | |
| | TGAGCGAGCGAGCGCGCAGAGAGGGAGTGGCCAA | |
| 1TetP1_1X_ | TCTGCGCGCTCGCTCGCTCACTGAGGCCGGGCGACCAAAGGTCGCCCG | 205 |
| CCT2.10_ | ACGCCCGGGCTTTGCCCGGGCGGCCTCAGTGAGCGAGCGAGCGCGCAG | |
| pscAAV_ | AGGATCCTATCAAGAACCAACCTAGAACAGGATACCACAGTGAATATC | |
| scITR2_CMV- | ACTGTTAGATCCCTATCAGTGATAGAGAGCCCCAGTGGCCTAATGGAT | |
| Thy1.1-pA | AAGGCACTGGCCTTCAAAGCCAGGGATTGCGGGTTCGAGTCCCGCCTG | |
| | GGGTGTTTTTTTTGGACCGTTATAACCGGTATTATCTTCGTGTTATTG | |
| | CCGTCGTAGCAGACCTGGCATTATGCCCAGTACATGACCTTATGGGAC | |
| | TTTCCTACTTGGCAGTACATCTACGTATTAGTCATCGCTATTACCATG | |
| | GTGATGCGGTTTTGGCAGTACATCAATGGGCGTGGATAGCGGTTTGAC | |
| | TCACGGGGATTTCCAAGTCTCCACCCCATTGACGTCAATGGGAGTTTG | |
| | TTTTGGCACCAAAATCAACGGGACTTTCCAAAATGTCGTAACAACTCC | |
| | GCCCCATTGACGCAAATGGGCGGTAGGCGTGTACGGTGGGAGGTCTAT | |
| | ATAAGCAGAGCTCGTTTAGTGAACCGTCAGATCGCCTGGAGACGCCAT | |
| | CCACGCTGTTTTGACCTCCATAGTAGAGCCGCCACCATGGCCACAACC | |
| | ATGGGTCTTTTCTGCAATCACCGTCCTCGAGGCACCATGAACCCAGCC | |
| | ATCAGCGTCGCTCTCCTGCTCTCAGTCTTGCGGGTGTCCCGAGGGCAG | |
| | AAGGTGACCAGCCTGACAGCCTGCCTGGTGAACCAAAACCTTCGCCTG | |
| | GACTGCCGCCATGAGAATAACACCAAGGATAACTCCATCCAGCATGAG | |
| | TTCAGCCTGACCCGAGAGAAGAGGAAGCACGTGCTCTCAGGCACCCTT | |
| | GGGATACCCGAGCACACGTACCGCTCCCGCGTCACCCTCTCCAACCAG | |
| | CCCTATATCAAGGTCCTTACCCTAGCCAACTTCACCACCAAGGATGAG | |
| | GGCGACTACTTTTGTGAGCTTCGCGTCTCGGGCGCGAATCCCATGAGC | |
| | TCCAATAAAAGTATCAGTGTGTATAGAGACAAGCTGGTCAAGTGTGGC | |
| | GGCATAAGCCTGCTGGTTCAGAACACATCCTGGATGCTGCTGCTGCTG | |
| | CTTTCCCTCTCCCTCCTCCAAGCCCTGGACTTCATTTCTCTGTAAAAT | |
| | AAAGGAAATTTATTTTCATTGCAATAGTGTGTTGGAATTTTTTGTGTC | |
| | TCTCACAAGCGGCGCTCACTGTAGACCTTGACACACCACTGATCGGCA | |
| | GATTCCTTAACTGTAATCAAGAGTTCTTAGAGGAGACAATCCAACTCC | |
| | TTGGACTCCTCGAATAACTCTACTCGGTTCACAGTGGAACACTATTGA | |
| | TTGTAGATTACGGTGAGGCCGTGCTCAGACTTCGAGATCGATCACAAG | |
| | ATTGTATCCAAGCGGAAGACTTAGAACAATCTGCAAGCTAATACCACG | |
| | GCAGATCGTATTGGAGTATAGGATTCTACCTAATACGGTATTCAGTCA | |
| | GGAGATTAAGATTGCGCAGTGCAGATTCTGAACTTGTCAAGTTCCTTA | |
| | CTCGCAGCGTACTGGCCTAGCTATCGTGCTCACACTGCTATCTTGGTG | |
| | CGACTATTGTTCTGTTGCAGTCACCGTGTGTAGTGTATCTGCAGTCAG | |
| | ACTACTCTCTACGGAGGCTGCTTAAGTATCAGCGCTGAGTTGTCGCTT | |
| | GATTGTGCTGGTGACTATAAGTCCTGAAGTCCGATTGTTGCCTCAATT | |
| | GTTACTGTAGCGATACGATTAAGCGACGATATAGGCGACGGAAGATAC | |
| | AGCTTGCTATTGAGTGCGCGTTCTATCGTGACAGATTATTAGAAGTGT | |
| | AGACCGGAGGATCAGATAGATCGGCCTACCGCACACCTGAGATCAATA | |
| | CGTCGGTAAGAGCGCTGTAAGGACTAATACAATAGTAATAAGACCTAT | |
| | TATAGAGGCTGACTGGTACAGGAAGTATCTGATCTAGACGAATTGGCA | |
| | CACAAGTACTAAGATTGCGGCCTGGCCACTGACTAAGATAGAAGCTGT | |
| | AACTTGCCTCAGATTGTGGCCGCTGATCTGCAGAGGAACCCCTAGTGA | |
| | TGGAGTTGGCCACTCCCTCTCTGCGCGCTCGCTCGCTCACTGAGGCCG | |
| | CCCGGGCAAAGCCCGGGCGTCGGGCGACCTTTGGTCGCCCGGCCTCAG | |
| | TGAGCGAGCGAGCGCGCAGAGAGGGAGTGGCCAA | |
| 1TetP1_3X_ | TCTGCGCGCTCGCTCGCTCACTGAGGCCGGGCGACCAAAGGTCGCCCG | 206 |
| CCT2.10_ | ACGCCCGGGCTTTGCCCGGGCGGCCTCAGTGAGCGAGCGAGCGCGCAG | |
| pscAAV_ | AGGATCCTATCAAGAACCAACCTAGAACAGGATACCACAGTGAATATC | |
| scITR2_CMV- | ACTGTTAGATCCCTATCAGTGATAGAGAGCCCCAGTGGCCTAATGGAT | |
| Thy1.1-pA | AAGGCACTGGCCTTCAAAGCCAGGGATTGCGGGTTCGAGTCCCGCCTG | |
| | GGGTGTTTTTTCGATAGGCTGTTGTAAGGCGATTGGTGAAGGAATACG | |
| | CGGTACAAGTACACTCTCCCTATCAGTGATAGAGAGCCCCAGTGGCCT | |
| | AATGGATAAGGCACTGGCCTTCAAAGCCAGGGATTGCGGGTTCGAGTC | |
| | CCGCCTGGGGTGTTTTTTGAATAGTTACAGCGATTACCGCCTATTACC | |
| | AACACGGAAGCACTAGACTAATATCCCTATCAGTGATAGAGAGCCCCA | |
| | GTGGCCTAATGGATAAGGCACTGGCCTTCAAAGCCAGGGATTGCGGGT | |
| | TCGAGTCCCGCCTGGGGTGTTTTTTTTGGACCGTTATAACCGGTATTA | |
| | TCTTCGTGTTATTGCCGTCGTAGCAGACCTGGCATTATGCCCAGTACA | |
| | TGACCTTATGGGACTTTCCTACTTGGCAGTACATCTACGTATTAGTCA | |
| | TCGCTATTACCATGGTGATGCGGTTTTGGCAGTACATCAATGGGCGTG | |
| | GATAGCGGTTTGACTCACGGGGATTTCCAAGTCTCCACCCCATTGACG | |
| | TCAATGGGAGTTTGTTTTGGCACCAAAATCAACGGGACTTTCCAAAAT | |
| | GTCGTAACAACTCCGCCCCATTGACGCAAATGGGCGGTAGGCGTGTAC | |
| | GGTGGGAGGTCTATATAAGCAGAGCTCGTTTAGTGAACCGTCAGATCG | |
| | CCTGGAGACGCCATCCACGCTGTTTTGACCTCCATAGTAGAGCCGCCA | |
| | CCATGGCCACAACCATGGGTCTTTTCTGCAATCACCGTCCTCGAGGCA | |
| | CCATGAACCCAGCCATCAGCGTCGCTCTCCTGCTCTCAGTCTTGCGGG | |
| | TGTCCCGAGGGCAGAAGGTGACCAGCCTGACAGCCTGCCTGGTGAACC | |
| | AAAACCTTCGCCTGGACTGCCGCCATGAGAATAACACCAAGGATAACT | |
| | CCATCCAGCATGAGTTCAGCCTGACCCGAGAGAAGAGGAAGCACGTGC | |
| | TCTCAGGCACCCTTGGGATACCCGAGCACACGTACCGCTCCCGCGTCA | |
| | CCCTCTCCAACCAGCCCTATATCAAGGTCCTTACCCTAGCCAACTTCA | |
| | CCACCAAGGATGAGGGCGACTACTTTTGTGAGCTTCGCGTCTCGGGCG | |
| | CGAATCCCATGAGCTCCAATAAAAGTATCAGTGTGTATAGAGACAAGC | |
| | TGGTCAAGTGTGGCGGCATAAGCCTGCTGGTTCAGAACACATCCTGGA | |
| | TGCTGCTGCTGCTGCTTTCCCTCTCCCTCCTCCAAGCCCTGGACTTCA | |
| | TTTCTCTGTAAAATAAAGGAAATTTATTTTCATTGCAATAGTGTGTTG | |
| | GAATTTTTTGTGTCTCTCACAAGCGGCGCTCACTGTAGACCTTGACAC | |
| | ACCACTGATCGGCAGATTCCTTAACTGTAATCAAGAGTTCTTAGAGGA | |
| | GACAATCCAACTCCTTGGACTCCTCGAATAACTCTACTCGGTTCACAG | |
| | TGGAACACTATTGATTGTAGATTACGGTGAGGCCGTGCTCAGACTTCG | |
| | AGATCGATCACAAGATTGTATCCAAGCGGAAGACTTAGAACAATCTGC | |
| | AAGCTAATACCACGGCAGATCGTATTGGAGTATAGGATTCTACCTAAT | |
| | ACGGTATTCAGTCAGGAGATTAAGATTGCGCAGTGCAGATTCTGAACT | |
| | TGTCAAGTTCCTTACTCGCAGCGTACTGGCCTAGCTATCGTGCTCACA | |
| | CTGCTATCTTGGTGCGACTATTGTTCTGTTGCAGTCACCGTGTGTAGT | |
| | GTATCTGCAGTCAGACTACTCTCTACGGAGGCTGCTTAAGTATCAGCG | |
| | CTGAGGTACTAAGATTGCGGCCTGGCCACTGACTAAGATAGAAGCTGT | |
| | AACTTGCCTCAGATTGTGGCCGCTGATCTGCAGAGGAACCCCTAGTGA | |
| | TGGAGTTGGCCACTCCCTCTCTGCGCGCTCGCTCGCTCACTGAGGCCG | |
| | CCCGGGCAAAGCCCGGGCGTCGGGCGACCTTTGGTCGCCCGGCCTCAG | |
| | TGAGCGAGCGAGCGCGCAGAGAGGGAGTGGCCAA | |
| 1TetP1_3X_ | TCTGCGCGCTCGCTCGCTCACTGAGGCCGGGCGACCAAAGGTCGCCCG | 207 |
| CCT2.10- | ACGCCCGGGCTTTGCCCGGGCGGCCTCAGTGAGCGAGCGAGCGCGCAG | |
| WT- | AGGATCCTATCAAGAACCAACCTAGAACAGGATACCACAGTGAATATC | |
| AC_pscAAV_ | ACTGTTAGATCCCTATCAGTGATAGAGAGCCCCAGTGGCCTAATGGAT | |
| scITR2 | AAGGCACTGGCCTCCTAAGCCAGGGATTGCGGGTTCGAGTCCCGCCTG | |
| CMV- | GGGTGTTTTTTCGATAGGCTGTTGTAAGGCGATTGGTGAAGGAATACG | |
| Thy1.1-pA | CGGTACAAGTACACTCTCCCTATCAGTGATAGAGAGCCCCAGTGGCCT | |
| | AATGGATAAGGCACTGGCCTCCTAAGCCAGGGATTGCGGGTTCGAGTC | |
| | CCGCCTGGGGTGTTTTTTGAATAGTTACAGCGATTACCGCCTATTACC | |
| | AACACGGAAGCACTAGACTAATATCCCTATCAGTGATAGAGAGCCCCA | |
| | GTGGCCTAATGGATAAGGCACTGGCCTCCTAAGCCAGGGATTGCGGGT | |
| | TCGAGTCCCGCCTGGGGTGTTTTTTTTGGACCGTTATAACCGGTATTA | |
| | TCTTCGTGTTATTGCCGTCGTAGCAGACCTGGCATTATGCCCAGTACA | |
| | TGACCTTATGGGACTTTCCTACTTGGCAGTACATCTACGTATTAGTCA | |
| | TCGCTATTACCATGGTGATGCGGTTTTGGCAGTACATCAATGGGCGTG | |
| | GATAGCGGTTTGACTCACGGGGATTTCCAAGTCTCCACCCCATTGACG | |
| | TCAATGGGAGTTTGTTTTGGCACCAAAATCAACGGGACTTTCCAAAAT | |
| | GTCGTAACAACTCCGCCCCATTGACGCAAATGGGCGGTAGGCGTGTAC | |
| | GGTGGGAGGTCTATATAAGCAGAGCTCGTTTAGTGAACCGTCAGATCG | |
| | CCTGGAGACGCCATCCACGCTGTTTTGACCTCCATAGTAGAGCCGCCA | |
| | CCATGGCCACAACCATGGGTCTTTTCTGCAATCACCGTCCTCGAGGCA | |
| | CCATGAACCCAGCCATCAGCGTCGCTCTCCTGCTCTCAGTCTTGCGGG | |
| | TGTCCCGAGGGCAGAAGGTGACCAGCCTGACAGCCTGCCTGGTGAACC | |
| | AAAACCTTCGCCTGGACTGCCGCCATGAGAATAACACCAAGGATAACT | |
| | CCATCCAGCATGAGTTCAGCCTGACCCGAGAGAAGAGGAAGCACGTGC | |
| | TCTCAGGCACCCTTGGGATACCCGAGCACACGTACCGCTCCCGCGTCA | |
| | CCCTCTCCAACCAGCCCTATATCAAGGTCCTTACCCTAGCCAACTTCA | |
| | CCACCAAGGATGAGGGCGACTACTTTTGTGAGCTTCGCGTCTCGGGCG | |
| | CGAATCCCATGAGCTCCAATAAAAGTATCAGTGTGTATAGAGACAAGC | |
| | TGGTCAAGTGTGGCGGCATAAGCCTGCTGGTTCAGAACACATCCTGGA | |
| | TGCTGCTGCTGCTGCTTTCCCTCTCCCTCCTCCAAGCCCTGGACTTCA | |
| | TTTCTCTGTAAAATAAAGGAAATTTATTTTCATTGCAATAGTGTGTTG | |
| | GAATTTTTTGTGTCTCTCACAAGCGGCGCTCACTGTAGACCTTGACAC | |
| | ACCACTGATCGGCAGATTCCTTAACTGTAATCAAGAGTTCTTAGAGGA | |
| | GACAATCCAACTCCTTGGACTCCTCGAATAACTCTACTCGGTTCACAG | |
| | TGGAACACTATTGATTGTAGATTACGGTGAGGCCGTGCTCAGACTTCG | |
| | AGATCGATCACAAGATTGTATCCAAGCGGAAGACTTAGAACAATCTGC | |
| | AAGCTAATACCACGGCAGATCGTATTGGAGTATAGGATTCTACCTAAT | |
| | ACGGTATTCAGTCAGGAGATTAAGATTGCGCAGTGCAGATTCTGAACT | |
| | TGTCAAGTTCCTTACTCGCAGCGTACTGGCCTAGCTATCGTGCTCACA | |
| | CTGCTATCTTGGTGCGACTATTGTTCTGTTGCAGTCACCGTGTGTAGT | |
| | GTATCTGCAGTCAGACTACTCTCTACGGAGGCTGCTTAAGTATCAGCG | |
| | CTGAGGTACTAAGATTGCGGCCTGGCCACTGACTAAGATAGAAGCTGT | |
| | AACTTGCCTCAGATTGTGGCCGCTGATCTGCAGAGGAACCCCTAGTGA | |
| | TGGAGTTGGCCACTCCCTCTCTGCGCGCTCGCTCGCTCACTGAGGCCG | |
| | CCCGGGCAAAGCCCGGGCGTCGGGCGACCTTTGGTCGCCCGGCCTCAG | |
| | TGAGCGAGCGAGCGCGCAGAGAGGGAGTGGCCAA | |
| 2TetP1_1X_ | TCTGCGCGCTCGCTCGCTCACTGAGGCCGGGCGACCAAAGGTCGCCCG | 208 |
| CCT2.10_ | ACGCCCGGGCTTTGCCCGGGCGGCCTCAGTGAGCGAGCGAGCGCGCAG | |
| pscAAV_ | AGGATCCTATCAAGAACCAACCTAGAACAGGATACCACAGTGAATATC | |
| scITR2_CMV- | ACTGTTAGATCCCTATCAGTGATAGAGATCTCCCTATCAGTGATAGAG | |
| Thy1.1-pA | AGCCCCAGTGGCCTAATGGATAAGGCACTGGCCTTCAAAGCCAGGGAT | |
| | TGCGGGTTCGAGTCCCGCCTGGGGTGTTTTTTTTGGACCGTTATAACC | |
| | GGTATTATCTTCGTGTTATTGCCGTCGTAGCAGACCTGGCATTATGCC | |
| | CAGTACATGACCTTATGGGACTTTCCTACTTGGCAGTACATCTACGTA | |
| | TTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTT | |
| | CCAAAATGTCGTAACAACTCCGCCCCATTGACGCAAATGGGCGGTAGG | |
| | CGTGTACGGTGGGAGGTCTATATAAGCAGAGCTCGTTTAGTGAACCGT | |
| | CAGATCGCCTGGAGACGCCATCCACGCTGTTTTGACCTCCATAGTAGA | |
| | GCCGCCACCATGGCCACAACCATGGGTCTTTTCTGCAATCACCGTCCT | |
| | CGAGGCACCATGAACCCAGCCATCAGCGTCGCTCTCCTGCTCTCAGTC | |
| | TTGCGGGTGTCCCGAGGGCAGAAGGTGACCAGCCTGACAGCCTGCCTG | |
| | GTGAACCAAAACCTTCGCCTGGACTGCCGCCATGAGAATAACACCAAG | |
| | GATAACTCCATCCAGCATGAGTTCAGCCTGACCCGAGAGAAGAGGAAG | |
| | CACGTGCTCTCAGGCACCCTTGGGATACCCGAGCACACGTACCGCTCC | |
| | CGCGTCACCCTCTCCAACCAGCCCTATATCAAGGTCCTTACCCTAGCC | |
| | AACTTCACCACCAAGGATGAGGGCGACTACTTTTGTGAGCTTCGCGTC | |
| | TCGGGCGCGAATCCCATGAGCTCCAATAAAAGTATCAGTGTGTATAGA | |
| | GACAAGCTGGTCAAGTGTGGCGGCATAAGCCTGCTGGTTCAGAACACA | |
| | TCCTGGATGCTGCTGCTGCTGCTTTCCCTCTCCCTCCTCCAAGCCCTG | |
| | GACTTCATTTCTCTGTAAAATAAAGGAAATTTATTTTCATTGCAATAG | |
| | TGTGTTGGAATTTTTTGTGTCTCTCACAAGCGGCGCTCACTGTAGACC | |
| | TTGACACACCACTGATCGGCAGATTCCTTAACTGTAATCAAGAGTTCT | |
| | TAGAGGAGACAATCCAACTCCTTGGACTCCTCGAATAACTCTACTCGG | |
| | TTCACAGTGGAACACTATTGATTGTAGATTACGGTGAGGCCGTGCTCA | |
| | GACTTCGAGATCGATCACAAGATTGTATCCAAGCGGAAGACTTAGAAC | |
| | AATCTGCAAGCTAATACCACGGCAGATCGTATTGGAGTATAGGATTCT | |
| | ACCTAATACGGTATTCAGTCAGGAGATTAAGATTGCGCAGTGCAGATT | |
| | CTGAACTTGTCAAGTTCCTTACTCGCAGCGTACTGGCCTAGCTATCGT | |
| | GCTCACACTGCTATCTTGGTGCGACTATTGTTCTGTTGCAGTCACCGT | |
| | GTGTAGTGTATCTGCAGTCAGACTACTCTCTACGGAGGCTGCTTAAGT | |
| | ATCAGCGCTGAGTTGTCGCTTGATTGTGCTGGTGACTATAAGTCCTGA | |
| | AGTCCGATTGTTGCCTCAATTGTTACTGTAGCGATACGATTAAGCGAC | |
| | GATATAGGCGACGGAAGATACAGCTTGCTATTGAGTGCGCGTTCTATC | |
| | GTGACAGATTATTAGAAGTGTAGACCGGAGGATCAGATAGATCGGCCT | |
| | ACCGCACACCTGAGATCAATACGTCGGTAAGAGCGCTGTAAGGACTAA | |
| | TACAATAGTAATAAGACCTATTATAGAGGCTGACTGGTACAGGAAGTA | |
| | TCTGAGTACTAAGATTGCGGCCTGGCCACTGACTAAGATAGAAGCTGT | |
| | AACTTGCCTCAGATTGTGGCCGCTGATCTGCAGAGGAACCCCTAGTGA | |
| | TGGAGTTGGCCACTCCCTCTCTGCGCGCTCGCTCGCTCACTGAGGCCG | |
| | CCCGGGCAAAGCCCGGGCGTCGGGCGACCTTTGGTCGCCCGGCCTCAG | |
| | TGAGCGAGCGAGCGCGCAGAGAGGGAGTGGCCAA | |
| 2TetP1_3X_ | TCTGCGCGCTCGCTCGCTCACTGAGGCCGGGCGACCAAAGGTCGCCCG | 209 |
| CCT2.10_ | ACGCCCGGGCTTTGCCCGGGCGGCCTCAGTGAGCGAGCGAGCGCGCAG | |
| pscAAV_ | AGGATCCTATCAAGAACCAACCTAGAACAGGATACCACAGTGAATATC | |
| scITR2_CMV- | ACTGTTAGATCCCTATCAGTGATAGAGATCTCCCTATCAGTGATAGAG | |
| Thy1.1-pA | AGCCCCAGTGGCCTAATGGATAAGGCACTGGCCTTCAAAGCCAGGGAT | |
| | TGCGGGTTCGAGTCCCGCCTGGGGTGTTTTTTCGATAGGCTGTTGTAA | |
| | GGCGATTGGTGAAGGAATACGCGGTACAAGTACACTCTCCCTATCAGT | |
| | GATAGAGATCTCCCTATCAGTGATAGAGAGCCCCAGTGGCCTAATGGA | |
| | TAAGGCACTGGCCTTCAAAGCCAGGGATTGCGGGTTCGAGTCCCGCCT | |
| | GGGGTGTTTTTTGAATAGTTACAGCGATTACCGCCTATTACCAACACG | |
| | GAAGCACTAGACTAATATCCCTATCAGTGATAGAGATCTCCCTATCAG | |
| | TGATAGAGAGCCCCAGTGGCCTAATGGATAAGGCACTGGCCTTCAAAG | |
| | CCAGGGATTGCGGGTTCGAGTCCCGCCTGGGGTGTTTTTTTTGGACCG | |
| | TTATAACCGGTATTATCTTCGTGTTATTGCCGTCGTAGCAGACCTGGC | |
| | ATTATGCCCAGTACATGACCTTATGGGACTTTCCTACTTGGCAGTACA | |
| | TCTACGTATTAGTCATCGCTATTACCATGGTGATGCGGTTTTGGCAGT | |
| | ACATCAATGGGCGTGGATAGCGGTTTGACTCACGGGGATTTCCAAGTC | |
| | TCCACCCCATTGACGTCAATGGGAGTTTGTTTTGGCACCAAAATCAAC | |
| | GGGACTTTCCAAAATGTCGTAACAACTCCGCCCCATTGACGCAAATGG | |
| | GCGGTAGGCGTGTACGGTGGGAGGTCTATATAAGCAGAGCTCGTTTAG | |
| | TGAACCGTCAGATCGCCTGGAGACGCCATCCACGCTGTTTTGACCTCC | |
| | ATAGTAGAGCCGCCACCATGGCCACAACCATGGGTCTTTTCTGCAATC | |
| | ACCGTCCTCGAGGCACCATGAACCCAGCCATCAGCGTCGCTCTCCTGC | |
| | TCTCAGTCTTGCGGGTGTCCCGAGGGCAGAAGGTGACCAGCCTGACAG | |
| | CCTGCCTGGTGAACCAAAACCTTCGCCTGGACTGCCGCCATGAGAATA | |
| | ACACCAAGGATAACTCCATCCAGCATGAGTTCAGCCTGACCCGAGAGA | |
| | AGAGGAAGCACGTGCTCTCAGGCACCCTTGGGATACCCGAGCACACGT | |
| | ACCGCTCCCGCGTCACCCTCTCCAACCAGCCCTATATCAAGGTCCTTA | |
| | CCCTAGCCAACTTCACCACCAAGGATGAGGGCGACTACTTTTGTGAGC | |
| | TTCGCGTCTCGGGCGCGAATCCCATGAGCTCCAATAAAAGTATCAGTG | |
| | TGTATAGAGACAAGCTGGTCAAGTGTGGCGGCATAAGCCTGCTGGTTC | |
| | AGAACACATCCTGGATGCTGCTGCTGCTGCTTTCCCTCTCCCTCCTCC | |
| | AAGCCCTGGACTTCATTTCTCTGTAAAATAAAGGAAATTTATTTTCAT | |
| | TGCAATAGTGTGTTGGAATTTTTTGTGTCTCTCACAAGCGGCGCTCAC | |
| | TGTAGACCTTGACACACCACTGATCGGCAGATTCCTTAACTGTAATCA | |
| | AGAGTTCTTAGAGGAGACAATCCAACTCCTTGGACTCCTCGAATAACT | |
| | CTACTCGGTTCACAGTGGAACACTATTGATTGTAGATTACGGTGAGGC | |
| | CGTGCTCAGACTTCGAGATCGATCACAAGATTGTATCCAAGCGGAAGA | |
| | CTTAGAACAATCTGCAAGCTAATACCACGGCAGATCGTATTGGAGTAT | |
| | AGGATTCTACCTAATACGGTATTCAGTCAGGAGATTAAGATTGCGCAG | |
| | TGCAGATTCTGAACTTGTCAAGTTCCTTACTCGCAGCGTACTGGCCTA | |
| | GCTATCGTGCTCACACTGCTATCTTGGTGCGACTATTGTTCTGTTGCA | |
| | GTCACCGTGTGTAGTGTATCTGCAGTCAGACTACTCTCTACGGAGGCT | |
| | GCTTAAGTATCAGCGCTGAGGTACTAAGATTGCGGCCTGGCCACTGAC | |
| | TAAGATAGAAGCTGTAACTTGCCTCAGATTGTGGCCGCTGATCTGCAG | |
| | AGGAACCCCTAGTGATGGAGTTGGCCACTCCCTCTCTGCGCGCTCGCT | |
| | CGCTCACTGAGGCCGCCCGGGCAAAGCCCGGGCGTCGGGCGACCTTTG | |
| | GTCGCCCGGCCTCAGTGAGCGAGCGAGCGCGCAGAGAGGGAGTGGCCA | |
| | A | |
| 1Tetm45_3X_ | CCTTAATTAGGCTGCGCGCTCGCTCGCTCACTGAGGCCGCCCGGGCAA | 210 |
| CCT2.10_ | AGCCCGGGCGTCGGGCGACCTTTGGTCGCCCGGCCTCAGTGAGCGAGC | |
| pssAAV_ | GAGCGCGCAGAGAGGGAGTGGCCAACTCCATCACTAGGGGTTCCTTGT | |
| scITR2_CMV- | AGTTAATGATTAACCCGCCATGCTACTTATCTACGTAGCCATGCTCTA | |
| Thy1.1-pA | GGAAGATCGGAATTCGGATCCTATCAAGAACCAACCTAGAACAGGATA | |
| | CCACAGTGAATATCACTGTTAGATCCCTATCAGTGATAGAGATGGCAA | |
| | TTCCACGTTATAGCCGTTGATCAGATAAGAGTTGCAGAAGCCCCAGTG | |
| | GCCTAATGGATAAGGCACTGGCCTTCAAAGCCAGGGATTGCGGGTTCG | |
| | AGTCCCGCCTGGGGTGTTTTTTCGATAGGCTGTTGTAAGGCGATTGGT | |
| | GAAGGAATACGCGGTACAAGTACACTCTCCCTATCAGTGATAGAGAAC | |
| | TGTTAACAAGACACAGTGTGTTGTCTATTCGGTTGGTGCAGAAGCCCC | |
| | AGTGGCCTAATGGATAAGGCACTGGCCTTCAAAGCCAGGGATTGCGGG | |
| | TTCGAGTCCCGCCTGGGGTGTTTTTTGAATAGTTACAGCGATTACCGC | |
| | CTATTACCAACACGGAAGCACTAGACTAATATCCCTATCAGTGATAGA | |
| | GACTGACTGGTACAGGAAGTATCTGATCTAGACGAATTGGTGCAGAAG | |
| | CCCCAGTGGCCTAATGGATAAGGCACTGGCCTTCAAAGCCAGGGATTG | |
| | CGGGTTCGAGTCCCGCCTGGGGTGTTTTTTTTGGACCGTTATAACCGG | |
| | TATTATCTTCGTGTTATTGCCGTCGTAGCAGACCTGGCATTATGCCCA | |
| | GTACATGACCTTATGGGACTTTCCTACTTGGCAGTACATCTACGTATT | |
| | AGTCATCGCTATTACCATGGTGATGCGGTTTTGGCAGTACATCAATGG | |
| | GCGTGGATAGCGGTTTGACTCACGGGGATTTCCAAGTCTCCACCCCAT | |
| | TGACGTCAATGGGAGTTTGTTTTGGCACCAAAATCAACGGGACTTTCC | |
| | AAAATGTCGTAACAACTCCGCCCCATTGACGCAAATGGGCGGTAGGCG | |
| | TGTACGGTGGGAGGTCTATATAAGCAGAGCTCGTTTAGTGAACCGTCA | |
| | GATCGCCTGGAGACGCCATCCACGCTGTTTTGACCTCCATAGTAGAGC | |
| | CGCCACCATGGCCACAACCATGGGTCTTTTCTGCAATCACCGTCCTCG | |
| | AGGCACCATGAACCCAGCCATCAGCGTCGCTCTCCTGCTCTCAGTCTT | |
| | GCGGGTGTCCCGAGGGCAGAAGGTGACCAGCCTGACAGCCTGCCTGGT | |
| | GAACCAAAACCTTCGCCTGGACTGCCGCCATGAGAATAACACCAAGGA | |
| | TAACTCCATCCAGCATGAGTTCAGCCTGACCCGAGAGAAGAGGAAGCA | |
| | CGTGCTCTCAGGCACCCTTGGGATACCCGAGCACACGTACCGCTCCCG | |
| | CGTCACCCTCTCCAACCAGCCCTATATCAAGGTCCTTACCCTAGCCAA | |
| | CTTCACCACCAAGGATGAGGGCGACTACTTTTGTGAGCTTCGCGTCTC | |
| | GGGCGCGAATCCCATGAGCTCCAATAAAAGTATCAGTGTGTATAGAGA | |
| | CAAGCTGGTCAAGTGTGGCGGCATAAGCCTGCTGGTTCAGAACACATC | |
| | CTGGATGCTGCTGCTGCTGCTTTCCCTCTCCCTCCTCCAAGCCCTGGA | |
| | CTTCATTTCTCTGTAAAATAAAGGAAATTTATTTTCATTGCAATAGTG | |
| | TGTTGGAATTTTTTGTGTCTCTCACAAGCGGCGCTCACTGTAGACCTT | |
| | GACACACCACTGATCGGCAGATTCCTTAACTGTAATCAAGAGTTCTTA | |
| | GAGGAGACAATCCAACTCCTTGGACTCCTCGAATAACTCTACTCGGTT | |
| | CACAGTGGAACACTATTGATTGTAGATTACGGTGAGGCCGTGCTCAGA | |
| | CTTCGAGATCGATCACAAGATTGTATCCAAGCGGAAGACTTAGAACAA | |
| | TCTGCAAGCTAATACCACGGCAGATCGTATTGGAGTATAGGATTCTAC | |
| | CTAATACGGTATTCAGTCAGGAGATTAAGATTGCGCAGTGCAGATTCT | |
| | GAACTTGTCAAGTTCCTTACTCGCAGCGTACTGGCCTAGCTATCGTGC | |
| | TCACACTGCTATCTTGGTGCGACTATTGTTCTGTTGCAGTCACCGTGT | |
| | GTAGTGTATCTGCAGTCAGACTACTCTCTACGGAGGCTGCTTAAGTAT | |
| | CAGCGCTGAGTTGTCGCTTGATTGTGCTGGTGACTATAAGTCCTGAAG | |
| | TCCGATTGTTGCCTCAATTGTTACTGTAGCGATACGATTAAGCGACGA | |
| | TATAGGCGACGGAAGATACAGCTTGCTATTGAGTGCGCGTTCTATCGT | |
| | GACAGATTATTAGAAGTGTAGACCGGAGGATCAGATAGATCGGCCTAC | |
| | CGCACACCTGAGATCAATACGTCGGTAAGAGCGCTGTAAGGACTAATA | |
| | CAATAGTAATAAGACCTATTATAGAGGATTCGTTGTCGAGTAGAATTA | |
| | TTCCTATACAATCGCAGGTTCGATTGAGATTCTACTTCGCGTTCTTGA | |
| | CAGCTGAAGTCCTTGTGAACAATAGCGCGGCGCAATTGAAGACGTACT | |
| | TGACAAGCGTATCGATCTCTGTGAAGCACCGAATTGGCAAGAAGTATT | |
| | GTATTATAATTACCGTCTGGTTAACAGAGTGTTCACTACTCCGCCAAT | |
| | CGAGGTTATTACTTGAATCAACCGTCAGTACAACTTCGACACACTAAC | |
| | TACTAGCCAATCGAGATACGTAAGACACGTGTGTATACCTCTCCTATC | |
| | AGTGCGTTCAAGTGTTACACGATAGTTCGCTTAATTGACTCGGTCAAG | |
| | ATTATCACGTAGCTATTGTTACAATACTTGTAGAATCTAAGCCGTATC | |
| | GCTATCGGAATTAGCCGCTGTCACCTATGACACTATCCGCACTGTAAG | |
| | ATCGCTGCAGTTCGCAGCCTCCGAACGATTGAGGTGAATAAGGTTATT | |
| | CTTGCAGTTGCACCAGGAACCTGCGTTATAATTAGTCCTTGGCGTCAC | |
| | TTGCGAAGATTAGGATCAGATTGCAGCACTCGTAGGACTTGACTTCAC | |
| | TACAGTGAGCAGTATCTAGACGCCAAGGTAGCTTCCAGTACGCCTCGT | |
| | TACTTCGGAGTTACGTATACTCACGCGTAAGTTGCCGAATAGGTGCAC | |
| | TATGACTGGAGTGCTTAGCGCGTGATTACTGCTGGAGGATTGGAATTG | |
| | GCGATTCTTACGCGGAACCACGATAACGAGATAACGTTAAGTCGCTAG | |
| | ACTTAAGGCGGCACGCGATCGAGCAACTCCTCACTACGATAGGTGAGC | |
| | GCAGTCCAGTGTAGCTCAGTCGGCCAATTACGGTTCTATTAGACAGTT | |
| | GCACGTTGACATCACACTCGTTCCTATAGTGTATAGACTCAATTGTAG | |
| | TTCACCGCTGAGTATCACTCACAGCACGATCTACTCAGACTTAATCGC | |
| | ACTACTGGACGGAATCGCGTCGTCGGCCAATAAGCCTGTGTCCAGAGC | |
| | TGCAGTAGTAACTCCACTGTAGCTGTTAGACAATAAGTACTAAGATTG | |
| | CGGCCTGGCCACTGACTAAGATAGAAGCTGTAACTTGCCTCAGATTGT | |
| | GGCCGCTGATCTGCAGGAATTCCCGATAAGGATCTTCCTAGAGCATGG | |
| | CTACGTAGATAAGTAGCATGGCGGGTTAATCATTAACTACAAGGAACC | |
| | CCTAGTGATGGAGTTGGCCACTCCCTCTCTGCGCGCTCGCTCGCTCAC | |
| | TGAGGCCGGGCGACCAAAGGTCGCCCGACGCCCGGGCTTTGCCCGGGC | |
| | GGCCTCAGTGAGCGAGCGAGCGCGCAG | |
| 1TetP1_3X_ | CCTTAATTAGGCTGCGCGCTCGCTCGCTCACTGAGGCCGCCCGGGCAA | 211 |
| CCT2.10_ | AGCCCGGGCGTCGGGCGACCTTTGGTCGCCCGGCCTCAGTGAGCGAGC | |
| pssAAV_ | GAGCGCGCAGAGAGGGAGTGGCCAACTCCATCACTAGGGGTTCCTTGT | |
| scITR2_CMV- | AGTTAATGATTAACCCGCCATGCTACTTATCTACGTAGCCATGCTCTA | |
| Thy1.1-pA | GGAAGATCGGAATTCGGATCCTATCAAGAACCAACCTAGAACAGGATA | |
| | CCACAGTGAATATCACTGTTAGATCCCTATCAGTGATAGAGAGCCCCA | |
| | GTGGCCTAATGGATAAGGCACTGGCCTTCAAAGCCAGGGATTGCGGGT | |
| | TCGAGTCCCGCCTGGGGTGTTTTTTCGATAGGCTGTTGTAAGGCGATT | |
| | GGTGAAGGAATACGCGGTACAAGTACACTCTCCCTATCAGTGATAGAG | |
| | AGCCCCAGTGGCCTAATGGATAAGGCACTGGCCTTCAAAGCCAGGGAT | |
| | TGCGGGTTCGAGTCCCGCCTGGGGTGTTTTTTGAATAGTTACAGCGAT | |
| | TACCGCCTATTACCAACACGGAAGCACTAGACTAATATCCCTATCAGT | |
| | GATAGAGAGCCCCAGTGGCCTAATGGATAAGGCACTGGCCTTCAAAGC | |
| | CAGGGATTGCGGGTTCGAGTCCCGCCTGGGGTGTTTTTTTTGGACCGT | |
| | TATAACCGGTATTATCTTCGTGTTATTGCCGTCGTAGCAGACCTGGCA | |
| | TTATGCCCAGTACATGACCTTATGGGACTTTCCTACTTGGCAGTACAT | |
| | CTACGTATTAGTCATCGCTATTACCATGGTGATGCGGTTTTGGCAGTA | |
| | CATCAATGGGCGTGGATAGCGGTTTGACTCACGGGGATTTCCAAGTCT | |
| | CCACCCCATTGACGTCAATGGGAGTTTGTTTTGGCACCAAAATCAACG | |
| | GGACTTTCCAAAATGTCGTAACAACTCCGCCCCATTGACGCAAATGGG | |
| | CGGTAGGCGTGTACGGTGGGAGGTCTATATAAGCAGAGCTCGTTTAGT | |
| | GAACCGTCAGATCGCCTGGAGACGCCATCCACGCTGTTTTGACCTCCA | |
| | TAGTAGAGCCGCCACCATGGCCACAACCATGGGTCTTTTCTGCAATCA | |
| | CCGTCCTCGAGGCACCATGAACCCAGCCATCAGCGTCGCTCTCCTGCT | |
| | CTCAGTCTTGCGGGTGTCCCGAGGGCAGAAGGTGACCAGCCTGACAGC | |
| | CTGCCTGGTGAACCAAAACCTTCGCCTGGACTGCCGCCATGAGAATAA | |
| | CACCAAGGATAACTCCATCCAGCATGAGTTCAGCCTGACCCGAGAGAA | |
| | GAGGAAGCACGTGCTCTCAGGCACCCTTGGGATACCCGAGCACACGTA | |
| | CCGCTCCCGCGTCACCCTCTCCAACCAGCCCTATATCAAGGTCCTTAC | |
| | CCTAGCCAACTTCACCACCAAGGATGAGGGCGACTACTTTTGTGAGCT | |
| | TCGCGTCTCGGGCGCGAATCCCATGAGCTCCAATAAAAGTATCAGTGT | |
| | GTATAGAGACAAGCTGGTCAAGTGTGGCGGCATAAGCCTGCTGGTTCA | |
| | GAACACATCCTGGATGCTGCTGCTGCTGCTTTCCCTCTCCCTCCTCCA | |
| | AGCCCTGGACTTCATTTCTCTGTAAAATAAAGGAAATTTATTTTCATT | |
| | GCAATAGTGTGTTGGAATTTTTTGTGTCTCTCACAAGCGGCGCTCACT | |
| | GTAGACCTTGACACACCACTGATCGGCAGATTCCTTAACTGTAATCAA | |
| | GAGTTCTTAGAGGAGACAATCCAACTCCTTGGACTCCTCGAATAACTC | |
| | TACTCGGTTCACAGTGGAACACTATTGATTGTAGATTACGGTGAGGCC | |
| | GTGCTCAGACTTCGAGATCGATCACAAGATTGTATCCAAGCGGAAGAC | |
| | TTAGAACAATCTGCAAGCTAATACCACGGCAGATCGTATTGGAGTATA | |
| | GGATTCTACCTAATACGGTATTCAGTCAGGAGATTAAGATTGCGCAGT | |
| | GCAGATTCTGAACTTGTCAAGTTCCTTACTCGCAGCGTACTGGCCTAG | |
| | CTATCGTGCTCACACTGCTATCTTGGTGCGACTATTGTTCTGTTGCAG | |
| | TCACCGTGTGTAGTGTATCTGCAGTCAGACTACTCTCTACGGAGGCTG | |
| | CTTAAGTATCAGCGCTGAGTTGTCGCTTGATTGTGCTGGTGACTATAA | |
| | GTCCTGAAGTCCGATTGTTGCCTCAATTGTTACTGTAGCGATACGATT | |
| | AAGCGACGATATAGGCGACGGAAGATACAGCTTGCTATTGAGTGCGCG | |
| | TTCTATCGTGACAGATTATTAGAAGTGTAGACCGGAGGATCAGATAGA | |
| | TCGGCCTACCGCACACCTGAGATCAATACGTCGGTAAGAGCGCTGTAA | |
| | GGACTAATACAATAGTAATAAGACCTATTATAGAGGATTCGTTGTCGA | |
| | GTAGAATTATTCCTATACAATCGCAGGTTCGATTGAGATTCTACTTCG | |
| | CGTTCTTGACAGCTGAAGTCCTTGTGAACAATAGCGCGGCGCAATTGA | |
| | AGACGTACTTGACAAGCGTATCGATCTCTGTGAAGCACCGAATTGGCA | |
| | AGAAGTATTGTATTATAATTACCGTCTGGTTAACAGAGTGTTCACTAC | |
| | TCCGCCAATCGAGGTTATTACTTGAATCAACCGTCAGTACAACTTCGA | |
| | CACACTAACTACTAGCCAATCGAGATACGTAAGACACGTGTGTATACC | |
| | TCTCCTATCAGTGCGTTCAAGTGTTACACGATAGTTCGCTTAATTGAC | |
| | TCGGTCAAGATTATCACGTAGCTATTGTTACAATACTTGTAGAATCTA | |
| | AGCCGTATCGCTATCGGAATTAGCCGCTGTCACCTATGACACTATCCG | |
| | CACTGTAAGATCGCTGCAGTTCGCAGCCTCCGAACGATTGAGGTGAAT | |
| | AAGGTTATTCTTGCAGTTGCACCAGGAACCTGCGTTATAATTAGTCCT | |
| | TGGCGTCACTTGCGAAGATTAGGATCAGATTGCAGCACTCGTAGGACT | |
| | TGACTTCACTACAGTGAGCAGTATCTAGACGCCAAGGTAGCTTCCAGT | |
| | ACGCCTCGTTACTTCGGAGTTACGTATACTCACGCGTAAGTTGCCGAA | |
| | TAGGTGCACTATGACTGGAGTGCTTAGCGCGTGATTACTGCTGGAGGA | |
| | TTGGAATTGGCGATTCTTACGCGGAACCACGATAACGAGATAACGTTA | |
| | AGTCGCTAGACTTAAGGCGGCACGCGATCGAGCAACTCCTCACTACGA | |
| | TAGGTGAGCGCAGTCCAGTGTAGCTCAGTCGGCCAATTACGGTTCTAT | |
| | TAGACAGTTGCACGTTGACATCACACTCGTTCCTATAGTGTATAGACT | |
| | CAATTGTAGTTCACCGCTGAGTATCACTCACAGCACGATCTACTCAGA | |
| | CTTAATCGCACTACTGGACGGAATCGCGTCGTCGGCCAATAAGCCTGT | |
| | GTCCAGAGCTGCAGTAGTAACTCCACTGTAGCTGTTAGACAATAAGAC | |
| | GAGTGATACTGGCTAGCGAGGAGCGCGAGGACACTTAGATCCACAGTC | |
| | TAACTGACAAGGACGAGTGAGCAACTAGCAGTCTGCGGTGATTGAGTC | |
| | TCGCGGCCGCTGTTATCGCCAGTACGGAATAGGTCGGTACTAAGATTG | |
| | CGGCCTGGCCACTGACTAAGATAGAAGCTGTAACTTGCCTCAGATTGT | |
| | GGCCGCTGATCTGCAGGAATTCCCGATAAGGATCTTCCTAGAGCATGG | |
| | CTACGTAGATAAGTAGCATGGCGGGTTAATCATTAACTACAAGGAACC | |
| | CCTAGTGATGGAGTTGGCCACTCCCTCTCTGCGCGCTCGCTCGCTCAC | |
| | TGAGGCCGGGCGACCAAAGGTCGCCCGACGCCCGGGCTTTGCCCGGGC | |
| | GGCCTCAGTGAGCGAGCGAGCGCGCAG | |
| 1TetP1_3X_ | CCTTAATTAGGCTGCGCGCTCGCTCGCTCACTGAGGCCGCCCGGGCAA | 212 |
| CCT2.10- | AGCCCGGGCGTCGGGCGACCTTTGGTCGCCCGGCCTCAGTGAGCGAGC | |
| WT- | GAGCGCGCAGAGAGGGAGTGGCCAACTCCATCACTAGGGGTTCCTTGT | |
| AC_pssAAV_ | AGTTAATGATTAACCCGCCATGCTACTTATCTACGTAGCCATGCTCTA | |
| scITR2_ | GGAAGATCGGAATTCGGATCCTATCAAGAACCAACCTAGAACAGGATA | |
| CMV- | CCACAGTGAATATCACTGTTAGATCCCTATCAGTGATAGAGAGCCCCA | |
| Thy1.1-pA | GTGGCCTAATGGATAAGGCACTGGCCTCCTAAGCCAGGGATTGCGGGT | |
| | TCGAGTCCCGCCTGGGGTGTTTTTTCGATAGGCTGTTGTAAGGCGATT | |
| | GGTGAAGGAATACGCGGTACAAGTACACTCTCCCTATCAGTGATAGAG | |
| | AGCCCCAGTGGCCTAATGGATAAGGCACTGGCCTCCTAAGCCAGGGAT | |
| | TGCGGGTTCGAGTCCCGCCTGGGGTGTTTTTTGAATAGTTACAGCGAT | |
| | TACCGCCTATTACCAACACGGAAGCACTAGACTAATATCCCTATCAGT | |
| | GATAGAGAGCCCCAGTGGCCTAATGGATAAGGCACTGGCCTCCTAAGC | |
| | CAGGGATTGCGGGTTCGAGTCCCGCCTGGGGTGTTTTTTTTGGACCGT | |
| | TATAACCGGTATTATCTTCGTGTTATTGCCGTCGTAGCAGACCTGGCA | |
| | TTATGCCCAGTACATGACCTTATGGGACTTTCCTACTTGGCAGTACAT | |
| | CTACGTATTAGTCATCGCTATTACCATGGTGATGCGGTTTTGGCAGTA | |
| | CATCAATGGGCGTGGATAGCGGTTTGACTCACGGGGATTTCCAAGTCT | |
| | CCACCCCATTGACGTCAATGGGAGTTTGTTTTGGCACCAAAATCAACG | |
| | GGACTTTCCAAAATGTCGTAACAACTCCGCCCCATTGACGCAAATGGG | |
| | CGGTAGGCGTGTACGGTGGGAGGTCTATATAAGCAGAGCTCGTTTAGT | |
| | GAACCGTCAGATCGCCTGGAGACGCCATCCACGCTGTTTTGACCTCCA | |
| | TAGTAGAGCCGCCACCATGGCCACAACCATGGGTCTTTTCTGCAATCA | |
| | CCGTCCTCGAGGCACCATGAACCCAGCCATCAGCGTCGCTCTCCTGCT | |
| | CTCAGTCTTGCGGGTGTCCCGAGGGCAGAAGGTGACCAGCCTGACAGC | |
| | CTGCCTGGTGAACCAAAACCTTCGCCTGGACTGCCGCCATGAGAATAA | |
| | CACCAAGGATAACTCCATCCAGCATGAGTTCAGCCTGACCCGAGAGAA | |
| | GAGGAAGCACGTGCTCTCAGGCACCCTTGGGATACCCGAGCACACGTA | |
| | CCGCTCCCGCGTCACCCTCTCCAACCAGCCCTATATCAAGGTCCTTAC | |
| | CCTAGCCAACTTCACCACCAAGGATGAGGGCGACTACTTTTGTGAGCT | |
| | TCGCGTCTCGGGCGCGAATCCCATGAGCTCCAATAAAAGTATCAGTGT | |
| | GTATAGAGACAAGCTGGTCAAGTGTGGCGGCATAAGCCTGCTGGTTCA | |
| | GAACACATCCTGGATGCTGCTGCTGCTGCTTTCCCTCTCCCTCCTCCA | |
| | AGCCCTGGACTTCATTTCTCTGTAAAATAAAGGAAATTTATTTTCATT | |
| | GCAATAGTGTGTTGGAATTTTTTGTGTCTCTCACAAGCGGCGCTCACT | |
| | GTAGACCTTGACACACCACTGATCGGCAGATTCCTTAACTGTAATCAA | |
| | GAGTTCTTAGAGGAGACAATCCAACTCCTTGGACTCCTCGAATAACTC | |
| | TACTCGGTTCACAGTGGAACACTATTGATTGTAGATTACGGTGAGGCC | |
| | GTGCTCAGACTTCGAGATCGATCACAAGATTGTATCCAAGCGGAAGAC | |
| | TTAGAACAATCTGCAAGCTAATACCACGGCAGATCGTATTGGAGTATA | |
| | GGATTCTACCTAATACGGTATTCAGTCAGGAGATTAAGATTGCGCAGT | |
| | GCAGATTCTGAACTTGTCAAGTTCCTTACTCGCAGCGTACTGGCCTAG | |
| | CTATCGTGCTCACACTGCTATCTTGGTGCGACTATTGTTCTGTTGCAG | |
| | TCACCGTGTGTAGTGTATCTGCAGTCAGACTACTCTCTACGGAGGCTG | |
| | CTTAAGTATCAGCGCTGAGTTGTCGCTTGATTGTGCTGGTGACTATAA | |
| | GTCCTGAAGTCCGATTGTTGCCTCAATTGTTACTGTAGCGATACGATT | |
| | AAGCGACGATATAGGCGACGGAAGATACAGCTTGCTATTGAGTGCGCG | |
| | TTCTATCGTGACAGATTATTAGAAGTGTAGACCGGAGGATCAGATAGA | |
| | TCGGCCTACCGCACACCTGAGATCAATACGTCGGTAAGAGCGCTGTAA | |
| | GGACTAATACAATAGTAATAAGACCTATTATAGAGGATTCGTTGTCGA | |
| | GTAGAATTATTCCTATACAATCGCAGGTTCGATTGAGATTCTACTTCG | |
| | CGTTCTTGACAGCTGAAGTCCTTGTGAACAATAGCGCGGCGCAATTGA | |
| | AGACGTACTTGACAAGCGTATCGATCTCTGTGAAGCACCGAATTGGCA | |
| | AGAAGTATTGTATTATAATTACCGTCTGGTTAACAGAGTGTTCACTAC | |
| | TCCGCCAATCGAGGTTATTACTTGAATCAACCGTCAGTACAACTTCGA | |
| | CACACTAACTACTAGCCAATCGAGATACGTAAGACACGTGTGTATACC | |
| | TCTCCTATCAGTGCGTTCAAGTGTTACACGATAGTTCGCTTAATTGAC | |
| | TCGGTCAAGATTATCACGTAGCTATTGTTACAATACTTGTAGAATCTA | |
| | AGCCGTATCGCTATCGGAATTAGCCGCTGTCACCTATGACACTATCCG | |
| | CACTGTAAGATCGCTGCAGTTCGCAGCCTCCGAACGATTGAGGTGAAT | |
| | AAGGTTATTCTTGCAGTTGCACCAGGAACCTGCGTTATAATTAGTCCT | |
| | TGGCGTCACTTGCGAAGATTAGGATCAGATTGCAGCACTCGTAGGACT | |
| | TGACTTCACTACAGTGAGCAGTATCTAGACGCCAAGGTAGCTTCCAGT | |
| | ACGCCTCGTTACTTCGGAGTTACGTATACTCACGCGTAAGTTGCCGAA | |
| | TAGGTGCACTATGACTGGAGTGCTTAGCGCGTGATTACTGCTGGAGGA | |
| | TTGGAATTGGCGATTCTTACGCGGAACCACGATAACGAGATAACGTTA | |
| | AGTCGCTAGACTTAAGGCGGCACGCGATCGAGCAACTCCTCACTACGA | |
| | TAGGTGAGCGCAGTCCAGTGTAGCTCAGTCGGCCAATTACGGTTCTAT | |
| | TAGACAGTTGCACGTTGACATCACACTCGTTCCTATAGTGTATAGACT | |
| | CAATTGTAGTTCACCGCTGAGTATCACTCACAGCACGATCTACTCAGA | |
| | CTTAATCGCACTACTGGACGGAATCGCGTCGTCGGCCAATAAGCCTGT | |
| | GTCCAGAGCTGCAGTAGTAACTCCACTGTAGCTGTTAGACAATAAGAC | |
| | GAGTGATACTGGCTAGCGAGGAGCGCGAGGACACTTAGATCCACAGTC | |
| | TAACTGACAAGGACGAGTGAGCAACTAGCAGTCTGCGGTGATTGAGTC | |
| | TCGCGGCCGCTGTTATCGCCAGTACGGAATAGGTCGGTACTAAGATTG | |
| | CGGCCTGGCCACTGACTAAGATAGAAGCTGTAACTTGCCTCAGATTGT | |
| | GGCCGCTGATCTGCAGGAATTCCCGATAAGGATCTTCCTAGAGCATGG | |
| | CTACGTAGATAAGTAGCATGGCGGGTTAATCATTAACTACAAGGAACC | |
| | CCTAGTGATGGAGTTGGCCACTCCCTCTCTGCGCGCTCGCTCGCTCAC | |
| | TGAGGCCGGGCGACCAAAGGTCGCCCGACGCCCGGGCTTTGCCCGGGC | |
| | GGCCTCAGTGAGCGAGCGAGCGCGCAG | |
| pUC57_GG_ | TCGCGCGTTTCGGTGATGACGGTGAAAACCTCTGACACATGCAGCTCC | 213 |
| CMV- | CGGAGACGGTCACAGCTTGTCTGTAAGCGGATGCCGGGAGCAGACAAG | |
| Thy1.1_RBG- | CCCGTCAGGGCGCGTCAGCGGGTGTTGGCGGGTGTCGGGGCTGGCTTA | |
| pA | ACTATGCGGCATCAGAGCAGATTGTACTGAGAGTGCACCATATGCGGT | |
| | GTGAAATACCGCACAGATGCGTAAGGAGAAAATACCGCATCAGGCGCC | |
| | ATTCGCCATTCAGGCTGCGCAACTGTTGGGAAGGGCGATCGGTGCGGG | |
| | CCTCTTCGCTATTACGCCAGCTGGCGAAAGGGGGATGTGCTGCAAGGC | |
| | GATTAAGTTGGGTAACGCCAGGGTTTTCCCAGTCACGACGTTGTAAAA | |
| | CGACGGCCAGTGAATTGACGCGTGGATCCTATCAAGAACCAACCTAGA | |
| | ACAGGATACCACAGTGAATATCACTGTTAGAGTTCAGAGACCATGACA | |
| | CTGAGGTCTCAGGCCTTGGACCGTTATAACCGGTATTATCTTCGTGTT | |
| | ATTGCCGTCGTAGCAGACCTGGCATTATGCCCAGTACATGACCTTATG | |
| | GGACTTTCCTACTTGGCAGTACATCTACGTATTAGTCATCGCTATTAC | |
| | CATGGTGATGCGGTTTTGGCAGTACATCAATGGGCGTGGATAGCGGTT | |
| | TGACTCACGGGGATTTCCAAGTCTCCACCCCATTGACGTCAATGGGAG | |
| | TTTGTTTTGGCACCAAAATCAACGGGACTTTCCAAAATGTCGTAACAA | |
| | CTCCGCCCCATTGACGCAAATGGGCGGTAGGCGTGTACGGTGGGAGGT | |
| | CTATATAAGCAGAGCTCGTTTAGTGAACCGTCAGATCGCCTGGAGACG | |
| | CCATCCACGCTGTTTTGACCTCCATAGTAGAGCCGCCACCATGGCCAC | |
| | AACCATGGGTCTTTTCTGCAATCACCGTCCTCGGGGCACCATGAACCC | |
| | AGCCATCAGCGTCGCTCTCCTGCTCTCAGTCTTGCGGGTGTCCCGAGG | |
| | GCAGAAGGTGACCAGCCTGACAGCCTGCCTGGTGAACCAAAACCTTCG | |
| | CCTGGACTGCCGCCATGAGAATAACACCAAGGATAACTCCATCCAGCA | |
| | TGAGTTCAGCCTGACCCGAGAGAAGAGGAAGCACGTGCTCTCAGGCAC | |
| | CCTTGGGATACCCGAGCACACGTACCGCTCCCGCGTCACCCTCTCCAA | |
| | CCAGCCCTATATCAAGGTCCTTACCCTAGCCAACTTCACCACCAAGGA | |
| | TGAGGGCGACTACTTTTGTGAGCTTCGCGTCTCGGGCGCGAATCCCAT | |
| | GAGCTCCAATAAAAGTATCAGTGTGTATAGAGACAAGCTGGTCAAGTG | |
| | TGGCGGCATAAGCCTGCTGGTTCAGAACACATCCTGGATGCTGCTGCT | |
| | GCTGCTTTCCCTCTCCCTCCTCCAAGCCCTGGACTTCATTTCTCTGTA | |
| | AAATAAAGGAAATTTATTTTCATTGCAATAGTGTGTTGGAATTTTTTG | |
| | TGTCTCTCAGTACTAAGATTGCGGCCTGGCCACTGACTAAGATAGAAG | |
| | CTGTAACTTGCCTCAGATTGTGGCCGCTGATCTGCAGCTCGAGCATGG | |
| | TCATAGCTGTTTCCTGTGTGAAATTGTTATCCGCTCACAATTCCACAC | |
| | AACATACGAGCCGGAAGCATAAAGTGTAAAGCCTGGGGTGCCTAATGA | |
| | GTGAGCTAACTCACATTAATTGCGTTGCGCTCACTGCCCGCTTTCCAG | |
| | TCGGGAAACCTGTCGTGCCAGCTGCATTAATGAATCGGCCAACGCGCG | |
| | GGGAGAGGCGGTTTGCGTATTGGGCGCTCTTCCGCTTCCTCGCTCACT | |
| | GACTCGCTGCGCTCGGTCGTTCGGCTGCGGCGAGCGGTATCAGCTCAC | |
| | TCAAAGGCGGTAATACGGTTATCCACAGAATCAGGGGATAACGCAGGA | |
| | AAGAACATGTGAGCAAAAGGCCAGCAAAAGGCCAGGAACCGTAAAAAG | |
| | GCCGCGTTGCTGGCGTTTTTCCATAGGCTCCGCCCCCCTGACGAGCAT | |
| | CACAAAAATCGACGCTCAAGTCAGAGGTGGCGAAACCCGACAGGACTA | |
| | TAAAGATACCAGGCGTTTCCCCCTGGAAGCTCCCTCGTGCGCTCTCCT | |
| | GTTCCGACCCTGCCGCTTACCGGATACCTGTCCGCCTTTCTCCCTTCG | |
| | GGAAGCGTGGCGCTTTCTCATAGCTCACGCTGTAGGTATCTCAGTTCG | |
| | GTGTAGGTCGTTCGCTCCAAGCTGGGCTGTGTGCACGAACCCCCCGTT | |
| | CAGCCCGACCGCTGCGCCTTATCCGGTAACTATCGTCTTGAGTCCAAC | |
| | CCGGTAAGACACGACTTATCGCCACTGGCAGCAGCCACTGGTAACAGG | |
| | ATTAGCAGAGCGAGGTATGTAGGCGGTGCTACAGAGTTCTTGAAGTGG | |
| | TGGCCTAACTACGGCTACACTAGAAGAACAGTATTTGGTATCTGCGCT | |
| | CTGCTGAAGCCAGTTACCTTCGGAAAAAGAGTTGGTAGCTCTTGATCC | |
| | GGCAAACAAACCACCGCTGGTAGCGGTGGTTTTTTTGTTTGCAAGCAG | |
| | CAGATTACGCGCAGAAAAAAAGGATCTCAAGAAGATCCTTTGATCTTT | |
| | TCTACGGGGTCTGACGCTCAGTGGAACGAAAACTCACGTTAAGGGATT | |
| | TTGGTCATGAGATTATCAAAAAGGATCTTCACCTAGATCCTTTTAAAT | |
| | TAAAAATGAAGTTTTAAATCAATCTAAAGTATATATGAGTAAACTTGG | |
| | TCTGACAGTTAGAAAAACTCATCGAGCATCAAATGAAACTGCAATTTA | |
| | TTCATATCAGGATTATCAATACCATATTTTTGAAAAAGCCGTTTCTGT | |
| | AATGAAGGAGAAAACTCACCGAGGCAGTTCCATAGGATGGCAAGATCC | |
| | TGGTATCGGTCTGCGATTCCGACTCGTCCAACATCAATACAACCTATT | |
| | AATTTCCCCTCGTCAAAAATAAGGTTATCAAGTGAGAAATCACCATGA | |
| | GTGACGACTGAATCCGGTGAGAATGGCAAAAGTTTATGCATTTCTTTC | |
| | CAGACTTGTTCAACAGGCCAGCCATTACGCTCGTCATCAAAATCACTC | |
| | GCATCAACCAAACCGTTATTCATTCGTGATTGCGCCTGAGCGAGACGA | |
| | AATACGCGATCGCTGTTAAAAGGACAATTACAAACAGGAATCGAATGC | |
| | AACCGGCGCAGGAACACTGCCAGCGCATCAACAATATTTTCACCTGAA | |
| | TCAGGATATTCTTCTAATACCTGGAATGCTGTTTTCCCAGGGATCGCA | |
| | GTGGTGAGTAACCATGCATCATCAGGAGTACGGATAAAATGCTTGATG | |
| | GTCGGAAGAGGCATAAATTCCGTCAGCCAGTTTAGTCTGACCATCTCA | |
| | TCTGTAACATCATTGGCAACGCTACCTTTGCCATGTTTCAGAAACAAC | |
| | TCTGGCGCATCGGGCTTCCCATACAATCGATAGATTGTCGCACCTGAT | |
| | TGCCCGACATTATCGCGAGCCCATTTATACCCATATAAATCAGCATCC | |
| | ATGTTGGAATTTAATCGCGGCCTAGAGCAAGACGTTTCCCGTTGAATA | |
| | TGGCTCATACTCTTCCTTTTTCAATATTATTGAAGCATTTATCAGGGT | |
| | TATTGTCTCATGAGCGGATACATATTTGAATGTATTTAGAAAAATAAA | |
| | CAAATAGGGGTTCCGCGCACATTTCCCCGAAAAGTGCCACCTGACGTC | |
| | TAAGAAACCATTATTATCATGACATTAACCTATAAAAATAGGCGTATC | |
| | ACGAGGCCCTTTCGTC | |
| 23K | ATGGGCTCCAGTGAGCAGGAACTGAAAGCCATTGTCAAAGATCTTGGT | 214 |
| Endoprotease | TGTGGGCCATATTTTTTGGGCACCTATGACAAGCGCTTTCCAGGCTTT | |
| | GTTTCTCCACACAAGCTCGCCTGCGCCATAGTCAATACGGCCGGTCGC | |
| | GAGACTGGGGGCGTACACTGGATGGCCTTTGCCTGGAACCCGCACTCA | |
| | AAAACATGCTACCTCTTTGAGCCCTTTGGCTTTTCTGACCAGCGACTC | |
| | AAGCAGGTTTACCAGTTTGAGTACGAGTCACTCCTGCGCCGTAGCGCC | |
| | ATTGCTTCTTCCCCCGACCGCTGTATAACGCTGGAAAAGTCCACCCAA | |
| | AGCGTACAGGGGCCCAACTCGGCCGCCTGTGGACTATTCTGCTGCATG | |
| | TTTCTCCACGCCTTTGCCAACTGGCCCCAAACTCCCATGGATCACAAC | |
| | CCCACCATGAACCTTATTACCGGGGTACCCAACTCCATGCTCAACAGT | |
| | CCCCAGGTACAGCCCACCCTGCGTCGCAACCAGGAACAGCTCTACAGC | |
| | TTCCTGGAGCGCCACTCGCCCTACTTCCGCAGCCACAGTGCGCAGATT | |
| | AGGAGCGCCACTTCTTTTTGTCACTTGAAAAACATGTAA | |
| VP1 | ATGCCGGGGTTTTACGAGATTGTGATTAAGGTCCCCAGCGACCTTGAC | 215 |
| | GAGCATCTGCCCGGCATTTCTGACAGCTTTGTGAACTGGGTGGCCGAG | |
| | AAGGAATGGGAGTTGCCGCCAGATTCTGACATGGATCTGAATCTGATT | |
| | GAGCAGGCACCCCTGACCGTGGCCGAGAAGCTGCAGCGCGACTTTCTG | |
| | ACGGAATGGCGCCGTGTGAGTAAGGCCCCGGAGGCTCTTTTCTTTGTG | |
| | CAATTTGAGAAGGGAGAGAGCTACTTCCACATGCACGTGCTCGTGGAA | |
| | ACCACCGGGGTGAAATCCATGGTTTTGGGACGTTTCCTGAGTCAGATT | |
| | CGCGAAAAACTGATTCAGAGAATTTACCGCGGGATCGAGCCGACTTTG | |
| | CCAAACTGGTTCGCGGTCACAAAGACCAGAAATGGCGCCGGAGGCGGG | |
| | AACAAGGTGGTGGATGAGTGCTACATCCCCAATTACTTGCTCCCCAAA | |
| | ACCCAGCCTGAGCTCCAGTGGGCGTGGACTAATATGGAACAGTATTTA | |
| | AGCGCCTGTTTGAATCTCACGGAGCGTAAACGGTTGGTGGCGCAGCAT | |
| | CTGACGCACGTGTCGCAGACGCAGGAGCAGAACAAAGAGAATCAGAAT | |
| | CCCAATTCTGATGCGCCGGTGATCAGATCAAAAACTTCAGCCAGGTAC | |
| | ATGGAGCTGGTCGGGTGGCTCGTGGACAAGGGGATTACCTCGGAGAAG | |
| | CAGTGGATCCAGGAGGACCAGGCCTCATACATCTCCTTCAATGCGGCC | |
| | TCCAACTCGCGGTCCCAAATCAAGGCTGCCTTGGACAATGCGGGAAAG | |
| | ATTATGAGCCTGACTAAAACCGCCCCCGACTACCTGGTGGGCCAGCAG | |
| | CCCGTGGAGGACATTTCCAGCAATCGGATTTATAAAATTTTGGAACTA | |
| | AACGGGTACGATCCCCAATATGCGGCTTCCGTCTTTCTGGGATGGGCC | |
| | ACGAAAAAGTTCGGCAAGAGGAACACCATCTGGCTGTTTGGGCCTGCA | |
| | ACTACCGGGAAGACCAACATCGCGGAGGCCATAGCCCACACTGTGCCC | |
| | TTCTACGGGTGCGTAAACTGGACCAATGAGAACTTTCCCTTCAACGAC | |
| | TGTGTCGACAAGATGGTGATCTGGTGGGAGGAGGGGAAGATGACCGCC | |
| | AAGGTCGTGGAGTCGGCCAAAGCCATTCTCGGAGGAAGCAAGGTGCGC | |
| | GTGGACCAGAAATGCAAGTCCTCGGCCCAGATAGACCCGACTCCCGTG | |
| | ATCGTCACCTCCAACACCAACATGTGCGCCGTGATTGACGGGAACTCA | |
| | ACGACCTTCGAACACCAGCAGCCGTTGCAAGACCGGATGTTCAAATTT | |
| | GAACTCACCCGCCGTCTGGATCATGACTTTGGGAAGGTCACCAAGCAG | |
| | GAAGTCAAAGACTTTTTCCGGTGGGCAAAGGATCACGTGGTTGAGGTG | |
| | GAGCATGAATTCTACGTCAAAAAGGGTGGAGCCAAGAAAAGACCCGCC | |
| | CCCAGTGACGCAGATATAAGTGAGCCCAAACGGGTGCGCGAGTCAGTT | |
| | GCGCAGCCATCGACGTCAGACGCGGAAGCTTCGATCAACTACGCGGAC | |
| | AGGTACCAAAACAAATGTTCTCGTCACGTGGGCATGAATCTGATGCTG | |
| | TTTCCCTGCAGACAATGCGAGAGACTGAATCAGAATTCAAATATCTGC | |
| | TTCACTCACGGTGTCAAAGACTGTTTAGAGTGCTTTCCCGTGTCAGAA | |
| | TCTCAACCCGTTTCTGTCGTCAAAAAGGCGTATCAGAAACTGTGCTAC | |
| | ATTCATCACATCATGGGAAAGGTGCCAGACGCTTGCACTGCTTGCGAC | |
| | CTGGTCAATGTGGACTTGGATGACTGTGTTTCTGAACAATAAATGACT | |
| | TAAACCAGGT | |
| E1B 19K | ATGGAGGCTTGGGAGTGTTTGGAAGATTTTTCTGCTGTGCGTAACTTG | 216 |
| | CTGGAACAGAGCTCTAACAGTACCTCTTGGTTTTGGAGGTTTCTGTGG | |
| | GGCTCATCCCAGGCAAAGTTAGTCTGCAGAATTAAGGAGGATTACAAG | |
| | TGGGAATTTGAAGAGCTTTTGAAATCCTGTGGTGAGCTGTTTGATTCT | |
| | TTGAATCTGGGTCACCAGGCGCTTTTCCAAGAGAAGGTCATCAAGACT | |
| | TTGGATTTTTCCACACCGGGGCGCGCTGCGGCTGCTGTTGCTTTTTTG | |
| | AGTTTTATAAAGGATAAATGGAGCGAAGAAACCCATCTGAGCGGGGGG | |
| | TACCTGCTGGATTTTCTGGCCATGCATCTGTGGAGAGCGGTTGTGAGA | |
| | CACAAGAATCGCCTGCTACTGTTGTCTTCCGTCCGCCCGGCGATAATA | |
| | CCGACGGAGGAGCAGCAGCAGCAGCAGGAGGAAGCCAGGCGGCGGCGG | |
| | CAGGAGCAGAGCCCATGGAACCCGAGAGCCGGCCTGGACCCTCGGGAA | |
| | TGA | |
| E1B 55K | ATGGAGCGAAGAAACCCATCTGAGCGGGGGGTACCTGCTGGATTTTCT | 217 |
| | GGCCATGCATCTGTGGAGAGCGGTTGTGAGACACAAGAATCGCCTGCT | |
| | ACTGTTGTCTTCCGTCCGCCCGGCGATAATACCGACGGAGGAGCAGCA | |
| | GCAGCAGCAGGAGGAAGCCAGGCGGCGGCGGCAGGAGCAGAGCCCATG | |
| | GAACCCGAGAGCCGGCCTGGACCCTCGGGAATGAATGTTGTACAGGTG | |
| | GCTGAACTGTATCCAGAACTGAGACGCATTTTGACAATTACAGAGGAT | |
| | GGGCAGGGGCTAAAGGGGGTAAAGAGGGAGCGGGGGGCTTGTGAGGCT | |
| | ACAGAGGAGGCTAGGAATCTAGCTTTTAGCTTAATGACCAGACACCGT | |
| | CCTGAGTGTATTACTTTTCAACAGATCAAGGATAATTGCGCTAATGAG | |
| | CTTGATCTGCTGGCGCAGAAGTATTCCATAGAGCAGCTGACCACTTAC | |
| | TGGCTGCAGCCAGGGGATGATTTTGAGGAGGCTATTAGGGTATATGCA | |
| | AAGGTGGCACTTAGGCCAGATTGCAAGTACAAGATCAGCAAACTTGTA | |
| | AATATCAGGAATTGTTGCTACATTTCTGGGAACGGGGCCGAGGTGGAG | |
| | ATAGATACGGAGGATAGGGTGGCCTTTAGATGTAGCATGATAAATATG | |
| | TGGCCGGGGGTGCTTGGCATGGACGGGGTGGTTATTATGAATGTAAGG | |
| | TTTACTGGCCCCAATTTTAGCGGTACGGTTTTCCTGGCCAATACCAAC | |
| | CTTATCCTACACGGTGTAAGCTTCTATGGGTTTAACAATACCTGTGTG | |
| | GAAGCCTGGACCGATGTAAGGGTTCGGGGCTGTGCCTTTTACTGCTGC | |
| | TGGAAGGGGGTGGTGTGTCGCCCCAAAAGCAGGGCTTCAATTAAGAAA | |
| | TGCCTCTTTGAAAGGTGTACCTTGGGTATCCTGTCTGAGGGTAACTCC | |
| | AGGGTGCGCCACAATGTGGCCTCCGACTGTGGTTGCTTCATGCTAGTG | |
| | AAAAGCGTGGCTGTGATTAAGCATAACATGGTATGTGGCAACTGCGAG | |
| | GACAGGGCCTCTCAGATGCTGACCTGCTCGGACGGCAACTGTCACCTG | |
| | CTGAAGACCATTCACGTAGCCAGCCACTCTCGCAAGGCCTGGCCAGTG | |
| | TTTGAGCATAACATACTGACCCGCTGTTCCTTGCATTTGGGTAACAGG | |
| | AGGGGGGTGTTCCTACCTTACCAATGCAATTTGAGTCACACTAAGATA | |
| | TTGCTTGAGCCCGAGAGCATGTCCAAGGTGAACCTGAACGGGGTGTTT | |
| | GACATGACCATGAAGATCTGGAAGGTGCTGAGGTACGATGAGACCCGC | |
| | ACCAGGTGCAGACCCTGCGAGTGTGGCGGTAAACATATTAGGAACCAG | |
| | CCTGTGATGCTGGATGTGACCGAGGAGCTGAGGCCCGATCACTTGGTG | |
| | CTGGCCTGCACCCGCGCTGAGTTTGGCTCTAGCGATGAAGATACAGAT | |
| | TGA | |
| E2A | TTAAAAATCAAAGGGGTTCTGCCGCGCATCGCTATGCGCCACTGGCAG | 218 |
| | GGACACGTTGCGATACTGGTGTTTAGTGCTCCACTTAAACTCAGGCAC | |
| | AACCATCCGCGGCAGCTCGGTGAAGTTTTCACTCCACAGGCTGCGCAC | |
| | CATCACCAACGCGTTTAGCAGGTCGGGCGCCGATATCTTGAAGTCGCA | |
| | GTTGGGGCCTCCGCCCTGCGCGCGCGAGTTGCGATACACAGGGTTGCA | |
| | GCACTGGAACACTATCAGCGCCGGGTGGTGCACGCTGGCCAGCACGCT | |
| | CTTGTCGGAGATCAGATCCGCGTCCAGGTCCTCCGCGTTGCTCAGGGC | |
| | GAACGGAGTCAACTTTGGTAGCTGCCTTCCCAAAAAGGGCGCGTGCCC | |
| | AGGCTTTGAGTTGCACTCGCACCGTAGTGGCATCAAAAGGTGACCGTG | |
| | CCCGGTCTGGGCGTTAGGATACAGCGCCTGCATAAAAGCCTTGATCTG | |
| | CTTAAAAGCCACCTGAGCCTTTGCGCCTTCAGAGAAGAACATGCCGCA | |
| | AGACTTGCCGGAAAACTGATTGGCCGGACAGGCCGCGTCGTGCACGCA | |
| | GCACCTTGCGTCGGTGTTGGAGATCTGCACCACATTTCGGCCCCACCG | |
| | GTTCTTCACGATCTTGGCCTTGCTAGACTGCTCCTTCAGCGCGCGCTG | |
| | CCCGTTTTCGCTCGTCACATCCATTTCAATCACGTGCTCCTTATTTAT | |
| | CATAATGCTTCCGTGTAGACACTTAAGCTCGCCTTCGATCTCAGCGCA | |
| | GCGGTGCAGCCACAACGCGCAGCCCGTGGGCTCGTGATGCTTGTAGGT | |
| | CACCTCTGCAAACGACTGCAGGTACGCCTGCAGGAATCGCCCCATCAT | |
| | CGTCACAAAGGTCTTGTTGCTGGTGAAGGTCAGCTGCAACCCGCGGTG | |
| | CTCCTCGTTCAGCCAGGTCTTGCATACGGCCGCCAGAGCTTCCACTTG | |
| | GTCAGGCAGTAGTTTGAAGTTCGCCTTTAGATCGTTATCCACGTGGTA | |
| | CTTGTCCATCAGCGCGCGCGCAGCCTCCATGCCCTTCTCCCACGCAGA | |
| | CACGATCGGCACACTCAGCGGGTTCATCACCGTAATTTCACTTTCCGC | |
| | TTCGCTGGGCTCTTCCTCTTCCTCTTGCGTCCGCATACCACGCGCCAC | |
| | TGGGTCGTCTTCATTCAGCCGCCGCACTGTGCGCTTACCTCCTTTGCC | |
| | ATGCTTGATTAGCACCGGTGGGTTGCTGAAACCCACCATTTGTAGCGC | |
| | CACATCTTCTCTTTCTTCCTCGCTGTCCACGATTACCTCTGGTGATGG | |
| | CGGGCGCTCGGGCTTGGGAGAAGGGCGCTTCTTTTTCTTCTTGGGCGC | |
| | AATGGCCAAATCCGCCGCCGAGGTCGATGGCCGCGGGCTGGGTGTGCG | |
| | CGGCACCAGCGCGTCTTGTGATGAGTCTTCCTCGTCCTCGGACTCGAT | |
| | ACGCCGCCTCATCCGCTTTTTTGGGGGCGCCCGGGGAGGCGGCGGCGA | |
| | CGGGGACGGGGACGACACGTCCTCCATGGTTGGGGGACGTCGCGCCGC | |
| | ACCGCGTCCGCGCTCGGGGGTGGTTTCGCGCTGCTCCTCTTCCCGACT | |
| | GGCCAT | |
| Hexon- | ATGAGCAAGGAAATTCCCACGCCCTACATGTGGAGTTACCAGCCACAA | 219 |
| associated | ATGGGACTTGCGGCTGGAGCTGCCCAAGACTACTCAACCCGAATAAAC | |
| precursor | TACATGAGCGCGGGACCCCACATGATATCCCGGGTCAACGGAATACGC | |
| | GCCCACCGAAACCGAATTCTCCTGGAACAGGCGGCTATTACCACCACA | |
| | CCTCGTAATAACCTTAATCCCCGTAGTTGGCCCGCTGCCCTGGTGTAC | |
| | CAGGAAAGTCCCGCTCCCACCACTGTGGTACTTCCCAGAGACGCCCAG | |
| | GCCGAAGTTCAGATGACTAACTCAGGGGCGCAGCTTGCGGGCGGCTTT | |
| | CGTCACAGGGTGCGGTCGCCCGGGCAGGGTATAACTCACCTGACAATC | |
| | AGAGGGCGAGGTATTCAGCTCAACGACGAGTCGGTGAGCTCCTCGCTT | |
| | GGTCTCCGTCCGGACGGGACATTTCAGATCGGCGGCGCCGGCCGCTCT | |
| | TCATTCACGCCTCGTCAGGCAATCCTAACTCTGCAGACCTCGTCCTCT | |
| | GAGCCGCGCTCTGGAGGCATTGGAACTCTGCAATTTATTGAGGAGTTT | |
| | GTGCCATCGGTCTACTTTAACCCCTTCTCGGGACCTCCCGGCCACTAT | |
| | CCGGATCAATTTATTCCTAACTTTGACGCGGTAAAGGACTCGGCGGAC | |
| | GGCTACGACTGA | |
| Hexon | ATGGAGTCAGTCGAGAAGAAGGACAGCCTAACCGCCCCCTCTGAGTTC | 220 |
| Assembly | GCCACCACCGCCTCCACCGATGCCGCCAACGCGCCTACCACCTTCCCC | |
| | GTCGAGGCACCCCCGCTTGAGGAGGAGGAAGTGATTATCGAGCAGGAC | |
| | CCAGGTTTTGTAAGCGAAGACGACGAGGACCGCTCAGTACCAACAGAG | |
| | GATAAAAAGCAAGACCAGGACAACGCAGAGGCAAACGAGGAACAAGTC | |
| | GGGCGGGGGGACGAAAGGCATGGCGACTACCTAGATGTGGGAGACGAC | |
| | GTGCTGTTGAAGCATCTGCAGCGCCAGTGCGCCATTATCTGCGACGCG | |
| | TTGCAAGAGCGCAGCGATGTGCCCCTCGCCATAGCGGATGTCAGCCTT | |
| | GCCTACGAACGCCACCTATTCTCACCGCGCGTACCCCCCAAACGCCAA | |
| | GAAAACGGCACATGCGAGCCCAACCCGCGCCTCAACTTCTACCCCGTA | |
| | TTTGCCGTGCCAGAGGTGCTTGCCACCTATCACATCTTTTTCCAAAAC | |
| | TGCAAGATACCCCTATCCTGCCGTGCCAACCGCAGCCGAGCGGACAAG | |
| | CAGCTGGCCTTGCGGCAGGGCGCTGTCATACCTGATATCGCCTCGCTC | |
| | AACGAAGTGCCAAAAATCTTTGAGGGTCTTGGACGCGACGAGAAGCGC | |
| | GCGGCAAACGCTCTGCAACAGGAAAACAGCGAAAATGAAAGTCACTCT | |
| | GGAGTGTTGGTGGAACTCGAGGGTGACAACGCGCGCCTAGCCGTACTA | |
| | AAACGCAGCATCGAGGTCACCCACTTTGCCTACCCGGCACTTAACCTA | |
| | CCCCCCAAGGTCATGAGCACAGTCATGAGTGAGCTGATCGTGCGCCGT | |
| | GCGCAGCCCCTGGAGAGGGATGCAAATTTGCAAGAACAAACAGAGGAG | |
| | GGCCTACCCGCAGTTGGCGACGAGCAGCTAGCGCGCTGGCTTCAAACG | |
| | CGCGAGCCTGCCGACTTGGAGGAGCGACGCAAACTAATGATGGCCGCA | |
| | GTGCTCGTTACCGTGGAGCTTGAGTGCATGCAGCGGTTCTTTGCTGAC | |
| | CCGGAGATGCAGCGCAAGCTAGAGGAAACATTGCACTACACCTTTCGA | |
| | CAGGGCTACGTACGCCAGGCCTGCAAGATCTCCAACGTGGAGCTCTGC | |
| | AACCTGGTCTCCTACCTTGGAATTTTGCACGAAAACCGCCTTGGGCAA | |
| | AACGTGCTTCATTCCACGCTCAAGGGCGAGGCGCGCCGCGACTACGTC | |
| | CGCGACTGCGTTTACTTATTTCTATGCTACACCTGGCAGACGGCCATG | |
| | GGCGTTTGGCAGCAGTGCTTGGAGGAGTGCAACCTCAAGGAGCTGCAG | |
| | AAACTGCTAAAGCAAAACTTGAAGGACCTATGGACGGCCTTCAACGAG | |
| | CGCTCCGTGGCCGCGCACCTGGCGGACATCATTTTCCCCGAACGCCTG | |
| | CTTAAAACCCTGCAACAGGGTCTGCCAGACTTCACCAGTCAAAGCATG | |
| | TTGCAGAACTTTAGGAACTTTATCCTAGAGCGCTCAGGAATCTTGCCC | |
| | GCCACCTGCTGTGCACTTCCTAGCGACTTTGTGCCCATTAAGTACCGC | |
| | GAATGCCCTCCGCCGCTTTGGGGCCACTGCTACCTTCTGCAGCTAGCC | |
| | AACTACCTTGCCTACCACTCTGACATAATGGAAGACGTGAGCGGTGAC | |
| | GGTCTACTGGAGTGTCACTGTCGCTGCAACCTATGCACCCCGCACCGC | |
| | TCCCTGGTTTGCAATTCGCAGCTGCTTAACGAAAGTCAAATTATCGGT | |
| | ACCTTTGAGCTGCAGGGTCCCTCGCCTGACGAAAAGTCCGCGGCTCCG | |
| | GGGTTGAAACTCACTCCGGGGCTGTGGACGTCGGCTTACCTTCGCAAA | |
| | TTTGTACCTGAGGACTACCACGCCCACGAGATTAGGTTCTACGAAGAC | |
| | CAATCCCGCCCGCCTAATGCGGAGCTTACCGCCTGCGTCATTACCCAG | |
| | GGCCACATTCTTGGCCAATTGCAAGCCATCAACAAAGCCCGCCAAGAG | |
| | TTTCTGCTACGAAAGGGACGGGGGGTTTACTTGGACCCCCAGTCCGGC | |
| | GAGGAGCTCAACCCAATCCCCCCGCCGCCGCAGCCCTATCAGCAGCAG | |
| | CCGCGGGCCCTTGCTTCCCAGGATGGCACCCAAAAAGAAGCTGCAGCT | |
| | GCCGCCGCCACCCACGGACGAGGAGGAATACTGGGACAGTCAGGCAGA | |
| | GGAGGTTTTGGACGAGGAGGAGGAGGACATGATGGAAGACTGGGAGAG | |
| | CCTAGACGAGGAAGCTTCCGAGGTCGAAGAGGTGTCAGACGAAACACC | |
| | GTCACCCTCGGTCGCATTCCCCTCGCCGGCGCCCCAGAAATCGGCAAC | |
| | CGGTTCCAGCATGGCTACAACCTCCGCTCCTCAGGCGCCGCCGGCACT | |
| | GCCCGTTCGCCGACCCAACCGTAG | |
| Rep2 | ATGCCGGGGTTTTACGAGATTGTGATTAAGGTCCCCAGCGACCTTGAC | 221 |
| | GAGCATCTGCCCGGCATTTCTGACAGCTTTGTGAACTGGGTGGCCGAG | |
| | AAGGAATGGGAGTTGCCGCCAGATTCTGACATGGATCTGAATCTGATT | |
| | GAGCAGGCACCCCTGACCGTGGCCGAGAAGCTGCAGCGCGACTTTCTG | |
| | ACGGAATGGCGCCGTGTGAGTAAGGCCCCGGAGGCTCTTTTCTTTGTG | |
| | CAATTTGAGAAGGGAGAGAGCTACTTCCACATGCACGTGCTCGTGGAA | |
| | ACCACCGGGGTGAAATCCATGGTTTTGGGACGTTTCCTGAGTCAGATT | |
| | CGCGAAAAACTGATTCAGAGAATTTACCGCGGGATCGAGCCGACTTTG | |
| | CCAAACTGGTTCGCGGTCACAAAGACCAGAAATGGCGCCGGAGGCGGG | |
| | AACAAGGTGGTGGATGAGTGCTACATCCCCAATTACTTGCTCCCCAAA | |
| | ACCCAGCCTGAGCTCCAGTGGGCGTGGACTAATATGGAACAGTATTTA | |
| | AGCGCCTGTTTGAATCTCACGGAGCGTAAACGGTTGGTGGCGCAGCAT | |
| | CTGACGCACGTGTCGCAGACGCAGGAGCAGAACAAAGAGAATCAGAAT | |
| | CCCAATTCTGATGCGCCGGTGATCAGATCAAAAACTTCAGCCAGGTAC | |
| | ATGGAGCTGGTCGGGTGGCTCGTGGACAAGGGGATTACCTCGGAGAAG | |
| | CAGTGGATCCAGGAGGACCAGGCCTCATACATCTCCTTCAATGCGGCC | |
| | TCCAACTCGCGGTCCCAAATCAAGGCTGCCTTGGACAATGCGGGAAAG | |
| | ATTATGAGCCTGACTAAAACCGCCCCCGACTACCTGGTGGGCCAGCAG | |
| | CCCGTGGAGGACATTTCCAGCAATCGGATTTATAAAATTTTGGAACTA | |
| | AACGGGTACGATCCCCAATATGCGGCTTCCGTCTTTCTGGGATGGGCC | |
| | ACGAAAAAGTTCGGCAAGAGGAACACCATCTGGCTGTTTGGGCCTGCA | |
| | ACTACCGGGAAGACCAACATCGCGGAGGCCATAGCCCACACTGTGCCC | |
| | TTCTACGGGTGCGTAAACTGGACCAATGAGAACTTTCCCTTCAACGAC | |
| | TGTGTCGACAAGATGGTGATCTGGTGGGAGGAGGGGAAGATGACCGCC | |
| | AAGGTCGTGGAGTCGGCCAAAGCCATTCTCGGAGGAAGCAAGGTGCGC | |
| | GTGGACCAGAAATGCAAGTCCTCGGCCCAGATAGACCCGACTCCCGTG | |
| | ATCGTCACCTCCAACACCAACATGTGCGCCGTGATTGACGGGAACTCA | |
| | ACGACCTTCGAACACCAGCAGCCGTTGCAAGACCGGATGTTCAAATTT | |
| | GAACTCACCCGCCGTCTGGATCATGACTTTGGGAAGGTCACCAAGCAG | |
| | GAAGTCAAAGACTTTTTCCGGTGGGCAAAGGATCACGTGGTTGAGGTG | |
| | GAGCATGAATTCTACGTCAAAAAGGGTGGAGCCAAGAAAAGACCCGCC | |
| | CCCAGTGACGCAGATATAAGTGAGCCCAAACGGGTGCGCGAGTCAGTT | |
| | GCGCAGCCATCGACGTCAGACGCGGAAGCTTCGATCAACTACGCGGAC | |
| | AGGTACCAAAACAAATGTTCTCGTCACGTGGGCATGAATCTGATGCTG | |
| | TTTCCCTGCAGACAATGCGAGAGACTGAATCAGAATTCAAATATCTGC | |
| | TTCACTCACGGTGTCAAAGACTGTTTAGAGTGCTTTCCCGTGTCAGAA | |
| | TCTCAACCCGTTTCTGTCGTCAAAAAGGCGTATCAGAAACTGTGCTAC | |
| | ATTCATCACATCATGGGAAAGGTGCCAGACGCTTGCACTGCTTGCGAC | |
| | CTGGTCAATGTGGACTTGGATGACTGTGTTTCTGAACAATAAATGACT | |
| | TAAACCAGGT | |
| Tet | ATGTCTAGATTAGATAAAAGTAAAGTGATTAACAGCGCATTAGAGCTG | 222 |
| Repressor | CTTAATGAGGTCGGAATCGAAGGTTTAACAACCCGTAAACTCGCCCAG | |
| Gene | AAGCTAGGTGTAGAGCAGCCTACATTGTATTGGCATGTAAAAAATAAG | |
| | CGGGCTTTGCTCGACGCCTTAGCCATTGAGATGTTAGATAGGCACCAT | |
| | ACTCACTTTTGCCCTTTAGAAGGGGAAAGCTGGCAAGATTTTTTACGT | |
| | AATAACGCTAAAAGTTTTAGATGTGCTTTACTAAGTCATCGCGATGGA | |
| | GCAAAAGTACATTTAGGTACACGGCCTACAGAAAAACAGTATGAAACT | |
| | CTCGAAAATCAATTAGCCTTTTTATGCCAACAAGGTTTTTCACTAGAG | |
| | AATGCATTATATGCACTCAGCGCTGTGGGGCATTTTACTTTAGGTTGC | |
| | GTATTGGAAGATCAAGAGCATCAAGTCGCTAAAGAAGAAAGGGAAACA | |
| | CCTACTACTGATAGTATGCCGCCATTATTACGACAAGCTATCGAATTA | |
| | TTTGATCACCAAGGTGCAGAGCCAGCCTTCTTATTCGGCCTTGAATTG | |
| | ATCATATGCGGATTAGAAAAACAACTTAAATGTGAAAGTGGGTCCGCG | |
| | TACAGCGGATCCCGGGAATTCAGATCTTATTAA | |
| Tet | MSRLDKSKVINSALELLNEVGIEGLTTRKLAQKLGVEQPTLYWHVKNK | 223 |
| Repressor | RALLDALAIEMLDRHHTHFCPLEGESWQDELRNNAKSFRCALLSHRDG | |
| Protein | AKVHLGTRPTEKQYETLENQLAFLCQQGFSLENALYALSAVGHFTLGC | |
| | VLEDQEHQVAKEERETPTTDSMPPLLRQAIELFDHQGAEPAFLFGLEL | |
| | IICGLEKQLKCESGSAYSGSREFRSY* | |
| CMV- | CCTGGCATTATGCCCAGTACATGACCTTATGGGACTTTCCTACTTGGC | 224 |
| kozak- | AGTACATCTACGTATTAGTCATCGCTATTACCATGGTGATGCGGTTTT | |
| Thy1.1-pA | GGCAGTACATCAATGGGCGTGGATAGCGGTTTGACTCACGGGGATTTC | |
| | CAAGTCTCCACCCCATTGACGTCAATGGGAGTTTGTTTTGGCACCAAA | |
| | ATCAACGGGACTTTCCAAAATGTCGTAACAACTCCGCCCCATTGACGC | |
| | AAATGGGCGGTAGGCGTGTACGGTGGGAGGTCTATATAAGCAGAGCTC | |
| | GTTTAGTGAACCGTCAGATCGCCTGGAGACGCCATCCACGCTGTTTTG | |
| | ACCTCCATAGTAGAGCCGCCACCATGGCCACAACCATGGGTCTTTTCT | |
| | GCAATCACCGTCCTCGAGGCACCATGAACCCAGCCATCAGCGTCGCTC | |
| | TCCTGCTCTCAGTCTTGCGGGTGTCCCGAGGGCAGAAGGTGACCAGCC | |
| | TGACAGCCTGCCTGGTGAACCAAAACCTTCGCCTGGACTGCCGCCATG | |
| | AGAATAACACCAAGGATAACTCCATCCAGCATGAGTTCAGCCTGACCC | |
| | GAGAGAAGAGGAAGCACGTGCTCTCAGGCACCCTTGGGATACCCGAGC | |
| | ACACGTACCGCTCCCGCGTCACCCTCTCCAACCAGCCCTATATCAAGG | |
| | TCCTTACCCTAGCCAACTTCACCACCAAGGATGAGGGCGACTACTTTT | |
| | GTGAGCTTCGCGTCTCGGGCGCGAATCCCATGAGCTCCAATAAAAGTA | |
| | TCAGTGTGTATAGAGACAAGCTGGTCAAGTGTGGCGGCATAAGCCTGC | |
| | TGGTTCAGAACACATCCTGGATGCTGCTGCTGCTGCTTTCCCTCTCCC | |
| | TCCTCCAAGCCCTGGACTTCATTTCTCTGTAAAATAAAGGAAATTTAT | |
| | TTTCATTGCAATAGTGTGTTGGAATTTTTTGTGTCTCTCA | |
| 5′ITR | TCTGCGCGCTCGCTCGCTCACTGAGGCCGGGCGACCAAAGGTCGCCCG | 225 |
| (scAAV) | ACGCCCGGGCTTTGCCCGGGCGGCCTCAGTGAGCGAGCGAGCGCGCAG | |
| | A | |
| 5′ITR | CCTTAATTAGGCTGCGCGCTCGCTCGCTCACTGAGGCCGCCCGGGCAA | 226 |
| (SSAAV) | AGCCCGGGCGTCGGGCGACCTTTGGTCGCCCGGCCTCAGTGAGCGAGC | |
| | GAGCGCGCAGAGAGGGAGTGGCCAACTCCATCACTAGGGGTTCCT | |
| 3′ITR | AGGAACCCCTAGTGATGGAGTTGGCCACTCCCTCTCTGCGCGCTCGCT | 227 |
| (scAAV) | CGCTCACTGAGGCCGCCCGGGCAAAGCCCGGGCGTCGGGCGACCTTTG | |
| | GTCGCCCGGCCTCAGTGAGCGAGCGAGCGCGCAGAGAGGGAGTGGCCA | |
| | A | |
| 3′ITR | AGGAACCCCTAGTGATGGAGTTGGCCACTCCCTCTCTGCGCGCTCGCT | 228 |
| (scAAV) | CGCTCACTGAGGCCGGGCGACCAAAGGTCGCCCGACGCCCGGGCTTTG | |
| | CCCGGGCGGCCTCAGTGAGCGAGCGAGCGCGCAG | |

### EXEMPLARY EMBODIMENTS

Embodiment 1: A composition comprising a conditionally repressible suppressor tRNA coding sequence.

Embodiment 2: The composition of embodiment 1, wherein the conditionally repressible suppressor tRNA coding sequence comprises sequence coding for a repressor element and a sequence coding for a suppressor tRNA.

Embodiment 3: The composition of embodiment 1 or 2, wherein the conditionally repressible suppressor tRNA coding sequence comprises a sequence coding for a repressor element upstream or downstream of a sequence coding for a suppressor tRNA.

Embodiment 4: The composition of embodiment 2 or 3, wherein binding of a repressor to the repressor element represses expression of the suppressor tRNA.

Embodiment 5: The composition of embodiment 4, wherein the repressor is a Tet repressor protein.

Embodiment 6: The composition of any one of embodiments 2-5, wherein the sequence coding for the repressor element is positioned at +7, +12, +20, +30, or +45 of the sequence coding for the suppressor tRNA.

Embodiment 7: The composition of any one of embodiments 2-5, wherein the sequence coding for the repressor element is positioned at +1, −7, −12, −20, −30, or −45 of the sequence coding for the suppressor tRNA.

Embodiment 8: The composition of any one of embodiments 2-7, wherein the repressor element comprises an operon.

Embodiment 9: The composition of any one of embodiments 2-8, wherein the repressor element comprises a sequence of SEQ ID NO: 149 or SEQ ID NO: 150.

Embodiment 10: The composition of embodiment 9, wherein the operon comprises a Tet operator (TetO) or Lac operator (LacO).

Embodiment 11: The composition of embodiment 9, wherein the operon comprises a sequence of SEQ ID NO: 150.

Embodiment 12: The composition of any one of embodiments 2-11, wherein the repressor element comprises a tetracycline response element (TRE).

Embodiment 13: The composition of embodiment 12, wherein TRE comprises a TetO sequence.

Embodiment 14: The composition of embodiment 12 or 13, wherein the TRE comprises a concatemer of TetO sequences.

Embodiment 15: The composition of embodiment 14, wherein the concatemer of TetO sequences comprises 2, 3, 4, 5, 6, 7, 8, 9, or 10 TetO sequences.

Embodiment 16: The composition of embodiment 14 or 15, wherein two or more TetO sequences in the concatemer of TetO sequences are separated by a spacer sequence.

Embodiment 17: The composition of embodiment 16, wherein the spacer sequence is TC.

Embodiment 18: The composition of any one of embodiments 14-17, wherein the concatemer of TetO sequences is 2 TetO sequences.

Embodiment 19: The composition of any one of embodiments 14-18, wherein the concatemer of TetO sequences comprises 2 TetO sequences separated by a spacer sequence.

Embodiment 20: The composition of any one of embodiments 14-17 or 19, wherein the concatemer of TetO sequences comprises 2 TetO sequences separated by a TC spacer sequence.

Embodiment 21: The composition of any one of embodiments 14-17, 19, or 20, wherein the concatemer of TetO sequences is SEQ ID NO: 149.

Embodiment 22: The composition of any one of embodiments 12-21, wherein the TRE further comprises a promoter.

Embodiment 23: The composition of any one of embodiments 12-22, wherein TRE is a fusion of the TetO sequence and a promoter.

Embodiment 24: The composition of any one of embodiments 12-22, wherein TRE is a fusion of the concatemer of TetO sequence and a promoter.

Embodiment 25: The composition of any one of embodiments 22-24, wherein the promoter is a tRNA promoter or a fragment thereof.

Embodiment 26: The composition of any one of embodiments 22-25, wherein the promoter is downstream of the TetO sequence or the concatemer of TetO sequences.

Embodiment 27: The composition of any one of embodiments 22-25, wherein the promoter is upstream of the TetO sequence or the concatemer of TetO sequences.

Embodiment 28: The composition of any one of embodiments 1-27, wherein the conditionally repressible suppressor tRNA coding sequence is flanked by ITRs.

Embodiment 29: The composition of any one of embodiments 1-28 comprising one or more conditionally repressible suppressor tRNA coding sequences.

Embodiment 30: The composition of any one of embodiments 1-29 comprising two conditionally repressible suppressor tRNA coding sequences.

Embodiment 31: The composition of any one of embodiments 1-29 comprising three conditionally repressible suppressor tRNA coding sequences.

Embodiment 32: The composition of any one of embodiments 1-29 comprising six conditionally repressible suppressor tRNA coding sequences.

Embodiment 33: The composition of embodiment 29, wherein the one or more conditionally repressible suppressor tRNA coding sequence are flanked by inverted terminal repeats (ITRs).

Embodiment 34: The composition of any one of embodiments 30-32, wherein the two conditionally repressible suppressor tRNA coding sequences, the three conditionally repressible suppressor tRNA coding sequences, or the six conditionally repressible suppressor tRNA coding sequences are flanked by ITRs.

Embodiment 35: The composition of any one of embodiments 1-4, wherein the conditionally repressible suppressor tRNA coding sequence comprises a guide RNA binding sequence and encodes a suppressor tRNA.

Embodiment 36: The composition of embodiment 35, wherein the conditionally repressible suppressor tRNA coding sequence further comprises a protospacer adjacent motif (PAM).

Embodiment 37: The composition of embodiments 35 or 36, wherein the guide RNA binding sequence is from 15 to 30 nucleotides in length.

Embodiment 38: The composition of any one of embodiments 35-37, wherein the guide RNA binding sequence is positioned at +1 or −7 of the sequence coding for the suppressor tRNA.

Embodiment 39: The composition of any one of embodiments 35-38, wherein binding of the guide RNA binding sequence to a guide RNA complexed to a Cas protein that is linked or fused to a repressor domain represses expression of the suppressor tRNA.

Embodiment 40: The composition of embodiment 4, wherein the repressor is a guide RNA complexed to a Cas protein, wherein the Cas protein is linked or fused to a repressor domain.

Embodiment 41: The composition of embodiments 39 or 40, wherein the Cas protein is a catalytically dead Cas protein.

Embodiment 42: The composition of any one of embodiments 39-41, wherein the repressor domain is a krab domain.

Embodiment 43: The composition of any one of embodiments 35-42, wherein the conditionally repressible suppressor tRNA coding sequence is flanked by ITRs.

Embodiment 44: The composition of any one of embodiments 35-43 comprising one or more conditionally repressible suppressor tRNA coding sequences.

Embodiment 45: The composition of any one of embodiments 35-43 comprising two conditionally repressible suppressor tRNA coding sequences.

Embodiment 46: The composition of any one of embodiments 35-43 comprising three conditionally repressible suppressor tRNA coding sequences.

Embodiment 47: The composition of any one of embodiments 35-43 comprising six conditionally repressible suppressor tRNA coding sequences.

Embodiment 48: The composition of embodiment 35, wherein the one or more conditionally repressible suppressor tRNA coding sequence are flanked by ITRs.

Embodiment 49: The composition of any one of embodiments 35-47, wherein the two conditionally repressible suppressor tRNA coding sequences, the three conditionally repressible suppressor tRNA coding sequences, or the six conditionally repressible suppressor tRNA coding sequences are flanked by ITRs.

Embodiment 50: The composition of any one of embodiments 2-49, wherein the suppressor tRNA are capable of suppressing an opal stop codon, an ochre stop codon, or an amber stop codon.

Embodiment 51: The composition of any one of embodiments 2-50, wherein a sequence of the suppressor tRNA comprises 100%, at least 99%, at least 98%, at least 95%, or at least 90% sequence identity to any one of SEQ ID NO: 3-SEQ ID NO: 48 or SEQ ID NO: 103-SEQ ID NO: 148; optionally, wherein the anticodon is engineered to bind to an ochre stop codon or an amber stop codon.

Embodiment 52: The composition of any one of embodiments 2-50, wherein the conditionally repressible suppressor tRNA coding sequence comprises a sequence selected from SEQ ID NOs: 158-202.

Embodiment 53: A composition comprising one or more short interfering RNA (siRNA), one or more short hairpin RNA (shRNA), one or more dicer-substrate RNAs (DsRNA), or any combination thereof, wherein the one or more siRNA, the one or more shRNA, or the one or more DsRNA bind to a suppressor tRNA.

Embodiment 54: The composition of embodiment 53, wherein upon binding of the one or more siRNA, the one or more shRNA, or the one or more DsRNA, the suppressor tRNA is targeted for degradation.

Embodiment 55: The composition of embodiment 53 or 54, wherein upon binding of the one or more siRNA, the one or more shRNA, or the one or more DsRNA, the suppressor tRNA is degraded by an RNA induced silencing complex.

Embodiment 56: The composition of any one of embodiments 53-55, wherein the one or more short interfering RNA (siRNA), one or more short hairpin RNA (shRNA), one or more dicer-substrate RNAs (DsRNA), or any combination thereof, bind to an anticodon region of the suppressor tRNA.

Embodiment 57: The composition of any one of embodiments 53-56, wherein the one or more siRNA are from 15 nucleotides to 25 nucleotides in length.

Embodiment 58: The composition of any one of embodiments 53-57, wherein the one or more siRNA are from 19 nucleotides in length.

Embodiment 59: The composition of any one of embodiments 53-58, wherein the one or more DsRNA are from 25 to 35 nucleotides in length.

Embodiment 60: The composition of any one of embodiments 53-60, wherein the one or more DsRNA are 27 nucleotides in length.

Embodiment 61: The composition of any one of embodiments 53-61, further comprising the suppressor tRNA.

Embodiment 62: The composition of embodiment 61, wherein the suppressor tRNA is capable of suppressing an opal stop codon, an ochre stop codon, or an amber stop codon.

Embodiment 63: The composition of embodiment 61 or 62, wherein the suppressor tRNA comprises 100%, at least 99%, at least 98%, at least 95%, or at least 90% sequence identity to any one of SEQ ID NO: 3-SEQ ID NO: 48 or SEQ ID NO: 103-SEQ ID NO: 148; optionally, wherein the anticodon is engineered to bind to an ochre stop codon or an amber stop codon.

Embodiment 64: A composition comprising one or more stop codon engineered AAV nucleotide sequence(s).

Embodiment 65: The composition of embodiment 64 further comprising one or more suppressor tRNA coding sequence(s).

Embodiment 66: The composition of embodiment 64, further comprises one or more conditionally repressible suppressor tRNA coding sequence(s); optionally, wherein the one or more conditionally repressible suppressor tRNA coding sequences are any of embodiments 1-52.

Embodiment 67: The composition of embodiment 65 or 66, wherein the suppressor tRNA coding sequence(s) or the conditionally repressible suppressor tRNA coding sequence(s) are flanked by ITRs.

Embodiment 68: The composition of any one of embodiments 65-67, wherein the suppressor tRNA coding sequence(s) or the conditionally repressible suppressor tRNA coding sequence(s) independently encode a suppressor tRNA that is capable of suppressing an opal stop codon, an ochre stop codon, or an amber stop codon.

Embodiment 69: The composition of any one of embodiments 65-68, wherein the suppressor tRNA coding sequence comprises 100%, at least 99%, at least 98%, at least 95%, or at least 90% sequence identity to any one of SEQ ID NO: 3-SEQ ID NO: 48 or SEQ ID NO: 103-SEQ ID NO: 148; optionally, wherein the anticodon is engineered to bind to an ochre stop codon or an amber stop codon.

Embodiment 70: The composition of any one of embodiments 64-69, wherein the stop codon engineered AAV nucleotide sequence(s) are selected from the group consisting of a sequence coding for a Rep protein, a sequence coding for a Cap protein, a sequence coding for a helper protein, a sequence encoding VA RNA, a sequence encoding AAP, a sequence encoding MAAP, a sequence encoding Protein X, and any combinations thereof.

Embodiment 71: The composition of embodiment 70, wherein the Cap protein(s) are selected from the group consisting of VP1, VP2, VP3, and any combinations thereof.

Embodiment 72: The composition of embodiment 70 or 71, wherein the Rep protein(s) are selected from the group consisting of Rep78, Rep52, Rep68, Rep40, and any combinations thereof.

Embodiment 73: The composition of any one of embodiments 70-72, wherein the helper protein(s) are selected from the group consisting of E1a, E1b, E4, E2a, and any combinations thereof.

Embodiment 74: The composition of any one of embodiments 64-73, wherein one or more opal stop codons of the AAV nucleotide sequence(s) is engineered to an amber stop codon or ochre stop codon.

Embodiment 75: The composition of any one of embodiments 64-74, wherein one or more ochre stop codons of the AAV nucleotide sequence(s) is engineered to an amber stop codon or opal stop codon.

Embodiment 76: The composition of any one of embodiments 64-75, wherein one or more amber stop codons of the AAV nucleotide sequence(s) is engineered to an ochre stop codon or opal stop codon.

Embodiment 77: The composition of any one of embodiments 64-76, wherein the one or more stop codon engineered AAV nucleotide sequence(s) comprises one or more sequences selected from SEQ ID NOs: 214-221.

Embodiment 78: A cell comprising the composition of any one of embodiments 1-77.

Embodiment 79: The cell of embodiment 76 or the composition of any one of embodiments 1-63, further comprising a sequence coding for a Rep protein.

Embodiment 80: The cell or composition of embodiment 79, wherein the Rep protein is Rep78, Rep52, Rep68, Rep40, or any combination thereof.

Embodiment 81: The cell or composition of embodiment 80, wherein an opal stop codon of the sequence coding for the Rep68 is changed to an amber stop codon or an ochre stop codon.

Embodiment 82: The cell or composition of embodiment 80 or 81, wherein an opal stop codon of the sequence coding for the Rep40 is changed to an amber stop codon or an ochre stop codon.

Embodiment 83: The cell or composition of any one of embodiments 80-82, wherein an ochre stop codon of the sequence coding for the Rep78 is changed to an amber stop codon or an opal stop codon.

Embodiment 84: The cell or composition of any one of embodiments 80-83, wherein an ochre stop codon of the sequence coding for the Rep52 is changed to an amber stop codon or an opal stop codon.

Embodiment 85: The cell of any one of embodiments 78-84 or composition of any one of embodiments 1-62 or 77-82, further comprising a sequence coding for a Cap protein.

Embodiment 86: The cell or composition of embodiment 85, wherein the Cap protein is VP1, VP2, VP3, or any combination thereof.

Embodiment 87: The cell or composition of embodiment 86, wherein an ochre stop codon of the sequence coding for VP1 is changed to an amber stop codon or an opal stop codon.

Embodiment 88: The cell or composition of embodiment 86 or 87, wherein an ochre stop codon of the sequence coding for VP2 is changed to an amber stop codon or an opal stop codon.

Embodiment 89: The cell or composition of any one of embodiments 86-88, wherein an ochre stop codon of the sequence coding for VP3 is changed to an amber stop codon or an opal stop codon.

Embodiment 90: The cell of any one of embodiments 78-89 or composition of any one of embodiments 1-63 or 79-89, further comprising a sequence coding for a Helper protein or encoding VA RNA.

Embodiment 91: The cell or composition of embodiment 90, wherein the Helper protein is E1a, E1b, E4, E2a, or any combination thereof.

Embodiment 92: The cell of any one of embodiments 78-91 or composition of any one of embodiments 1-63 or 79-91, further comprising a sequence coding for AAP.

Embodiment 93: The cell or composition of embodiment 92, wherein an opal stop codon of the sequence coding for AAP is changed to an amber stop codon or an ochre stop codon.

Embodiment 94: The cell of any one of embodiments 78-93 or composition of any one of embodiments 1-63 or 79-93, further comprising a sequence coding for MAAP.

Embodiment 95: The cell or composition of embodiment 92, wherein an amber stop codon of the sequence coding for MAAP is changed to an opal stop codon or an ochre stop codon.

Embodiment 96: The cell of any one of embodiments 78-95 or composition of any one of embodiments 1-63 or 79-95, further comprising a sequence coding for Protein X.

Embodiment 97: The cell or composition of embodiment 96, wherein an opal stop codon of the sequence coding for Protein X is changed to an amber stop codon or an ochre stop codon.

Embodiment 98: The cell of any one of embodiments 78-97 or composition of any one of embodiments 1-97, wherein the AAV nucleotide sequence, the conditionally repressible tRNA sequence, the suppressor tRNA coding sequence, the sequence coding for a Rep protein, the sequence coding for a Cap protein, a sequence coding for a helper protein, a sequence encoding VA RNA, a sequence coding for AAP, a sequence coding for MAAP, a sequence coding for Protein X, a sequence coding for a repressor, or any combination of these sequences are in one or more plasmids.

Embodiment 99: The cell of any one of embodiments 78-97 or composition of any one of embodiments 1-97, wherein the AAV nucleotide sequence, the conditionally repressible tRNA sequence, the suppressor tRNA coding sequence, the sequence coding for a Rep protein, the sequence coding for a Cap protein, a sequence coding for a helper protein, a sequence encoding VA RNA, a sequence coding for AAP, a sequence coding for MAAP, a sequence coding for Protein X, a sequence coding for a repressor, or any combination of these sequences are stably integrated into the nuclear genome of the cell.

Embodiment 100: The cell of any one of embodiments 78-99, wherein the cell is a mammalian cell.

Embodiment 101: The cell of any one of embodiments 78-96, wherein the cell is an HEK293 cell or a CHO cell.

Embodiment 102: The cell of any one of embodiments 78-99, wherein the cell is an insect cell.

Embodiment 103: The cell of any one of embodiments 78-99 or 102, wherein the cell is an Sf9 cell.

Embodiment 104: The cell of any one of embodiments 78-103, wherein cell is capable of producing a virion encapsidating the suppressor tRNA.

Embodiment 105: The cell of any one of embodiments 78-104, wherein the cell is capable of producing a virion encapsidating the conditionally repressible suppressor tRNA coding sequence.

Embodiment 106: The cell of any one of embodiments 78-105 or composition of any one of embodiments 1-51 or 77-103, further comprising the repressor or a sequence coding for the repressor.

Embodiment 107: The cell or composition of embodiment 106, wherein the repressor is a Tet repressor protein or a guide RNA complexed to a catalytically dead Cas protein that is linked or fused to a repressor domain.

Embodiment 108: The cell or composition of embodiment 107, wherein the repressor domain is a krab domain.

Embodiment 109: A method of silencing a suppressor tRNA comprising:
providing a cell comprising a repressor; and
transfecting the cell with the composition of any one of embodiments 1-52,
wherein the repressor binds to the repressor element, thereby silencing expression of the suppressor tRNA.

Embodiment 110: A method of silencing a suppressor tRNA comprising:
providing a cell comprising the suppressor tRNA; and
transfecting the cell with the composition of any one of embodiments 53-63;
wherein the siRNA, shRNA, DsRNA or any combination thereof binds to the suppressor tRNA and targets the suppressor tRNA for degradation, thereby silencing the tRNA.

Embodiment 111: A method of silencing suppressor tRNA for virion production comprising:
providing a cell comprising a repressor;
transfecting the cell with a polynucleotide comprising a sequence coding for a helper protein, a polynucleotide comprising a sequence encoding VA RNA, a polynucleotide comprising a sequence coding for a Rep protein, and a polynucleotide comprising a sequence coding for a Cap protein;
transfecting the cell with the composition of any one of embodiments 1-52, wherein the conditionally repressible suppressor tRNA coding sequence is flanked by ITRs; and
culturing the cell under conditions suitable for expression of the helper protein, the VA RNA, the Rep protein, and the Cap protein and for encapsidation of the conditionally repressible suppressor tRNA coding sequence, thereby producing virions encapsidating the conditionally repressible suppressor tRNA coding sequence,
wherein the repressor binds to the repressor element, thereby silencing expression of the suppressor tRNA for improved/enhanced virion production.

Embodiment 112: A method of silencing suppressor tRNA for virion production comprising:
providing a cell comprising a suppressor tRNA flanked by ITRs;
transfecting the cell with a sequence coding for a helper protein, a sequence encoding VA RNA, a sequence coding for a Rep protein, and a sequence coding for a Cap protein;
transfecting the cell with the composition of any one of embodiments 53-63; and
culturing the cell under conditions suitable for expression of the helper protein, the VA RNA, the Rep protein, and the Cap protein and for encapsidation of the suppressor tRNA coding sequence,
wherein the siRNA, shRNA, DsRNA or any combination thereof binds to the suppressor tRNA and targets the suppressor tRNA for degradation, thereby silencing the suppressor tRNA for virion production.

Embodiment 113: A method of producing virion encapsidating a sequence coding for a suppressor tRNA comprising
providing a cell comprising a repressor;
transfecting the cell with a sequence coding for a helper protein, a sequence coding for a Rep protein, and a sequence coding for a Cap protein;
transfecting the cell with the composition of any one of embodiments 1-52, wherein the conditionally repressible suppressor tRNA coding sequence is flanked by ITRs; and
culturing the cell under conditions suitable for expression of the helper protein, the VA RNA, the Rep protein, and the Cap protein and for encapsidation of the conditionally repressible suppressor tRNA coding sequence, thereby producing virions encapsidating the sequence coding for a suppressor tRNA,
wherein the repressor binds to the repressor element, thereby silencing expression of the suppressor tRNA.

Embodiment 114: A method of producing virion encapsidating a sequence coding for a suppressor tRNA comprising
providing a cell comprising a suppressor tRNA coding sequence flanked by ITRs;
transfecting the cell with a sequence coding for a helper protein, a sequence coding for a Rep protein, and a sequence coding for a Cap protein;
transfecting the cell with the composition of any one of embodiments 53-63; and
culturing the cell under conditions suitable for expression of the helper protein, the VA RNA, the Rep protein, and the Cap protein and for encapsidation of the conditionally repressible suppressor tRNA coding sequence, thereby producing virions encapsidating the conditionally repressible suppressor tRNA coding sequence,
wherein the siRNA, shRNA, DsRNA or any combination thereof binds to the suppressor tRNA and targets the suppressor tRNA for degradation, thereby silencing the suppressor tRNA.

Embodiment 115: A method of producing virion encapsidating a sequence coding for a suppressor tRNA, the method comprising:
a) providing a cell comprising: a sequence encoding a suppressor tRNA flanked by ITRs, a sequence encoding a helper protein, a sequence encoding a VA RNA, a sequence encoding a Rep protein, a sequence encoding a Cap protein, and optionally a sequence coding for AAP, a sequence coding for MAAP, and/or a sequence coding for Protein X, wherein one or more of the sequence encoding the helper protein, the sequence encoding the VA RNA, the sequence encoding the Rep protein, the sequence coding for the Cap protein, the sequence coding for AAP, the sequence coding for MAAP, and/or the sequence coding for Protein X is stop codon engineered; and
b) culturing the cell under conditions suitable for expression and encapsidation of the suppressor tRNA.

Embodiment 116: A method of increasing titer of virions encapsidating a sequence coding for a suppressor tRNA by silencing the suppressor tRNA comprising
providing a cell comprising a repressor;
transfecting the cell with a sequence coding for a helper protein, a sequence coding for a Rep protein, and a sequence coding for a Cap protein;
transfecting the cell with the composition of any one of embodiments 1-52, wherein the conditionally repressible suppressor tRNA coding sequence is flanked by ITRs; and
culturing the cell under conditions suitable for expression of the helper protein, the VA RNA, the Rep protein, and the Cap protein and for encapsidation of the conditionally repressible suppressor tRNA coding sequence, thereby producing virions encapsidating the sequence coding for a suppressor tRNA,
wherein the repressor binds to the repressor element, thereby silencing expression of the suppressor tRNA for virion production, and
wherein the titer of virions encapsidating the sequence coding for the suppressor tRNA is increased compared to titer of virions encapsidating the sequence for the suppressor tRNA produced by steps b)-d) or produced by transfecting the cell with a sequence coding for a suppressor tRNA and transfecting the cell with a sequence coding for a helper protein, a sequence coding for a Rep protein, and a sequence coding for a Cap protein.

Embodiment 117: A method of increasing titer of virions encapsidating a sequence coding for a suppressor tRNA by silencing the suppressor tRNA comprising
transfecting a cell with the sequence coding for the suppressor tRNA flanked by ITRs;
transfecting the cell with a sequence coding for a helper protein, a sequence coding for a Rep protein, and a sequence coding for a Cap protein;
transfecting the cell with the composition of any one of embodiments 53-63; and
culturing the cell under conditions suitable for expression of the helper protein, the VA RNA, the Rep protein, and the Cap protein and for encapsidation of the conditionally repressible suppressor tRNA coding sequence, thereby producing virions encapsidating the sequence coding for the suppressor tRNA,
wherein the siRNA, shRNA, DsRNA or any combination thereof binds to the suppressor tRNA and targets the suppressor tRNA for degradation, thereby silencing the suppressor tRNA, and
wherein the titer of virions encapsidating the sequence coding for the suppressor tRNA is increased compared to titer of virions encapsidating the sequence for the suppressor tRNA produced by steps a), b), and d).

Embodiment 118: A method of increasing titer of virions encapsidating a sequence coding for a suppressor tRNA, the method comprising:
a) providing a cell comprising: a sequence encoding a suppressor tRNA flanked by ITRs, a sequence encoding a helper protein, a sequence encoding a VA RNA, a sequence encoding a Rep protein, a sequence encoding a Cap protein, and optionally a sequence coding for AAP, a sequence coding for MAAP, and/or a sequence coding for Protein X, wherein one or more of the sequence encoding the helper protein, the sequence encoding the VA RNA, the sequence encoding the Rep protein, the sequence coding for the Cap protein, the sequence coding for AAP, the sequence coding for MAAP, and/or the sequence coding for Protein X is stop codon engineered; and
b) culturing the cell under conditions suitable for expression and encapsidation of the suppressor tRNA.

Embodiment 119: The method of any one of embodiments 109, 111, 113, or 116, wherein the repressor is a Tet repressor protein or a guide RNA complexed to a catalytically dead Cas protein that is linked or fused to a repressor domain.

Embodiment 120: The method of embodiment 119, wherein the repressor domain is a krab domain.

Embodiment 121: The method of any one of embodiments 111-120, wherein the Rep protein is Rep78, Rep52, Rep68, Rep40, or any combination thereof.

Embodiment 122: The method of embodiment 121, wherein an opal stop codon of the sequence coding for the Rep68 is changed to an amber stop codon or an ochre stop codon.

Embodiment 123: The method of embodiment 121 or 122, wherein an opal stop codon of the sequence coding for the Rep40 is changed to an amber stop codon or an ochre stop codon.

Embodiment 124: The method of any one of embodiments 121-123, wherein an ochre stop codon of the sequence coding for the Rep78 is changed to an amber stop codon or an opal stop codon.

Embodiment 125: The method of any one of embodiments 121-124, wherein an ochre stop codon of the sequence coding for the Rep52 is changed to an amber stop codon or an opal stop codon.

Embodiment 126: The method of any one of embodiments 111-123, wherein the Cap protein is VP1, VP2, VP3, or any combination thereof.

Embodiment 127: The method of embodiment 126, wherein an ochre stop codon of the sequence coding for VP1 is changed to an amber stop codon or an opal stop codon.

Embodiment 128: The method of embodiment 126 or 127, wherein an ochre stop codon of the sequence coding for VP2 is changed to an amber stop codon or an opal stop codon.

Embodiment 129: The method of any one of embodiments 126-128, wherein an ochre stop codon of the sequence coding for VP3 is changed to an amber stop codon or an opal stop codon.

Embodiment 130: The method of any one of embodiments 124-129, the helper protein is E1a, E1b, E4, E2a, or any combination thereof.

Embodiment 131: The method of any one of embodiments 124-130, wherein the cell is a mammalian cell.

Embodiment 132: The method of any one of embodiments 124-131, wherein the cell is an HEK293 cell or a CHO cell.

Embodiment 133: The method of any one of embodiments 124-130, wherein the cell is an insect cell.

Embodiment 134: The method of any one of embodiments 124-130 or 133, wherein the cell is an Sf9 cell.

Embodiment 135: The method of any one of embodiments 124-134, wherein the virion is an AAV virion.

Embodiment 136: The method of embodiment 135, wherein a capsid of the AAV virion is selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combinations thereof.

Embodiment 137: A virion produced by the method of any one of embodiments 111-136.

Embodiment 138: A virion encapsidating the sequence coding for the conditionally repressible suppressor tRNA of any one of embodiments 1-52.

Embodiment 139: The virion of embodiment 137 or 138, wherein the virion is an AAV virion. Embodiment 140: The virion of any one of embodiments 137-139, wherein a capsid of the AAV virion is selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combinations thereof.

Embodiment 141: A method of treating a subject having a disease associated with a premature stop codon comprising administering the virion of any one of embodiments 137-140 to the subject.

Embodiment 143: The method of embodiment 141, wherein the disease associated with a premature stop codon is Rett syndrome, Dravet syndrome, or Duchenne Muscular Dystrophy.

Embodiment 144: A method of treating a subject having Rett syndrome comprising administering the virion of any one of embodiments 137-140 to the subject.

Embodiment 145: The method of any one of embodiments 141-143 wherein the subject is a human.

### OTHER EMBODIMENTS

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

What is claimed is:

1. A polynucleotide comprising, from 5′ to 3′:
a 5′ AAV inverted terminal repeat (ITR) sequence;
a polynucleotide sequence comprising one or more conditionally repressible suppressor tRNA coding sequence(s); and
a 3′ AAV inverted terminal repeat (ITR) sequence.

2. The polynucleotide of claim 1, wherein the conditionally repressible suppressor tRNA coding sequence(s) each comprise sequence(s) encoding repressor element(s) and sequence(s) encoding suppressor tRNA(s).

3. The polynucleotide of claim 2 or claim 3, wherein the sequence encoding the repressor element is upstream of the sequence encoding the suppressor tRNA.

4. The polynucleotide of claim 2 or claim 3, wherein the sequence encoding the repressor element is downstream of the sequence encoding the suppressor tRNA.

5. The polynucleotide of claim 3, wherein the sequence coding for the repressor element is positioned at +1, −7, −12, −20, −30, or −45 of the sequence coding for the suppressor tRNA.

6. The polynucleotide of claim 4, wherein the sequence encoding the repressor element is positioned at +7, +12, +20, +30, or +45 of the sequence encoding the suppressor tRNA.

7. The polynucleotide of any one of claims 2-6, wherein the repressor element comprises a sequence encoding an operator.

8. The polynucleotide of claim 7, wherein the operator is selected from the group consisting of a tetracycline operator (TetO) and a lac operator (LacO).

9. The polynucleotide of claim 8, wherein the tetracycline operator comprises SEQ ID NO: 150.

10. The polynucleotide of any one of claims 7-9, wherein the repressor element comprises a sequence encoding a tetracycline response element (TRE).

11. The polynucleotide of claim 10, wherein the sequence encoding the TRE comprises TetO sequence(s).

12. The polynucleotide of claim 10 or 11, wherein the TetO sequence(s) comprises a concatemer of TetO sequences.

13. The polynucleotide of claim 12, wherein the TetO sequence(s) comprises 2, 3, 4, 5, 6, 7, 8, 9, or 10 TetO sequences.

14. The polynucleotide of claim 12 or 13, wherein two or more TetO sequences in the concatemer of TetO sequences are separated by a spacer sequence.

15. The polynucleotide of claim 16, wherein the spacer sequence is TC.

16. The polynucleotide of any one of claims 12-15, wherein the concatemer of TetO sequences consists of 2 TetO sequences.

17. The polynucleotide of any one of claims 12-16, wherein the concatemer of TetO sequences comprises 2 TetO sequences separated by a spacer sequence.

18. The polynucleotide of any one of claim 12-15 or 17, wherein the concatemer of TetO sequences comprises 2 TetO sequences separated by a TC spacer sequence.

19. The polynucleotide of any one of claim 12-15, 17, or 18, wherein the concatemer of TetO sequences consists of SEQ ID NO: 149.

20. The polynucleotide of any one of claims 10-19, wherein the polynucleotide sequence comprising one or more conditionally repressible suppressor tRNA coding sequence(s) further comprises a promoter sequence.

21. The polynucleotide of claim 20, wherein the promoter sequence comprises a RNA polymerase type III promoter, optionally a U6 promoter sequence, optionally a hU6 promoter sequence.

22. The polynucleotide of claim 20 or 21, wherein the promoter sequence comprises a tRNA promoter sequence or fragment thereof.

23. The polynucleotide of any one of claims 20-22, wherein the promoter sequence is upstream of the conditionally repressible suppressor tRNA coding sequence(s).

24. The polynucleotide of any one of claims 20-22, wherein the promoter sequence is downstream of the conditionally repressible suppressor tRNA coding sequence(s).

25. The polynucleotide of any one of claims 20-22, wherein the promoter sequence is interspersed within the conditionally repressible tRNA coding sequence(s).

26. The polynucleotide of any one of claims 20-25, wherein the promoter sequence is an hU6 promoter sequence.

27. The polynucleotide of claim 26, wherein the hU6 promoter sequence comprises one or more promoter sequence element(s) selected from the group consisting of a SPH element, an Oct-1 element, a proximal sequence element (PSE), and a TATA element.

28. The polynucleotide of claim 27, wherein the promoter sequence element(s) are interspersed between repressor element sequence(s) and suppressor tRNA sequence(s).

29. The polynucleotide of claim 27 or 28, wherein the repressor element sequence(s) are downstream of the SPH and Oct-1 element(s).

30. The polynucleotide of any one of claims 27-29, wherein the repressor element sequence(s) are before the PSE element, between the PSE and TATA elements, and/or after the TATA element.

31. The polynucleotide of any one of claims 2-30, wherein the sequence(s) encoding the suppressor tRNA(s) are sequence(s) encoding for opal suppressor tRNA(s).

32. The polynucleotide of any one of claims 2-30, wherein the sequence(s) encoding the suppressor tRNA(s) are sequence(s) encoding for ochre suppressor tRNA(s).

33. The polynucleotide of any one of claims 2-30, wherein the sequence(s) encoding the suppressor tRNA(s) are sequence(s) encoding for amber suppressor tRNA(s).

34. The polynucleotide of any one of claims 2-30, wherein the sequences encoding the suppressor tRNAs are sequence(s) encoding for opal suppressor tRNA(s), sequence(s) encoding for ochre suppressor tRNA(s), sequence(s) encoding for amber suppressor tRNA(s), or any combination thereof.

35. The polynucleotide of any one of claims 2-34, wherein the sequence(s) encoding the suppressor tRNA(s) are each selected from the group consisting of any one of SEQ ID NOs: 3-48, 49-68, and 69-88.

36. The polynucleotide of any one of claims 2-34, wherein the sequence(s) encoding the suppressor tRNA(s) are each selected from the group consisting of any one of SEQ ID NOs: 103-148, 249-268, and 269-288.

37. The polynucleotide of any one of claims 1-36, wherein the sequence encoding the polynucleotide sequence comprising one or more conditionally repressible suppressor tRNA coding sequence(s) comprises at least 80%, 85%, 90%, 95%, 97%, 99%, or 100% sequence identity to any one of SEQ ID NOs: 158-162, 164-168, 171-175, 177-181, 182-194, or 199; optionally wherein the sequence encoding the polynucleotide sequence comprises multiple copies of the one or more conditionally repressible suppressor tRNA coding sequence(s) comprising at least 80%, 85%, 90%, 95%, 97%, 99%, or 100% sequence identity to any one of SEQ ID NOs: 158-162, 164-168, 171-175, 177-181, 182-194, or 199; optionally wherein the sequence encoding the polynucleotide sequence comprises one, two, three, four, five, six, seven, eight, nine, or ten of the one or more conditionally repressible suppressor tRNA coding sequence(s) comprising at least 80%, 85%, 90%, 95%, 97%, 99%, or 100% sequence identity to any one of SEQ ID NOs: 158-162, 164-168, 171-175, 177-181, 182-194, or 199.

38. The polynucleotide of any one of claims 1-36, wherein the sequence encoding the polynucleotide sequence comprising one or more conditionally repressible suppressor tRNA coding sequence(s) comprises or consists of at least 80%, 85%, 90%, 95%, 97%, 99%, or 100% sequence identity any one of SEQ ID NOs: 203-206 or 208-211, with or without the transduction marker; optionally, wherein the transduction marker comprises at least 80%, 85%, 90%, 95%, 97%, 99%, or 100% sequence identity to SEQ ID NO: 224.

39. The polynucleotide of claim 1, wherein the conditionally repressible suppressor tRNA comprises a guide RNA binding sequence and sequence encoding a suppressor tRNA.

40. The polynucleotide of claim 39, wherein the conditionally repressible suppressor tRNA coding sequence further comprises a protospacer adjacent motif (PAM).

41. The polynucleotide of claim 39 or 40, wherein the guide RNA binding sequence is from 15 to 30 nucleotides in length.

42. The polynucleotide of any one of claims 39-41, wherein the guide RNA binding sequence is positioned at +1 or −7 of the sequence encoding the suppressor tRNA.

43. The polynucleotide of claim 42, wherein the repressor is a guide RNA complexed to a Cas protein, wherein the Cas protein is linked or fused to a repressor domain.

44. The polynucleotide of claim 43, wherein the Cas protein is a catalytically dead Cas protein.

45. The polynucleotide of claim 43 or 44, wherein the repressor domain is a krab domain.

46. The polynucleotide of any one of claims 1-45, wherein the polynucleotide sequence comprising one or more conditionally repressible suppressor tRNA coding sequence(s) comprises two or more conditionally repressible suppressor tRNA coding sequences.

47. The polynucleotide of any one of claims 1-46, wherein the polynucleotide sequence comprising one or more conditionally repressible suppressor tRNA coding sequence(s) comprises two, three, four, five, or six conditionally repressible suppressor tRNA coding sequences.

48. The polynucleotide of claim 47, wherein the polynucleotide sequence comprising one or more conditionally repressible suppressor tRNA coding sequence(s) comprises two conditionally repressible suppressor tRNA coding sequences.

49. The polynucleotide of claim 47, wherein the polynucleotide sequence comprising one or more conditionally repressible suppressor tRNA coding sequence(s) comprises three conditionally repressible suppressor tRNA coding sequences.

50. The polynucleotide of claim 47, wherein the polynucleotide sequence comprising one or more conditionally repressible suppressor tRNA coding sequence(s) comprises six conditionally repressible suppressor tRNA coding sequences.

51. The polynucleotide of any one of claims 46-50, wherein the conditionally repressible suppressor tRNA coding sequences are the same.

52. The polynucleotide of any one of claims 46-50, wherein the conditionally repressible suppressor tRNA coding sequences are different.

53. The polynucleotide of any one of claims 46-52, wherein the conditionally repressible suppressor tRNA coding sequences are separated by filler sequence(s).

54. The polynucleotide of claim 53, wherein the filler sequence(s) are each, independently, from 1 to 500 nucleotides in length.

55. The polynucleotide of claim 54, wherein the filler sequence(s) are 100 nucleotides in length.

56. The polynucleotide of any one of claims 1-55, wherein the polynucleotide sequence comprising one or more conditionally repressible suppressor tRNA coding sequence(s) further comprises quantification tag sequence(s).

57. The polynucleotide of claim 56, comprising a quantification tag sequence 5′ of the conditionally repressible suppressor tRNA coding sequence(s).

58. The polynucleotide of claim 56 or 57, comprising a quantification tag sequence 3′ of the conditionally repressible suppressor tRNA coding sequence(s).

59. The polynucleotide of any one of claims 1-58, wherein the polynucleotide sequence comprising one or more conditionally repressible suppressor tRNA coding sequence(s) further comprises a transduction marker sequence.

60. The polynucleotide of claim 59, wherein the transduction marker sequence encodes a fluorescent protein.

61. The polynucleotide of any one of claims 1-52, wherein the 5′ and 3′ ITR sequences are selected from AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68 5′ and 3′ ITR sequences, or any combination thereof; optionally, wherein the 5′ ITR sequence comprises at least 80%, 85%, 90%, 95%, 97%, 99%, or 100% sequence identity to any one of SEQ ID NOs: 225-226; optionally, wherein the 3′ ITR comprises at least 80%, 85%, 90%, 95%, 97%, 99%, or 100% sequence identity to any one of SEQ ID NOs: 227-228.

62. A vector comprising the polynucleotide of any one of claims 1-61.

63. A system comprising the vector of claim 62; and
one or more vectors comprising polynucleotide sequences(s) encoding for one or more proteins selected from the group of: a Rep protein, a Cap protein, a Helper protein, VA RNA, an AAP protein, an MAAP protein, a Protein X protein, and combinations thereof.

64. Cell(s) comprising the vector of claim 62 or the system of claim 63.

65. The cell(s) of claim 64, wherein the cell(s) are engineered to express a repressor configured to bind to the repressor element of the polynucleotide of any one of claims 2-49.

66. The cell(s) of claim 65, wherein the repressor is Tet Repressor protein; optionally, wherein the Tet Repressor protein is TetR; further optionally, wherein the Tet Repressor protein comprises at least 80%, 85%, 90%, 95%, 97%, 99%, or 100% sequence identity to SEQ ID NO: 223.

67. The cell(s) of any one of claims 64-66, wherein the cell(s) comprise adenovirus gene E1; optionally, wherein the adenovirus gene E1 comprises ELA gene and/or E1B gene.

68. The cell(s) of any one of claims 65-67, wherein binding of the repressor to the repressor element represses expression of the suppressor tRNA, relative to a control, optionally wherein the repressor element represses expression of the suppressor tRNA at least 40%, at least 45%, at least 50%, at least 55%, at least 60%, at least 65%, at least 70%, at least 75%, at least 80%, at least 85%, or at least 90% relative to a control.

69. The cell(s) of claim 68, wherein the control is: a) expression of the suppressor tRNA in the cell(s) when the repressor is inhibited from binding to the repressor element, optionally wherein the repressor is inhibited from binding to the repressor element by binding to a switching agent; or b) expression of the suppressor tRNA from reference cell(s) comprising the vector of claim 62, wherein the reference cell(s) do not express the repressor.

70. A method for producing AAV genomes, the method comprising:
culturing the cell(s) of any one of claims 64-69 under conditions effective to replicate the polynucleotide sequence comprising one or more conditionally repressible suppressor tRNA coding sequence(s).

71. A method for producing virion encapsidating a polynucleotide coding for a suppressor tRNA, the method comprising:
culturing the cell(s) of any one of claims 64-69 under conditions effective to replicate the polynucleotide sequence comprising one or more conditionally repressible suppressor tRNA coding sequence(s) and express the Cap protein.

72. A composition comprising one or more stop codon engineered AAV nucleotide sequence(s).

73. The composition of claim 72, further comprising one or more suppressor tRNA coding sequence(s), optionally flanked by 5′ and 3′ ITR sequences.

74. The composition of claim 72 or 73, wherein the suppressor tRNA coding sequence(s) independently encode a suppressor tRNA that is capable of suppressing an opal stop codon, an ochre stop codon, or an amber stop codon.

75. The composition of claim 73 or 74, wherein the suppressor tRNA coding sequence comprises 100%, at least 99%, at least 98%, at least 95%, or at least 90% sequence identity to any one of SEQ ID NO: 3-SEQ ID NO: 48, SEQ ID NO: 103-SEQ ID NO: 148, SEQ ID NO: 49-SEQ ID NO: 88, or SEQ ID NO: 249-288; optionally, wherein the anticodon is engineered to bind to an ochre stop codon, an amber stop codon, or an opal stop codon.

76. The composition of any one of claims 72-75, wherein the stop codon engineered AAV nucleotide sequence(s) are selected from the group consisting of a sequence coding for a Rep protein, a sequence coding for a Cap protein, a sequence coding for a helper protein, a sequence encoding VA RNA, a sequence encoding AAP, a sequence encoding MAAP, a sequence encoding Protein X, and any combinations thereof.

77. The composition of any one of claims 72-76, wherein the stop codon engineered AAV nucleotide sequence(s) are selected from the group consisting of 23K Endoprotease, VP1, E1B 19K, E1B 55K, E2A, Hexon-associated precursor, Hexon assembly, Rep2, and combinations thereof.

78. The composition of any one of claims 72-77, wherein one or more opal stop codons of the AAV nucleotide sequence(s) is engineered to an amber stop codon or ochre stop codon.

79. The composition of any one of claims 72-77, wherein one or more ochre stop codons of the AAV nucleotide sequence(s) is engineered to an amber stop codon or opal stop codon.

80. The composition of any one of claims 72-79, wherein the one or more stop codon engineered AAV nucleotide sequence(s) comprises one or more sequences selected from SEQ ID NOs: 155-156, with one or more of the stop codons engineered to a different stop codon.

81. The composition of any one of claims 72-80, wherein the one or more stop codon engineered AAV nucleotide sequence(s) comprises one or more sequences selected from SEQ ID NOs: 214-221, with one or more of the stop codons engineered to a different stop codon.

82. Cell(s) comprising the composition of any one of claims 72-81.

83. A method for producing AAV genomes, the method comprising:
culturing the cell(s) of claim 82 under conditions effective to replicate the polynucleotide sequence comprising one or more conditionally repressible suppressor tRNA coding sequence(s).

84. A method for producing virion encapsidating a polynucleotide coding for a suppressor tRNA, the method comprising:
culturing the cell(s) of claim 82 under conditions effective to replicate the polynucleotide sequence comprising one or more conditionally repressible suppressor tRNA coding sequence(s) and express the Cap protein.

85. A virion produced by the method of claim 71 or 84.

86. The virion of claim 85, wherein a capsid of the AAV virion is selected from the group consisting of AAV1, AAV2, AAV3, AAV4, AAV5, AAV6, AAV7, AAV8, AAV9, AAV10, AAV11, AAV 12, AAV13, AAV14, AAV15, AAV16, AAV-DJ, AAV-DJ/8, AAV-DJ/9, AAV1/2, AAV.rh8, AAV.rh10, AAV.rh20, AAV.rh39, AAV.Rh43, AAV.v66, AAV.Rh74, AAV.Oligo001, AAV.SCH9, AAV.r3.45, AAV.RHM4-1, AAV.hu37, AAV.Anc80, AAV.Anc80L65, AAV.7m8, AAV.PhP.eB, AAV.PhP.V1, AAV.PHP.B, AAV.PhB.C1, AAV.PhB.C2, AAV.PhB.C3, AAV.PhB.C6, AAV.cy5, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, AAV.HSC16, AAV.HSC17, and AAVhu68, or any combination thereof.

87. A method of treating a subject having a disease associated with a premature stop codon comprising administering the virion of claim 85 or 86 to the subject.

88. The method of claim 87, wherein the disease associated with a premature stop codon is Rett syndrome, Dravet syndrome, or Duchenne Muscular Dystrophy.

89. A method of treating a subject having Rett syndrome comprising administering the virion of claim 85 or 86 to the subject.

90. The method of any one of claims 87-89 wherein the subject is a human.

91. A method for producing encapsidated virion, the method comprising:
culturing the cell(s) of any one of the preceding claims under conditions effective (a) to replicate the polynucleotide comprising one or more conditionally repressible suppressor tRNA coding sequence(s) and (b) express the Cap protein;
wherein expression of the suppressor tRNA is repressed compared to a control;
optionally wherein the expression of the suppressor tRNA is repressed at least 40%, at least 45%, at least 50%, at least 55%, at least 60%, at least 65%, at least 70%, at least 75%, at least 80%, at least 85%, or at least 90% relative to a control;
optionally wherein the control is: a) expression of the suppressor tRNA in the cell(s) when the repressor is inhibited from binding to the repressor element, optionally wherein the repressor is inhibited from binding to the repressor element by a switching agent that binds to the repressor; or b) expression of the suppressor tRNA from reference cell(s) comprising the vector of claim 62, wherein the reference cell(s) do not express the repressor; optionally, wherein the switching agent is doxycycline.

92. A method for readthrough of a premature stop codon in a subject having a disease associated with a premature stop codon, the method comprising:
producing an encapsidated virion according to the method of claim 91; and
administering the virion to a subject having a disease associated with a premature stop codon under conditions effective for expression of the suppressor tRNA and readthrough of the premature stop codon.

93. The method of claim 92, wherein the disease associated with a premature stop codon is Rett syndrome, Dravet syndrome, or Duchenne Muscular Dystrophy.

94. The method of claim 92, wherein the disease associated with a premature stop codin is Rett syndrome.

95. The method of any one of claims 92-94 wherein the subject is a human.
